(19) United States
(12) Patent Application Publication
(10) Pub. No.: US 2026/0167988 A1
(43) Pub. Date: Jun. 18, 2026

(54) MINISTRING DNA FOR PRODUCING ADENO-ASSOCIATED VIRUS

(71) Applicant: MEDIPHAGE BIOCEUTICALS, INC., Toronto (CA)

(72) Inventors: Roderick SLAVCEV, Waterloo, ON (CA); Nafiseh NAFISSI, Toronto, ON (CA)

(21) Appl. No.: 19/126,883

(22) PCT Filed: Nov. 2, 2023
(86) PCT No.: PCT/IB2023/061082

§ 371 Date: May 2, 2025

Related U.S. Application Data
(60) Provisional application No. 63/382,070, filed on Nov. 2, 2022.

(51) Int. Cl. C12N 15/86

(52) U.S. Cl. CPC C12N 15/86; C12N 2750/14143; C12N 2750/14152

(57) ABSTRACT

Provided herein are expression vectors, vector production systems, bacterial sequence-free vectors, and methods for producing adeno-associated viruses (AAVs). Also provided are AAVs as well as pharmaceutical compositions and uses thereof.

### CROSS REFERENCE TO RELATED APPLICATIONS

This PCT application claims the priority benefit of U.S. Provisional Application No. 63/382,070, filed Nov. 2, 2022, which is incorporated herein by reference in its entirety.

### REFERENCE TO SEQUENCE LISTING SUBMITTED ELECTRONICALLY VIA

The content of the electronically submitted sequence listing (Name: 4471_003PC03_Seqlisting_ST26; Size: 155,348 Bytes; and Date of Creation: Oct. 31, 2023) is herein incorporated by reference in its entirety.

### FIELD OF THE INVENTION

The present disclosure provides vectors and methods for producing adeno-associated virus (AAV) from ministring DNA.

### BACKGROUND OF THE INVENTION

AAV is a small, nonpathogenic virus containing a linear single-stranded DNA genome that is packaged into a non-enveloped viral capsid. It is a parvovirus of the family Parvoviridae and member of the genus Dependoparvovirus requiring functions provided by a co-infecting helper virus for efficient replication. See, e.g., Daya and Berms, Clin. Microbiol. Rev. 21(4): 583-593 (2008); Lisowski et al., Curr. Opin. Pharmacol. 24:59-67 (2015); Mary et al., Adeno-associated Virus Vectors in Gene Therapy, in Gene and Cell Therapy: Biology and Applications (Jayandharan G. eds, 2018).

The wild-type AAV genome is about 5 kilobases in length and encodes 8 proteins from the replication (rep) and capsid (cap) genes. Four Rep proteins (Rep40, Rep52, Rep68, and Rep78) as well as three Cap proteins (VP1, VP2, and VP3) and assembly-activating protein (AAP) are expressed by alternative promoters and as splice variants from the rep and cap genes. The Rep proteins are involved in replication and packaging, while the Cap proteins and AAP are involved in formation of the viral capsid. See, e.g., Lisowski et al.; Salganik et al., Microbiol. Spectrum 3(4):MDNA3-0052-2014 (2015).

Two inverted terminal repeats (ITRs) flank the coding sequences of the AAV genome and are required for replication and packaging. The terminus of each wild-type ITR contains palindromic regions that self-anneal, resulting in a double-stranded T-shaped hairpin structure on each end of the AAV genome. The hairpin acts as an origin for AAV DNA replication and complementary strand synthesis using an infected cell's DNA polymerase complex. Either the sense or antisense strand of the double-stranded replication intermediate can be packaged as the single-stranded genome into the viral capsid. See, e.g., Daya and Berns, Lisowski et al.; Ling et al., J. Mol. Genet. Med. 9(3):175 (2015); Salganik et al.

Recombinant AAV can be produced by replacing the AAV genome between the ITRs with a nucleic acid sequence of interest and has been widely used in clinical and research studies. The standard method of producing recombinant AAV requires transfection of a mammalian or insect AAV producer cell with a vector containing a nucleic acid sequence of interest flanked by ITRs and separate helper vectors or viruses that provide the necessary AAV rep/cap and helper virus functions. For example, a typical production method involves transfection of human embryonic kidney 293 (HEK293) cells with three plasmids: one that contains a nucleic acid of sequence between two ITRs, one containing AAV rep and cap genes, and one containing adenoviral helper genes. By supplying the rep/cap and helper sequences in trans, the nucleic acid of interest between the ITRs is packaged into a capsid to form the recombinant AAV.

The production of AAV with existing methods is highly variable, inefficient, and often results in encapsidation of undesirable DNA. See, e.g., Wright, J. F., Gene Therapy 15:840-848 (2008). For example, 50-95% of AAV particles produced with standard methods are empty AAV capsids that do not contain any packaged DNA. See, e.g., Sommer et al., Mol. Ther. 7(1): 122-128 (2003). Even when capsids are packaged, they can contain nucleic acids other than or in addition to the desired nucleic acid sequence of interest, such as helper sequences, producer cell sequences, and/or bacterial sequences from plasmids containing the ITRs. These contaminating sequences can range from 1% to 8% of total DNA in purified AAV particles and can result in potential immunogenic and/or oncogenic effects. See, e.g., Wright, J. F. As of yet, state-of-the art purification strategies have failed to remove these contaminating sequences from AAV vector preparations.

A need exists for improved AAV vectors and production systems.

### SUMMARY OF THE INVENTION

The present disclosure is directed to an expression vector comprising: (a) a first sequence comprising an inverted terminal repeat (ITR) and a multiple cloning site (MCS), wherein the ITR flanks at least one side of the MCS, and wherein the ITR comprises a sequence for adeno-associated virus (AAV) replication and an AAV packaging signal, (b) a target sequence for a first recombinase flanking each side of the first sequence, and (c) one or more additional target sequences for one or more additional recombinases integrated within non-binding regions of the target sequence for the first recombinase, wherein the expression vector is for producing a bacterial sequence-free vector having linear covalently closed ends (i.e., “expression vector A”). In some aspects, the ITR flanks only one side of the MCS. In some aspects, the ITR flanks each side of the MCS. In some aspects, the expression vector further comprises a spacer sequence between the target sequence for the first recombinase and the first sequence. In some aspects, the spacer sequence is 10 to 500 nucleotides. In some aspects, the expression vector further comprises an expression cassette comprising an AAV replication (rep) gene and an AAV capsid (cap) gene flanked on one side by the target sequence for the first recombinase and flanked on the other side by the first sequence.

The present disclosure is directed to an expression vector comprising: (a) first sequence comprising an ITR and an expression cassette comprising a nucleic acid sequence of interest, wherein the ITR flanks at least one side of the expression cassette comprising the nucleic acid sequence of interest, and wherein the ITR comprises a sequence for AAV replication and an AAV packaging signal, (b) a target sequence for a first recombinase flanking each side of the first sequence, and (c) one or more additional target sequences for one or more additional recombinases integrated within non-binding regions of the target sequence for the first recombinase, wherein the expression vector is for producing a bacterial sequence-free vector having linear covalently closed ends (i.e., “expression vector B”).

In some aspects, the ITR flanks only one side of the expression cassette comprising the nucleic acid sequence of interest in expression vector B (i.e., “expression vector B1”). In some aspects, the expression vector further comprises a spacer sequence between the target sequence for the first recombinase and the first sequence. In some aspects, the spacer sequence is 10 to 500 nucleotides.

In some aspects, expression vector B1 further comprises an expression cassette comprising an AAV rep gene and an AAV cap gene flanked on one side by the target sequence for the first recombinase and flanked on the other side by the first sequence (i.e., “expression vector B2”).

In some aspects, the ITR flanks each side of the expression cassette comprising the nucleic acid sequence of interest in expression vector B (i.e., “expression vector B3”). In some aspects, the expression vector further comprises a spacer sequence between the target sequence for the first recombinase and the first sequence. In some aspects, the spacer sequence is 10 to 500 nucleotides.

In some aspects, expression vector B3 further comprises an expression cassette comprising an AAV rep gene and an AAV cap gene flanked on one side by the target sequence for the first recombinase and flanked on the other side by the first sequence (i.e., “expression vector B4”).

The present disclosure is directed to an expression vector comprising: (a) a first sequence comprising an ITR and a palindromic sequence, wherein the ITR flanks each side of the palindromic sequence, wherein the palindromic sequence comprises an expression cassette comprising a nucleic acid sequence of interest and a complement of the expression cassette, and wherein the ITR comprises a sequence for AAV replication and an AAV packaging signal, (b) a target sequence for a first recombinase flanking each side of the first sequence, and (c) one or more additional target sequences for one or more additional recombinases integrated within non-binding regions of the target sequence for the first recombinase, wherein the expression vector is for producing a bacterial sequence-free vector having linear covalently closed ends (i.e., “expression vector C”). In some aspects, the complement is separated from the expression cassette comprising the nucleic acid sequence of interest by a non-complementary spacer sequence. In some aspects, the expression vector further comprises a spacer sequence between the target sequence for the first recombinase and the first sequence. In some aspects, the spacer sequence is 10 to 500 nucleotides.

In some aspects, expression vector C further comprises an expression cassette comprising an AAV rep gene and an AAV cap gene flanked on one side by the target sequence for the first recombinase and flanked on the other side by the first sequence (i.e., “expression vector C1”).

The present disclosure is directed to an expression vector comprising: (a) a first sequence comprising a portion of an expression cassette comprising a nucleic acid sequence of interest flanked on one side by a splicing sequence, an ITR flanking each side of the first sequence, wherein the ITR comprises a sequence for AAV replication and an AAV packaging signal, (b) a target sequence for a first recombinase flanking each ITR, and (c) one or more additional target sequences for one or more additional recombinases integrated within non-binding regions of the target sequence for the first recombinase, wherein the expression vector is for producing a bacterial sequence-free vector having linear covalently closed ends (i.e., “expression vector D”). In some aspects, the portion of the expression cassette comprises a 5′ portion that in combination with the remaining portion of the expression cassette provides the complete sequence of the expression cassette, and the splicing sequence flanks the 3′ end of the 5′ portion. In some aspects, the portion of the expression cassette comprises a 3′ portion that in combination with the remaining portion of the expression cassette provides the complete sequence of the expression cassette, and the splicing sequence flanks the 5′ end of the 3′ portion. In some aspects, the expression vector further comprises a spacer sequence between the target sequence for the first recombinase and the first sequence. In some aspects, the spacer sequence is 10 to 500 nucleotides.

In some aspects, the sequence for AAV replication in any of the above expression vectors comprises an AAV ITR Replication (Rep) protein binding element (RBE) and terminal resolution site (TRS).

In some aspects, the AAV packaging signal in any of the above expression vectors comprises an AAV ITR D-sequence.

The present disclosure is directed to an expression vector comprising: (a) an expression cassette comprising an AAV rep gene and an AAV cap gene, (b) a target sequence for a first recombinase flanking each side of the expression cassette, and (c) one or more additional target sequences for one or more additional recombinases integrated within non-binding regions of the target sequence for the first recombinase wherein the expression vector is for producing a bacterial sequence-free vector having linear covalently closed ends (i.e., “expression vector E”).

The present disclosure is directed to an expression vector comprising: (a) an expression cassette comprising one or more helper virus genes for production of AAV, (b) a target sequence for a first recombinase flanking each side of the expression cassette, and (c) one or more additional target sequences for one or more additional recombinases integrated within non-binding regions of the target sequence for the first recombinase, wherein the expression vector is for producing a bacterial sequence-free vector having linear covalently closed ends (i.e., “expression vector F”). In some aspects, the one or more helper virus genes are from an adenovirus, a herpesvirus, a retrovirus, a poxvirus, and/or a lentivirus. In some aspects, the one or more helper virus genes comprise an adenovirus Early 4 (E4) gene, adenovirus Early 2A (E2A) gene, and adenovirus Viral Associated (VA) gene.

In some aspects, the target sequence for the first recombinase and the one or more additional target sequences for the one or more additional recombinases in any of the above expression vectors are selected from the group consisting of the PY54 pal site, the N15 telRL site, and the qK02 telRL site. In some aspects, any of the above expression vectors comprises each of the target sequences. In some aspects, any of the expression vectors comprises the Tel recombinase pal site and the telRL recombinase target binding sequence integrated within the pal site.

In some aspects, the target sequence for the first recombinase in any of the above expression vectors is the phage PY54 Tel 142 base pair target site.

The present disclosure is directed to a vector production system comprising recombinant cells designed to encode at least a first recombinase under the control of an inducible promoter, wherein the cells comprise any of the above expression vectors B-F. In some aspects, the inducible promoter is thermally-regulated, chemically-regulated, IPTG regulated, glucose-regulated, arabinose inducible, T7 polymerase regulated, cold-shock inducible, pH inducible, or combinations thereof. In some aspects, the first recombinase is selected from TelN and Tel, and the expression vector incorporates the target sequence for at least the first recombinase. In some aspects, the recombinant cells have been further designed to encode a nuclease genome editing system, and wherein the expression vector further comprises a backbone sequence containing a cleavage site for the nuclease genome editing system. In some aspects, the nuclease genome editing system is a CRISPR nuclease system comprising a Cas nuclease and gRNA, and the expression vector comprises a target sequence for the gRNA within the backbone sequence.

The present disclosure is directed to a method of producing a bacterial sequence-free vector having linear covalently closed ends comprising incubating any of the above vector production systems under suitable conditions for expression of the first recombinase.

The present disclosure is directed to a method of producing a bacterial sequence-free vector having linear covalently closed ends comprising incubating any of the above vector production systems under suitable conditions for expression of the first recombinase and the nuclease genome editing system. In some aspects, the method further comprises harvesting the bacterial sequence-free vector.

The present disclosure is directed to a bacterial sequence-free vector produced by any of the above methods of producing a bacterial sequence-free vector having linear covalently closed ends. In some aspects, the bacterial sequence-free vector is produced from expression vector B1. In some aspects, the bacterial sequence-free vector is produced from expression vector B2. In some aspects, the bacterial sequence-free vector is produced from expression vector B3. In some aspects, the bacterial sequence-free vector is produced from the expression vector B4. In some aspects, the bacterial sequence-free vector is produced from expression vector C. In some aspects, the bacterial sequence-free vector is produced from expression vector C1. In some aspects, the bacterial sequence-free vector is produced from expression vector D. In some aspects, the bacterial sequence-free vector is produced from expression vector E. In some aspects, the bacterial sequence-free vector is produced from expression vector F.

The present disclosure is directed to a method for producing a single-stranded AAV, wherein the method comprises: (a) transfecting cells that are capable of producing an AAV with: (i) the bacterial sequence-free vector produced from expression vector B3, (ii) the bacterial sequence-free vector produced from expression vector E or an expression vector comprising an expression cassette comprising an AAV rep gene and an AAV cap gene, and (iii) the bacterial sequence-free vector produced from expression vector F or an expression vector comprising an expression cassette comprising one or more helper virus genes for production of AAV, and (b) incubating the cells under conditions suitable for production of an AAV.

The present disclosure is directed to a method for producing a single-stranded AAV, wherein the method comprises: (a) transfecting cells that are capable of producing an AAV with: (i) the bacterial sequence-free vector produced from expression vector B4, (ii) the bacterial sequence-free vector produced from expression vector F or an expression vector comprising an expression cassette comprising one or more helper virus genes for production of AAV, and (b) incubating the cells under conditions suitable for production of an AAV.

The present disclosure is directed to a method for producing a single-stranded AAV, wherein the method comprises: (a) transfecting cells that are capable of producing an AAV with the bacterial sequence-free vector produced from expression vector B3, wherein each of an AAV rep gene, an AAV cap gene, and one or more helper virus genes for production of AAV is encoded by the cells or a vector, (b) incubating the cells under conditions suitable for expression of the rep gene, the cap gene, and the one or more helper virus genes and for production of an AAV.

The present disclosure is directed to a method for producing a self-complementary AAV, wherein the method comprises: (a) transfecting cells that are capable of producing an AAV with: (i) the bacterial sequence-free vector produced from expression vector B1, (ii) the bacterial sequence-free vector produced from expression vector E or an expression vector comprising an expression cassette comprising an AAV rep gene and an AAV cap gene, and (iii) the bacterial sequence-free vector produced from expression vector F or an expression vector comprising an expression cassette comprising one or more helper virus genes for production of AAV, and (b) incubating the cells under conditions suitable for production of an AAV.

The present disclosure is directed to a method for producing a self-complementary AAV, wherein the method comprises: (a) transfecting cells that are capable of producing an AAV with: (i) the bacterial sequence-free vector produced from expression vector B2, (ii) the bacterial sequence-free vector produced from expression vector F or an expression vector comprising an expression cassette comprising one or more helper virus genes for production of AAV, and (b) incubating the cells under conditions suitable for production of an AAV.

The present disclosure is directed to a method for producing a self-complementary AAV, wherein the method comprises: (a) transfecting cells that are capable of producing an AAV with: (i) the bacterial sequence-free vector produced from expression vector C, (ii) the bacterial sequence-free vector produced from expression vector E or an expression vector comprising an expression cassette comprising an AAV rep gene and an AAV cap gene, and (iii) the bacterial sequence-free vector produced from expression vector F or an expression vector comprising an expression cassette comprising one or more helper virus genes for production of AAV, and (b) incubating the cells under conditions suitable for production of an AAV.

The present disclosure is directed to a method for producing a self-complementary AAV, wherein the method comprises: (a) transfecting cells that are capable of producing an AAV with: (i) the bacterial sequence-free vector produced from expression vector C1, (ii) the bacterial sequence-free vector produced from expression vector F or an expression vector comprising an expression cassette comprising one or more helper virus genes for production of AAV, and (b) incubating the cells under conditions suitable for production of an AAV.

The present disclosure is directed to a method for producing a self-complementary AAV, wherein the method comprises: (a) transfecting cells that are capable of producing an AAV with the bacterial sequence-free vector produced from expression vector B1 or C, wherein each of an AAV rep gene, an AAV cap gene, and one or more helper virus genes for production of AAV is encoded by the cells or a vector, and (b) incubating the cells under conditions suitable for expression of the rep gene, the cap gene, and the one or more helper virus genes and for production of an AAV.

In some aspects, the cells in any of the above methods for producing a single-stranded AAV or self-complementary AAV are HEK293T cells.

In some aspects, any of the above methods for producing a single-stranded AAV or self-complementary AAV further comprise harvesting the AAV.

The present disclosure is directed to an AAV produced by any of the above methods for producing a single-stranded AAV or self-complementary AAV.

The present disclosure is directed to a pharmaceutical composition comprising the above AAV.

The present disclosure is directed to a method of treating a disease or disorder in a subject in need thereof, comprising administering the above AAV or the above pharmaceutical composition to the subject.

### BRIEF DESCRIPTION OF THE DRAWINGS

FIG. 1 shows a map of an exemplary expression vector containing a sequence of an inverted terminal repeat (ITR) flanking each side of an expression cassette encoding green fluorescent protein (GFP), and a specialized Super Sequence site (interchangeably designated in the figures as SS or SSeq) containing recombinase target sequences flanking each side of the ITR-expression cassette-ITR sequence.

FIG. 2 shows a map of an exemplary ministring DNA (msDNA) encoding GFP that is produced from the expression vector shown in FIG. 1.

FIG. 3 shows a map of an exemplary msDNA in which only the 3′ side of the expression cassette is flanked by an ITR.

FIG. 4 shows a map of an exemplary expression vector containing a SS flanking each side of an expression cassette encoding AAV Replication (Rep) and Capsid (Cap) sequences.

FIG. 5 shows a map for an exemplary msDNA encoding Rep and Cap sequences that is produced from the expression vector shown in FIG. 4.

FIG. 6 shows a map for an exemplary msDNA encoding helper sequences.

FIG. 7 shows a map of an exemplary expression vector containing a sequence of an ITR flanking each side of an expression cassette encoding GFP, and a SSeq flanking each side of the ITR-expression cassette-ITR sequence.

FIG. 8 shows a map of an exemplary msDNA that is produced from the expression vector shown in FIG. 7.

FIGS. 9A-9D show bar graphs of transfection efficiency (TE) (FIG. 9A and FIG. 9C), median fluorescence intensity (FIG. 9B), and cell viability (FIG. 9D) at day 2 (FIG. 9A and FIG. 9B) and day 6 (FIG. 9C and FIG. 9D) after transfection of HEK293 cells with the expression vector of FIG. 7 (AAV ITR pDNA) or the msDNA of FIG. 8 (AAV ITR msDNA). Arrows in FIG. 9A-9C indicate the highest values observed for msDNA. *=P<0.05, **=P<0.01, ***=P<0.001, and ns=not significant.

FIG. 10 shows photomicrographs of GFP expression in the transfected cells described in FIG. 9A-9D. Nuclei are indicated by staining with diamidino-2-phenylindole (DAPI).

FIG. 11 shows a map of an exemplary ITR-SacB-CmR-ITR expression cassette encoding the SacB protein and chloramphenicol acetyltransferase.

FIGS. 12A-12B show representative images of sucrose plates (FIG. 12A) and a bar graph of mutation rates (FIG. 12B) in cells transformed with ITR-sacB-CmR-ITR LCC DNA generated in vitro by PCR (Taq and Q5) or RCA (Phi29), or msDNA generated in vivo in E. coli (MBI2). Bars in (FIG. 9B) show the average of three biological replicates and error bars show one standard deviation. One-way ANOVA with Dunnett's test (compared to MBI2): ****=p<0.0001.

FIG. 13 shows a diagram of AAV production in which a conventional GOI-containing plasmid is replaced with msDNA.

FIG. 14 shows a map of an exemplary plasmid without SSeq containing a sequence of an ITR flanking each side of an expression cassette encoding GFP.

FIG. 15 shows photomicrographs of GFP expression in samples from 35 mL cultures 72 hours after transfection with a mixture of a conventional Helper plasmid, a conventional Rep2/Cap2 plasmid, and the msDNA shown in FIG. 8 (“msDNA”) or the plasmid shown in FIG. 14 (“pDNA”) in a molar ratio of 1:2:1, 2:1.5:1, or 1.4:1.5:1.

FIG. 16 shows photomicrographs of GFP expression in samples from 150 mL cultures for producing AAV1 or AAV2 72 hours after transfection with a mixture of a conventional Helper plasmid, a conventional Rep2/Cap1 plasmid (for AAV1 production) or a conventional Rep2/Cap2 plasmid (for AAV2 production), and the msDNA shown in FIG. 8 (“msDNA”) or the plasmid shown in FIG. 14 (“pDNA”) in 1.4:1.5:1 molar ratios for transfectants containing the msDNA or 2:1.5:1 ratios for transfectants containing the pDNA.

FIGS. 17A and 17B show cell viabilities in the samples described in FIG. 16 as % viable cells (FIG. 17A) and viable cell density (VCD) as concentrations in 10⁶ cells/mL (FIG. 17B).

FIG. 18 shows titers of AAV2 vector genome/mL (VG/mL) determined by droplet digital PCT (ddPCR) of the GOI after harvest of the cultures described in FIG. 15 at 72 hours after transfection.

FIGS. 19A and 19B show chromatograms from affinity chromatography of cultures transfected with msDNA for production of AAV1 (FIG. 19A) and AAV2 (FIG. 19B) as described in FIG. 16 following harvest at 72 hours. The upper line in each figure is the absorbance at 280 nm, while the lower line is the absorbance at 260 nm. The amount of “VP/mL” indicates the concentration of vector particles per milliliter in the eluate, and the percentage indicates the proportion of particles that are packaged with DNA.

FIGS. 20A and 20B show chromatograms from affinity chromatography of cultures transfected with pDNA for production of AAV1 (FIG. 20A) and AAV2 (FIG. 20B) as described in FIG. 16 following harvest at 72 hours. The lines, “VP/mL,” and percentage are as described for FIGS. 19A and 19B.

FIG. 21A shows a photomicrograph of an electrophoresis gel indicating capsid proteins VP1, VP2, and VP3 as the three respective bands from the top to the bottom of each lane as detected by ddPCR following affinity chromatography of the msDNA and pDNA AAV2 cultures as shown in FIGS. 19B and 20B, respectively.

FIG. 21B shows a bar graph of AAV1 and AAV2 titers in vector genome/mL (VG/mL) following affinity chromatography of the msDNA and pDNA AAV cultures described in FIGS. 19A-19B and 20A-20B.

FIGS. 22-23 show chromatograms from anion exchange (AEX) chromatography of the affinity chromatography captures shown in FIGS. 19A and 20A, respectively. The VP/mL and percentage are as described for FIGS. 19A-19B. Peak #1 in each figure includes particles that are primarily packaged with DNA, while peak #2 shows includes empty particles as well as particles with packaged DNA.

FIG. 24A shows a photomicrograph of an electrophoresis gel with bands as described for FIG. 21A following the AEX chromatography described for FIGS. 22-23, with pk #1 and pK #2 referring to peaks #1 and #2, respectively, of the AEX chomatograms.

FIGS. 24B and 24C show bar graphs of AAV1 titers (VG/mL) as determined by ddPCR from peaks #1 and #2 from the chromatograms of FIGS. 22-23, respectively, associated with the GOI or backbone elements from the conventional Rep/Cap and Helper plasmids (origin of replication (Ori), kanamycin resistance gene (KanR), and ampicillin resistance gene (AmpR)).

FIG. 25 shows a bar graph of AAV2 titers (VG/mL) as determined by ddPCR for GOI and backbone (Ori) sequences after harvest of the cultures described in FIG. 15 at 72 hours after transfection with the different ratios of msDNA or pDNA and conventional Rep/Cap and Helper plasmids.

FIG. 26 shows a next-generation sequencing (NGS) coverage map of packaged genomes in relation to plasmid map positions from the msDNA and pDNA AAV cultures after affinity chromatography capture as described in FIGS. 19A-19B and 20A-20B, respectively.

FIG. 27 shows a map of an exemplary msDNA containing an expression cassette encoding Rep2 and Cap2 and a SSeq flanking each side of the expression cassette.

FIGS. 28A and 28B show chromatograms from affinity chromatography of cultures transfected with a 1.4:1.5:1 molar ratio (FIG. 28A) of a conventional Helper plasmid (“pDNA-helper”), the msDNA shown in FIG. 27 (“msDNA-Rep2Cap2”), and the msDNA shown in FIG. 8 (“msDNA-cis”) and a 1:2:1 molar ratio (FIG. 29A) of the conventional Helper plasmid, a conventional Rep2/Cap2 plasmid (“pDNA-Rep2Cap2”), and the plasmid shown in FIG. 14 (“pDNA-cis”). The upper line, lower line, VP/mL, and percentage are as described for FIGS. 19A-19B. VG/mL indicates the number of particles/mL packaged with DNA in the eluate.

FIGS. 29A-29C show chromatograms from affinity chromatography. FIGS. 29A and 29C show chromatograms from affinity chromatography from independent repeats of the cultures described in FIGS. 28A and 28B, respectively. FIG. 29B shows a chromatogram from affinity chromatography of a culture transfected with a 1.4:1.5:1 molar ratio of pDNA-helper:pDNA-RepCap2:msDNA-cis as described in FIGS. 28A and 28B. The upper line, lower line, VP/mL, and percentage are as described for FIGS. 19A-19B. VG/mL is as described for FIGS. 28A and 28B. VP is the total number of viral particles calculated by multiplying the value VP/mL by the total volume of the eluate.

FIG. 29D shows a photomicrograph of an electrophoresis gel with bands as described for FIG. 21A, with lanes (a)-(c) corresponding to eluates from the chromatograms described in FIGS. 29A-29C, respectively.

FIGS. 30-32 show chromatograms from AEX chromatography of the affinity chromatography captures shown in FIGS. 29A-29C, respectively. The percentage is as described for FIGS. 19A-19B. VG/mL is as described for FIGS. 28A and 28B.

FIG. 33A shows titers of AAV2 vector genome/mL (VG/mL) based on presence of the GOI as determined by ddPCR for samples from initial harvest of the AAV2 after lysis of the cell cultures (“Harvest”), affinity chromatography (“Capture”), and AEX chromatography (“AEX”). The two Harvest and two Capture values for msDNA and pDNA samples are from independent repeats on different days as described in FIGS. 28A-28B and 29A-C, respectively. msDNA=transfection with pDNA-helper, msDNA-Rep2Cap2, and msDNA-cis; pDNA=transfection with pDNA-helper, pDNA-Rep2Cap2, and pDNA-cis; mixed-msDNA=transfection with pDNA-helper, pDNA-Rep2Cap2, msDNA-cis.

FIG. 33B shows titers of AAV2 (VG, mass balance) determined for each sample from the corresponding VG/mL titer in FIG. 33A.

FIG. 34A shows a bar graph of full particle percentages determined from the ratio of A260/A280 for the samples described in FIG. 33A as calculated from affinity chromatography captures shown in FIGS. 28A-28B (the first “msDNA” and “pDNA” bars graphs on the x-axis) as well as from the affinity chromatography captures shown in FIGS. 29A-29C and the AEX chromatography peaks shown in FIGS. 30-32 (the following “msDNA,” “mixed-msDNA,” and “pDNA” bar graphs).

FIG. 34B shows bar graphs of full particle percentages determined by mass photometry for the samples described in FIG. 33A from affinity chromatography captures shown in FIGS. 28A-28B and AEX chromatography peaks shown in FIGS. 30-32.

FIG. 35 shows a diagram of AAV production in which all three conventional plasmids are replaced with msDNA.

FIG. 36 shows a map of an exemplary msDNA containing an expression cassette encoding helper virus genes for AAV production.

FIGS. 37A-37D show chromatograms from affinity chromatography of cultures transfected with all msDNAs (FIGS. 37A-37C) or all pDNA (FIG. 37D). The msDNA cultures were transfected with the msDNAs of FIGS. 8, 27, and 36 in a 1:2:1 molar ratio and a 1:1 ratio of transfection agent:total DNA (FIG. 37A), a 1:2:1 molar ratio and 2:1 transfection agent:total DNA ratio (FIG. 37B), and a 1:1:1 molar ratio and 2:1 transfection agent:total DNA ratio (FIG. 37C). FIG. 37D shows the chromatogram of the all pDNA sample from FIG. 29C. The upper line, lower line, VP/mL, and percentages are as described for FIGS. 19A-19B. VG/L is the concentration of vector genomes in the culture.

FIG. 38 includes a summary of the sample characteristics and data from FIG. 37A-37D along with photomicrographs of electrophoresis gels indicating capsid proteins VP1, VP2, and VP3 with bands as described for FIG. 21A.

FIGS. 39-42 shows chromatograms from AEX chromatography of the affinity chromatography captures shown in FIGS. 37A-37D, respectively. Percentages and VG/mL are are as described for FIGS. 19A-19B and 28A-28B, respectively. Inserted photomicrographs in each figure show electrophoresis gels indicating capsid proteins VP1, VP2, and VP3 with bands as described for FIG. 21A.

FIG. 43A shows a bar graph of full particle percentages determined from the ratio of A260/A280 calculated from the affinity chromatography captures shown in FIGS. 37A-37D and the AEX chromatography peaks shown in FIGS. 39-42.

FIG. 43B shows a bar graph of full particle percentages determined by mass photometry from affinity chromatography captures shown in FIGS. 37A-37D and the AEX chromatography peaks shown in FIGS. 39-42.

FIG. 44A shows titers of AAV2 vector genome/mL (VG/mL) based on presence of the GOI as determined by ddPCR from initial harvest after lysis of the cultures described in FIGS. 37A-37D (“Harvest”), affinity chromatography shown in FIGS. 37A-37D (“Capture”), and AEX chromatography peak #1 shown in FIGS. 39-42 (“AEX”).

FIGS. 44B and 44C show the titers of AAV2 (VG, mass balance) determined for each sample from the corresponding VG/mL titer in FIG. 44A, with FIG. 44C including the sum of both peaks #1 and 2 of the AEX chromatography.

FIG. 45 shows an NGS coverage map of packaged genomes in relation to plasmid map positions from AAV cultures containing 1, 2, or 3 msDNAs for AAV production as described in the preceding figures and as compared to all pDNAs for AAV production.

FIG. 46 shows a bar graph of transfection efficiencies at 48 or 72 hours post-transfection with the msDNA of FIG. 8 (msDNA) or the plasmid of FIG. 14 (AAV PP) in a 1:1:1 molar ratio with bacterial-sequence minimized/reduced plasmids for Rep2/Cap9 and helper sequences for AAV9 production. msDNA1-9 indicate varying amounts of total transfected DNA and ratios of transfection agent (PEI):DNA as follows: (msDNA1) 1.0 μg/mL DNA and 1.5:1 PEI:DNA, (msDNA2) 1.0 g/mL DNA and 2:1 PEI:DNA, (msDNA3) 1.0 μg/mL DNA and 2.5:1 PEI:DNA, (msDNA4) 1.75 μg/mL DNA and 1.5:1 PEI:DNA, (msDNA5) 1.75 μg/mL DNA and 2:1 PEI:DNA, (msDNA6) 1.75 μg/mL DNA and 2.5:1 PEI:DNA, (msDNA7) 2.5 μg/mL DNA and 1.5:1 PEI:DNA, (msDNA8) 2.5 μg/mL DNA and 2:1 PEI:DNA, and (msDNA9) 2.5 μg/mL DNA and 2.5:1 PEI:DNA.

FIG. 47 shows bar graphs of cell viabilities as viable cell density (VCD, cells/mL) and % viable cells at 48 or 72 hours following the transfections described in FIG. 46.

FIG. 48 shows a bar graph of capsid titers for the samples described in FIG. 46 as determined by ELISA specific for AAV9 at 72 hours post-transfection.

FIG. 49 shows a bar graph of AAV titers determined for the samples described in FIG. 46 by ddPCR with primers specific to the ITR region at 72 hours post-transfection.

FIG. 50 shows a bar graph of total AAV9 titers determined by ddPCR after AEX chromatography for a 10 L culture of the transfectant msDNA5 (“msDNA”) and AAV PP (“pDNA”) as described in FIG. 46.

### DETAILED DESCRIPTION OF THE INVENTION

The present disclosure provides expression vectors, vector production systems, methods of producing bacterial sequence-free vectors, and bacterial sequence-free vectors for producing AAV as well as methods for producing the AAV, the AAV, compositions comprising the AAV, and methods of using the AAV.

All publications cited herein are hereby incorporated by reference in their entireties, including without limitation all journal articles, books, manuals, patent applications, and patents cited herein, to the same extent as if each individual publication was specifically and individually indicated to be incorporated by reference.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range, or integer range is to be understood to include the value of any integer within the recited range and, when appropriate, fractions thereof (such as one tenth and one hundredth of an integer), unless otherwise indicated. Numeric ranges are inclusive of the numbers defining the range.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Système International de Unites (SI) accepted form.

Unless otherwise indicated, nucleotide sequences are written left to right in 5′ to 3′ orientation. Amino acid sequences are written left to right in amino to carboxy orientation.

The headings provided herein are not limitations of the various aspects of the disclosure, which can be had by reference to the specification as a whole. Accordingly, the terms defined immediately below are more fully defined by reference to the specification in its entirety.

An “adeno-associated virus,” i.e., “AAV,” as used herein refers to a parvovirus of the family Parvoviridae that is a member of the genus Dependoparvovirus (formerly Dependovirus). An AAV containing a nucleic acid sequence of interest as disclosed herein can be interchangeably referred to as an “AAV,” “recombinant AAV,” “rAAV,” or “AAV vector.”

An “inverted terminal repeat,” i.e., “ITR,” as used herein refers to a single-stranded polynucleotide or a sense or antisense strand (i.e., + or − strand, respectively) of a double-stranded polynucleotide that contains a sequence for AAV replication and a non-palindromic packaging signal. An “ITR” as disclosed herein includes a wild-type AAV 5′ ITR and/or 3′ ITR sequence, a portion thereof, or an artificial sequence.

A “sequence for AAV replication” as used herein refers to a sequence within an AAV ITR associated with AAV replication, and includes the Rep protein binding element (RBE), RBE′, terminal resolution site (TRS), or any combination thereof. The RBE can also be referred to interchangeably herein as the Rep protein binding site (RBS).

An “AAV packaging signal” as used herein refers to a non-palindromic sequence in an ITR associated with AAV encapsidation, and comprises the “D region” of a 5′ or 3′ AAV ITR or a functional portion thereof.

“Protein” or “polypeptide” refers to any polymer of two or more individual amino acids (whether or not naturally occurring) linked via a peptide bond, and occurs when the carboxyl carbon atom of the carboxylic acid group bonded to the alpha-carbon of one amino acid (or amino acid residue) becomes covalently bound to the amino nitrogen atom of amino group bonded to the non alpha-carbon of an adjacent amino acid. The term “protein” is understood to include the terms “polypeptide” and “peptide” (which, at times may be used interchangeably herein) within its meaning. In addition, proteins comprising multiple polypeptide subunits will also be understood to be included within the meaning of “protein” as used herein. Similarly, fragments of proteins and polypeptides are also within the scope of the disclosure and may be referred to herein as “proteins.” In one aspect of the disclosure, a polypeptide comprises a chimera of two or more parental peptide segments. The term “polypeptide” is also intended to refer to and encompass the products of post-translation modification (“PTM”) of the polypeptide, including without limitation disulfide bond formation, glycosylation, carbamylation, lipidation, acetylation, phosphorylation, amidation, derivatization by known protecting/blocking groups, proteolytic cleavage, modification by non-naturally occurring amino acids, or any other manipulation or modification, such as conjugation with a labeling component. A polypeptide can be derived from a natural biological source or produced by recombinant technology, but is not necessarily translated from a designated nucleic acid sequence. It can be generated in any manner, including by chemical synthesis. An “isolated” polypeptide or a fragment, variant, or derivative thereof refers to a polypeptide that is not in its natural milieu. No particular level of purification is required. For example, an isolated polypeptide can simply be removed from its native or natural environment. Recombinantly produced polypeptides and proteins expressed in host cells are considered isolated for the purpose of the disclosure, as are native or recombinant polypeptides which have been separated, fractionated, or partially or substantially purified by any suitable technique.

“Polynucleotide” or “nucleic acid” as used herein refers to a polymeric form of nucleotides. In some instances, a polynucleotide comprises a sequence that is either not immediately contiguous with the coding sequences or is immediately contiguous (on the 5′ end or on the 3′ end) with the coding sequences in the naturally occurring genome of the organism from which it is derived. The term therefore includes, for example, a recombinant DNA which is incorporated into a vector, into an autonomously replicating plasmid or virus, or into the genomic DNA of a prokaryote or eukaryote, or which exists as a separate molecule (e.g., a cDNA) independent of other sequences. The nucleotides of the disclosure can be ribonucleotides, deoxyribonucleotides, or modified forms of either nucleotide. A polynucleotide as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The term polynucleotide encompasses genomic DNA or RNA (depending upon the organism, i.e., RNA genome of viruses), as well as mRNA encoded by the genomic DNA, and cDNA. In certain embodiments, a polynucleotide comprises a conventional phosphodiester bond or a non-conventional bond (e.g., an amide bond, such as found in peptide nucleic acids (PNA)). By “isolated” nucleic acid or polynucleotide is intended a nucleic acid molecule, e.g., DNA or RNA, which has been removed from its native environment. For example, a nucleic acid molecule comprising a polynucleotide encoding a recombinant polypeptide contained in a vector is considered “isolated” for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vitro RNA transcripts of polynucleotides of the present disclosure. Isolated polynucleotides or nucleic acids according to the present disclosure further include polynucleotides and nucleic acids (e.g., nucleic acid molecules) produced synthetically.

As used herein, an “expression cassette” comprises a nucleic acid sequence of interest (e.g., a nucleic acid sequence for expression of a polypeptide, DNA, or RNA) and an expression control region.

As used herein, a “transgene” can be used interchangeably with “gene of interest” or “GOI” to refer to a portion of a polynucleotide that contains codons translatable into amino acids. Although a “stop codon” (TAG, TGA, or TAA) is typically not translated into an amino acid, it may be considered to be part of a transgene, but any flanking sequences, for example promoters, ribosome binding sites, transcriptional terminators, introns, and the like, are not part of the transgene. The boundaries of a transgene are typically determined by a start codon at the 5′ terminus, encoding the amino-terminus of the resultant polypeptide, and a translation stop codon at the 3′ terminus, encoding the carboxyl-terminus of the resulting polypeptide.

As used herein, the term “expression control region” refers to a transcription control element that is operably associated with a nucleic acid sequence of interest to direct or control expression of the expression product of the nucleic acid sequence of interest, including, for example, cis-regulatory modules (CRMs), promoters (e.g., a tissue specific promoter and/or an inducible promoter), enhancers, operators, repressors, ribosome binding sites, translation leader sequences, introns, post-transcriptional elements, polyadenylation recognition sequences, RNA processing sites, effector binding sites, stem-loop structures, transcription termination signals, miRNA binding sites, and combinations thereof. Expression control regions include nucleotide sequences located upstream (5′), within, or downstream (3′) of a nucleic acid sequence of interest, and which influence the transcription, RNA processing, stability, or translation of the associated nucleic acid sequence of interest. If a transgene is intended for expression in a eukaryotic cell, a polyadenylation signal and transcription termination sequence will usually be located 3′ to the transgene.

As used herein, the terms “host cell” and “cell” can be used interchangeably and can refer to any type of cell or a population of cells, e.g., a primary cell, a cell in culture, or a cell from a cell line, that harbors or is capable of harboring a nucleic acid molecule (e.g., a recombinant nucleic acid molecule). Host cells can be a prokaryotic cell, or alternatively, the host cells can be eukaryotic, for example, fungal cells, such as yeast cells, and various animal cells, such as insect cells or mammalian cells.

“Culture,” “to culture” and “culturing,” as used herein, means to incubate cells under in vitro conditions that allow for cell growth or division or to maintain cells in a living state. “Cultured cells,” as used herein, means cells that are propagated in vitro.

A “subject” includes any human or nonhuman animal. The term “nonhuman animal” includes, but is not limited to, vertebrates such as nonhuman primates, sheep, dogs, and rodents such as mice, rats and guinea pigs. In preferred aspects, the subject is a human. The terms, “subject” and “patient” are used interchangeably herein.

“Administering” refers to the physical introduction of a composition comprising a therapeutic agent to a subject, using any of the various methods and delivery systems known to those skilled in the art.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease, condition, or disorder.

As used herein, “effective treatment” refers to treatment producing a beneficial effect, e.g., amelioration of at least one symptom of a disease, condition, or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of a disease, condition, or disorder. Effective treatment can refer to alleviation of at least one symptom of a disease, condition, or disorder.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some aspects, an effective amount is an amount sufficient to prevent or delay recurrence of a symptom of a disease, condition, or disorder. An effective amount can be administered in one or more

Various aspects of the invention are described in further detail in the following subsections.

### II. Production of AAV from msDNA

Ministring DNA vectors (interchangeably referred to herein as “msDNA vectors” or “msDNA”) are bacterial sequence-free vectors having linear covalently closed (LCC) ends. See U.S. Pat. Nos. 9,290,778 and 9,862,954, and International Publication No. WO 2022/264095; Nafissi and Slavcev, Microbial Cell Factories 11:154 (2012); and Nafissi et al., Nucleic Acids 3(6):e165 (2014), incorporated by reference herein in their entireties. msDNA is a “bacterial sequence-free vector” because it lacks any bacterial backbone sequences, such as antibiotic resistance genes, bacterial origin of replication, or immunostimulatory un-methylated CpG motifs typical of plasmid-based vectors. Integration of msDNA into a cell's chromosome results in a chromosomal break and elimination of the cell through apoptotic cell death. Thus, msDNA eliminates any risk of insertional mutagenesis, avoiding potential genotoxicity and oncogenic events associated with integration when using other delivery vectors. See Nafissi et al.

msDNA is produced from an expression vector (e.g., a plasmid) that contains specialized “Super Sequence” (“SS” or “SSeq,” as used interchangeably herein) sites comprising target sequences for recombinases. The SS sites flank an expression cassette containing a nucleic acid of interest. When the expression vector is present in a recombinant cell that expresses an appropriate recombinase, an msDNA containing the expression cassette is separated from the backbone DNA of the expression vector. The msDNA can then be purified and used directly as a delivery vector. See U.S. Pat. Nos. 9,290,778 and 9,862,954, International Publication No. WO 2022/264095, Nafissi and Slavcev, and Nafissi et al.

A. Expression Vectors, Vector Production Systems, and msDNA

Provided herein are expression vectors for producing msDNA comprising sequences that can be used to produce AAV.

In one aspect, the expression vector comprises: (a) a first sequence comprising an inverted terminal repeat (ITR) that flanks at least one side of a desired sequence, wherein the ITR comprises a sequence for adeno-associated virus (AAV) replication and an AAV packaging signal, (b) a target sequence for a first recombinase flanking each side of the first sequence, and (c) one or more additional target sequences for one or more additional recombinases integrated within non-binding regions of the target sequence for the first recombinase, wherein the expression vector is for producing a bacterial sequence-free vector having linear covalently closed ends (i.e., an msDNA). In some aspects, the desired sequence is a multiple cloning site (MCS), an expression cassette comprising a nucleic acid sequence of interest, a palindromic sequence comprising an expression cassette comprising a nucleic acid sequence of interest and a complement of the expression cassette, or a portion of an expression cassette comprising a nucleic acid sequence of interest flanked on one side by a splicing sequence.

Any ITR sequence containing a sequence for AAV replication and an AAV packaging signal as disclosed herein can be used in the aspects of the invention disclosed herein.

The terminus of each wild-type AAV ITR contains palindromic regions (A and A′, B and B′, and C and C′) that self-anneal to form a double-stranded T-shaped hairpin structure. Self-annealed B-B′ and C-C′ palindromes form the cross arm of the hairpin while the self-annealed A-A′ palindrome forms the stem of the hairpin. The hairpin is followed by a short non-palindromic region (D) in the ITR that provides a packaging signal. A, A′, B, B′, C, C′, and D can be interchangeably referred to herein as “sequences” or “regions” (e.g., A sequence, sequence A, A region, region A, etc.). Studies have shown that packaging and replication of AAV can occur in ITRs lacking the B-B′ and C-C′ regions. See, e.g., Zhou et al., Scientific Reports 7:5432 (2017).

The ITR contains sequences associated with Rep protein functions. A 16-nucleotide tetrameric repeat within A-A′ known as the Rep protein binding element (RBE) is bound by Rep68/Rep78, which has helicase activity and unwinds the RBE sequence. As used herein, RBE includes the double-stranded structure formed when the palindromic A-RBE and A′-RBE sequences self-anneal. A sequence at one tip of one of the internal palindromic B-B′ region termed RBE′ orients Rep68/Rep78 towards a terminal resolution site (TRS). Rep68/Rep78 endonuclease activity cleaves the TRS to resolve the double-stranded sequence during replication and produce the single-stranded genome for packaging. See, e.g., Daya and Berns, Lisowski et al.; Ling et al., J. Mol. Genet. Med. 9(3):175 (2015); Salganik et al., Microbiol. Spectrum 3(4):MDNA3-0052-2014.

Currently, over 100 human and non-human primate AAVs have been identified, including 13 serotypes. See, e.g., Daya and Berns; Lisowski et al.; and Mary et al. Sequences for the ITRs of each serotype are known in the art or can be readily determined by skilled artisans, including from exemplary accession numbers for AAV1 (NC_002077.1; AF063497.1), AAV2 (J01901.1; NC_001401.2), AAV3 (AAV3A, NC_001729.1; AAV3B, AF028705.1), AAV4 (NC_001829.1), AAV5 (NC_006152.1; AF085716.1), AAV6 (AF028704.1), AAV7 (NC_006260.1), AAV8 (NC_006261.1), AAV9 (AX753250.1), AAV10 (AY631965.1), AAV11 (AY631966.1), AAV12 (DQ813647.1), and AAV13 (EU285562.1).

In some aspects, the ITR flanks only one side (i.e., 5′ITR or 3′ ITR) of the MCS, expression cassette, palindromic sequence, or portion of an expression cassette in an expression vector disclosed herein.

In some aspects, the ITR flanks each side (i.e., 5′ ITR and 3′ ITR) of the MCS, expression cassette, palindromic sequence, or portion of an expression cassette in an expression vector disclosed herein.

In some aspects, an expression vector, msDNA, or AAV as disclosed herein comprises a wild-type AAV 5′ ITR and/or 3′ ITR sequence. In some aspects, an expression vector, msDNA, or AAV as disclosed herein comprises a portion of a wild-type AAV ITR sequence or an artificial sequence, which contains a sequence for AAV replication and an AAV packaging signal.

In some aspects, the ITR is a wild-type AAV ITR.

In some aspects, the ITR is a portion of a wild-type AAV ITR comprising a sequence for AAV replication and an AAV packaging signal.

In some aspects, the ITR is an artificial ITR comprising a sequence for AAV replication and an AAV packaging signal.

In some aspects, the ITR comprises A, A′, and D sequences.

In some aspects, the ITR comprises a RBE and a D sequence of an AAV ITR.

In some aspects, the ITR flanks each side of the MCS, expression cassette, palindromic sequence, or portion of an expression cassette and the ITR on each side is identical.

In some aspects, the ITR flanks each side of the MCS, expression cassette, palindromic sequence, or portion of an expression cassette and the ITR on each side is different.

In some aspects, any of the expression vectors, msDNAs, or AAVs as disclosed herein comprise a 5′ ITR and a 3′ ITR flanking the MCS, expression cassette, palindromic sequence, or portion of an expression cassette, and the 5′ and 3′ ITRs are from the same serotype or different serotypes.

In some aspects, an ITR as disclosed herein is a chimeric ITR comprising sequences from different AAV serotypes.

Exemplary AAV ITR sequences are shown in Table 1.

TABLE 1
Exemplary AAV ITR sequences

| AAV region | sequence in + strand AAV genome |
|---|---|
| wild-type 5′ ITR | ttggccactccctctctgcgcgctcgctcgctcactgaggccgggcgaccaaaggtcgcc |
| | cgacgcccgggctttgcccgggggcctcagtgagcgagcgagcgcgcagagaggg |
| | agtggccaactccatcactaggggttcct (SEQ ID NO: 1) |
| wild-type 3′ ITR | aggaacccctagtgatggagttggccactccctctctgcgcgctcgctcgctcactgagg |
| | ccgggcgaccaaaggtcgcccgacgcccgggctttgcccgggcggcctcagtgagcg |
| | agcgagcgcgcagagagggagtggccaa (SEQ ID NO: 2) |
| A | ttggccactccctctctgegcgctcgctcgctcactgaggc (SEQ ID NO: 3) |
| A′ | gcctcagtgagcgagcgagcgcgcagagagggagtggccaa (SEQ ID NO: 4) |
| B | cgggcgacca (SEQ ID NO: 5) |
| B′ | aaggtcgcccg (SEQ ID NO: 6) |
| C | cgcccgggct (SEQ ID NO: 7) |
| C′ | ttgcccgggcg (SEQ ID NO: 8) |
| 5′ ITR D | ctccatcactaggggttcct (SEQ ID NO: 9) |
| 3′ ITR D | aggaacccctagtgatggag (SEQ ID NO: 10) |
| A-RBE | gegcgctcgctegctc (SEQ ID NO: 11) |
| A′-RBE | gagcgagcgagegcgc (SEQ ID NO: 12) |
| RBE′ | caaag (SEQ ID NO: 13) |
| 5′ ITR TRS | ccaact (SEQ ID NO: 14) |
| 3′ ITR TRS | agttgg (SEQ ID NO: 15) |
| artificial 5′ ITR | ctgcgcgctcgctcgctcactgaggccgcccgggcaaagcccgggcgtcgggcgacct |
| | ttggtcgcccggcctcagtgagcgagcgagcgcgcagagagggagtggccaactccat |
| | cactaggggttcct (SEQ ID NO: 16) |
| artificial 3′ ITR | aggaacccctagtgatggagttggccactccctctctgcgcgctcgctcgctcactgagg |
| | ccgggcgaccaaaggtcgcccgacgcccgggctttgcccgggcggcctcagtgagcg |
| | agcgagcgcgcag (SEQ ID NO: 17) |
| artificial 5′ ITR | ttggccactccctctctgcgcgctcgctcgctcactgaggccgcccgggcaaagcccgg |
| | gcgtcgggcgacctttggtcgcccggcctcagtgagcgagcgagcgcgcagagaggg |
| | agtggccaactccatcactaggggttcct (SEQ ID NO: 38) |
| artificial 3′ ITR | aggaacccctagtgatggagttggccactccctctctgcgcgctcgctcgctcactgagg |
| | ccgggcgaccaaaggtcgcccgacgcccgggctttgcccgggcggcctcagtgagcg |
| | agcgagcgcgcagagagggagtggccaa (SEQ ID NO: 39) |

In some aspects, an ITR for any of the expression vectors, msDNAs, or AAVs as disclosed herein comprises one or more sequences from Table 1.

In some aspects, an ITR for any of the expression vectors, msDNAs, or AAVs as disclosed herein comprises one or more sequences from a minus strand (i.e., − strand or antisense strand) AAV genome corresponding to the plus strand (i.e., + strand or sense strand) AAV genome sequences in Table 1.

In some aspects, an ITR sequence for any of the expression vectors, msDNAs, or AAVs as disclosed herein comprises one or more 5′ ITR, 3′ ITR, A, A′, B, B′, C, C′, D, A-RBE, A′-RBE, RBE′, 5′ ITR D, 3′ ITR D, 5′ ITR TRS, or 3′ ITR TRS sequences at least about 90%, at least about 91%, at least about 92%, at least about 93%, at least about 94%, at least about 95%, at least about 96%, at least about 97%, at least about 98%, or at least about 99% identical to a corresponding sequence in Table 1.

In some aspects, an ITR sequence for any of the expression vectors, msDNAs, or AAVs as disclosed herein comprises one or more 5′ ITR, 3′ ITR, A, A′, B, B′, C, C′, D, A-RBE, A′-RBE, RBE′, 5′ ITR D, 3′ ITR D, 5′ ITR TRS, or 3′ ITR TRS sequences at least about 90%, at least about 91%, at least about 92%, at least about 93%, at least about 94%, at least about 95%, at least about 96%, at least about 97%, at least about 98%, or at least about 99% identical to a corresponding-strand (i.e., antisense strand) AAV genome sequence of the + strand (i.e., sense strand) AAV genome sequence in Table 1.

In some aspects, the ITRs for any of the expression vectors, msDNAs, or AAVs as disclosed herein comprise a 5′ ITR having the polynucleotide sequence of SEQ ID NO: 16 and/or a 3′ ITR the polynucleotide sequence of SEQ ID NO: 17. In some aspects, the ITRs for any of the expression vectors, msDNAs, or AAVs as disclosed herein comprise a 5′ ITR having the polynucleotide sequence of SEQ ID NO: 16 and a 3′ ITR the polynucleotide sequence of SEQ ID NO: 17.

In some aspects, the ITRs for any of the expression vectors, msDNAs, or AAVs as disclosed herein comprise a 5′ ITR having the polynucleotide sequence of SEQ ID NO: 38 and/or a 3′ ITR the polynucleotide sequence of SEQ ID NO: 39. In some aspects, the ITRs for any of the expression vectors, msDNAs, or AAVs as disclosed herein comprise a 5′ ITR having the polynucleotide sequence of SEQ ID NO: 38 and a 3′ ITR the polynucleotide sequence of SEQ ID NO: 39.

In some aspects, an expression vector as disclosed herein further comprises a spacer sequence between the target sequence for the first recombinase and the first sequence (e.g., between the target sequence for the first recombinase and the ITR such as between the 5′ target sequence for the first recombinase and the 5′ ITR and/or between the 3′ target sequence for the first recombinase and the 3′ ITR). In some aspects, an msDNA or AAV as described herein further comprises a spacer sequence between a portion of a SSeq (e.g., the portion remaining after Tel recombination of an expression vector as described herein comprising a SSeq, e.g., the portion provided in the polynucleotide sequence of SEQ ID NO: 37) and the ITR (i.e., the 5′ ITR and/or the 3′ ITR). In some aspects, the spacer sequence is about 10 to about 500 nucleotides. In some aspects, the spacer sequence is about 1 to about 10 nucleotides, about 10 to about 50 nucleotides, about 50 to about 100 nucleotides, about 100 to about 250 nucleotides, or about 250 to about 500 nucleotides.

In some aspects, the 5′ spacer sequence is the polynucleotide sequence of SEQ ID NO: 40.

In some aspects, the 3′ spacer sequence is the polynucleotide sequence of SEQ ID NO: 41.

In some aspects, any of the expression vectors, msDNAs, or AAVs as disclosed herein comprise a 5′ spacer sequence having the polynucleotide sequence of SEQ ID NO: 40 and 3′ spacer sequence having the polynucleotide sequence of SEQ ID NO: 41.

In some aspects, the 5′ spacer sequence is the polynucleotide sequence of SEQ ID NO: 40 and the 5′ ITR is the polynucleotide sequence of SEQ ID NO: 38 and/or the 3′ spacer sequence is the polynucleotide sequence of SEQ ID NO: 41 and the 3′ ITR the polynucleotide sequence of SEQ ID NO: 39.

In some aspects, any of the expression vectors, msDNAs, or AAVs as disclosed herein comprise a 5′ spacer sequence having the polynucleotide sequence of SEQ ID NO: 40, a 5′ ITR having the polynucleotide sequence of SEQ ID NO: 38, a 3′ spacer sequence having the polynucleotide sequence of SEQ ID NO: 41, and a 3′ ITR having the polynucleotide sequence of SEQ ID NO: 39.

In some aspects, the expression vector lacks any spacer sequence between the target sequence for the first recombinase and the first sequence.

In some aspects, the expression vector further comprises an expression cassette comprising an AAV replication (rep) gene and/or an AAV capsid (cap) gene flanked on one side by the target sequence for the first recombinase and flanked on the other side by the first sequence.

The AAV rep and cap genes in any of the expression vectors or msDNAs disclosed herein can be from any AAV, including any AAV serotype, disclosed herein. The ITR, rep gene, and/or cap gene can be from the same AAV or different AAVs, including the same AAV serotype or different AAV serotypes. The rep gene and/or cap gene can also be a hybrid sequence containing sequences from different AAVs such that any of Rep40, Rep52, Rep68, Rep78, VP1, VP2, VP3, and AAP, or portions thereof, can be encoded by sequences from different AAVs.

Sequences for the rep and cap genes for each of the 13 identified serotypes are known in the art or could be readily determined by those of skill in the art, including from the exemplary accession numbers for the 13 serotypes disclosed herein. For examples, AAV2 rep and cap genes are provided herein as SEQ ID NOs: 21 and 22, respectively, or SEQ ID NOs: 45 and 22, respectively. As used herein, a “serotype” refers to an AAV with a capsid that is serologically distinct from other AAVs as shown, for example, by lack of cross-reactivity between antibodies to one AAV and another AAV due to differences in capsid proteins.

The serotypes differ in their tissue tropism (i.e., the types of cells that they infect) based on their capsid. See, e.g., Likowski et al.; Daya and Berns.

In some aspects, an AAV is targeted to a tissue or cell comprising a cell surface receptor for an AAV serotype. In some aspects, the cell surface receptor is heparan sulfate proteoglycan (e.g., a cell surface receptor for AAV-3), O-linked sialic acid (e.g., a cell surface receptor for AAV-4), platelet-derived growth factor receptor (e.g., a cell surface receptor for AAV-5), or a 37-kDa/67-kDa laminin receptor (e.g. a cell surface receptor for AAV-2, AAV-3, AAV-8, or AAV-9).

The mixing of genomic sequences from one AAV serotype with a capsid from another AAV serotype is known as pseudotyping, and is denoted herein with a slash. For example, a recombinant AAV having ITR sequences from AAV2 in combination with a capsid from AAV5 is denoted herein as AAV2/5.

In some aspects, an expression vector, an msDNA, a combination of expression vectors (i.e., the ITR and cap sequences are located on separate expression vectors), a combination of msDNAs, or an AAV as disclosed herein comprises an ITR/cap pseudotype of 1/2, 1/3, 1/4, 1/5, 1/6, 1/7, 1/8, 1/9, 1/10, 1/11, 1/12, or 1/13. In some aspects, a pseudotyped AAV as disclosed herein contains an ITR/cap gene of 2/1, 2/3, 2/4, 2/5, 2/6, 2/7, 2/8, 2/9, 2/10, 2/11, 2/12, or 2/13. In some aspects, a pseudotyped AAV as disclosed herein contains an ITR/cap gene of 3/1, 3/2, 3/4, 3/5, 3/6, 3/7, 3/8, 3/9, 3/10, 3/11, 3/12, or 3/13. In some aspects, a pseudotyped AAV as disclosed herein contains an ITR/cap gene of 4/1, 4/2, 4/3, 4/5, 4/6, 4/7, 4/8, 4/9, 4/10, 4/11, 4/12, or 4/13. In some aspects, a pseudotyped AAV as disclosed herein contains an ITR/cap gene of 5/1, 5/2, 5/3, 5/4, 5/6, 5/7, 5/8, 5/9, 5/10, 5/11, 5/12, or 5/13. In some aspects, a pseudotyped AAV as disclosed herein contains an ITR/cap gene of 6/1, 6/2, 6/3, 6/4, 6/5, 6/7, 6/8, 6/9, 6/10, 6/11, 6/12, or 6/13. In some aspects, a pseudotyped AAV as disclosed herein contains an ITR/cap gene of 7/1, 7/2, 7/3, 7/4, 7/5, 7/6, 7/8, 7/9, 7/10, 7/11, 7/12, or 7/13. In some aspects, a pseudotyped AAV as disclosed herein contains an ITR/cap gene of 8/1, 8/2, 8/3, 8/4, 8/5, 8/6, 8/7, 8/9, 8/10, 8/11, 8/12, or 8/13. In some aspects, a pseudotyped AAV as disclosed herein contains an ITR/cap gene of 9/1, 9/2, 9/3, 9/4, 9/5, 9/6, 9/7, 9/8, 9/10, 9/11, 9/12, or 9/13. In some aspects, a pseudotyped AAV as disclosed herein contains an ITR/cap gene of 10/1, 10/2, 10/3, 10/4, 10/5, 10/6, 10/7, 10/8, 10/9, 10/11, 10/12, or 10/13. In some aspects, a pseudotyped AAV as disclosed herein contains an ITR/cap gene of 11/1, 11/2, 11/3, 11/4, 11/5, 11/6, 11/7, 11/8, 11/9, 11/10, 11/12, or 11/13. In some aspects, a pseudotyped AAV as disclosed herein contains an ITR/cap gene of 12/1, 12/2, 12/3, 12/4, 12/5, 12/6, 12/7, 12/8, 12/9, 12/10, 12/11, 12/12, or 12/13. In some aspects, a pseudotyped AAV as disclosed herein contains an ITR/cap gene of 13/1, 13/2, 13/3, 13/4, 13/5, 13/6, 13/7, 13/8, 13/9, 13/10, 13/11, or 13/12.

A capsid as disclosed herein can also be a hybrid capsid produced with capsid proteins from multiple serotypes. For example, AAV-DJ has a hybrid capsid derived from 8 serotypes. In some aspects, an expression vector or an msDNA as disclosed herein encodes, or an AAV as disclosed herein comprises, a hybrid capsid. In some aspects, the hybrid capsid comprises capsid proteins from any two or more of the AAV1-AAV13 serotypes.

Table 2 provides an exemplary listing of tissue tropism for selected AAV serotypes, strains, and recombinant AAVs.

TABLE 2
Exemplary AAV tissue tropism

| Tissue | AAV |
|---|---|
| Central Nervous System | AAV1, AAV2, AAV4, AAV5, AAV7, |
| | AAV8, AAV9, AAVrh10, AAV-PHP.eB |
| Peripheral Nervous System | AAV-PHP.S |
| Brain | AAV1, AAV2, AAV5, AAV7, AAV8, |
| | AAV-DJ/8 |
| Heart | AAV1, AAV4, AAV5, AAV6, AAV8, |
| | AAV9, AAVrh10, AAV-DJ |
| Inner Ear | AAV1, AAV2, AAV6.2, AAV8, AAV9, |
| | AAV2.7m8 |
| Kidney | AAV2, AAV4, AAV8, AAV9, AAVrh10, |
| | AAV-DJ, AAV-DJ/8 |
| Liver | AAV1, AAV2, AAV3, AAV6, AAV6.2, |
| | AAV7, AAV8, AAV9, AAVrh10, AAV- |
| | DJ, AAV-DJ/8 |
| Lung | AAV1, AAV3, AAV4, AAV5, AAV6, |
| | AAV6.2, AAV9, AAVrh10 |
| Pancreas | AAV1, AAV2, AAV6, AAV8, AAV9, |
| | AAVrh10 |
| Photoreceptor Cells | AAV2, AAV5, AAV8 |
| RPE (Retinal Pigment | AAV1, AAV2, AAV4, AAV5, AAV8 |
| Epithelium) | |
| Retina | AAV1, AAV2, AAV4, AAV5, AAV7, |
| | AAV8, AAV9, AAVrh10, AAV2.7m8 |
| Skeletal Muscle | AAV1, AAV6, AAV7, AAV8, AAV9 |
| Smooth Muscle | AAV1, AAV2, AAV3, AAV5, AAV6, |
| | AAV7, AAV8, AAV9, AAVrh10 |
| Adipose | AAV6, AAV8, AAV9 |
| Testes | AAV2, AAV9 |
| Spleen | AAV-DJ, AAV-DJ/8 |
| Spinal nerves | AAV2-retro |
| Endothelial cells | AAV2-QuadYF |

In some aspects, any of the expression vectors, msDNAs, or AAVs disclosed herein comprise an ITR, rep gene, or cap gene from any of the AAVs, or any combination of AAVs, listed in Table 2. In some aspects, any of the AAVs disclosed herein comprise a capsid from any of the AAVs listed in Table 2.

In some aspects, any of the expression vectors, msDNAs, or AAVs as disclosed herein comprising a rep gene and a cap gene comprise a rep2 gene in combination with a cap gene from any of the AAV1-AAV13 serotypes for production of the serotype.

In some aspects, any of the expression vectors, msDNAs, or AAVs as disclosed herein comprising a rep gene and a cap gene comprise a rep2 gene and a cap1 gene for AAV1 production.

In some aspects, any of the expression vectors, msDNAs, or AAVs as disclosed herein comprising a rep gene and a cap gene comprise a rep2 gene and a cap2 gene for AAV2 production.

In some aspects, an msDNA for expression of a rep gene and a cap gene in AAV2 production as disclosed herein comprises the polynucleotide sequence of SEQ ID NO: 24.

In some aspects, an msDNA for expression of a rep gene and a cap gene in AAV2 production as disclosed herein comprises the polynucleotide sequence of SEQ ID NO: 48.

In some aspects, an msDNA for expression of a rep gene and a cap gene in AAV5 production as disclosed herein comprises the polynucleotide sequence of SEQ ID NO: 49.

In some aspects, an msDNA for expression of a rep gene and a cap gene in AAV9 production as disclosed herein comprises the polynucleotide sequence of SEQ ID NO: 50.

In some aspects, any of the expression vectors, msDNAs, or AAVs as disclosed herein comprising a rep gene and a cap gene comprise a rep2 gene and a cap5 gene for AAV5 production.

In some aspects, any of the expression vectors, msDNAs, or AAVs as disclosed herein comprising a rep gene and a cap gene comprise a rep2 gene and a cap9 gene for AAV9 production.

In some aspects, the cap gene comprises a sequence encoding a small peptide or ligand for targeting an AAV as disclosed herein to a cell and/or tissue type (i.e., the cap gene is a recombinant sequence). In some aspects, the sequence encoding a small peptide or ligand is for targeting an AAV to a tumor cell or tumor tissue. In some aspects, the cap gene comprises a sequence for targeting AAV to tumor tissue. In some aspects, the cap gene comprises a sequence encoding an NGR peptide motif. In some aspects, the cap gene comprises a sequence encoding a RGD peptide motif (e.g., a 4C-RGD peptide). In some aspects, the cap gene comprises a sequence encoding a designed ankyrin repeat protein (DARPin). In some aspects, the cap gene comprises mutations that enhance transduction efficiencies. In some aspects, the cap gene is from AAV3 and encodes a capsid protein with Y701F, Y705F, Y731F, S663V, T492V, and/or K533R mutations. In some aspects, the cap gene is from any other serotype and encodes a capsid protein with a mutation corresponding to Y701F, Y705F, Y731F, S663V, T492V, and/or K533R numbered according to the AAV3 capsid protein. In some aspects, the mutation comprises a combination of Y705F and Y731F. In some aspects, the mutation comprises S663V, T492V, and K533R. In some aspects, the mutation comprises S663V and T492V. In some aspects, the cap gene comprises a sequence encoding a protease recognition sequence (e.g., a protease recognition sequence recognized by a matrix metalloproteinase (MMP). See, e.g., Santiago-Oritz et al., J. Control Release (2016), http://dx.doi.org/10.1016/j.jconrel.2016.01.001

In some aspects, an expression vector or msDNA disclosed herein comprises an MCS. The MCS comprises restriction sites for insertion of a nucleic acid sequence of interest (e.g., a gene of interest) into the expression vector. The MCS can be operably linked to any appropriate expression control region known to those of skill in the art.

Provided herein is an expression vector comprising: (a) a first sequence comprising an inverted terminal repeat (ITR) and a multiple cloning site (MCS), wherein the ITR flanks at least one side of the MCS, and wherein the ITR comprises a sequence for adeno-associated virus (AAV) replication and an AAV packaging signal, (b) a target sequence for a first recombinase flanking each side of the first sequence, and (c) one or more additional target sequences for one or more additional recombinases integrated within non-binding regions of the target sequence for the first recombinase, wherein the expression vector is for producing a bacterial sequence-free vector having linear covalently closed ends (i.e., “expression vector A”). In some aspects, the ITR flanks only one side of the MCS. In some aspects, the ITR flanks each side of the MCS. In some aspects, the expression vector further comprises a spacer sequence between the target sequence for the first recombinase and the first sequence. In some aspects, the spacer sequence is 10 to 500 nucleotides. In some aspects, the expression vector further comprises an expression cassette comprising an AAV replication (rep) gene and an AAV capsid (cap) gene flanked on one side by the target sequence for the first recombinase and flanked on the other side by the first sequence.

Provided herein is an expression vector comprising: (a) first sequence comprising an ITR and an expression cassette comprising a nucleic acid sequence of interest, wherein the ITR flanks at least one side of the expression cassette comprising the nucleic acid sequence of interest, and wherein the ITR comprises a sequence for AAV replication and an AAV packaging signal, (b) a target sequence for a first recombinase flanking each side of the first sequence, and (c) one or more additional target sequences for one or more additional recombinases integrated within non-binding regions of the target sequence for the first recombinase, wherein the expression vector is for producing a bacterial sequence-free vector having linear covalently closed ends (i.e., “expression vector B”).

In some aspects, the ITR flanks only one side of the expression cassette comprising the nucleic acid sequence of interest in expression vector B (i.e., “expression vector B1”). In some aspects, the expression vector further comprises a spacer sequence between the target sequence for the first recombinase and the first sequence. In some aspects, the spacer sequence is 10 to 500 nucleotides.

In some aspects, expression vector B1 further comprises an expression cassette comprising an AAV rep gene and an AAV cap gene flanked on one side by the target sequence for the first recombinase and flanked on the other side by the first sequence (i.e., “expression vector B2”).

In some aspects, the ITR flanks each side of the expression cassette comprising the nucleic acid sequence of interest in expression vector B (i.e., “expression vector B3”). In some aspects, the expression vector further comprises a spacer sequence between the target sequence for the first recombinase and the first sequence. In some aspects, the spacer sequence is 10 to 500 nucleotides.

In some aspects, expression vector B3 further comprises an expression cassette comprising an AAV rep gene and an AAV cap gene flanked on one side by the target sequence for the first recombinase and flanked on the other side by the first sequence (i.e., “expression vector B4”).

Provided herein is an expression vector comprising: (a) a first sequence comprising an ITR and a palindromic sequence, wherein the ITR flanks each side of the palindromic sequence, wherein the palindromic sequence comprises an expression cassette comprising a nucleic acid sequence of interest and a complement of the expression cassette, and wherein the ITR comprises a sequence for AAV replication and an AAV packaging signal, (b) a target sequence for a first recombinase flanking each side of the first sequence, and (c) one or more additional target sequences for one or more additional recombinases integrated within non-binding regions of the target sequence for the first recombinase, wherein the expression vector is for producing a bacterial sequence-free vector having linear covalently closed ends (i.e., “expression vector C”). In some aspects, the complement is separated from the expression cassette comprising the nucleic acid sequence of interest by a non-complementary spacer sequence. In some aspects, the expression vector further comprises a spacer sequence between the target sequence for the first recombinase and the first sequence. In some aspects, the spacer sequence is 10 to 500 nucleotides.

In some aspects, expression vector C further comprises an expression cassette comprising an AAV rep gene and an AAV cap gene flanked on one side by the target sequence for the first recombinase and flanked on the other side by the first sequence (i.e., “expression vector C1”).

Provided herein is an expression vector comprising: (a) a first sequence comprising a portion of an expression cassette comprising a nucleic acid sequence of interest flanked on one side by a splicing sequence, an ITR flanking each side of the first sequence, wherein the ITR comprises a sequence for AAV replication and an AAV packaging signal, (b) a target sequence for a first recombinase flanking each ITR, and (c) one or more additional target sequences for one or more additional recombinases integrated within non-binding regions of the target sequence for the first recombinase, wherein the expression vector is for producing a bacterial sequence-free vector having linear covalently closed ends (i.e., “expression vector D”). In some aspects, the portion of the expression cassette comprises a 5′ portion that in combination with the remaining portion of the expression cassette provides the complete sequence of the expression cassette, and the splicing sequence flanks the 3′ end of the 5′ portion. In some aspects, the portion of the expression cassette comprises a 3′ portion that in combination with the remaining portion of the expression cassette provides the complete sequence of the expression cassette, and the splicing sequence flanks the 5′ end of the 3′ portion. In some aspects, the expression vector further comprises a spacer sequence between the target sequence for the first recombinase and the first sequence. In some aspects, the spacer sequence is 10 to 500 nucleotides.

The expression cassette in any of the expression vectors disclosed herein can include any appropriate expression control region known to those of skill in the art.

In some aspects, the expression control region is a cis-regulatory module (CRM), promoter, enhancer, operator, repressor, ribosome binding site, translation leader sequence, intron, post-transcriptional element, polyadenylation recognition sequence, RNA processing site, effector binding site, stem-loop structure, transcription termination signal, an miRNA binding site, or combination thereof. See, e.g., Domenger and Grimm, Hum. Mol. Genet. 28(R1):R3-R14 (2019).

In some aspects, the promoter is a mammalian, viral, wild-type, or synthetic promoter, including, e.g., a tissue and/or cell-specific promoter.

In some aspects, the post-transcriptional element is a woodchuck hepatitis virus posttranscriptional regulatory element (WPRE).

In some aspects, the 3′ end of the expression cassette includes an miRNA binding site for control of vector expression, e.g., tissue and/or cell-specific expression.

The nucleic acid sequence of interest in any of the expression vectors disclosed herein can be any desired sequence and is not limited by any specific requirement other than packaging constraints imposed by the AAV capsid. Specifically, single-stranded DNA sequences up to about 5 kilobases can be packaged into a single AAV capsid, including any ITR sequences, while double-stranded DNA sequences up to about half of that size can be packaged.

In some aspects, the nucleic acid sequence of interest comprises a sequence encoding: a polypeptide, an RNA (messenger RNA (mRNA), micro-RNA (miRNA), small interfering RNA (siRNA), small hairpin RNA (shRNA), ribozyme, or antisense RNA), or a non-coding DNA (e.g., an antisense oligonucleotide). In some aspects, the nucleic acid sequence of interest comprises a sequence encoding; an anti-cancer agent, a tumor suppressor, an apoptotic agent, an anti-angiogenesis agent, an enzyme, a cytotoxic agent, a suicide gene, a cytokine, an interferon, an interleukin, an immunomodulatory agent, an immunostimulatory agent, an immunoinhibitory agent, a chemokine, an antigen for stimulating an antigen-presenting cell, an antibody (e.g., a monoclonal, chimeric, humanized, or human antibody, or an antigen-binding fragment thereof), or an immunogenic agent (e.g., as a vaccine).

Exemplary nucleic acid sequences of interest and exemplary associated therapies include: surfactant protein B (SP-B, for treating surfactant dysfunction disorder), surfactant protein C (SP-C, for treating surfactant dysfunction disorder), ATP-binding cassette sub-family A member 3 (ABCA3, for treating surfactant dysfunction disorder), solute carrier family 34 member 2 (SLC34A2, for treating pulmonary alveolar microlithiasis and/or testicular microlithiasis), cystic fibrosis transmembrane conductance regulator (CFTR, for treating cystic fivrosis), glutamate decarboxylase (GAD, e.g., GAD65 or GAD67, for treating Parkinson's disease), aspartoacylase gene (ASPA, also known as aminoacylase (AAC), for treating Canavan disease), aromatic L-amino acid decarboxylase (AADC, for treating Parkinson's disease and/or for treating AADC deficiency), neurturin (NRTN, for treating Parkinson's disease), glial cell line-derived neurotrophic factor (GDNF, for treating Parkinson's disease), nerve growth factor (NGF, for treating Alzheimer's disease), tripeptidyl peptidase I (TPP1, also known as ceroid lipofuscinosis neuronal-2 (CLN2), for treating Batten disease), arylsulfatase A (ARSA, for treating metachromatic leukodystrophy), N-sulphoglucosamine sulphohydrolase (SGSH, for treating Sanfilippo syndrome, Type A), Sulfatase-modifying factor 1 (SUMF1, for treating Sanfilippo syndrome, Type A), N-acetyl-alpha-glucosaminidase (NAGLU, for treating Sanfilippo syndrome, Type B), survival of motor neuron 1 (SMN1, for treating spinal muscular atrophy 1), retinal pigment epithelium-specific 65 kDa protein (RPE65, also known as retinoid isomerohydrolase, for treating Leber's congenital amaurosis), Rab escort protein 1 (REP1, for treating choroideremia), retinoschisin 1 (RS1, for treating X-linked juvenile retinoschisis), alpha-1 antitrypsin (AAT, for treating hereditary emphysema or AAT deficiency), minidystrophin (for treating Duchenne's muscular dystrophy), α-sarcoglycan (αSG, for treating Duchenne's muscular dystrophy or limb girdle muscular dystrophy type 2), β-sarcoglycan (BSG), γ-sarcoglycan (γSG, for treating limb girdle muscular dystrophy type 2), δ-sarcoglycan (γSG), ipoprotein lipase (LPL, for treating familial LPL deficiency), acid alpha-glucosidase (GAA, for treating Pompe disease), tumor necrosis factor receptor: Fc (TNFR: Fc, for treating arthritis, e.g., inflammatory arthritis), sarcoplasmic/endoplasmic reticulum Ca(2+)ATPase 2a (SERCA2a, for treating congestive heart failure), Factor VIII, Factor IX (FIX, for treating hemophilia B), porphobilinogen deaminase gene (PBGD, for treating acute intermittent porphyria), soluble fms-like tyrosine kinase-1 (sFLT1, for treating age-related macular degeneration or cancer, e.g., ovarian cancer), a soluble chimeric vascular endothelial growth factor (VEGF) receptor comprising domains of VEGFR-1 and VEGF-R2 (for treating cancer, e.g., melanoma or colon cancer), soluble VEGFR3 (for treating cancer, e.g., endometrial cancer), a soluble VEGF-C decoy receptor (sVEGFR3-Fc, for treating cancer, e.g., melanoma, renal cell carcinoma, or prostate cancer), pigment epithelium-derived growth factor (PEDF, for treating cancer, e.g., Lewis lung carcinoma), a neutralizing monoclonal antibody against VEGFR2 (e.g., DC101, for treating cancer, e.g., melanoma or glioblastoma), endostatin (for treating cancer, e.g., bladder or pancreatic cancer), angiostatin (for treating cancer, e.g., liver cancer), both endostatin and angiostatin (i.e., as a bicistronic sequence, for treating cancer, e.g., ovarian or prostate cancer), an endostatin mutant (i.e., P1254A-endostatin, for treating cancer, e.g., ovarian cancer), antiangiogenic domain of TSP-1 (3TSR, for treating cancer, e.g., pancreatic cancer), tissue factor pathway inhibitor-2 (TFPI-2, for treating cancer, e.g., glioblastoma), a fragment of plasminogen (e.g., kringle 5, for treating cancer, e.g., ovarian cancer), plasminogen kringle 1-5 (for treating cancer, e.g., melanoma or lung cancer), siRNA against an unfolded protein response protein (UPR; e.g., IRE1α, XBP-1, or ATF6, for treating cancer, e.g., breast cancer), vasostatin (for treating cancer, e.g., lung cancer), herpes simplex virus type 1 thymidine kinase (HSV-TK, for treating cancer, e.g., breast cancer), sc39TK (for treating cancer, e.g., cervical cancer), diphtheria toxin A (DTA, for treating cancer, e.g., cervical cancer or myeloma), p53 upregulated modulator of apoptosis (PUMA, for treating cancer, e.g., cervical cancer or myeloma), tumor necrosis factor (TNF)-related apoptosis-inducing ligand (TRAIL, for treating cancer, e.g., lymphoma, hepatocellular carcinoma, head and neck squamous cell carcinoma (i.e., head and neck cancer), or glioblastoma), soluble TRAIL (for treating cancer, e.g., liver cancer or lung adenocarcinoma), IFN-β (for treating cancer, e.g., colorectal cancer, lung cancer, neuroblastoma, or glioblastoma multiforme), IFN-α (for treating cancer, e.g., metastatic melanoma), a CD-40 ligand (CD40L) or CD40L mutant (for treating cancer, e.g., lung cancer), melanoma differentiation-associated gene-7 and interleukin 24 (mda-7 and IL24, for treating cancer, e.g., Ehrlich ascites tumor), apoptotin and IL24 (for treating cancer, e.g., liver cancer), IL24 (for treating cancer, e.g., mixed-lineage leukemia (MLL)/AF4 positive acute lymphoblastic leukemia (ALL)), IL15 (for treating cancer, e.g., metastatic hepatocellular carcinoma), secondary lymphoid tissue chemokine (SLC, for treating cancer, e.g., liver cancer), Nk4 (the N-terminal hairpin and subsequent four kringle domains of hepatocyte growth factor (HGF) for treating cancer, e.g., metastatic Lewis lung carcinoma), tumor necrosis factor superfamily member 14 (TNFSF14, also known as LIGHT, for treating cancer, e.g., cervical cancer), Granulocyte-macrophage colony-stimulating factor (GM-CSF, for treating cancer), TNF-α (for treating cancer, e.g., glioma), a dominant negative mutant of survivin (e.g., C84A or T34A, for treating cancer, e.g., colon or gastric cancer), the C-terminal fragment of the human telomerase reverse transcriptase (hTERTC27, for treating cancer, e.g., glioblastoma multiforme), maspin (for treating cancer, e.g., prostate cancer), nm23H1 (for treating cancer, e.g., metastatic ovarian cancer), kringle 1 domain of human hepatocyte growth factor (HGFK1, for treating cancer, e.g., colorectal carcinoma), anti-calcitonin ribozyme (for treating cancer, e.g., prostate cancer), eukaryotic translation initiation factor 4E-binding protein 1 (4EBP1, for treating cancer, e.g., lung cancer), C-X-C motif chemokine receptor 2 (CXCR2) C-tail sequence (for treating cancer, e.g., pancreatic cancer), alpha-tocopherol-associated protein (TAP, for treating cancer, e.g., prostate cancer), trichosanthin (for treating cancer, e.g., hepatocellular carcinoma), decorin (for treating cancer, e.g., glioblastoma multiforme), cathelicidin (for treating cancer, e.g., colon cancer), Niemann-Pcik type C2 (NPC2, for treating cancer, e.g., hepatocellular carcinoma), Mullerian inhibiting substance (MIS, for treating cancer, e.g., ovarian cancer), p53 (for treating cancer, e.g., bronchioalveolar cancer), shRNA against highly expressed in cancer 1 (Hec1, for treating cancer, e.g., glioma), shRNA against Epstein-Barr virus latent membrane protein-1 (EBV LMP-1, for treating cancer, e.g., nasopharyngeal cancer), anti-sense RNA against human papilloma virus 16 E7 oncogene (HPV16-E7, for treating cancer, e.g., cervical cancer), shRNA against androgen receptor (AR, for treating cancer, e.g., prostate cancer), siRNA against Snail (also known as SNA1, for treating cancer, e.g., pancreatic cancer), siRNA against Slug (i.e., the protein product of SNAI2, for treating cancer, e.g., cholangiocarcinoma (liver cancer)), shRNA against Four and a half LIM-only protein 2 (FHL2, for treating cancer, e.g., colon cancer), miR-26a (for treating cancer, e.g., hepatocellular carcinoma), HPV 16 structural protein L1 (HPV16-L1, for treating cancer, e.g., cervical cancer), HPV 16 E5, E6, and E7 oncogenes (HPV16 E5/E6/E7, for treating cancer, e.g., cervical cancer), B-cell leukemia/lymphoma 1 (BLC1) idiotype (for treating cancer, e.g., B cell leukemia/lymphoma 1), EBV LMP1 and LMP2 fused to heat shock protein (EBV LMP2/1-hsp, for treating cancer, e.g., nasopharyngeal carcinoma), carcinoembryonic antigen (CEA, for treating cancer, e.g., colon cancer), soluble form of B and T lymphocyte attenuator in combination with a heat shock protein (BTLA and HSP70, for treating cancer, e.g., melanoma pulmonary metastasis), HPV16-L1/E7 (for treating cancer, e.g., cervical cancer), HPV16-L1 (for treating cancer, e.g., cervical cancer), an anti-EGFR antibody (e.g., 14D1, for treating cancer, e.g., vulvar carcinoma), an anti-death receptor 5 (DR5) antibody (e.g., adximab, for treating cancer, e.g., liver or colon cancer), an anti-Enolase 1 (ENOI1) antibody (for treating cancer, e.g., pancreatic ductal adenocarcinoma), an anti-VEGFA antibody (e.g., bevaciumab, for treating cancer, e.g., metastatic lung cancer or ovarian cancer), the Mucin 1 (MUC1) antigen (for treating cancer, e.g., gastric cancer), or an aquaporin (e.g., hAQP1, for treating irradiation induced parotid salivary hypofunction, i.e., xerostomia). See, e.g., Lisowski et al., Santiago-Oritz et al.

In some aspects, the sequence for AAV replication in any of the above expression vectors comprises an AAV ITR Replication (Rep) protein binding element (RBE) and terminal resolution site (TRS).

In some aspects, the AAV packaging signal in any of the above expression vectors comprises an AAV ITR D-sequence.

Provided herein is an expression vector for producing a bacterial sequence-free vector having linear covalently closed ends (i.e., an msDNA) comprising sequences that encode AAV rep, AAV cap, and/or helper virus genes required for production of AAV. In some aspects, the expression vector comprises AAV rep. In some aspects, the expression vector comprises AAV cap. In some aspects, the expression vector comprises AAV rep and AAV cap. In some aspects, the expression vector comprises helper virus genes. In some aspect, the expression vector comprises AAV rep and helper virus genes. In some aspects, the expression vector comprises AAV cap and helper virus genes. In some aspects, the expression vector comprises AAV rep, AAV cap, and helper virus genes. Expression vectors comprising AAV rep, AAV cap, and/or helper virus genes lack (i.e., do not include) an ITR flanking either end of the AAV rep, AAV cap, and/or helper virus genes to avoid packaging AAV rep, AAV cap, and/or helper virus genes into an AAV capsid. In some aspects, the coding sequence(s) of an expression vector as disclosed herein consists essentially of AAV rep, AAV cap, and/or helper virus genes.

Helper virus genes as disclosed herein comprise one or more genes from a virus that supplies a function required for replication of AAV. In some aspects, the helper virus genes comprise one or more genes from an adenovirus, a herpesvirus (e.g., a herpes simplex virus (HSV), an Epstein-Barr virus (EBV), a cytomegalovirus (CMV), or a pseudorabies virus (PRV)), a retrovirus, a poxvirus (e.g., a vaccinia virus), and/or a lentivirus. In some aspects, the helper virus genes comprise one or more of adenovirus Early 4 (E4) gene, adenovirus Early 2A (E2A) gene, or adenovirus Viral Associated (VA) gene. In some aspects, the helper virus genes comprise the adenovirus E4, E2A, and VA genes.

In some aspects, an msDNA for expression of helper genes as disclosed herein comprises the polynucleotide sequence of SEQ ID NO: 25.

In some aspects, an msDNA for expression of helper genes as disclosed herein comprises the polynucleotide sequence of SEQ ID NO: 51.

Provided herein is an expression vector comprising: (a) an expression cassette comprising an AAV rep gene and an AAV cap gene, (b) a target sequence for a first recombinase flanking each side of the expression cassette, and (c) one or more additional target sequences for one or more additional recombinases integrated within non-binding regions of the target sequence for the first recombinase wherein the expression vector is for producing a bacterial sequence-free vector having linear covalently closed ends (i.e., “expression vector E”).

Provided herein is an expression vector comprising: (a) an expression cassette comprising one or more helper virus genes for production of AAV, (b) a target sequence for a first recombinase flanking each side of the expression cassette, and (c) one or more additional target sequences for one or more additional recombinases integrated within non-binding regions of the target sequence for the first recombinase, wherein the expression vector is for producing a bacterial sequence-free vector having linear covalently closed ends (i.e., “expression vector F”). In some aspects, the one or more helper virus genes are from an adenovirus, a herpesvirus, a retrovirus, a poxvirus, and/or a lentivirus. In some aspects, the one or more helper virus genes comprise an adenovirus E4 gene, adenovirus E2A gene, and adenovirus VA gene.

In some aspects, the target sequence for the first recombinase and the one or more additional target sequences for the one or more additional recombinases in any of the expression vectors disclosed herein are selected from the group consisting of the PY54 pal site, the N15 telRL site, and the qK02 telRL site. In some aspects, any of the expression vectors disclosed herein comprises each of the target sequences. In some aspects, any of the expression vectors disclosed herein comprises the Tel recombinase pal site and the telRL recombinase target binding sequence integrated within the pal site.

In some aspects, the target sequence for the first recombinase in any of the expression vectors disclosed herein is the phage PY54 Tel 142 base pair target site.

Provided herein is a vector production system comprising recombinant cells designed to encode at least a first recombinase under the control of an inducible promoter, wherein the cells comprise any of the expression vectors disclosed herein. In some aspects, the cells comprise any of expression vectors B-F as disclosed herein. In some aspects, the recombinant cell is an Escherichia coli cell, a yeast cell such as Saccharomyces cerevisiae, or a mammalian cell as disclosed in U.S. Pat. No. 9,862,954. In some aspects, the inducible promoter is thermally-regulated, chemically-regulated, IPTG regulated, glucose-regulated, arabinose inducible, T7 polymerase regulated, cold-shock inducible, pH inducible, or combinations thereof. In some aspects, the first recombinase is selected from TelN and Tel, and the expression vector incorporates the target sequence for at least the first recombinase. In some aspects, the recombinant cells have been further designed to encode a nuclease genome editing system, and wherein the expression vector further comprises a backbone sequence containing a cleavage site for the nuclease genome editing system. In some aspects, the nuclease genome editing system is a CRISPR nuclease system comprising a Cas nuclease and gRNA, and the expression vector comprises a target sequence for the gRNA within the backbone sequence.

Provided herein is a method of producing a bacterial sequence-free vector having linear covalently closed ends (i.e., an msDNA or msDNA vector) comprising incubating any of the vector production systems disclosed herein under suitable conditions for expression of the first recombinase.

Provided herein is a method of producing a bacterial sequence-free vector having linear covalently closed ends comprising incubating any of the vector production systems disclosed herein under suitable conditions for expression of the first recombinase and the nuclease genome editing system. In some aspects, the method further comprises harvesting the bacterial sequence-free vector.

Provided herein is a method a producing a bacterial sequence-free vector having linear covalently closed ends comprising incubating any of the expression vectors disclosed herein in vitro with a bacteriophage PY54-derived Tel/Pal recombination system. In some aspects, the method further comprises harvesting the bacterial sequence-free vector.

Provided herein is a bacterial sequence-free vector produced by any of the methods of producing a bacterial sequence-free vector having linear covalently closed ends as disclosed herein. In some aspects, the bacterial sequence-free vector is produced from expression vector B1. In some aspects, the bacterial sequence-free vector is produced from expression vector B2. In some aspects, the bacterial sequence-free vector is produced from expression vector B3. In some aspects, the bacterial sequence-free vector is produced from the expression vector B4. In some aspects, the bacterial sequence-free vector is produced from expression vector C. In some aspects, the bacterial sequence-free vector is produced from expression vector C1. In some aspects, the bacterial sequence-free vector is produced from expression vector D. In some aspects, the bacterial sequence-free vector is produced from expression vector E. In some aspects, the bacterial sequence-free vector is produced from expression vector F.

B. Methods of Producing AAV from msDNA

Wild-type AAV is packaged as a single-stranded genome (i.e., single-stranded AAV, “ssAAV”). An msDNA as disclosed herein comprising an expression cassette flanked on each side by an ITR can produce a ssAAV in the presence of rep, cap, and helper virus genes.

The packaging capacity of an AAV capsid is about 5 kb. Nucleic acid sequences of interest larger than about 5 kb and up to about 10 kb can be delivered by co-infection of cells with two separate ssAAVs that each carry a portion of the nucleic acid sequence of interest. The portions can be joined together in the co-infected cell either through trans-splicing or homologous recombination to reproduce the complete nucleic acid sequence of interest.

Trans-splicing takes advantage of the ability of AAV genomes to form head-to-tail concatemers via recombination in the ITRs after infecting a cell. See, e.g., Daya and Berns; Yan et al., PNAS 97(12): 6716-6721 (2000). Transcription from the recombined AAVs followed by splicing of the mRNA transcripts can join the separate 5′ and 3′ portions. Specifically, a 5′ portion of a nucleic acid sequence of interest along with a splicing sequence (e.g., a 3′ splice donor) is flanked on each side by an ITR in a first AAV, while the remaining 3′ portion of the nucleic acid sequence of interest along with a splicing sequence (e.g., a 5′ splice acceptor) is flanked on each side by an ITR in the second AAV. Upon infection, the 5′ and 3′ portions from each AAV are spliced together to form the complete nucleic acid sequence of interest.

Alternatively, a nucleic acid sequence of interest can be split as two portions with substantial overlap in sequence between two separate ssAAVs. Co-expression in an infected cell induces homologous recombination and formation of the complete nucleic acid sequence of interest.

msDNAs as disclosed herein can be used to produce ssAAVs comprising portions of a nucleic acid sequence of interest for delivering sequences up to about 10 kb to target cells and tissues through co-infection and trans-splicing or homologous recombination.

Because AAV depends on the cell's DNA replication machinery to synthesize the complementary strand to the ssAAV genome, expression of a nucleic acid sequence of interest (e.g., a transgene) from ssAAV can be delayed following infection of a cell or tissue. To overcome delayed expression of a nucleic acid sequence of interest, a self-complementary AAV (scAAV) can be produced that contains complementary sequences capable of spontaneously annealing upon infection to form transcriptionally competent double-stranded DNA. See, e.g., Daya and Berns. An msDNA as disclosed herein comprising a palindromic sequence (e.g., an expression cassette comprising a nucleic sequence of interest and a complement of the expression cassette) that is flanked on each side by an ITR is packaged as scAAV in the presence of rep, cap, and helper virus genes.

An scAAV can also be formed using msDNA as disclosed herein containing only a single ITR flanking one side of an expression cassette. Without both ITRs, the msDNA is not replicated as an ssAAV intermediate. Instead, the sequence is directly packaged as double-stranded DNA due to the packaging signal in the ITR in the presence of rep, cap, and helper virus genes. The nucleic acid sequence of interest is then available as transcriptionally competent double-stranded DNA at the time of infection.

AAV rep and cap sequences can be provided in a standard plasmid for producing AAV or can be provided in one or more msDNAs as disclosed herein.

Helper virus genes can be provided in a standard plasmid, as helper virus, or in one or more msDNAs as disclosed herein.

In some aspects, production of AAV2 as disclosed herein comprises: an msDNA encoding a GOI as disclosed herein, an msDNA comprising rep2 and cap2 genes comprising the polynucleotide sequence of SEQ ID NO: 24, and/or an msDNA comprising helper virus genes comprising the polynucleotide sequence of SEQ ID NO: 25.

In some aspects, production of AAV2 as disclosed herein comprises: an msDNA encoding a GOI as disclosed herein, an msDNA comprising rep2 and cap2 genes comprising the polynucleotide sequence of SEQ ID NO: 48, and/or an msDNA comprising helper virus genes comprising the polynucleotide sequence of SEQ ID NO: 51.

In some aspects, production of AAV5 as disclosed herein comprises: an msDNA encoding a GOI as disclosed herein, an msDNA comprising rep2 and cap5 genes comprising the polynucleotide sequence of SEQ ID NO: 49, and/or an msDNA comprising helper virus genes comprising the polynucleotide sequence of SEQ ID NO: 51.

In some aspects, production of AAV9 as disclosed herein comprises: an msDNA encoding a GOI as disclosed herein, an msDNA comprising rep2 and cap9 genes comprising the polynucleotide sequence of SEQ ID NO: 50. and/or an msDNA comprising helper virus genes comprising the polynucleotide sequence of SEQ ID NO: 51.

Alternatively, AAV producer cell lines that contain integrated rep, cap, and/or helper virus genes in the genome of the producer cell can be used in production of AAV according to the methods disclosed herein to provide consistent and stable expression of AAV replication and packaging proteins. AAV producer cell lines can be generated to contain integrated rep, cap, and/or helper virus genes by homologous recombination with corresponding msDNAs as disclosed herein that comprise homology arms for recombination.

An AAV producer cell can be any cell capable of producing AAV. In some aspects, the producer cell is a mammalian cell (e.g., HEK293, COS, HeLa, or KB). In some aspect, the producer cell is HEK293. In some aspects, the producer cell is an insect cell (e.g., expresSF+®, Drosophila Schneider 2 (S2), Se301, SeIZD2109, SeUCRI, Sf9, Sf900+, Sf21, BTI-TN-5B1-4, MG-I, 5 Tn368, HzAml, Ha2302, or Hz2E5). In some aspects, when the producer cell is an insect cell, the expression vector for producing an msDNA as disclosed herein is a baculoviral vector.

Provided herein is a method for producing a single-stranded AAV, wherein the method comprises: (a) transfecting cells that are capable of producing an AAV with: (i) the bacterial sequence-free vector produced from expression vector B3, (ii) the bacterial sequence-free vector produced from expression vector E or an expression vector comprising an expression cassette comprising an AAV rep gene and an AAV cap gene, and (iii) the bacterial sequence-free vector produced from expression vector F or an expression vector comprising an expression cassette comprising one or more helper virus genes for production of AAV, and (b) incubating the cells under conditions suitable for production of an AAV.

Provided herein is a method for producing a single-stranded AAV, wherein the method comprises: (a) transfecting cells that are capable of producing an AAV with: (i) the bacterial sequence-free vector produced from expression vector B4, (ii) the bacterial sequence-free vector produced from expression vector F or an expression vector comprising an expression cassette comprising one or more helper virus genes for production of AAV, and (b) incubating the cells under conditions suitable for production of an AAV.

Provided herein is a method for producing a single-stranded AAV, wherein the method comprises: (a) transfecting cells that are capable of producing an AAV with the bacterial sequence-free vector produced from expression vector B3, wherein each of an AAV rep gene, an AAV cap gene, and one or more helper virus genes for production of AAV is encoded by the cells or a vector, (b) incubating the cells under conditions suitable for expression of the rep gene, the cap gene, and the one or more helper virus genes and for production of an AAV.

Provided herein is a method for producing a self-complementary AAV, wherein the method comprises: (a) transfecting cells that are capable of producing an AAV with: (i) the bacterial sequence-free vector produced from expression vector B1, (ii) the bacterial sequence-free vector produced from expression vector E or an expression vector comprising an expression cassette comprising an AAV rep gene and an AAV cap gene, and (iii) the bacterial sequence-free vector produced from expression vector F or an expression vector comprising an expression cassette comprising one or more helper virus genes for production of AAV, and (b) incubating the cells under conditions suitable for production of an AAV.

Provided herein is a method for producing a self-complementary AAV, wherein the method comprises: (a) transfecting cells that are capable of producing an AAV with: (i) the bacterial sequence-free vector produced from expression vector B2, (ii) the bacterial sequence-free vector produced from expression vector F or an expression vector comprising an expression cassette comprising one or more helper virus genes for production of AAV, and (b) incubating the cells under conditions suitable for production of an AAV.

Provided herein is a method for producing a self-complementary AAV, wherein the method comprises: (a) transfecting cells that are capable of producing an AAV with: (i) the bacterial sequence-free vector produced from expression vector C, (ii) the bacterial sequence-free vector produced from expression vector E or an expression vector comprising an expression cassette comprising an AAV rep gene and an AAV cap gene, and (iii) the bacterial sequence-free vector produced from expression vector F or an expression vector comprising an expression cassette comprising one or more helper virus genes for production of AAV, and (b) incubating the cells under conditions suitable for production of an AAV.

Provided herein is a method for producing a self-complementary AAV, wherein the method comprises: (a) transfecting cells that are capable of producing an AAV with: (i) the bacterial sequence-free vector produced from expression vector C1, (ii) the bacterial sequence-free vector produced from expression vector F or an expression vector comprising an expression cassette comprising one or more helper virus genes for production of AAV, and (b) incubating the cells under conditions suitable for production of an AAV.

Provided herein is a method for producing a self-complementary AAV, wherein the method comprises: (a) transfecting cells that are capable of producing an AAV with the bacterial sequence-free vector produced from expression vector B1 or C, wherein each of an AAV rep gene, an AAV cap gene, and one or more helper virus genes for production of AAV is encoded by the cells or a vector, and (b) incubating the cells under conditions suitable for expression of the rep gene, the cap gene, and the one or more helper virus genes and for production of an AAV.

In some aspects, the cells in any of the methods for producing a single-stranded AAV or self-complementary AAV as disclosed herein are HEK293T cells.

In some aspects, any of the methods for producing a single-stranded AAV or self-complementary AAV as disclosed herein further comprise harvesting the AAV.

In one aspect, the present disclosure is directed to an AAV produced by any of the methods for producing a single-stranded AAV or self-complementary AAV as disclosed herein.

In some aspects, AAV produced according to the methods disclosed herein have a reduction in the number of contaminating bacterial sequences when compared to AAV produced using another method (e.g., a system in which three plasmids comprise the nucleic acid sequence of interest, rep/cap, and the helper virus genes, respectively (i.e., a three-plasmid system)). In some aspects, the reduction in the number of contaminating bacterial sequences is below a detection limit (i.e., contaminating bacterial sequences are undetectable in the AAV). In some aspects, the reduction in the number of contaminating bacterial sequences is determined by Real-Time quantitative PCR against bacterial backbone and/or other plasmid associated impurities such as, for example, ampicillin resistance (ampR) gene, an origin or replication (ori, e.g., F1 ori), kanamycin resistance (kanR) gene, or sequence for chromatography, affinity and recombination (SCAR).

In some aspects, AAV produced according to the methods disclosed herein provide a higher number of AAV comprising the nucleic acid sequence of interest when compared to AAV produced using another method.

In some aspects, AAV produced according to the methods disclosed herein comprise a reduced number of empty capsids when compared to AAV produced using another method.

The number of empty capsids, including the ratio of full to empty AAV particles, can be assessed by any known method in the art, including, for example, analytical ultracentrifugation, transmission electron microscopy, anion-exchange high-performance liquid chromatography assay, and/or capillary isoelectric focusing. See, e.g., Burnham et al., Hum. Gene Ther. Methods 26(6): 228-242(2015); Chen, Microsc. Microanal. 13(5), 384-389(2007); Fu et al., Hum. Gene Ther. Methods 30(4): 144-152(2019); Li et al., Curr. Mol. Med. doi: 10.2174/1566524020666200915105456 (2020).

In some aspects, AAV produced according to the methods disclosed herein provide a higher transfection efficiency when compared to AAV produced using another method.

In some aspects, AAV produced according to the methods disclosed herein provide a higher copy number per unit of transfection when compared to AAV produced using another method.

In some aspects, AAV produced according to the methods disclosed herein provide a greater amount of nuclear localization of the AAV when compared to AAV produced using another method.

In some aspects, AAV produced according to the methods disclosed herein result in a decreased immune response, fewer neutralizing antibodies, less risk of genomic integration, less silencing of the nucleic acid of interest, and/or less risk of antibiotic resistance following administration to a subject, or as measured in vitro, when compared to AAV produced using another method.

### III. Pharmaceutical Compositions and Therapeutic Uses

Provided herein is a pharmaceutical composition comprising an AAV as disclosed herein.

In certain aspects, the composition further comprises a physiologically acceptable carrier, excipient, or stabilizer. See, e.g., Remington: The Science and Practice of Pharmacy, 22^{nd} ed. (2013). Acceptable carriers, excipients, or stabilizers can include those that are nontoxic to a subject. In certain embodiments, the composition or one or more components of the composition are sterile. A sterile component can be prepared, for example, by filtration (e.g., by a sterile filtration membrane) or by irradiation (e.g., by gamma irradiation).

An excipient of the present invention can be described as a “pharmaceutically acceptable” excipient when added to a pharmaceutical composition, meaning that the excipient is a compound, material, composition, salt, and/or dosage form which is, within the scope of sound medical judgment, suitable for contact with the tissues of human beings and animals without excessive toxicity, irritation, allergic response, or other problematic complications over the desired duration of contact commensurate with a reasonable benefit/risk ratio. In some embodiments, the term “pharmaceutically acceptable” means approved by a regulatory agency of the Federal or a state government or listed in the U.S. Pharmacopeia or other generally recognized international pharmacopeia for use in animals, and more particularly in humans. Various excipients can be used. In some embodiments, the excipient can be, but is not limited to, an alkaline agent, a stabilizer, an antioxidant, an adhesion agent, a separating agent, a coating agent, an exterior phase component, a controlled-release component, a solvent, a surfactant, a humectant, a buffering agent, a filler, an emollient, or combinations thereof. Excipients in addition to those discussed herein can include excipients listed in, though not limited to, Remington: The Science and Practice of Pharmacy, 22^{nd} ed. (2013). Inclusion of an excipient in a particular classification herein (e.g., “solvent”) is intended to illustrate rather than limit the role of the excipient. A particular excipient can fall within multiple classifications.

A pharmaceutical composition of the disclosure is formulated to be compatible with its intended route of administration. Examples of routes of administration include Routes of administration for the compositions disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the composition is administered via a non-parenteral route, in some aspects, orally. Other non-parenteral routes include a topical, epidermal, or mucosal route of administration, for example, intranasally, sublingually, or topically. Administering can also be performed, for example, once, a plurality of times, and/or over one or more extended periods. In some aspects, a pharmaceutical composition comprising an AAV as disclosed herein further comprises a delivery agent. In some aspects, the delivery agent comprises a nanoparticle. In some aspects, the delivery agent is selected from the group consisting of liposomes, non-lipid polymeric molecules, endosomes, and any combination thereof. In some aspects, the delivery agent (e.g., a nanoparticle) comprises a targeting ligand.

Provided herein is a method of treating a disease or disorder in a subject in need thereof, comprising administering an AAV or pharmaceutical composition as disclosed herein to the subject.

Treatment is continued as long as clinical benefit is observed or until unacceptable toxicity or disease progression occurs. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels can be varied so as to obtain an amount of a nucleic acid sequence of interest as disclosed herein which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

All of the references cited above, as well as all references cited herein, are incorporated herein by reference in their entireties.

The following examples are offered by way of illustration and not by way of limitation.

### EXAMPLES

### Example 1

### Production of Expression Vectors and Ministring DNA

Ministring DNAs (msDNAs) are produced that contain a gene of interest, Rep/Cap sequences for AAV replication/packaging, or adenovirus helper sequences according to the methods disclosed herein as well as in U.S. Pat. Nos. 9,290,778 and 9,862,954, and International Publication No. WO 2022/264095, incorporated by reference herein in their entireties.

### A. Production of Expression Vector Containing GFP

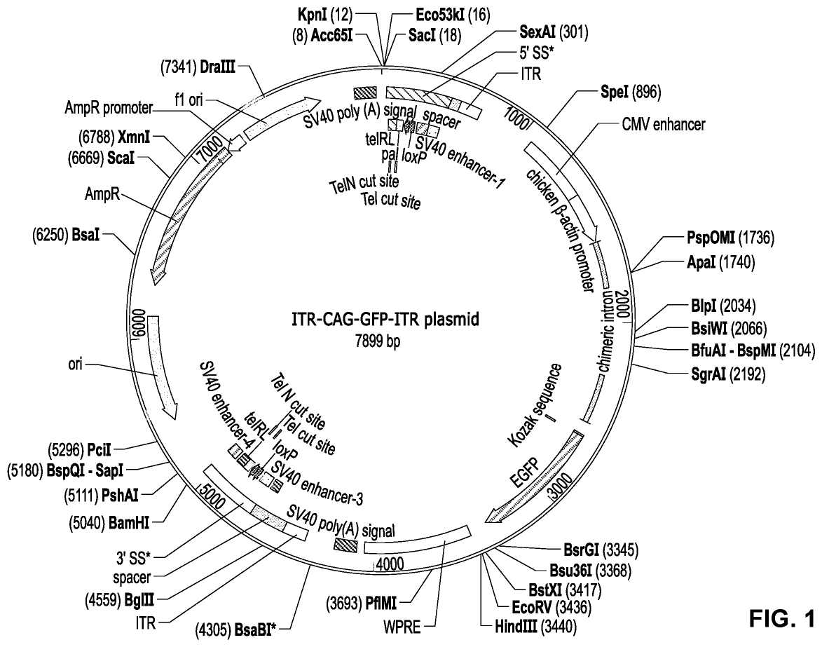

An expression vector is prepared containing green fluorescent protein (GFP) as an exemplary gene of interest flanked by a 5′ITR and a 3′ ITR (“ITR-GFP-ITR,” see, e.g., FIG. 1) or flanked only by a 3′ ITR (“GFP-ITR”).

The ITR-GFP-ITR and GFP-ITR sequences are obtained by restriction digestion or polymerase chain reaction (PCR) amplification from an AAV-GFP vector or the GFP sequence is cloned into plasmids carrying the appropriate ITR(s).

ITR-GFP-ITR and GFP-ITR are each inserted into the multicloning site between two specialized Super Sequence (“SS” or “SSeq” as used interchangeably herein) sites in separate expression vectors (pMinistring, Mediphage Bioceuticals, Inc., Toronto, CA; U.S. Pat. Nos. 9,290,778 and 9,862,954, and International Publication No. WO 2022/264095).

A map for an exemplary expression vector encoding an ITR-GFP-ITR msDNA is shown as “ITR-CAG-GFP-ITR plasmid” in FIG. 1 and the nucleic acid sequence for the vector is provided as SEQ ID NO:18.

The map and sequence for an exemplary expression vector encoding GFP-ITR are identical to those of ITR-CAG-GFP-ITR plasmid, except that the expression cassette in the expression vector encoding GFP-ITR is only flanked by a 3′ ITR and not by a 5′ ITR.

Additional expression vectors are prepared with ITR-GFP-ITR and GFP-ITR in which spacer sequences of different lengths are included between the 5′ SS and the 5′ ITR and the 3′ ITR and the 3′ SS for ITR-GFP-ITR, or between the 3′ ITR and the 3′ SS for GFP-ITR. Exemplary spacer sequence lengths are 0-10, 10-15, 50-100, 100-250, and 250-500 nucleotides.

Expression vectors also are prepared in which the ITRs in ITR-GFP-ITR and GFP-ITR are minimal ITRs lacking the B-B′ and C-C′ palindromic sequences (i.e., the ITRs only contain the A-A′ palindromic sequences and the D-sequence).

### B. Production of Expression Vector Containing Rep and Cap Sequences

A sequence comprising rep and cap is obtained by restriction digestion or PCR amplification from a plasmid containing the genes and inserted between two SS sites in an expression vector.

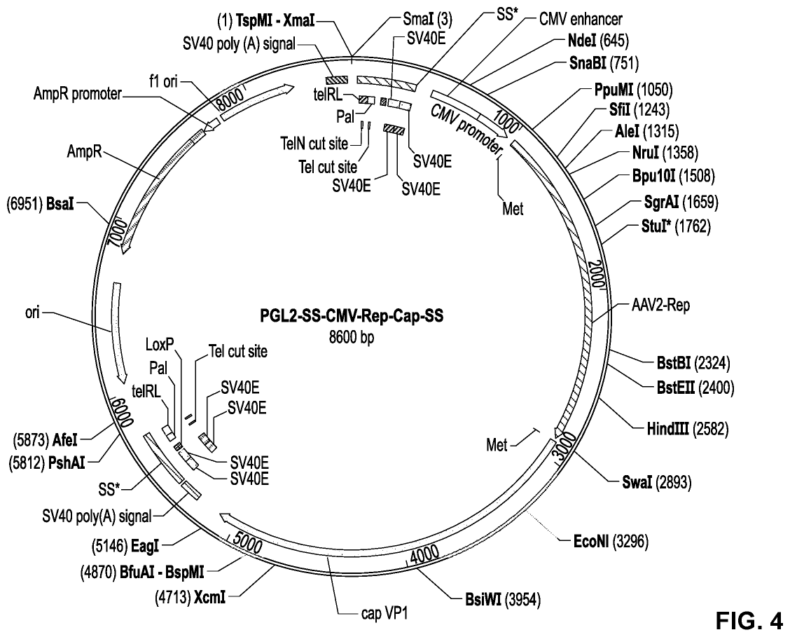

A map for an exemplary expression vector encoding a Rep-Cap msDNA is shown in FIG. 4 as “PGL2-SS-CMV-Rep-Cap-SS,” and the nucleic acid sequence for the vector is provided as SEQ ID NO:23.

Additional expression vectors are prepared in which the rep and cap sequences are combined into a single expression vector with a GOI, for example, expression vectors in which rep and cap are flanked on one side by a SS and the other side by the ITR-GFP-ITR or GFP-ITR (e.g., SS-Rep-Cap-ITR-GFP-ITR-SS and SS-Rep-Cap-GFP-ITR-SS).

### C. Production of Expression Vector Containing Adenovirus Helper Sequences

Adenovirus helper sequences for AAV replication are obtained by restriction digestion or PCR amplification from an AAV helper plasmid and inserted into the multicloning site between two SS sites in a ministring vector.

D. Production of msDNA from Expression Vectors

DNA ministrings (msDNA) are produced in inducible E. coli cells according to methods described herein as well as in U.S. Pat. Nos. 9,290,778 and 9,862,954, and International Publication No. WO 2022/264095, incorporated by reference herein in their entireties.

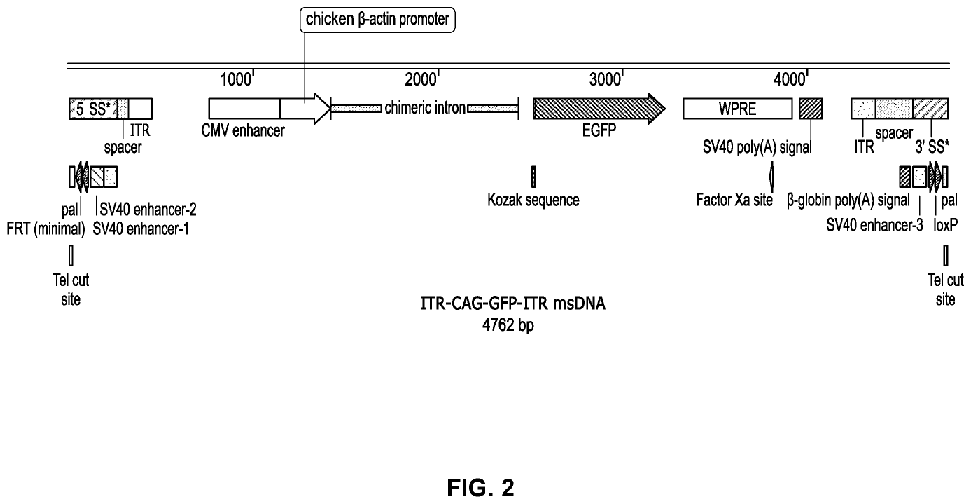

FIG. 2 shows a map for an exemplary ITR-GFP-ITR msDNA, “ITR-CAG-GFP-ITR msDNA,” that is produced from the expression vector shown in FIG. 1. A nucleic acid sequence for ITR-CAG-GFP-ITR msDNA is provided as SEQ ID NO:19.

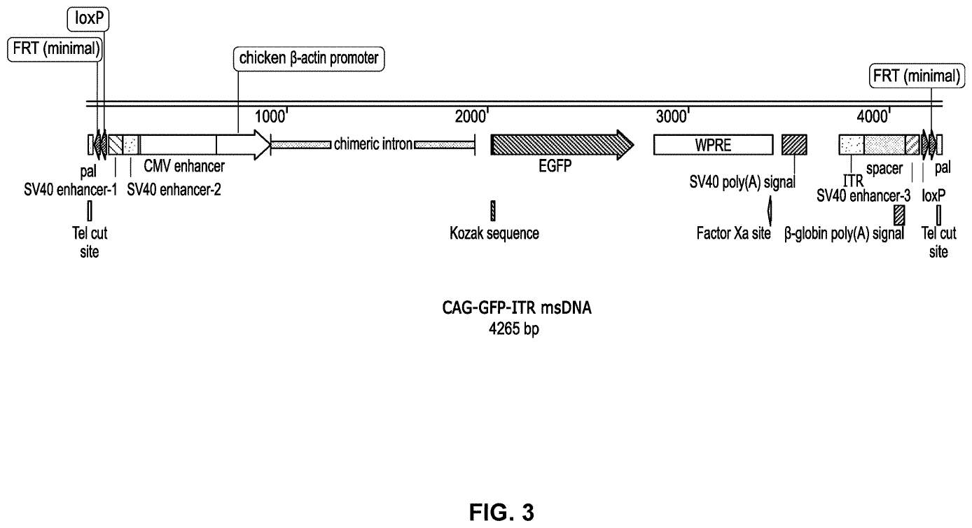

FIG. 3 shows a map for an exemplary GFP-ITR msDNA, “CAG-GFP-ITR msDNA.” A nucleic acid sequence for CAG-GFP-ITR msDNA is provided as SEQ ID NO: 20.

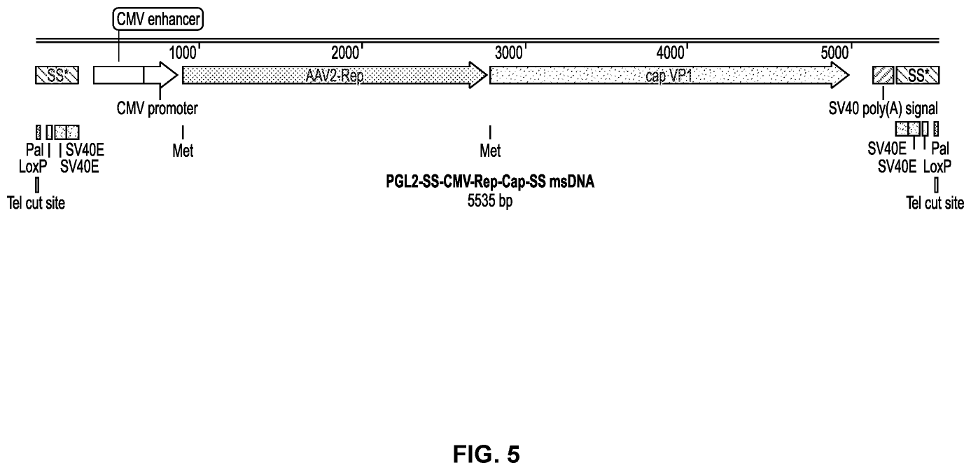

FIG. 5 shows a map for an exemplary Rep-Cap msDNA, “PGL2-SS-CMV-Rep-Cap-SS msDNA,” that is produced from the expression vector shown in FIG. 4. A nucleic acid sequence for PGL2-SS-CMV-Rep-Cap-SS msDNA is provided as SEQ ID NO:24.

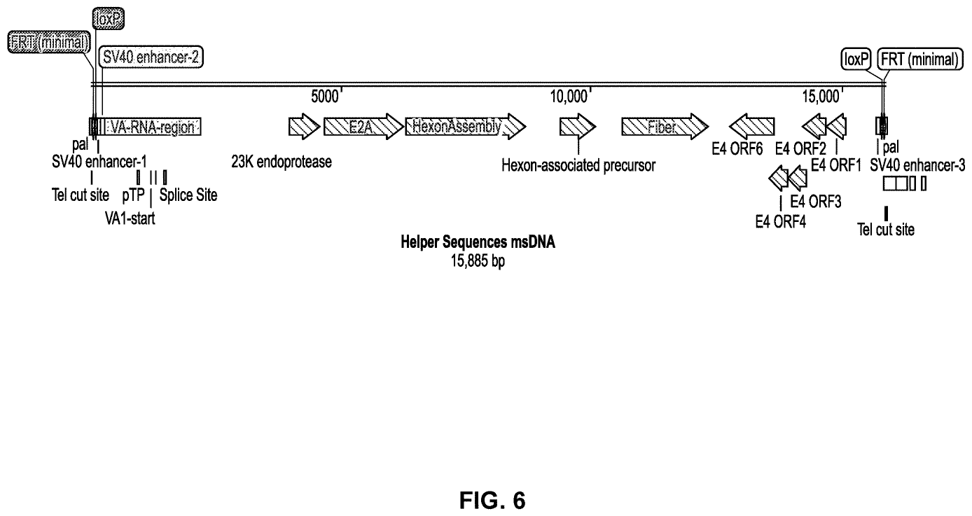

FIG. 6 shows a map for an exemplary “Helper Sequences msDNA.” A nucleic acid sequence for Helper Sequences msDNA is provided as SEQ ID NO:25.

### Example 2

Production and Characterization of AAV Produced with msDNA

An objective of this study is to evaluate AAV produced using the msDNAs described in Example 1 as compared to AAV produced using conventional plasmids.

### A. AAV Production

Conventional plasmids and the msDNAs of Example 1 are used to produce AAV. The conventional plasmids are: a plasmid containing GFP flanked by ITRs (i.e., pITR-GFP-ITR), a plasmid containing AAV rep and cap genes (e.g., pRep-Cap, such as pRep2-Cap1 for production of AAV1, pRep2-Cap2 for production of AAV2, pRep2-Cap5 for production of AAV5, pRep2-Cap9 for production of AAV9, etc.), and a plasmid containing adenovirus helper genes (i.e., pHelper).

The combination of three conventional plasmids for AAV production (i.e., pITR-GFP-ITR, pRep-Cap, and pHelper, shown below as Combination 1 in Table 3) serves as the baseline for comparisons with AAV produced with msDNA as the carrier of the GOI in combination with either conventional plasmids as carriers of the Rep/Cap and Helper sequences or with msDNA providing the Rep/Cap and helper sequences.

TABLE 3
Combinations of msDNA and plasmids for producing AAV

| Combina- | | | |
|---|---|---|---|
| tion | GOI | Rep-Cap | Helper |
| 1 | pITR-GFP-ITR | pRep-Cap | pHelper |
| 2 | ITR-GFP-ITR msDNA | pRep-Cap | pHelper |
| 3 | GFP-ITR msDNA | pRep-Cap | pHelper |
| 4 | Rep-Cap-ITR-GFP-ITR msDNA | | pHelper |
| 5 | Rep-Cap-GFP-ITR msDNA | | pHelper |
| 6 | ITR-GFP-ITR msDNA | Rep-Cap msDNA | Helper msDNA |
| 7 | GFP-ITR msDNA | Rep-Cap msDNA | Helper msDNA |

| 8 | Rep-Cap-ITR-GFP-ITR msDNA | Helper msDNA |
|---|---|---|
| 9 | Rep-Cap-GFP-ITR msDNA | Helper msDNA |
| 10 | ITR-GFP-ITR msDNA | Rep-Cap-Helper |
| | | msDNA |
| 11 | ITR-GOI-ITR-Helper msDNA | Rep-Cap msDNA |

| | |
|---|---|
| 12 | ITR-GOI-ITR-Helper-RepCap msDNA |

Combinations also will include ITR-GFP-ITR msDNA and GFP-ITR msDNA with varying lengths of spacer sequences between the SS and ITR as well as minimal ITRs as described in Example 1.

The combinations in Table 3 are separately transfected into mammalian production cells (e.g., HEK293T, ATCC® CRL-3216™), the cells are incubated for production of AAV, and AAV is purified, according to standard procedures.

B. Generation of Stable AAV Production Cell Line with msDNA

A stable mammalian AAV production cell line (e.g., HEK-293) is generated through nuclease-mediated homologous recombination of Rep-Cap msDNA and/or Helper msDNA as described in Example 1 to integrate Rep/Cap and/or Helper genes, respectively, into the cell line genome. This results in consistent and stable expression of AAV replication and packaging proteins by the production cell lines such that AAV can be produced by transfection with a single vector carrying a GOI.

AAV production cells with stably integrated Rep/Cap and/or Helper genes are transfected with either the ITR-GFP-ITR msDNA or GFP-ITR msDNA from Example 1, the cells are incubated for production of AAV, and AAV is purified, according to standard procedures.

### C. Characterization of AAVs

AAVs produced by each combination in Table 3 are characterized as well as AAVs produced from production cells with stably integrated Rep/Cap and Helper genes.

Capsid composition is analyzed by Western blotting using capsid protein-specific primary mouse antibodies and secondary peroxidase-conjugated donkey anti-mouse IgG.

Transducing titers are determined by transduction of HeLa cells with serially diluted vectors.

Transduction efficiencies are evaluated by applying identical viral titers to cells and evaluating transgene expression by flow cytometry at 72 hours post transduction. For packaging efficiency, the number of fully loaded AAV particles relative to empty capsids is determined. For transfection efficiency, the levels and durability of GFP expression is determined.

Viral vector genomic particles (genomic titers) are evaluated by Real-Time quantitative PCR (qPCR). Total DNA is isolated from AAV preparations followed by Real-Time qPCR using transgene-specific primers.

Bacterial backbone and other plasmid-associated impurities are quantified by Real-Time qPCR using primers listed in Table 4.

TABLE 4
Primers for qPCR Analysis

| | | | Range of |
|---|---|---|---|
| | | | Detection |
| Target | Forward Primer | Reverse Primer | (# particles) |
| ampR | ATCCCGTATTGACGCC | CGCTCGTCGTTTGGTA | 200-2 × 10⁸ |
| | (SEQ ID NO: 26) | (SEQ ID NO: 27) | |
| F1 ori | GCGTGATGGACAGACT | GTCGAGGTGCCGTAAAG | 200-2 × 10⁸ |
| | (SEQ ID NO: 28) | (SEQ ID NO: 29) | |
| ori | TACCGGGTTGGACTCA | CCCGACAGGACTATAAAG | 200-2 × 10⁸ |
| | (SEQ ID NO: 30) | (SEQ ID NO: 31) | |
| SCAR | AACCCATAATTGTGAGCG | CATTAAGGTCGGGAAAATGC | 2 × 10³- |
| | (SEQ ID NO: 32) | (SEQ ID NO: 33) | 2 × 10⁸ |
| kanR | GCCCTGAATGAACTGC | CCATCCGAGTACGTGC | 200-2 × 10⁹ |
| | (SEQ ID NO: 4 | (SEQ ID NO: 35) | |

Multiple t-test or ANOVA is used for statistical analysis, followed by Tukey test, with P values below 0.05 considered statistically significant.

### Example 3

Production of ITR-GOI-ITR msDNA

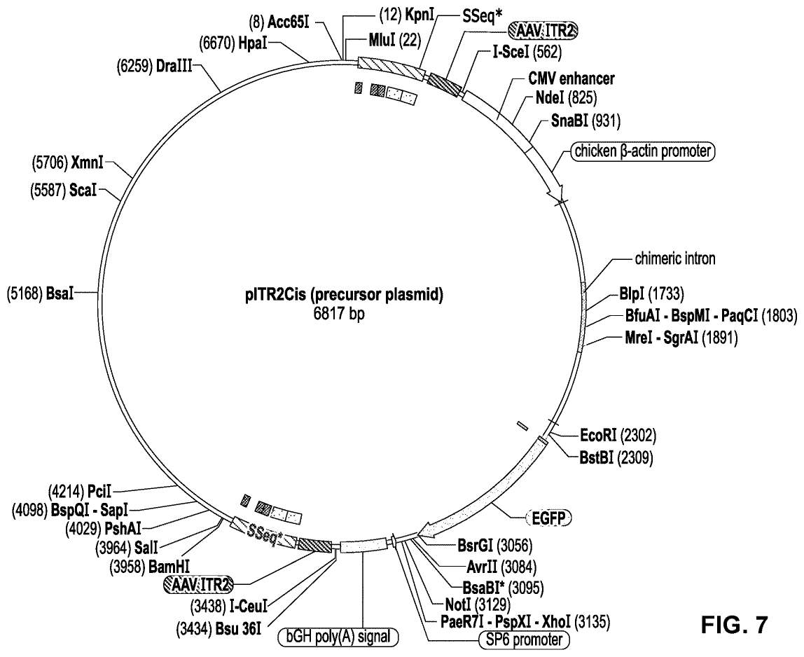

An expression vector for producing msDNA encoding enhanced green fluorescent protein (eGFP) as the gene of interest (GOI) flanked by artificial AAV2 ITRs was produced as described in Example 1, U.S. Pat. Nos. 9,290,778 and 9,862,954, and International Publication No. WO 2022/264095. FIG. 7 shows a map of the expression vector (pITR2Cis (precursor plasmid)), which includes a specialized Super Sequence site (“SSeq*” (SEQ ID NO: 36)) having recombinase target sequences (telL, FRT (minimal), and loxP) flanking artificial AAV2 ITR sequences (5′ “AAV ITR2” (SEQ ID NO: 38) and 3′ “AAV ITR2” (SEQ ID NO: 39)) that in turn flank an expression cassette containing a synthetic promoter that includes a cytomegalovirus (CMV) enhancer, a promoter from chicken β-actin, and a chimeric intron, sequences encoding enhanced green fluorescent protein (eGFP), and a bovine growth hormone polyadenylation signal (bGHpA). The SSeq is separated from each of the 5′ and 3′ ITRs by an artificial spacer sequence (SEQ ID NOs: 40 and 41, respectively). A nucleic acid sequence for pITR2Cis (precursor plasmid) is provided as SEQ ID NO:42.

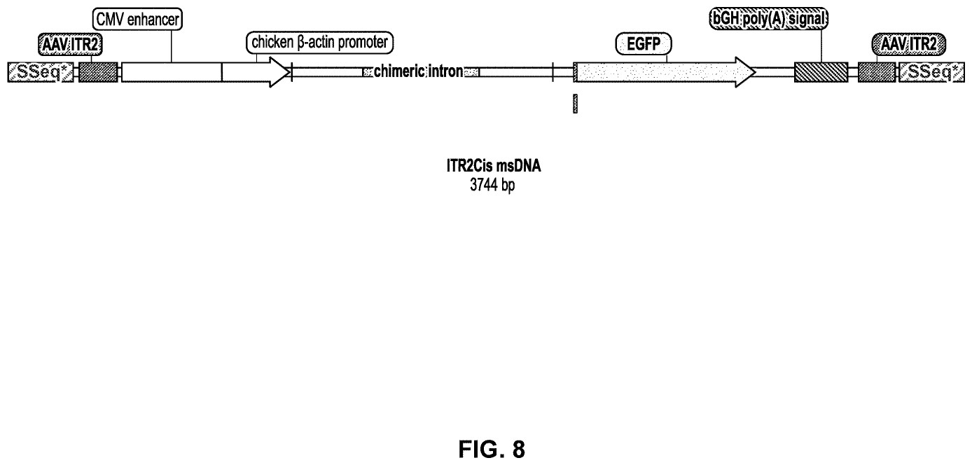

An msDNA was produced from the precursor plasmid shown in FIG. 7 as described in Example 1, U.S. Pat. Nos. 9,290,778 and 9,862,954, and International Publication No. WO 2022/264095. FIG. 8 shows a map of the msDNA (ITR2Cis msDNA), which includes a portion of the SSeq after Tel recombination (SEQ ID NO: 37) at the 5′ and 3′ ends. A nucleic acid sequence for ITR2Cis msDNA is provided as SEQ ID NO: 43.

Production of ITR-GOI-ITR msDNA

Transfection efficiency of the msDNA (ITR2Cis msDNA) was compared to its parent plasmid (pITR2Cis).

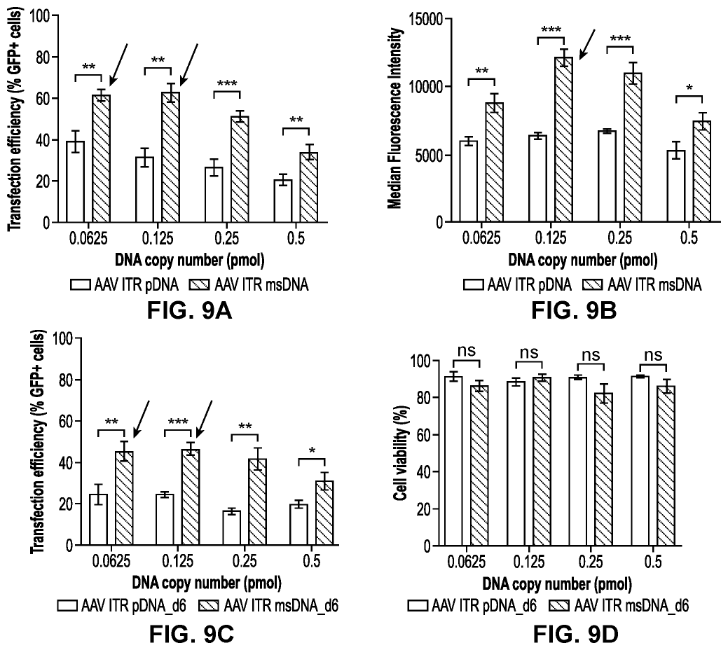

Briefly, equal molarities of ITR2Cis msDNA and pITR2Cis were formulated with LIPOFECTAMINE 3000 and separately transfected into adherent HEK293 cells. Transfection efficiency (TE, percentage of GFP-positive cells) and median fluorescence intensity (MFI) were evaluated by flow cytometry at days 2 and 6 post-transfection. FIGS. 9A and 9B show the results at day 2 for TE and MFI, respectively, while FIGS. 9C and 9D show the results at day 6 for TE and MFI, respectively.

The results show that lower doses of the msDNA offered greater TE and GFP expression levels, with the 0.125 pMol dose of msDNA (0.38 μg) showing the highest TE and MFI. The greatest transfection efficiency and transgene expression was observed at 2 days post-transfection. The results also show that AAV ITR-GOI-ITR msDNA significantly outperformed equimolar quantities of ITR-GOI-ITR precursor plasmid DNA.

Cell viability was not impacted by ITR-GOI-ITR msDNA (FIG. 9D).

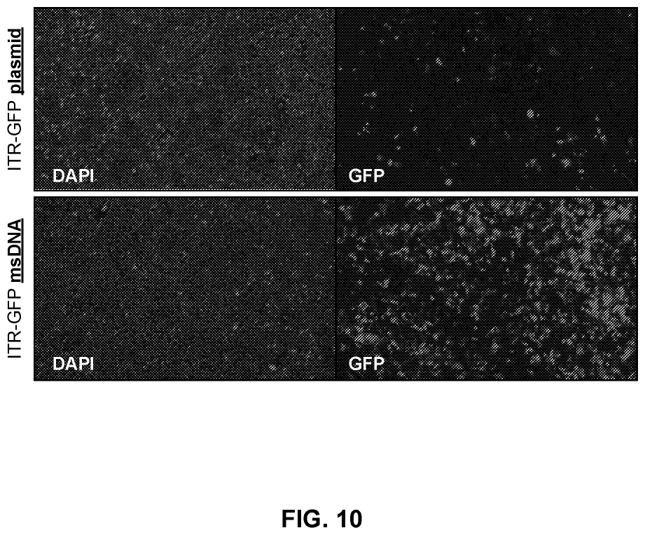

Additionally, live imaging was performed at day 3 post-transfection of HEK293 cells with 0.25 pMol msDNA (0.58 μg) or 0.25 pMol precursor plasmid DNA (1.05 μg) in a 1:4 ratio with LIPOFECTAMINE 3000. FIG. 10 shows a photomicrograph of GFP expression in the transfected cells. Nuclei are indicated by staining with diamidino-2-phenylindole (DAPI). The msDNA demonstrated much stronger TE than the parent plasmid.

### Example 4

### Sucrose Toxicity Assay

A Sucrose Toxicity (SuTox) fidelity assay was used to assess the accuracy of in vivo versus in vitro msDNA synthesis based on loss-of-function (LOF) mutations in the conditionally toxic sacB gene. Specifically, the SacB protein is toxic to bacteria in the presence of sucrose, allowing for positive selection of mutants. Faithful replication of the sacB gene results in bacterial cell death, whereas a colony will grow on sucrose if a LOF mutation in sacB occurs during DNA synthesis.

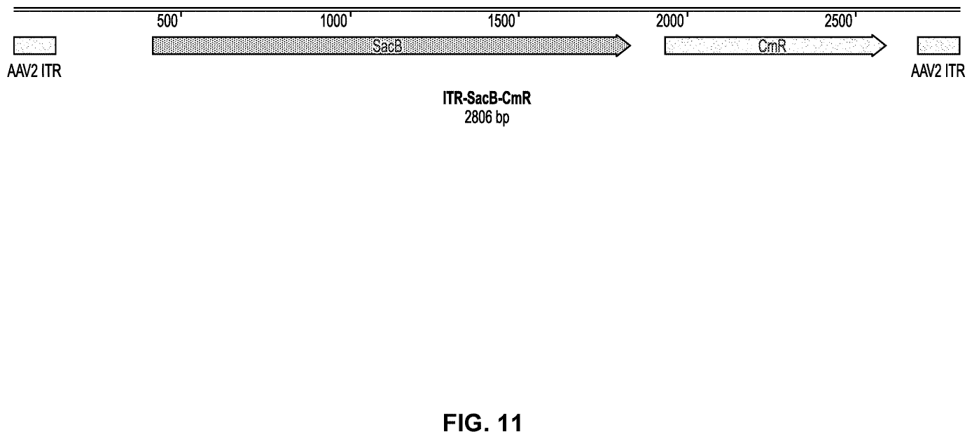

A polygenic expression vector for msDNA was produced containing an expression cassette with a sacB gene and a chloramphenicol resistance gene (encoding chloramphenicol acetyl transferase) flanked by 5′ and 3′ AAV ITR2 sequences in turn flanked by a 5′ and 3′ SSeq. See Examples 1 and 3 as well as U.S. Pat. Nos. 9,290,778 and 9,862,954, and International Publication No. WO 2022/264095. FIG. 11 shows the ITR-sacB-CmR-ITR cassette contained in the expression vector.

msDNA was expressed from the expression vector in vivo in E. coli cells (MBI2 and MBI3 strains, Mediphage Bioceuticals, Inc., Toronto, CA) using the methods described in Example 1, U.S. Pat. Nos. 9,290,778 and 9,862,954, and International Publication No. WO 2022/264095.

For in vitro replication, primers were designed that contained either a SacI or SalI restriction enzyme site for binding just outside of the ITR-sacB-cmR-ITR cassette in the expression vector. Using these primers, the cassette was amplified by PCR using Taq (FroggaBio T-500) or Q5 (New England Biolabs M0491) polymerases, applying their respective manufacturer-suggested buffers and thermocycling conditions. The same primers were used to guide rolling circle amplification (RCA) with Phi29 polymerase (New England Biolabs M0269). Following PCR or RCA, the enzymes and buffer reagents were removed using a commercial PCR purification kit (Thermo Fisher K0702) to obtain in vitro-synthesized DNA. Since Phi29 produces multimers that are difficult to purify, the completed Phi29 reaction was digested with SacI and SalI restriction enzymes prior to the PCR purification protocol. The DNA input was calculated as the quantity of starter plasmid added to the reaction as template multiplied by the length of the amplified region as a fraction of the total plasmid size. The DNA output was calculated as the concentration of the PCR purification (obtained using a nanodrop spectrophotometer) multiplied by the elution volume.

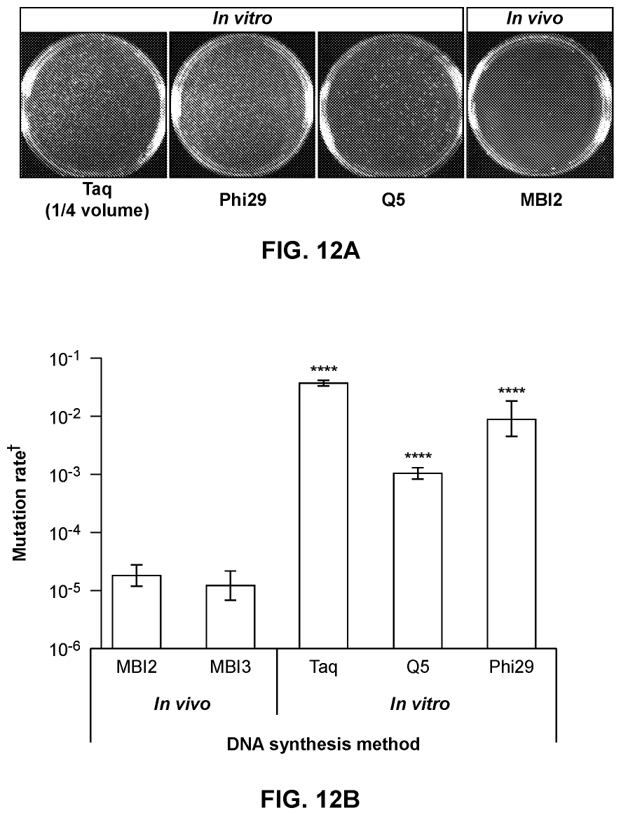

The in vivo and in vitro synthesized DNA were digested with SacI and SalI restriction enzymes (New England Biolabs R3156, R3138), as was a pUC19 vector with ampicillin resistance. A commercial gel extraction kit (Thermo Fisher K0691) was used to isolate the sacB-CmR fragment and each insert:vector combination was ligated overnight with T4 ligase (New England Biolabs M0202). A no-insert reaction also was included as a negative control. The ligations were transformed into high efficiency (1-3×10⁹ CFU/ug pUC19 DNA) competent cells (New England Biolabs C3040). The transformations were serially diluted and plated on LB with ampicillin (100 μg/ml), chloramphenicol (25 μg/ml), and either 1% NaCl (standard Miller LB) or 6% sucrose. The plates were grown at 37° C. for 16-24 h. Colony forming units (CFU) were counted for each sample with and without sucrose. Any CFU counted from the negative control transformation were subtracted as background for all samples. Samples with no CFU on sucrose after background subtraction were treated as below detection limit (BDL) and calculated as if 0.5 CFU were on the sucrose plate. CFU from the sucrose plates were counted as sacB mutants. FIG. 12A shows representative images of sucrose plates with transformations of ITR-sacB-CmR-ITR LCC DNA generated by PCR (Taq and Q5), RCA (Phi29), or in vivo in E. coli (MBI2). For Taq, one-fourth of the volume was plated relative to the other images.

CFU on standard LB plates were counted as total transformants. The number of DNA doublings was calculated as Log 2 (DNA output/input). Finally, the mutation rate was calculated as the sacB mutants/total transformants/DNA doubling as shown in FIG. 12B. Paraphrased, this calculates the fraction of DNA molecules that contain a LOF sacB mutation, normalized to how many times the original template was replicated. The mutation rate calculated from the SuTox method describes LOF mutations in the sacB gene.

To obtain an estimate of the number of mutations per bp replicated, the mutation rate was divided by the length of the sacB ORF and promoter (1533 bp) and then multiplied by 1000 bp to yield mutations/kb replicated, followed by multiplying by 100% to express the values as a percentage. Mutation rates are shown below in Table 5.

TABLE 5
Approximate LOF mutation rates per kilobase synthesized

| DNA Synthesis Method | | Mutation Rate/kb (%) |
|---|---|---|
| In | MB12 | 0.0012 |
| vivo | MB13 | 0.00079 |
| In | Taq | 2.3 |
| vitro | Q5 | 0.066 |
| | Phi29 | 0.58 |

The data in FIG. 12B and Table 5 show that in vitro replication with Taq, Phi29, or Q5 results in about 3000, 750, and 85 times more errors, respectively, than with in vivo replication of msDNA. Thus, the data show that DNA produced in vivo in E. coli cells significantly surpassed the accuracy of PCR or RCA methods. Employing E. coli-based in vivo generated DNA, such as msDNA, can effectively reduce mutations, thereby mitigating risk and enhancing the overall quality of the final product.

### Example 5

Production of AAV with msDNA

AAV was produced by replacing one, two, or all three components of a conventional plasmid AAV production system with msDNA.

Test 1—One msDNA, Two Conventional Plasmids

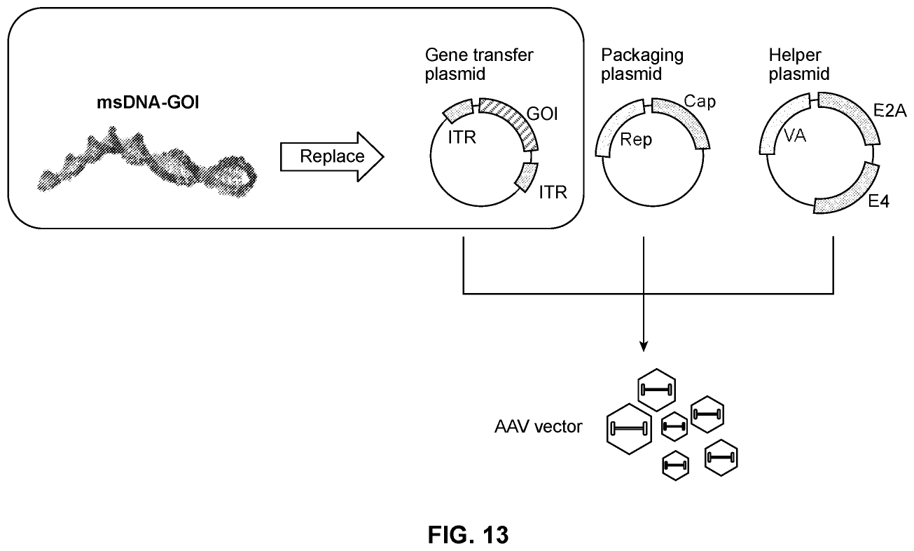

FIG. 13 shows a diagram of AAV production in which a conventional GOI-containing plasmid is replaced with msDNA.

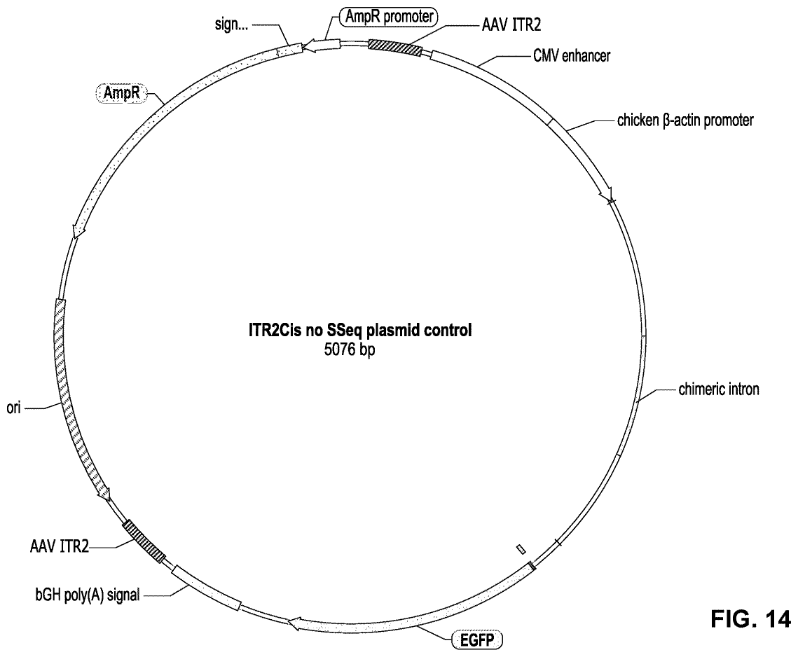

AAV1 and AAV2 serotypes were produced using the ITR-GOI-ITR msDNA described in Example 3 (i.e., ITR2Cis msDNA) or a plasmid encoding GFP and containing no SSeq (i.e., ITR2Cis no SSeq plasmid control as shown in FIG. 14) in combination with a conventional Rep/Cap plasmid (i.e., pDNA-Rep2/Cap1 for AAV1 production or pDNA-Rep2/Cap2 for AAV2 production) and a conventional helper plasmid (i.e., pDNA-helper) at different molar ratios at constant mass. A nucleic acid sequence for ITR2Cis no SSeq plasmid control is provided as SEQ ID NO:44.

pDNA-helper, pDNA-Rep/Cap, and ITR2Cis msDNA or ITR2Cis no SSeq plasmid control were mixed in molar ratios of 1:2:1, 2:1.5:1, or 1.4:1.5:1 and then each mixture was complexed in a 1:1 ratio with FECTOVIR-AAV transfection reagent for 15 minutes in 5% high glucose DMEM media. Following complexation, 2 μg/mL of each mixture was separately transfected into either 35 mL or 150 mL cultures of GIBCO VCP2.0 cells (Thermo Fisher Scientific), a clonal cell line derived from the HEK293F parental cell line, having a density of 2×10⁶ cells/mL for a total concentration of 1 μg DNA/1×10⁶ cells. DNA was selected for AAV2 production in 35 mL cultures, while DNA was selected for both AAV1 and AAV2 serotypes in 150 mL cultures.

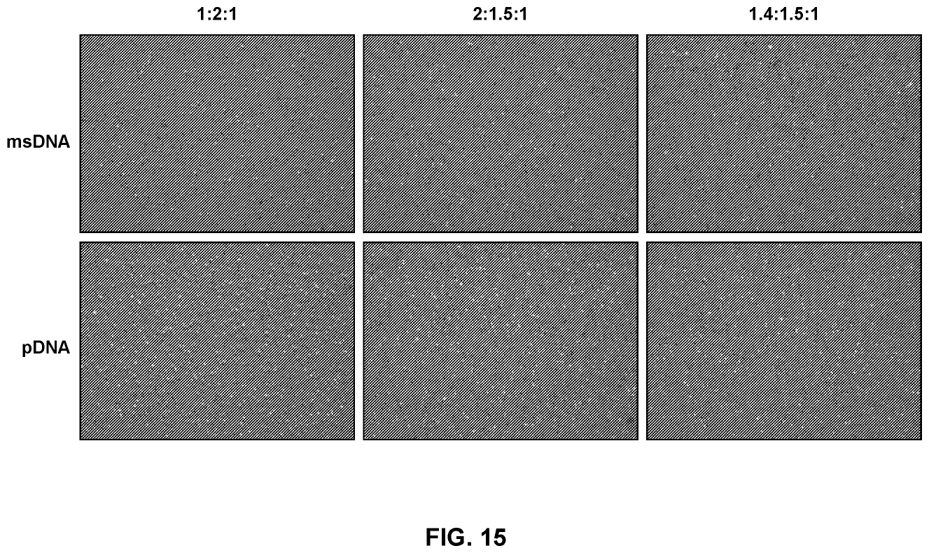

FIG. 15 shows GFP expression from samples of the 35 mL cultures for all three ratios.

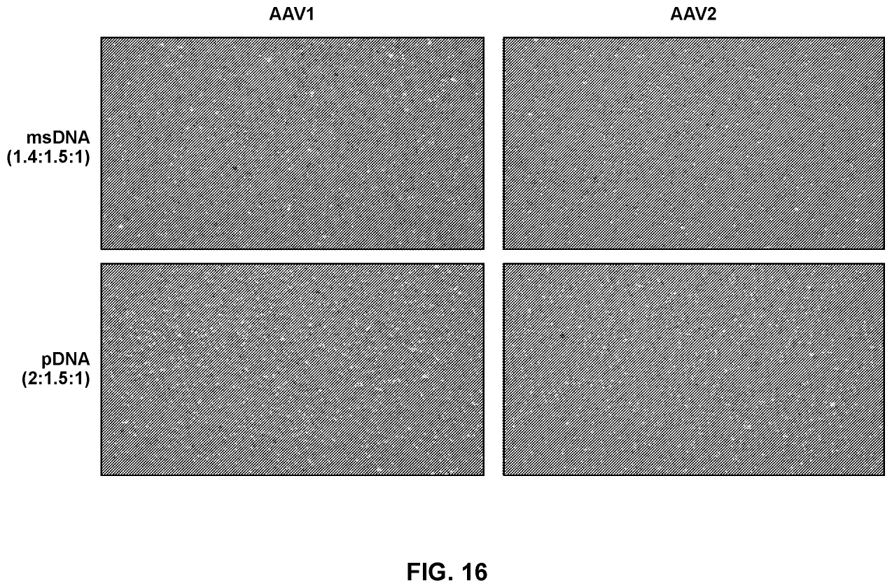

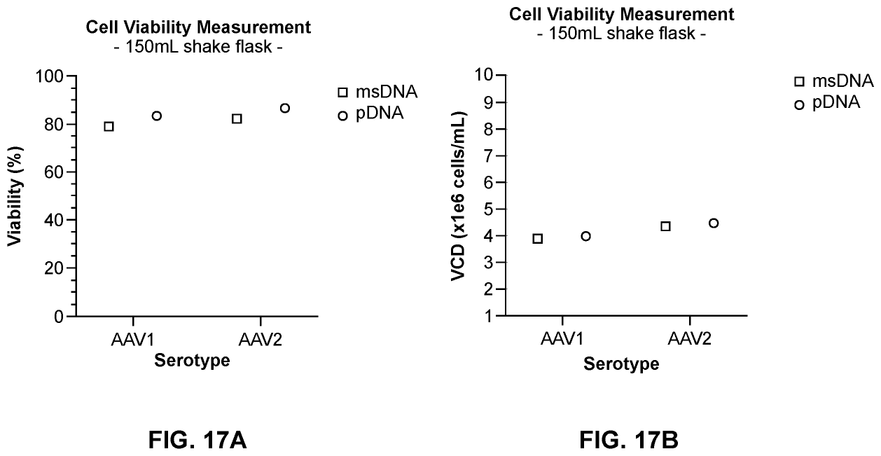

FIG. 16 shows GFP expression from samples of the 150 mL cultures for the AAV1 and AAV2 serotypes produced from a 1.4:1.5:1 molar ratio of pDNA-helper:pDNA-Rep/Cap:ITR2Cis msDNA (“msDNA”) or 2:1.5:1 pDNA-helper:pDNA-Rep/Cap:ITR2Cis no SSeq plasmid control (“pDNA”). Cell viabilities associated with these transfections are shown in FIGS. 17A (% viable cells) and 17B (viable cell density (VCD), ×10⁶ cells/mL).

After 72 hours, cells in the 35 mL and 150 mL cultures were lysed for 2 hours at 37° C. on a shaker in a lysis buffer containing 1% Tween, 500 mM NaCl, 2 mM MgCl2 buffer, and 20 U/mL DENARASE.

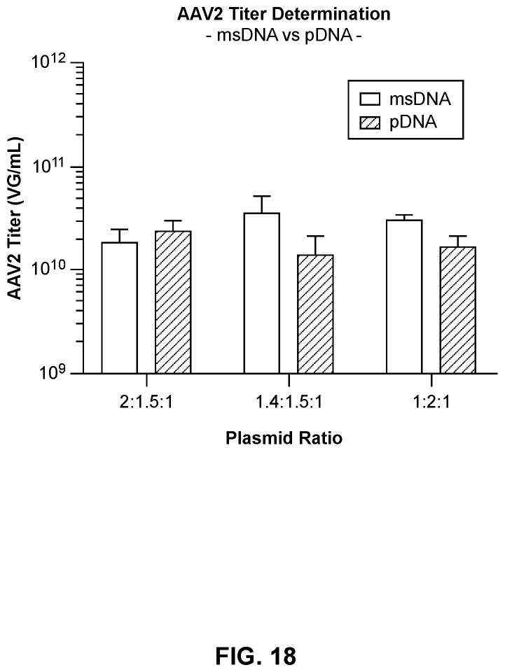

Droplet digital PCT (ddPCR) was conducted to determine titers of AAV2 vector genome/mL (VG/mL) based on presence of the GOI in the 35 mL cultures for the different ratios using either ITR2Cis msDNA (“msDNA”) or ITR2Cis no SSeq plasmid control (“pDNA”). Results are shown in FIG. 18 and below in Table 6.

TABLE 6
ddPCR Titer of Bulk AAV2 Harvest (30 mL)

| Sample (ratio of vectors) | Titer of AAV2 (VG/mL) |
|---|---|
| msDNA (1:2:1) | 3.00 × 10¹⁰ |
| msDNA (2:1.5:1) | 1.85 × 10¹⁰ |
| msDNA (1.4:1.5:1) | 3.55 × 10¹⁰ |
| pDNA (1:2:1) | 1.65 × 10¹⁰ |
| pDNA (2:1.5:1) | 2.34 × 10¹⁰ |
| pDNA (1.4:1.5:1) | 1.38 × 10¹⁰ |

msDNA = AAV produced from the noted ratio pDNA-helper:pDNA-Rep/Cap:ITR2Cis msDNA
pDNA = AAV produced from the noted ratio pDNA-helper:pDNA-Rep/Cap:ITR2Cis no SSeq plasmid control

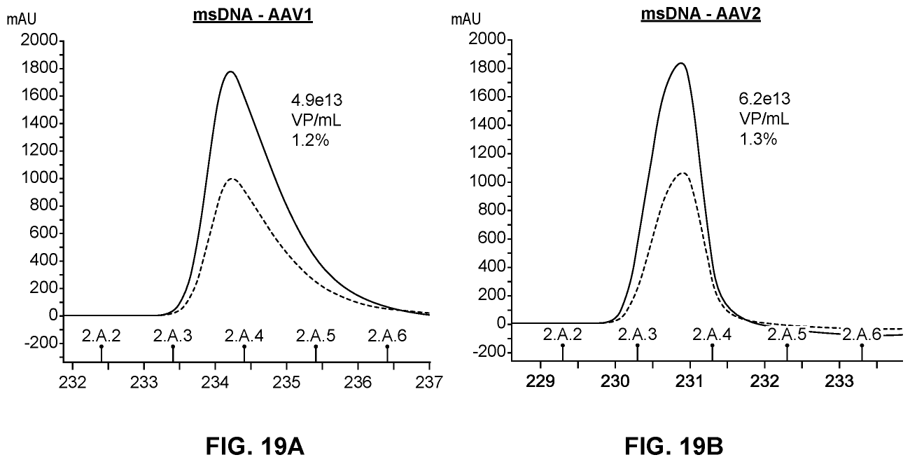

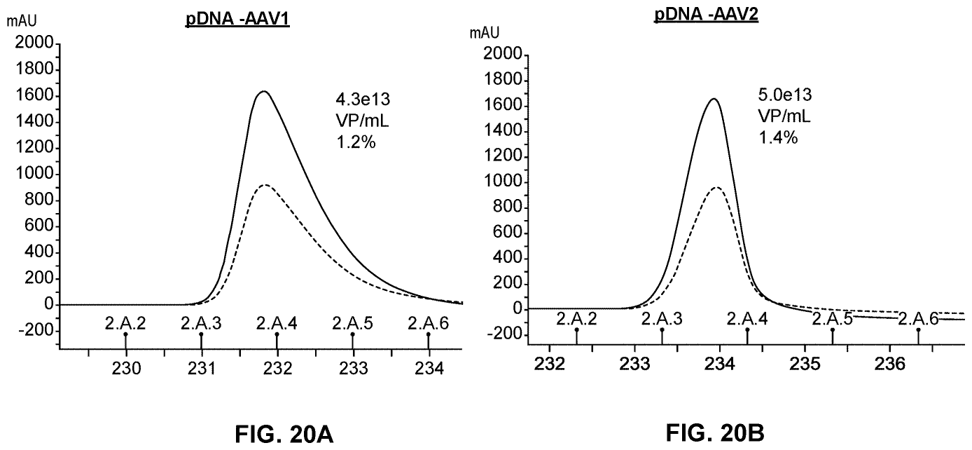

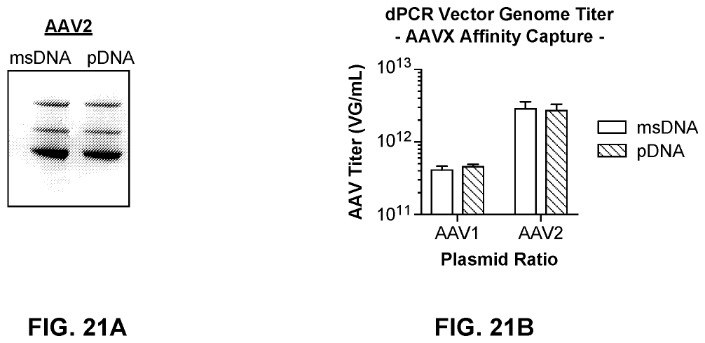

AAV1 and AAV2 were purified from 150 mL shake flask cultures with affinity chromatography. FIGS. 19A-19B and 20A-20B show chromatograms from affinity chromatography of cultures produced using either ITR2Cis msDNA (msDNA (1.4:1.5:1), FIGS. 19A (AAV1) and 19B (AAV2)) or ITR2Cis no SSeq plasmid control (pDNA (2:1.5:1), FIGS. 20A (AAV1) and 20B (AAV2)). The upper line in each figure is the absorbance at 280 nm, which indicates AAV1 or AAV2 empty capsids (i.e., they do not include encapsulated DNA), while the lower line is the absorbance at 260 nm, which indicates AAV1 or AAV2 capsids that contain encapsulated DNA. The amount of “VP/mL” indicates the concentration of vector particles per milliliter in the eluate, and the percentage indicates the proportion of particles that are packaged with DNA (i.e., % full as calculated by either A260/A280 (see, e.g., Werle et al., Mol. Ther. Methods Clin. Dev. 23:254-262 (December 2021)), directly reported from the AKTA chromatography trace, or mass photometry (REFEYN)). The percentage of full particles can include particles packaged with the GOI as well as aberrantly packaged DNA such as conventional plasmid backbone. FIG. 21A shows a photomicrograph of an electrophoresis gel indicating capsid proteins VP1, VP2, and VP3 as the three respective bands from the top to the bottom of each lane as detected by ddPCR following affinity chromatography of the msDNA (1.4:1.5:1) and pDNA (2:1.5:1) samples from the AAV2 harvest. FIG. 21B shows vector genome titers of the AAV1 and AAV2 harvest from the msDNA (1.4:1.5:1) and pDNA (2:1.5:1) samples as determined by ddPCR of the GOI.

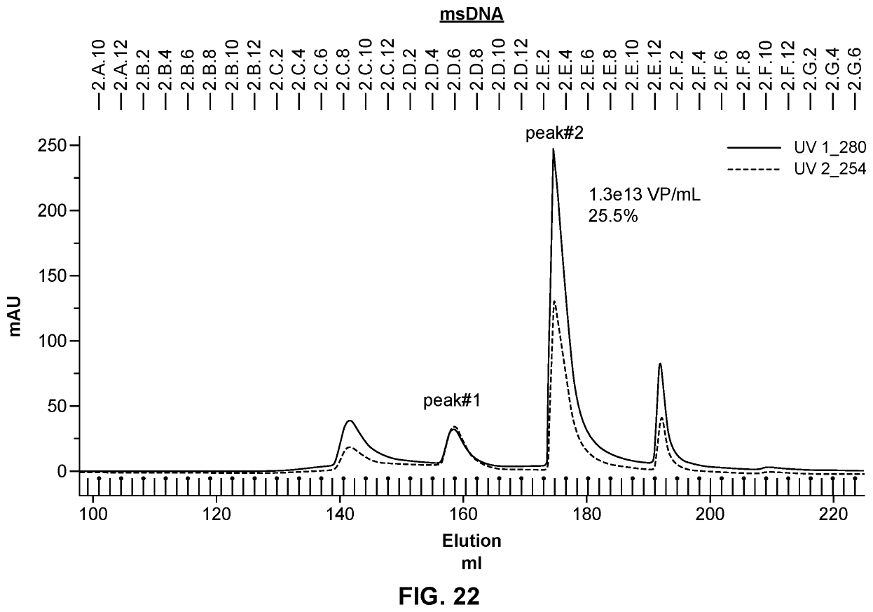

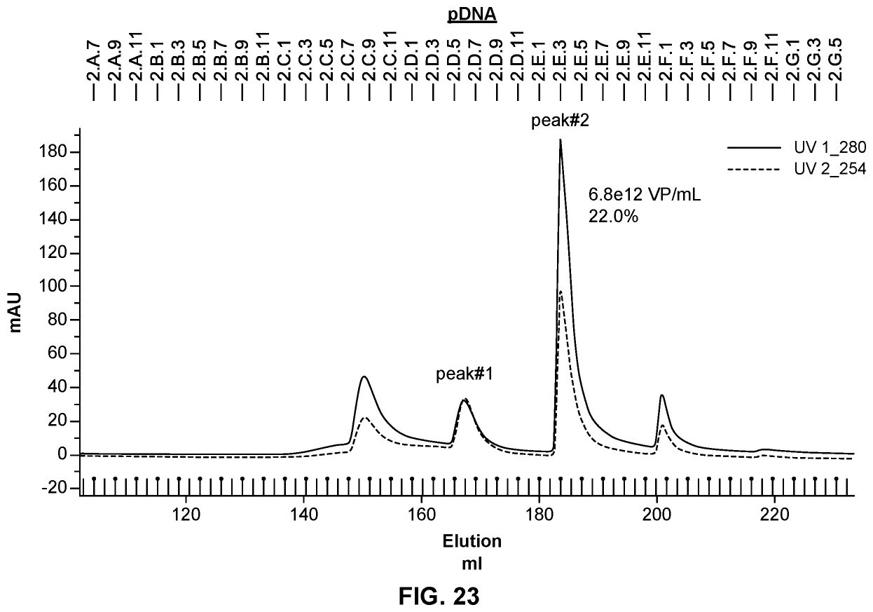

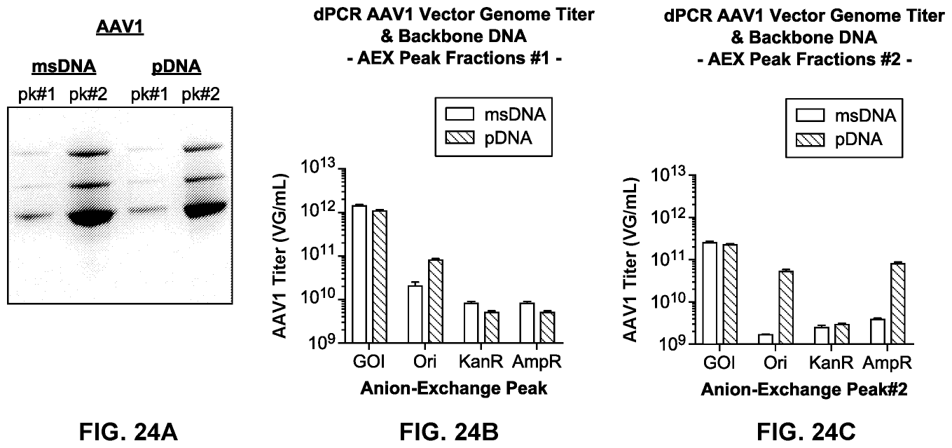

AAV1 purified by affinity chromatography from 150 mL shake flask cultures was further purified with MUSTANG Q anion exchange (AEX) chromatography. FIGS. 22 and 23 show associated chromatograms from cultures produced using either ITR2Cis msDNA (msDNA (1.4:1.5:1), FIG. 22) or ITR2Cis no SSeq plasmid control (pDNA (2:1.5:1), FIG. 23). The upper line, lower line, VP/mL, and percentage are as described for FIGS. 19-20. Peak #1 in each figure includes particles that are primarily packaged with DNA, while peak #2 shows includes empty particles as well as particles with packaged DNA. FIG. 24A shows a photomicrograph of an electrophoresis gel indicating capsid proteins VP1, VP2, and VP3 as the three respective bands from the top to the bottom of each lane as detected by ddPCR from peaks #1 and #2 of the MUSTANG Q AEX chromatography. FIGS. 24B and 24C show titers for peaks #1 and #2, respectively, associated with packaged DNA containing the gene of interest (GOI) or conventional plasmid DNA such as the origin of replication (Ori), the KanR gene, or the AmpR gene. These data show that msDNA generated a 1.9 fold higher yield and 3.5% higher full:empty ratio.

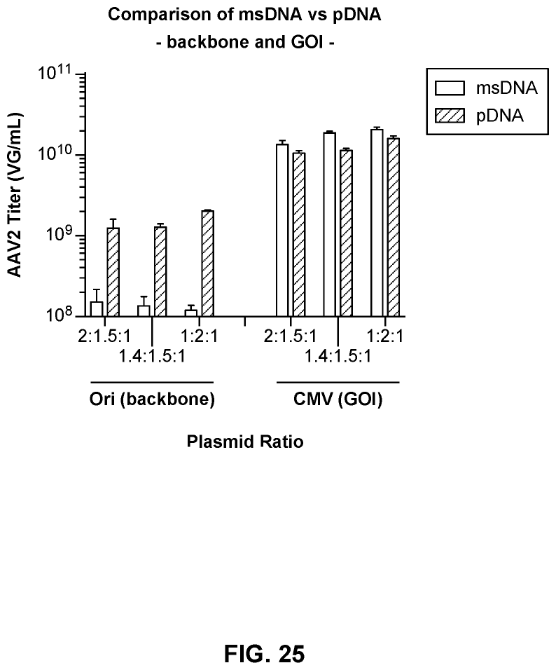

FIG. 25 compares the titers for AAV2 produced from different msDNA and pDNA ratios as described above for the 35 mL cultures, containing encapsulated conventional plasmid DNA (“Ori (backbone)”) or the expression cassette containing the GOI (“CMV (GOI)”). The figure shows much lower packaging of plasmid backbone sequences when using even a single msDNA.

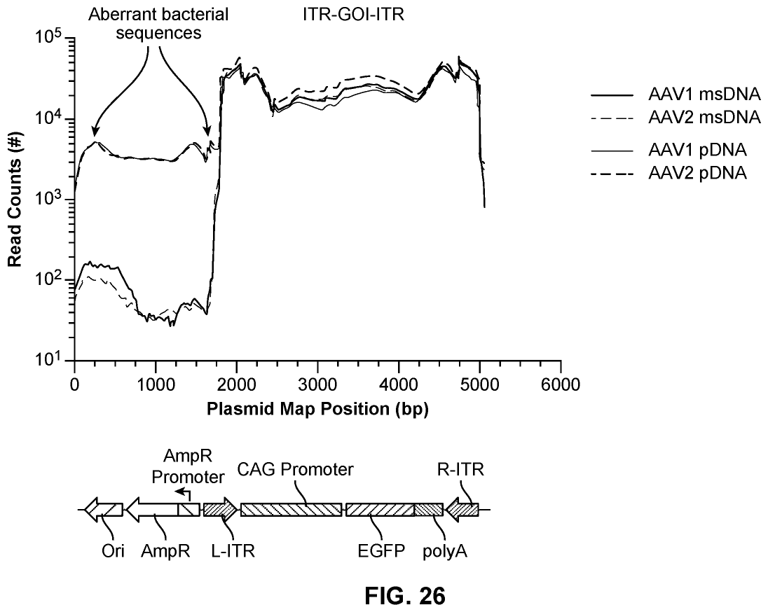

FIG. 26 shows a next-generation sequencing (NGS) coverage map of AAV1 and AAV2 packaged genomes produced using either ITR2Cis msDNA (msDNA (1.4:1.5:1) as described above) or ITR2Cis no SSeq plasmid control (pDNA (2:1.5:1) as described above) in relation to the plasmid map positions. Based on the lack of backbone sequences in the msDNA vector, FIG. 26 shows that aberrant packaging of sequences outside of the ITR-GOI-ITR region is almost negligible when using a single msDNA. Thus, aberrant backbone plasmid sequences can be eliminated when using msDNA as the carrier of the GOI versus a conventional plasmid in production of AAV.

Test 2—Two msDNAs, One Conventional Plasmid

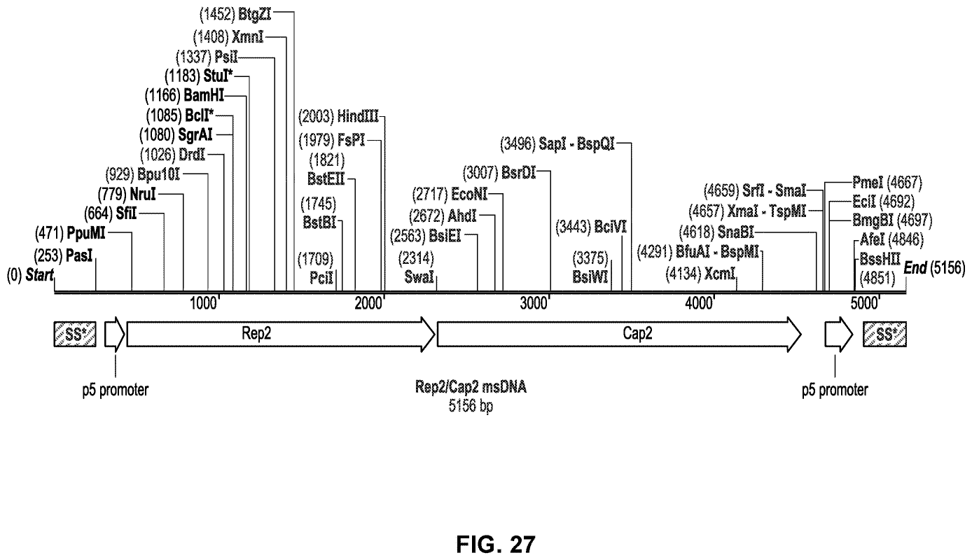

AAV2 was produced using the ITR-GOI-ITR msDNA described in Example 3 (i.e., ITR2Cis msDNA) or a conventional plasmid encoding GFP and containing no SSeq as described in Test 1 (i.e., ITR2Cis no SSeq plasmid control) in combination with a Rep2/Cap2 msDNA as shown in FIG. 27 or a conventional Rep2/Cap2 plasmid, and a conventional helper plasmid (i.e., pDNA-helper from Applied Viromics). A nucleic acid sequence for Rep2/Cap2 msDNA is provided as SEQ ID NO:48.

pDNA-helper, Rep2/Cap2 msDNA, and ITR2Cis msDNA were mixed in a 1.4:1.5:1 molar ratio (“msDNA”). pDNA-helper, pDNA-Rep2/Cap2, and ITR2Cis msDNA were mixed in a 1.4:1.5:1 molar ratio (“mixed-msDNA”). pDNA-helper, pDNA-Rep/Cap, and ITR2Cis no SSeq plasmid control were mixed a 1:2:1 molar ratio (“pDNA”). Each mixture was then individually complexed in a 1:1 ratio with FECTOVIR-AAV transfection reagent for 15 minutes in 5% high glucose DMEM media. Following complexation, each mixture was separately transfected into 150 mL cultures of GIBCO VCP2.0 cells (passage 37). 2% Glutamax was added to VPC media before use, and all media components were warmed before culture. Cells were grown at 37° C. with shaking at 130 rpm with 7% CO₂, and harvested 72 hours after transfection. Cells were then lysed by adding 1% Tween, 500 mM NaCl, and 25 U/mL DENARASE with mixing for 2 hours. Lysate was then clarified by spinning at 4000 rpm for 40 minutes before purification. Table 7 below shows viability of the samples after 72 hours of culture.

TABLE 7
Viability of Test 2 Samples

| | Total cells | Viable cells | Viability |
|---|---|---|---|
| Sample | (×10⁶ cells/mL) | (×10⁶ cells/mL) | (%) |
| msDNA | 6.8 | 6.2 | 91.0 |
| mixed-msDNA | 6.8 | 6.4 | 93.8 |
| pDNA | 7.3 | 7.1 | 96.9 |

msDNA = pDNA-helper, Rep2/Cap2 msDNA, and ITR2Cis msDNA
mixed msDNA = pDNA-helper, pDNA-Rep2/Cap2, and ITR2Cis msDNA
pDNA = pDNA-helper, pDNA-Rep/Cap, and ITR2Cis no SSeq plasmid control

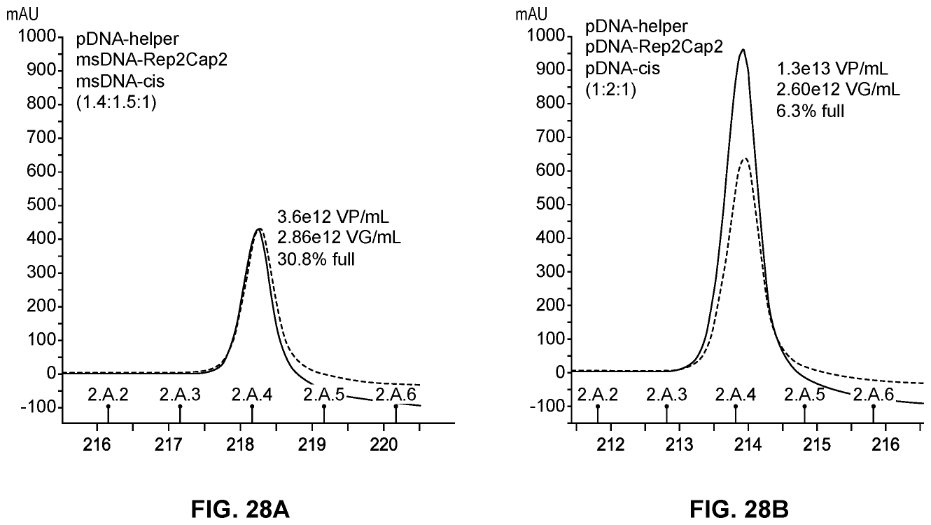

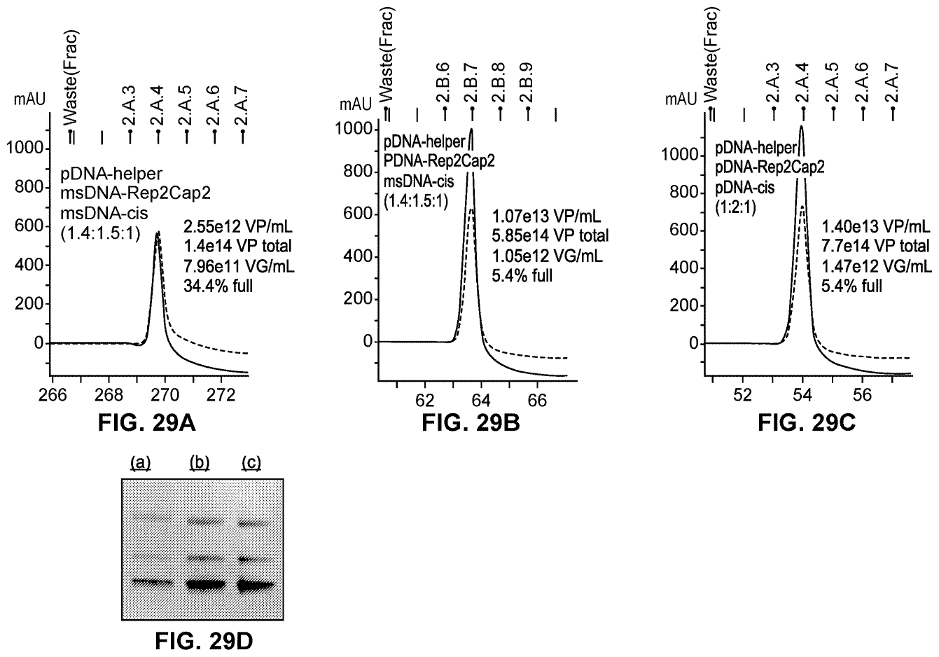

AAV2 was purified from the cultures using affinity chromatography. FIGS. 28A and 28B show chromatograms for AAV produced from the msDNA and pDNA samples, respectively. The upper line, lower line, VP/mL, and percentage are as described for FIGS. 19-20. VG/mL indicates the number of particles/mL packaged with DNA in the eluate. Consistent production was observed from an independent repeat, with chromatograms for AAV produced from the msDNA, mixed-msDNA, and pDNA samples from that repeat shown in FIGS. 29A-29C, respectively. FIG. 29D shows a photomicrograph of an electrophoresis gel indicating capsid proteins VP1, VP2, and VP3 as the three respective bands from the top to the bottom of each lane as detected by ddPCR following affinity chromatography of the msDNA sample in lane (a), the mixed-msDNA sample in lane (b), and the pDNA sample in lane (c).

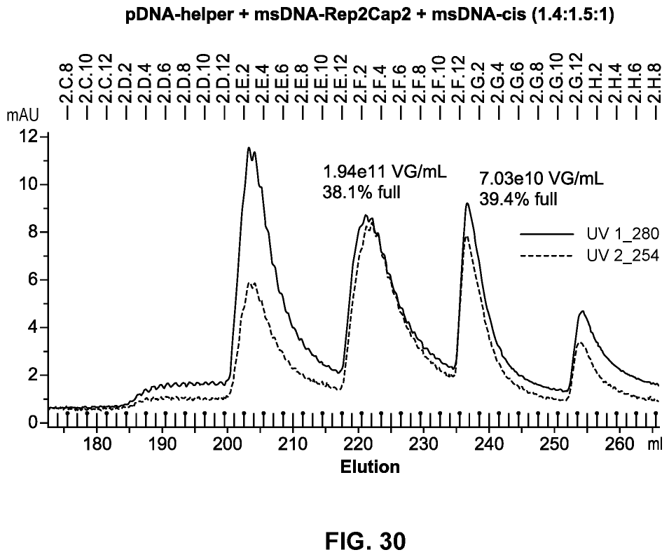

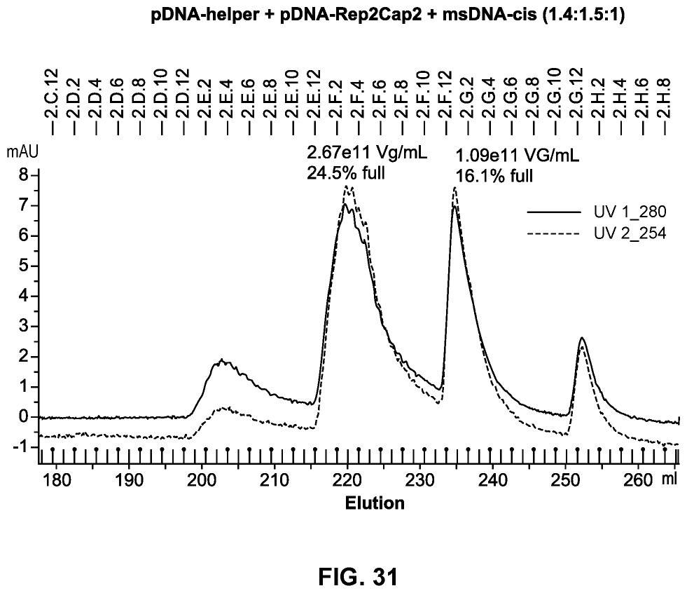

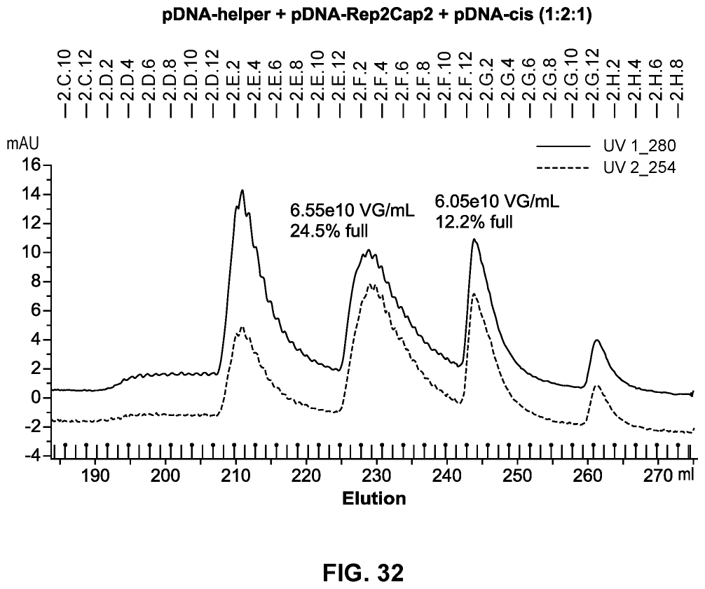

About 87.5% of the capture from affinity chromatography of the repeat shown in FIGS. 29A-29C was used for further AEX purification, resulting in the chromatograms shown in FIGS. 30-32, respectively. The peaks labeled with VG/ml and % full amounts in each figure indicate the number of vector particles containing encapsulated DNA, and the percentage of vector particles containing encapsulated DNA, respectively, as discussed above. Those peaks represent the eluates with the highest proportion of particles containing encapsulated DNA.

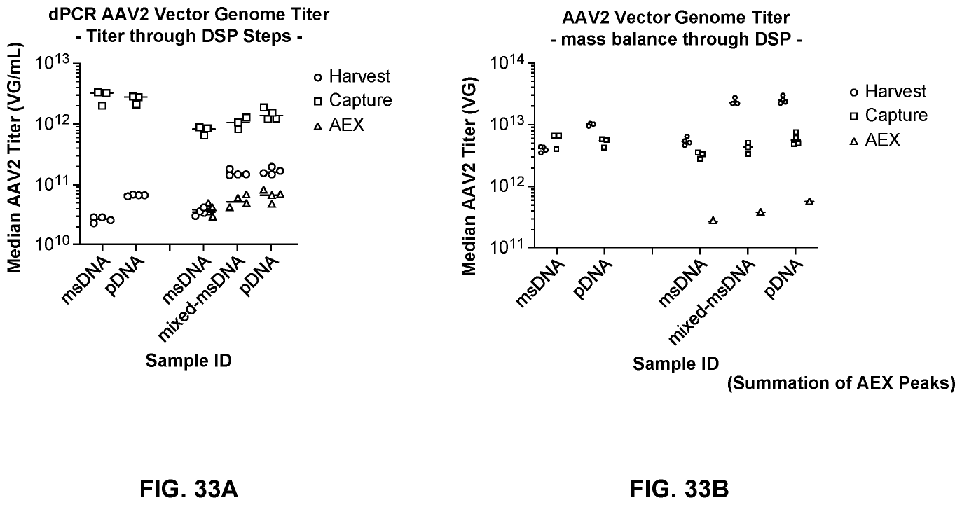

FIG. 33A shows titers of AAV2 vector genome/mL (VG/mL) based on presence of the GOI as determined by ddPCR for samples from initial harvest of the AAV2 after lysis of the cell cultures (“Harvest”), affinity chromatography (“Capture”), and AEX chromatography (“AEX”). The two Harvest and two Capture values for msDNA and pDNA samples are from independent repeats on different days as described in FIGS. 28 and 29, respectively.

FIG. 33B shows titers of AAV2 (VG, mass balance) determined for each sample by multiplying the corresponding VG/mL titer in FIG. 33A by the total volume of the sample.

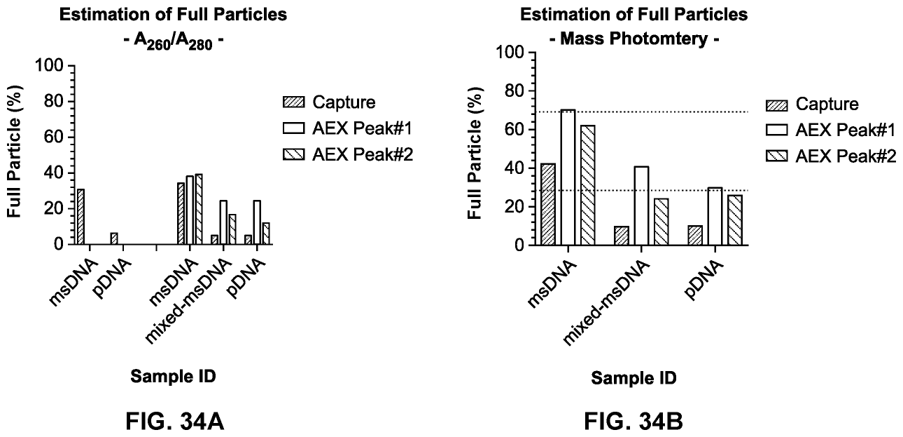

FIG. 34A shows estimation of full particles by the ratio of A260/A280 for the samples described in FIG. 33A, while FIG. 34B shows estimation of full particles by mass photometry for samples in the independent repeat as described above.

The data show a greater number of full particles using 2 msDNAs in place of 2 of the 3 conventional plasmids typically used in manufacture of AAV as well a large improvement in the avoidance of aberrant packaging of the backbone plasmid DNA.

Test 2—Three msDNAs

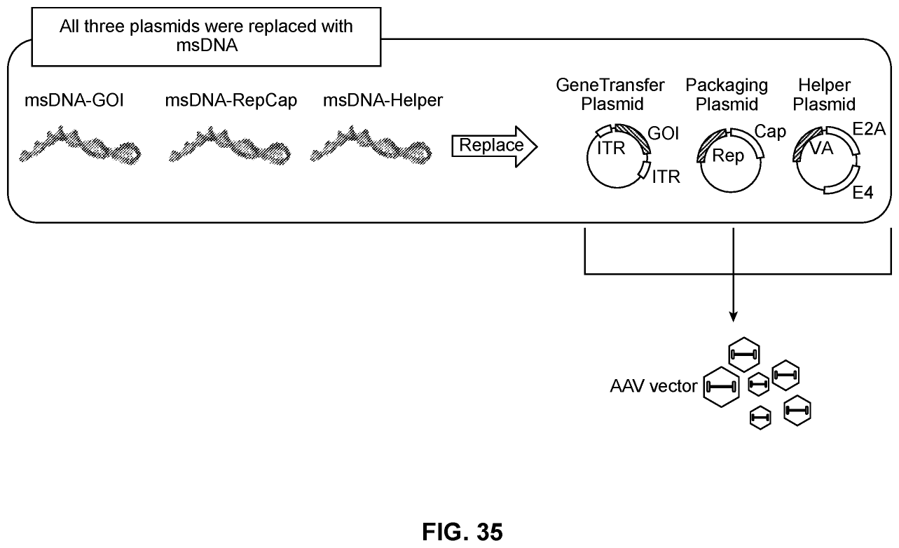

FIG. 35 shows a diagram of AAV production in which all three conventional plasmids are replaced with msDNA.

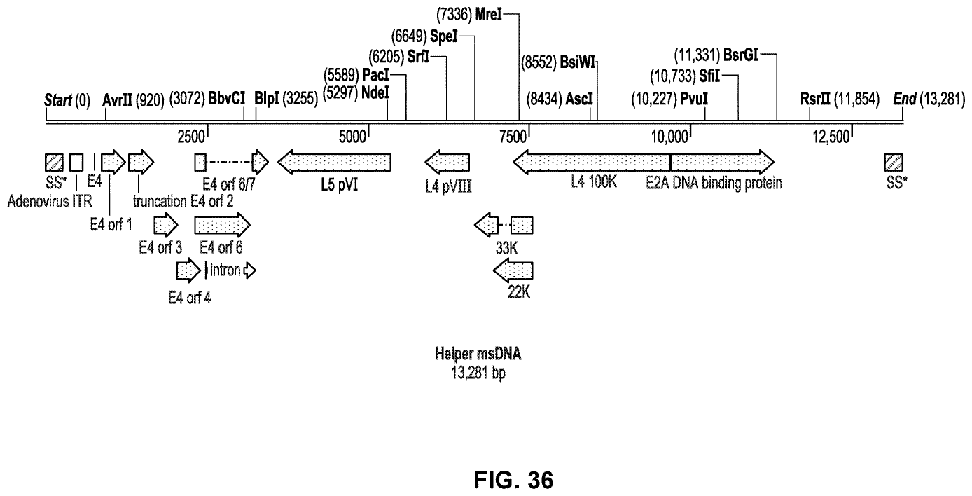

AAV2 was produced using all msDNAs, i.e., the ITR-GOI-ITR msDNA described in Example 3 (i.e., ITR2Cis msDNA), the Rep2/Cap2 msDNA described in Test 2, and the Helper msDNA shown in FIG. 36, the nucleic acid sequence for which is provided as SEQ ID NO: 51. AAV produced using all pDNAs from the independent repeat in Test 2 as shown for FIG. 29C, for example, was used as a comparison.

ITR2Cis msDNA, Rep2/Cap2 msDNA, and Helper msDNA were mixed in 1:2:1 or 1:1:1 molar ratios. Individual mixtures of the 1:2:1 ratio were complexed with either a 1:1 or 2:1 ratio of FECTOVIR-AAV transfection reagent:DNA, while the 1:1:1 ratio was complexed with a 2:1 ratio of FECTOVIR-AAV transfection reagent:DNA. Complexation occurred for 15 minutes in 5% high glucose DMEM media. Following complexation, each mixture was separately transfected into 150 mL cultures of GIBCO VCP2.0 cells. 2% Glutamax was added to VPC media before use, and all media components were warmed before culture. Cells were grown at 37° C. with shaking at 130 rpm with 7% CO₂, and harvested 72 hours after transfection. Cells were then lysed by adding 1% Tween, 500 mM NaCl, and 25 U/mL DENARASE with mixing for 2 hours. Lysate was then clarified by spinning at 4000 rpm for 40 minutes before purification.

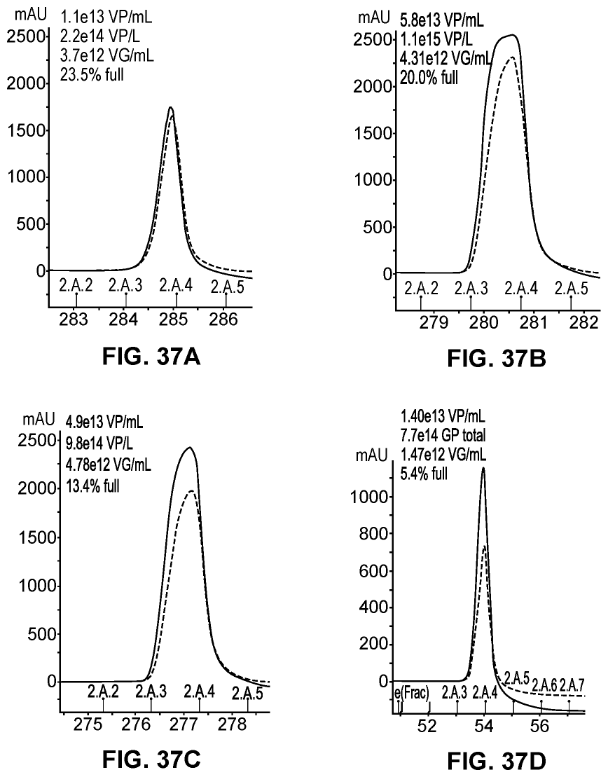

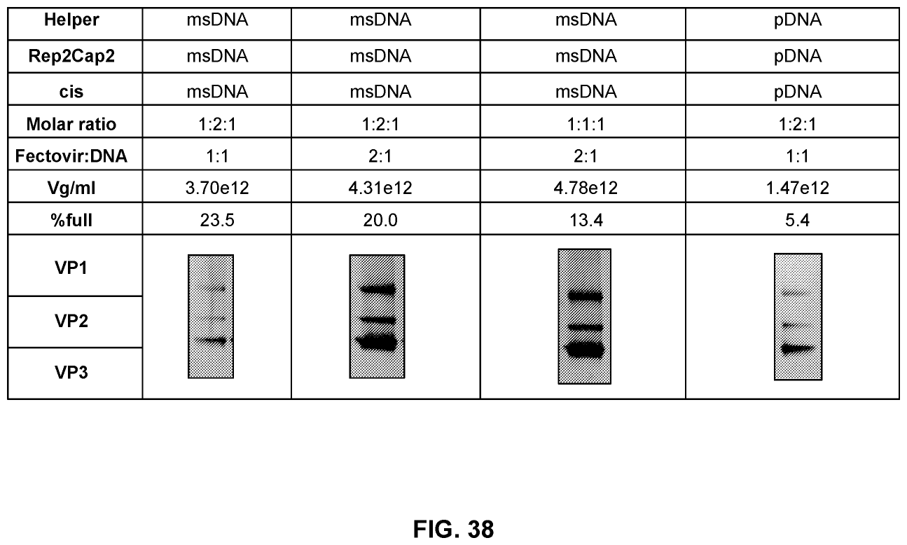

AAV2 was purified from the cultures using affinity chromatography. FIGS. 37A-37D show chromatograms for AAV produced from the msDNA 1:2:1 molar ratio 1:1 FECTOVIR:DNA sample (A), the msDNA 1:2:1 molar ratio 2:1 FECTOVIR:DNA sample (B), and the msDNA 1:1:1 molar ratio 2:1 FECTOVIR:DNA sample (C). FIG. 37D shows the chromatogram of the all pDNA sample from FIG. 29C (1:2:1 molar ratio of pDNA-helper, pDNA-Rep/Cap, and ITR2Cis no SSeq plasmid control and 1:1 FECTOVIR:DNA). The upper line, lower line, VP/mL, VG/mL and % full values are as described above. VG/L is the concentration of vector genomes in the culture. FIG. 38 includes a summary of the sample characteristics from FIGS. 37A-37D and includes photomicrographs of electrophoresis gels indicating capsid proteins VP1, VP2, and VP3 as the three respective bands from the top to the bottom of each lane as detected by ddPCR following the affinity chromatography of the samples.

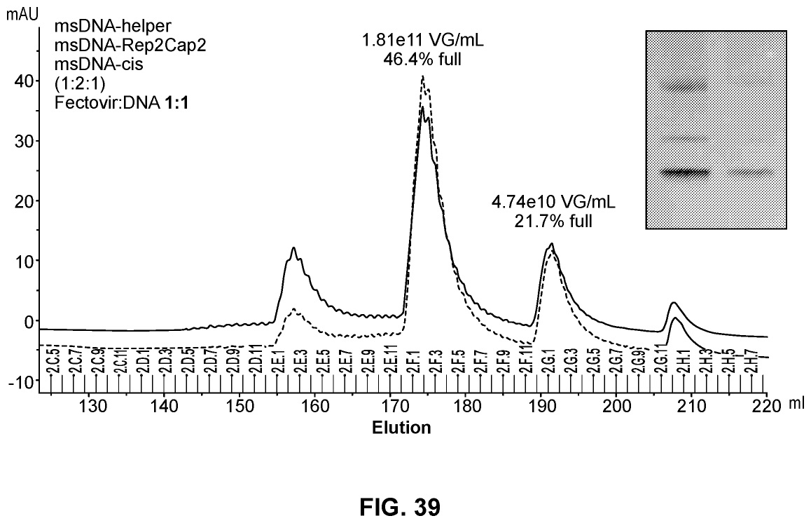

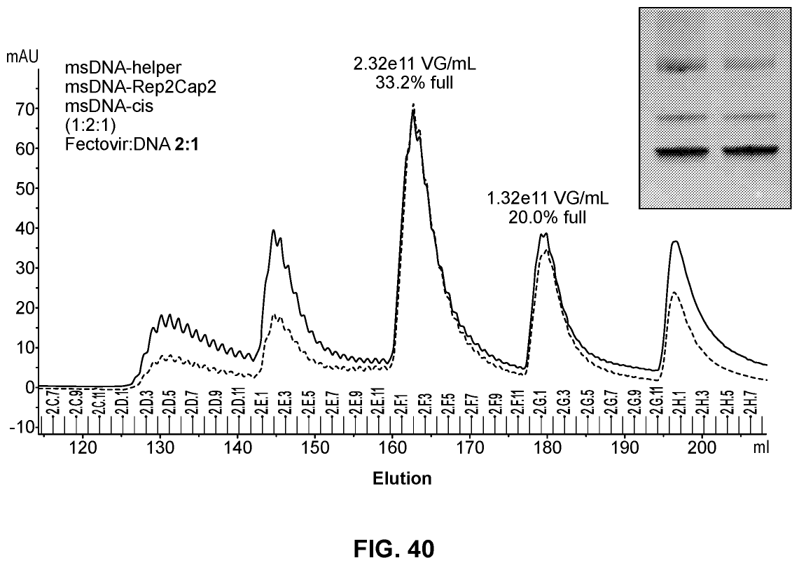

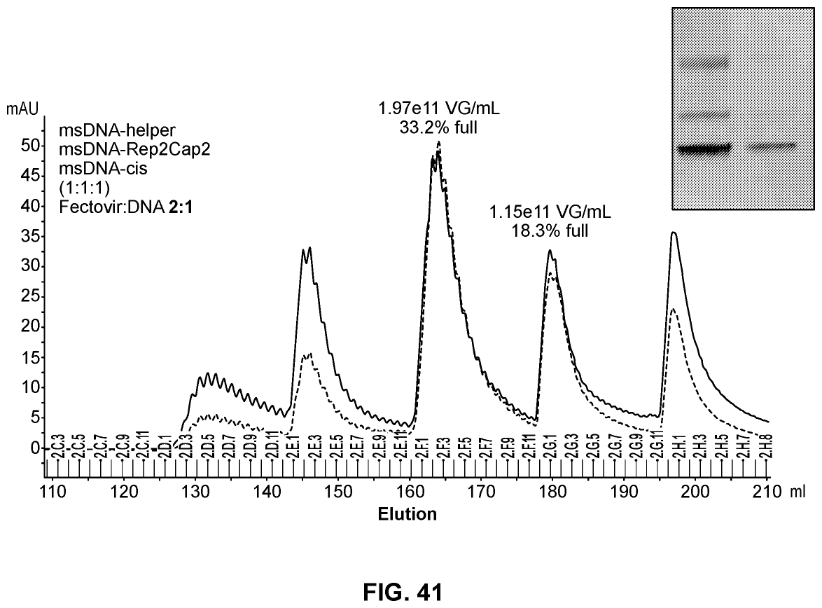

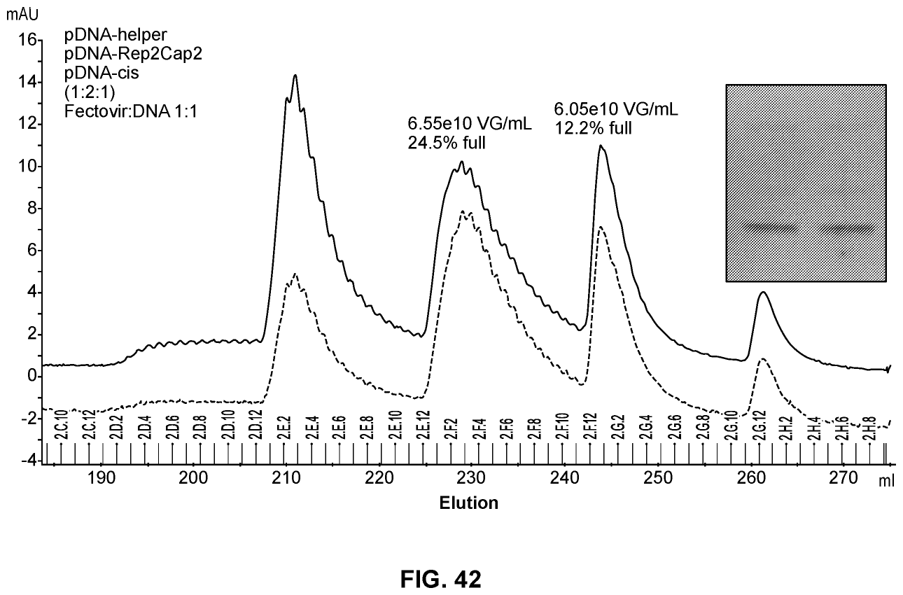

About 87.5% of the capture from the affinity chromatography shown in FIG. 37A-37D was used for further AEX purification, resulting in the chromatograms shown in FIGS. 39-42, respectively. The peaks labeled with VG/ml and % full amounts in each figure indicate the number of vector particles containing encapsulated DNA, and the percentage of vector particles containing encapsulated DNA, respectively, as discussed above. Those peaks represent the eluates with the highest proportion of particles containing encapsulated DNA.

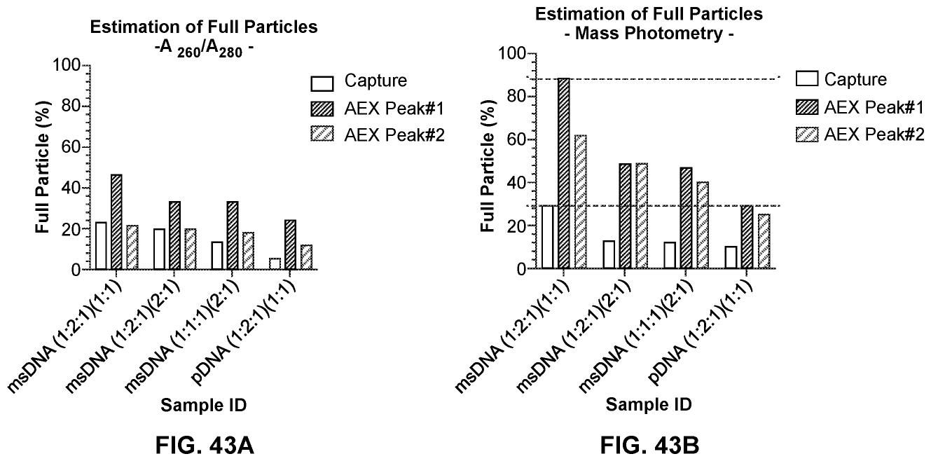

FIG. 43A shows estimation of full particles by the ratio of A260/A280 for the samples from affinity chromatography (“Capture”), and AEX chromatography (“AEX Peak #1” and “AEX Peak #2”). FIG. 43B shows estimation of full particles by mass photometry for the samples.

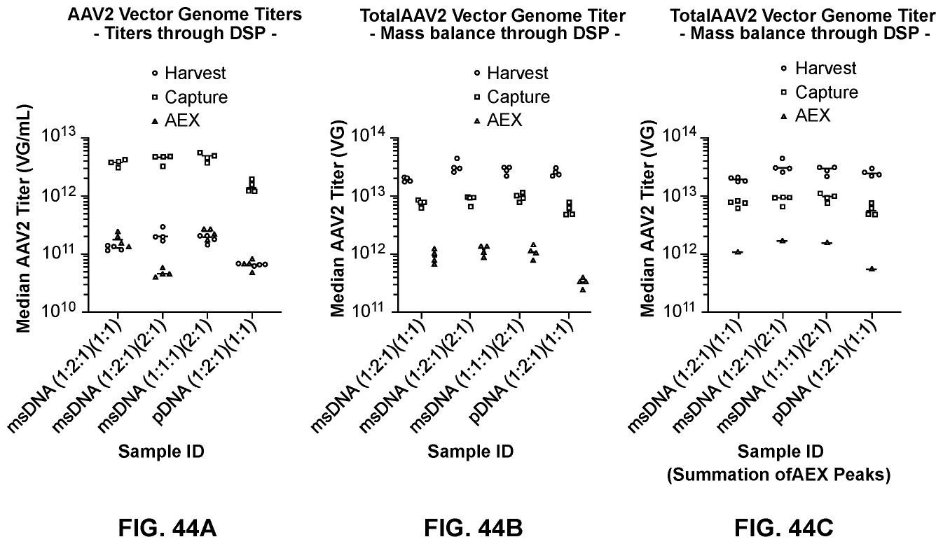

FIG. 44A shows titers of AAV2 vector genome/mL (VG/mL) based on presence of the GOI as determined by ddPCR for the samples from initial harvest of the AAV2 after lysis of the cell cultures (“Harvest”), affinity chromatography (“Capture”), and peak #1 of AEX chromatography (“AEX”). FIG. 44B shows the titers of AAV2 (VG, mass balance) determined for each sample by multiplying the corresponding VG/mL titer in FIG. 44A by the total volume of the sample. FIG. 44C show titers of AAV2 (VG, mass balance) determined for each sample, including both peaks #1 and 2 of the AEX chromatography.

The data show that the Full:Empty capsid ratios dramatically improved from 5.4% full (triple pDNA conventional plasmid transfection) to >20% full with semi-optimized conditions using msDNA in place of all three conventional plasmids. The culture and other process-specific parameters were not optimized for msDNA and were simply plug-and-play based on the existing SOPs for plasmid.

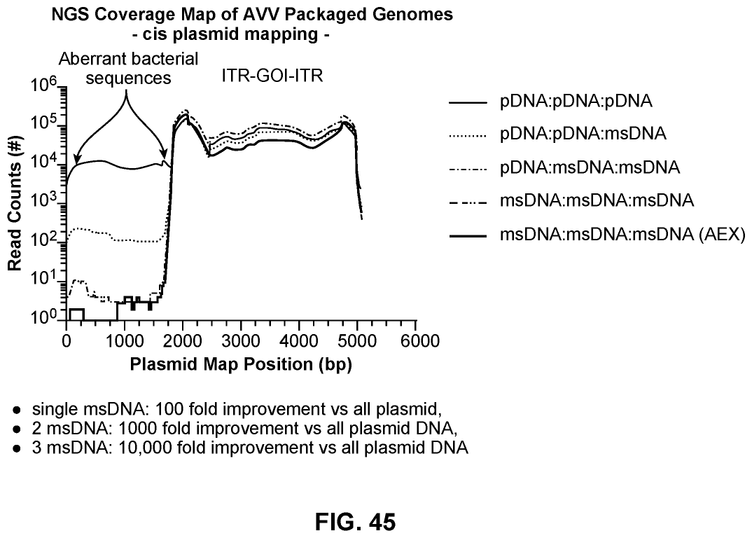

FIG. 45 shows a NGS coverage map of AAV2 packaged genomes produced with one, two, or three msDNAs in place of the three conventional plasmids.

FIG. 45 also shows that replacing a conventional plasmid encoding the GOI with msDNA resulted in 100-fold improvement of aberrant packaging versus use of all three plasmids. Replacing the conventional plasmids encoding the GOI and Rep2/Cap2 with msDNAs resulted in 1000-fold improvement. And, replacing all conventional plasmids with msDNAs resulted in a 10,000-fold improvement.

### Example 6

Production of AAV9 with msDNA

### Test 1

AAV9 was produced using the ITR-GOI-ITR msDNA described in Example 3 (i.e., ITR2Cis msDNA) or a plasmid encoding GFP and containing no SSeq as described in Example 5 (i.e., ITR2Cis no SSeq plasmid control) in combination with bacterial-sequence minimized/reduced plasmids for Rep2/Cap9 and Helper sequences, respectively.

ITR2Cis msDNA or ITR2Cis no SSeq plasmid control were mixed in a 1:1:1 molar ratio with the Rep2/Cap9 and Helper plasmids and the individual mixtures were complexed with polyethylenimine (PEI) at PEI:DNA ratios of 1.5:1, 2:1 and 2.5:1.

For combinations with the msDNA, total DNA concentrations of 1.0 μg/mL (0.156 μg/mL msDNA, 0.379 μg/mL Rep2/Cap9 plasmid, and 0.465 μg/mL Helper plasmid), 1.75 μg/mL (0.273 μg/mL msDNA, 0.663 μg/mL Rep2/Cap9 plasmid, and 0.813 μg/mL Helper plasmid), and 2.5 μg/mL (0.39 μg/mL msDNA, 0.948 μg/mL Rep2/Cap9 plasmid, and 1.162 μg/mL Helper plasmid) at each ratio were transfected into separate 150 mL cultures of HEK-293 cells to yield 9 different msDNA transfectants: (msDNA1) 1.0 μg/mL DNA and 1.5:1 PEI:DNA, (msDNA2) 1.0 g/mL DNA and 2:1 PEI:DNA, (msDNA3) 1.0 μg/mL DNA and 2.5:1 PEI:DNA, (msDNA4) 1.75 μg/mL DNA and 1.5:1 PEI:DNA, (msDNA5) 1.75 μg/mL DNA and 2:1 PEI:DNA, (msDNA6) 1.75 μg/mL DNA and 2.5:1 PEI:DNA, (msDNA7) 2.5 μg/mL DNA and 1.5:1 PEI:DNA, (msDNA8) 2.5 μg/mL DNA and 2:1 PEI:DNA, and (msDNA9) 2.5 μg/mL DNA and 2.5:1 PEI:DNA. A summary of the

ITR2Cis no SSeq plasmid control and the Rep2/Cap9 and Helper bacterial-sequence minimized/reduced plasmids were complexed with PEI at a PEI:DNA ratio of 2:1 and a total DNA concentration of 2 μg/mL (0.36 μg/mL ITR2Cis plasmid control, 0.74 μg/mL Rep2/Cap9 plasmid, and 0.9 μg/mL Helper plasmid).

A summary of the DNA concentrations and PEI:DNA ratios for the transfectants is provided below in Table 8.

TABLE 8
Summary of Transfectant DNA concentrations and PEI:DNA ratios

| | | msDNA | | | | | | | | |
|---|---|---|---|---|---|---|---|---|---|---|
| Sample | AAV PP | 1 | 2 | 3 | 4 | 5 | 6 | 7 | 8 | 9 |
| ITR DNA | 2 | 1 | 1 | 1 | 1.75 | 1.75 | 1.75 | 2.5 | 2.5 | 2.5 |
| (μg/mL) | | | | | | | | | | |
| PEI:DNA | 2:1 | 1.5:1 | 2:1 | 2.5:1 | 1.5:1 | 2:1 | 2.5:1 | 1.5:1 | 2:1 | 2.5:1 |

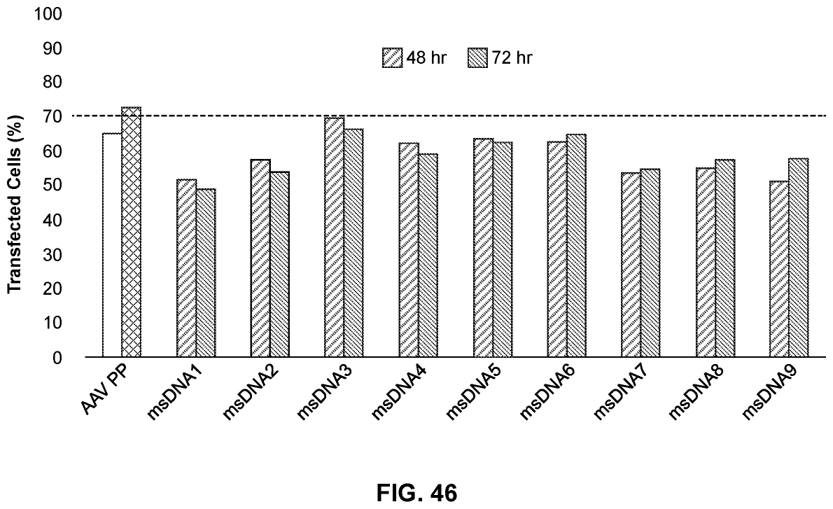

FIG. 46 shows transfection efficiencies at 48 hours and 72 hours post-transfection with the three plasmids (“AAV PP”) and the nine separate msDNA transfectants, evaluated using flow cytometry for the GFP GOI.

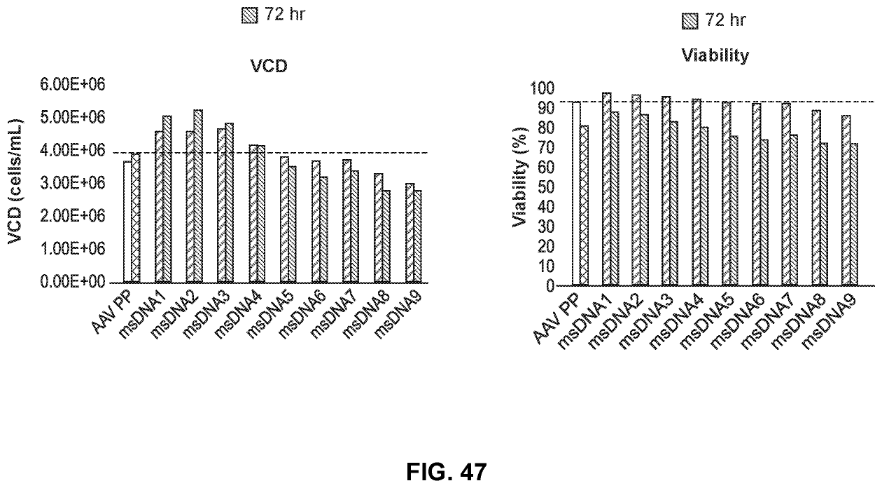

FIG. 47 shows viable cell density (VCD, viable cells/mL) and viability (% live cells) for samples at 48 hours and 72 hours post-transfection as analyzed with a Vi-CELL XR HEK293 profile.

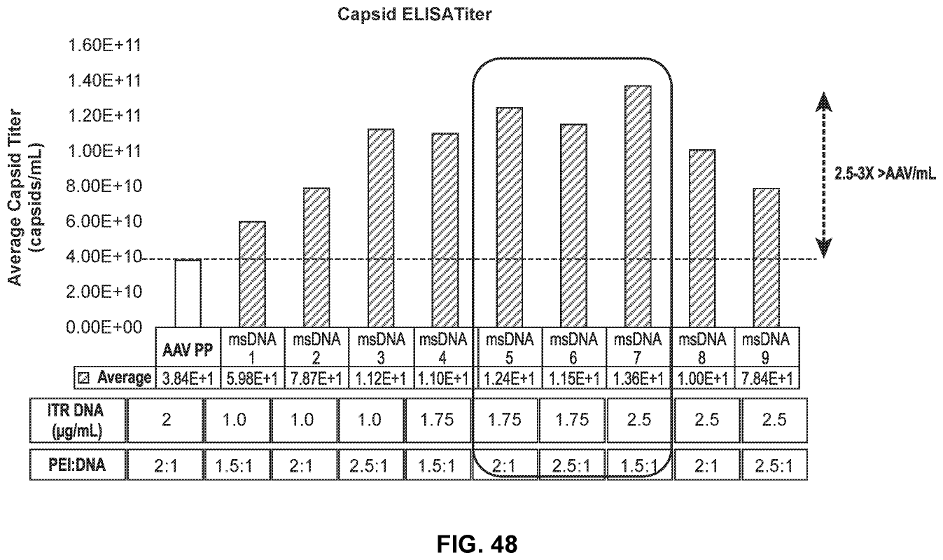

FIG. 48 shows capsid titers for the samples as determined by ELISA specific for AAV9 at 72 hours post-transfection. Capsid ELISA does not differentiate empty versus full capsids. The figure shows that 2.5-3 times higher AAV/mL were obtained by replacing the conventional plasmid carrying the GOI with msDNA.

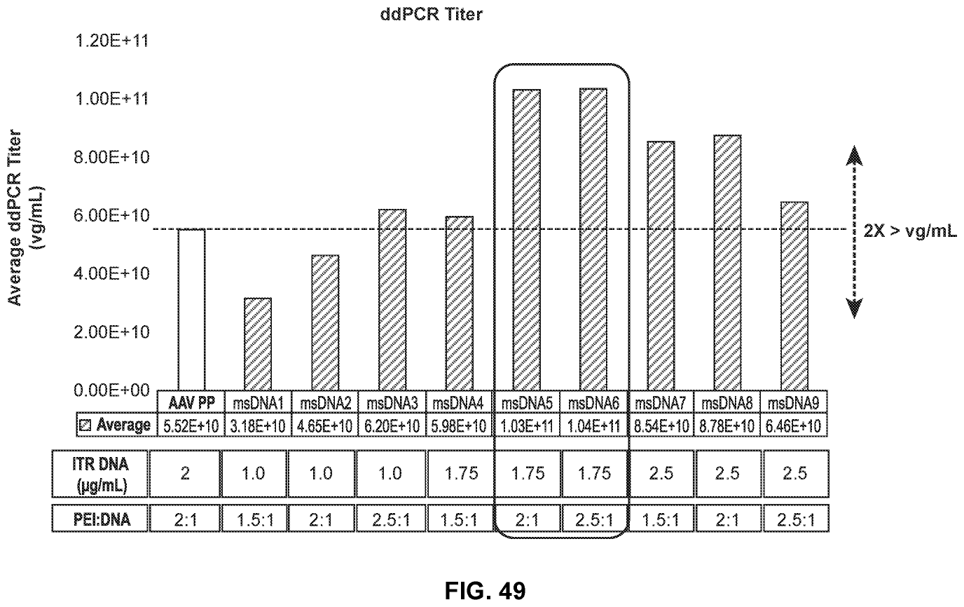

FIG. 49 shows AAV titers determined for the samples by ddPCR with primers specific to the ITR region at 72 hours post-transfection. Compared to the standard plasmid condition (2 μg/mL total DNA and 2:1 ratio of PEI:DNA), half the starting mass when substituting msDNA for the plasmid containing the GOI resulted in similar VG/mL while a similar starting mass resulted in twice the VG/mL.

### Test 2

msDNA5 (1.75 μg/mL total DNA and 2:1 ratio of PEI:DNA) and the standard plasmid condition (2 μg/mL total DNA and 2:1 ratio of PEI:DNA) were scaled to 10 L AAV cultures.

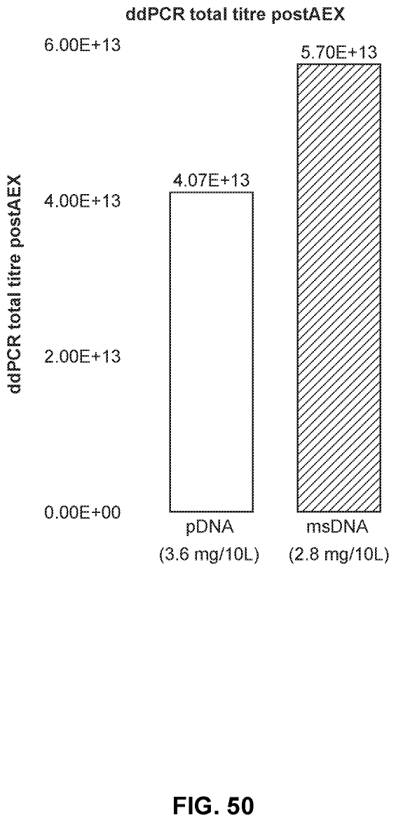

FIG. 50 shows that a similar mass of transfected DNA including msDNA in place of the conventional plasmid containing the GOI produced higher AAV titers in the 10 L cultures as determined by ddPCR following AEX chromatography.

Full capsids were also measured by mass photometry with msDNA5 resulting in 46.1% full capsids versus 40.2% for the standard plasmid condition.

SEQUENCES
AAV-2 wild-type 5′ ITR (+ strand)
SEQ ID NO: 1
ttggccactccctctctgcgcgctcgctcgctcactgaggccgggcgaccaaaggtcgcccgacg
cccgggctttgcccgggcggcctcagtgagcgagcgagcgcgcagagagggagtggccaactcca
tcactaggggttcct
AAV-2 wild-type 3′ ITR (+ strand)
SEQ ID NO: 2
aggaacccctagtgatggagttggccactccctctctgcgcgctcgctcgctcactgaggccggg
cgaccaaaggtcgcccgacgcccgggctttgcccgggcggcctcagtgagcgagcgagcgcgcag
agagggagtggccaa
AAV-2 wild-type ITR A-sequence (+ strand)
SEQ ID NO: 3
ttggccactccctctctgcgcgctcgctcgctcactgaggc
AAV-2 wild-type ITR A′-sequence (+ strand)
SEQ ID NO: 4
gcctcagtgagcgagcgagcgcgcagagagggagtggccaa
AAV-2 wild-type ITR B-sequence (+ strand)
SEQ ID NO: 5
cgggcgacca
AAV-2 wild-type ITR B′-sequence (+ strand)
SEQ ID NO: 6
aaggtcgcccg
AAV-2 wild-type ITR C-sequence (+ strand)
SEQ ID NO: 7
cgcccgggct
AAV-2 wild-type ITR C′-sequence (+ strand)
SEQ ID NO: 8
ttgcccgggcg
AAV-2 wild-type 5′ ITR D-sequence (+ strand)
SEQ ID NO: 9
ctccatcactaggggttcct
AAV-2 wild-type 3′ ITR D-sequence (+ strand)
SEQ ID NO: 10
aggaacccctagtgatggag
AAV-2 wild-type ITR A-RBS (+ strand)
SEQ ID NO: 11
gcgcgctcgctcgctc
AAV-2 wild-type ITR A′-RBS (+ strand)
SEQ ID NO: 12
gagcgagcgagcgcgc
AAV-2 wild-type ITR RBE′ (+ strand)
SEQ ID NO: 13
caaag
AAV-2 wild-type 5′ ITR TRS (+ strand)
SEQ ID NO: 14
ccaact
AAV-2 wild-type 3′ ITR TRS (+ strand)
SEQ ID NO: 15
agttgg
AAV artificial 5′ ITR (+ strand)
SEQ ID NO: 16
ctgcgcgctcgctcgctcactgaggccgcccgggcaaagcccgggcgtcgggcgacctttggtcg
cccggcctcagtgagcgagcgagcgcgcagagagggagtggccaactccatcactaggggttcct
AAV artificial 3′ ITR (+ strand)
SEQ ID NO: 17
aggaacccctagtgatggagttggccactccctctctgcgcgctcgctcgctcactgaggccggg
cgaccaaaggtcgcccgacgcccgggctttgcccgggcggcctcagtgagcgagcgagcgcgcag
ITR-CAG-GFP-ITR plasmid
SEQ ID NO: 18
cccgggaggtaccgagctcttacgcgtgctagaattaaagtaacccaatcagcacacaattgcca
ttatacgcgcgtataatggactattgtgtgctgataaacctatttcagcatactacgcgcgtagt
atgctgaaataggtgactagaagttcctatactttctagagaataggaacttcataacttcgtat
aatgtatgctatacgaagttatgggttactttaatttggttgctgactaattgagatgcatgctt
tgcatacttctgcctgctggggagcctggggactttccacacctggttgctgactaattgagatg
catgctttgcatacttctgcctgctggggagcctggggactttccacaccccaggctgcgcaact
gttgggaagggcgatcggtgcgggcctcttcgctattacgccagctgcgcgctcgctcgctcact
gaggccgcccgggcaaagcccgggcgtcgggcgacctttggtcgcccggcctcagtgagcgagcg
agcgcgcagagagggagtggccaactccatcactaggggttccttgtagttaatgattaacccgc
catgctacttatctacgtagccatgctctaggaagagtaccattgacgtcaataatgacgtatgt
tcccatagtaacgccaatagggactttccattgacgtcaatgggtggagtatttacggtaaactg
cccacttggcagtacatcaagtgtatcatatgccaagtacgccccctattgacgtcaatgacggt
aaatggcccgcctggcattatgcccagtacatgaccttatgggactttcctacttggcagtacat
ctacgtattagtcatcgctattaccatggctgggtcgacattgattattgactagttattaatag
taatcaattacggggtcattagttcatagcccatatatggagttccgcgttacataacttacggt
aaatggcccgcctggctgaccgcccaacgacccccgcccattgacgtcaataatgacgtatgttc
ccatagtaacgccaatagggactttccattgacgtcaatgggtggagtatttacggtaaactgcc
cacttggcagtacatcaagtgtatcatatgccaagtacgccccctattgacgtcaatgacggtaa
atggcccgcctggcattatgcccagtacatgaccttatgggactttcctacttggcagtacatct
acgtattagtcatcgctattaccatggtcgaggtgagccccacgttctgcttcactctccccatc
tcccccccctccccacccccaattttgtatttatttattttttaattattttgtgcagcgatggg
ggcggggggggggggggcgcgcgccaggcggggcggggcggggcgaggggcggggcggggcgagg
cggaaaggtgcggcggcagccaatcagagcggcgcgctccgaaagtttccttttatggcgaggcg
gcggcggcggcggccctataaaaagcgaagcgcgcggcgggcgggagtcgctgcgttgccttcgc
cccgtgccccgctccgcgccgcctcgcgccgcccgccccggctctgactgaccgcgttactccca
caggtgagcgggcgggacggcccttctcctccgggctgtaattagcgcttggtttaatgacggct
cgtttcttttctgtggctgcgtgaaagccttaaagggctccgggagggccctttgtgcggggggg
agcggctcggggggtgcgtgcgtgtgtgtgtgcgtggggagcgccgcgtgcggctccgcgctgcc
cggcggctgtgagcgctgcgggcgcggcgcggggctttgtgcgctccgcagtgtgcgcgagggga
gcgcggccgggggcggtgccccgcggtgcggggggggctgcgaggggaacaaaggctgcgtgcgg
ggtgtgtgcgtgggggggtgagcagggggtgtgggcgcggcggtcgggctgtaacccccccctgc
acccccctccccgagttgctgagcacggcccggcttcgggtgcggggctccgtacggggcgtggc
gcggggctcgccgtgccgggcggggggtggcggcaggtgggggtgccgggcggggcggggccgcc
tcgggccggggagggctcgggggaggggcgcggcggcccccggagcgccggcggctgtcgaggcg
cggcgagccgcagccattgccttttatggtaatcgtgcgagagggcgcagggacttcctttgtcc
caaatctgtgcggagccgaaatctgggaggcgccgccgcaccccctctagcgggcgcggggcgaa
gcggtgcggcgccggcaggaaggaaatgggcggggagggccttcgtgcgtcgccgcgccgccgtc
cccttctccctctccagcctcggggctgtccgcggggggacggctgccttcgggggggacggggc
agggcggggttcggcttctggcgtgtgaccggcggctctagagcctctgctaaccatgttcatgc
cttcttctttttcctacagctcctgggcaacgtgctggttattgtgctgtctcatcattttggca
aagaattgattaattcgagcgaacgcgtcgccaccatggtgagcaagggcgaggagctgttcacc
ggggtggtgcccatcctggtcgagctggacggcgacgtaaacggccacaagttcagcgtgtccgg
cgagggcgagggcgatgccacctacggcaagctgaccctgaagttcatctgcaccaccggcaagc
tgcccgtgccctggcccaccctcgtgaccaccctgacctacggcgtgcagtgcttcagccgctac
cccgaccacatgaagcagcacgacttcttcaagtccgccatgcccgaaggctacgtccaggagcg
caccatcttcttcaaggacgacggcaactacaagacccgcgccgaggtgaagttcgagggcgaca
ccctggtgaaccgcatcgagctgaagggcatcgacttcaaggaggacggcaacatcctggggcac
aagctggagtacaactacaacagccacaacgtctatatcatggccgacaagcagaagaacggcat
caaggtgaacttcaagatccgccacaacatcgaggacggcagcgtgcagctcgccgaccactacc
agcagaacacccccatcggcgacggccccgtgctgctgcccgacaaccactacctgagcacccag
tccgccctgagcaaagaccccaacgagaagcgcgatcacatggtcctgctggagttcgtgaccgc
cgccgggatcactctcggcatggacgagctgtacaagtaagcggccgcactcctcaggtgcaggc
tgcctatcagaaggtggtggctggtgtggccaatgccctggctcacaaaattcgatatcaagctt
atcgataatcaacctctggattacaaaatttgtgaaagattgactggtattcttaactatgttgc
tccttttacgctatgtggatacgctgctttaatgcctttgtatcatgctattgcttcccgtatgg
ctttcattttctcctccttgtataaatcctggttgctgtctctttatgaggagttgtggcccgtt
gtcaggcaacgtggcgtggtgtgcactgtgtttgctgacgcaacccccactggttggggcattgc
caccacctgtcagctcctttccgggactttcgctttccccctccctattgccacggcggaactca
tcgccgcctgccttgcccgctgctggacaggggctcggctgttgggcactgacaattccgtggtg
ttgtcggggaaatcatcgtcctttccttggctgctcgcctgtgttgccacctggattctgcgcgg
gacgtccttctgctacgtcccttcggccctcaatccagcggaccttccttcccgcggcctgctgc
cggctctgcggcctcttccgcgtcttcgccttcgccctcagacgagtcggatctccctttgggcc
gcctccccgcatcgataccgtcgacccgggcggccgcttcgagcagacatgataagatacattga
tgagtttggacaaaccacaactagaatgcagtgaaaaaaatgctttatttgtgaaatttgtgatg
ctattgctttatttgtaaccattataagctgcaataaacaagttaacaacaacaattgcattcat
tttatgtttcaggttcagggggagatgtgggaggttttttaaagcaagtaaaacctctacaaatg
tggtaaaatcgataaggatcttcctagagcatggctacgtagataagtagcatggcgggttaatc
attaactacaaggaacccctagtgatggagttggccactccctctctgcgcgctcgctcgctcac
tgaggccgggcgaccaaaggtcgcccgacgcccgggctttgcccgggcggcctcagtgagcgagc
gagcgcgcagctgcattaatgaatcggccaacgcgcggggagaggcggtttgcgtattgggcgct
taccactgagatctttttccctctgccaaaaattatggggacatcatgaagccccttgagcatct
gacttctggctaataaaggaaatttattttcattgcaatagtgtgttggaattttttgtgtctct
cactcggaaggacatggtgtggaaagtccccaggctccccagcaggcagaagtatgcaaagcatg
catctcaattagtcagcaaccaaattaaagtaacccataacttcgtatagcatacattatacgaa
gttatgaagttcctattctctagaaagtataggaacttctagtcacctatttcagcatactacgc
gcgtagtatgctgaaataggtttatcagcacacaatagtccattatacgcgcgtataatggcaat
tgtgtgctgattgggttactttaattggtgtggaaagtccccaggctccccagcaggcagaagta
tgcaaagcatgcatctcaattagtcagcaaccatggatccgtcgaccgatgcccttgagagcctt
caacccagtcagctccttccggtgggcgcggggcatgactatcgtcgccgcacttatgactgtct
tctttatcatgcaactcgtaggacaggtgccggcagcgctcttccgcttcctcgctcactgactc
gctgcgctcggtcgttcggctgcggcgagcggtatcagctcactcaaaggcggtaatacggttat
ccacagaatcaggggataacgcaggaaagaacatgtgagcaaaaggccagcaaaaggccaggaac
cgtaaaaaggccgcgttgctggcgtttttccataggctccgcccccctgacgagcatcacaaaaa
tcgacgctcaagtcagaggtggcgaaacccgacaggactataaagataccaggcgtttccccctg
gaagctccctcgtgcgctctcctgttccgaccctgccgcttaccggatacctgtccgcctttctc
ccttcgggaagcgtggcgctttctcatagctcacgctgtaggtatctcagttcggtgtaggtcgt
tcgctccaagctgggctgtgtgcacgaaccccccgttcagcccgaccgctgcgccttatccggta
actatcgtcttgagtccaacccggtaagacacgacttatcgccactggcagcagccactggtaac
aggattagcagagcgaggtatgtaggcggtgctacagagttcttgaagtggtggcctaactacgg
ctacactagaagaacagtatttggtatctgcgctctgctgaagccagttaccttccagggaaaaa
gagttggtagctcttgatccggcaaacaaaccaccgctggtagcggtggtttttttgtttgcaag
cagcagattacgcgcagaaaaaaaggatctcaagaagatcctttgatcttttctacggggtctga
cgctcagtggaacgaaaactcacgttaagggattttggtcatgagattatcaaaaaggatcttca
cctagatccttttaaattaaaaatgaagttttaaatcaatctaaagtatatatgagtaaacttgg
tctgacagttaccaatgcttaatcagtgaggcacctatctcagcgatctgtctatttcgttcatc
catagttgcctgactccccgtcgtgtagataactacgatacgggagggcttaccatctggcccca
gtgctgcaatgataccgcgagacccacgctcaccggctccagatttatcagcaataaaccagcca
gccggaagggccgagcgcagaagtggtcctgcaactttatccgcctccatccagtctattaattg
ttgccgggaagctagagtaagtagttcgccagttaatagtttgcgcaacgttgttgccattgcta
caggcatcgtggtgtcacgctcgtcgtttggtatggcttcattcagctccggttcccaacgatca
aggcgagttacatgatcccccatgttgtgcaaaaaagcggttagctccttcggtcctccgatcgt
tgtcagaagtaagttggccgcagtgttatcactcatggttatggcagcactgcataattctctta
ctgtcatgccatccgtaagatgcttttctgtgactggtgagtactcaaccaagtcattctgagaa
tagtgtatgcggcgaccgagttgctcttgcccggcgtcaatacgggataataccgcgccacatag
cagaactttaaaagtgctcatcattggaaaacgttcttcggggcgaaaactctcaaggatcttac
cgctgttgagatccagttcgatgtaacccactcgtgcacccaactgatcttcagcatcttttact
ttcaccagcgtttctgggtgagcaaaaacaggaaggcaaaatgccgcaaaaaagggaataagggc
gacacggaaatgttgaatactcatactcttcctttttcaatattattgaagcatttatcagggtt
attgtctcatgagcggatacatatttgaatgtatttagaaaaataaacaaataggggttccgcgc
acatttccccgaaaagtgccacctgacgcgccctgtagcggcgcattaagcgcggcgggtgtggt
ggttacgcgcagcgtgaccgctacacttgccagcgccctagcgcccgctcctttcgctttcttcc
cttcctttctcgccacgttcgccggctttccccgtcaagctctaaatcgggggctccctttaggg
ttccgatttagtgctttacggcacctcgaccccaaaaaacttgattagggtgatggttcacgtag
tgggccatcgccctgatagacggtttttcgccctttgacgttggagtccacgttctttaatagtg
gactcttgttccaaactggaacaacactcaaccctatctcggtctattcttttgatttataaggg
attttgccgatttcggcctattggttaaaaaatgagctgatttaacaaaaatttaacgcgaattt
taacaaaatattaacgcttacaatttgccattcgccattcaggctgcgcaactgttgggaagggc
gatcggtgcgggcctcttcgctattacgccagcccaagctaccatgataagtaagtaatattaag
gtacgtggaggttttacttgctttaaaaaacctcccacacctccccctgaacctgaaacataaaa
tgaatgcaattgttgttgttaacttgtttattgcagcttataatggttacaaataaagcaatagc
atcacaaatttcacaaataaagcatttttttcactgcattctagttgtggtttgtccaaactcat
caatgtatcttatggtactgtaactgagctaacataa
ITR-CAG-GFP-ITR msDNA
SEQ ID NO: 19
tacgcgcgtagtatgctgaaataggtgactagaagttcctatactttctagagaataggaacttc
ataacttcgtataatgtatgctatacgaagttatgggttactttaatttggttgctgactaattg
agatgcatgctttgcatacttctgcctgctggggagcctggggactttccacacctggttgctga
ctaattgagatgcatgctttgcatacttctgcctgctggggagcctggggactttccacacccca
ggctgcgcaactgttgggaagggcgatcggtgcgggcctcttcgctattacgccagctgcgcgct
cgctcgctcactgaggccgcccgggcaaagcccgggcgtcgggcgacctttggtcgcccggcctc
agtgagcgagcgagcgcgcagagagggagtggccaactccatcactaggggttccttgtagttaa
tgattaacccgccatgctacttatctacgtagccatgctctaggaagagtaccattgacgtcaat
aatgacgtatgttcccatagtaacgccaatagggactttccattgacgtcaatgggtggagtatt
tacggtaaactgcccacttggcagtacatcaagtgtatcatatgccaagtacgccccctattgac
gtcaatgacggtaaatggcccgcctggcattatgcccagtacatgaccttatgggactttcctac
ttggcagtacatctacgtattagtcatcgctattaccatggctgggtcgacattgattattgact
agttattaatagtaatcaattacggggtcattagttcatagcccatatatggagttccgcgttac
ataacttacggtaaatggcccgcctggctgaccgcccaacgacccccgcccattgacgtcaataa
tgacgtatgttcccatagtaacgccaatagggactttccattgacgtcaatgggtggagtattta
cggtaaactgcccacttggcagtacatcaagtgtatcatatgccaagtacgccccctattgacgt
caatgacggtaaatggcccgcctggcattatgcccagtacatgaccttatgggactttcctactt
ggcagtacatctacgtattagtcatcgctattaccatggtcgaggtgagccccacgttctgcttc
actctccccatctcccccccctccccacccccaattttgtatttatttattttttaattattttg
tgcagcgatgggggcggggggggggggggcgcgcgccaggcggggcggggcggggcgaggggcgg
ggcggggcgaggcggaaaggtgcggcggcagccaatcagagcggcgcgctccgaaagtttccttt
tatggcgaggcggcggcggcggcggccctataaaaagcgaagcgcgcggcgggcgggagtcgctg
cgttgccttcgccccgtgccccgctccgcgccgcctcgcgccgcccgccccggctctgactgacc
gcgttactcccacaggtgagcgggcgggacggcccttctcctccgggctgtaattagcgcttggt
ttaatgacggctcgtttcttttctgtggctgcgtgaaagccttaaagggctccgggagggccctt
tgtgcgggggggagcggctcggggggtgcgtgcgtgtgtgtgtgcgtggggagcgccgcgtgcgg
ctccgcgctgcccggcggctgtgagcgctgcgggcgcggcgcggggctttgtgcgctccgcagtg
tgcgcgaggggagcgcggccgggggcggtgccccgcggtgcggggggggctgcgaggggaacaaa
ggctgcgtgcggggtgtgtgcgtgggggggtgagcagggggtgtgggcgcggcggtcgggctgta
acccccccctgcacccccctccccgagttgctgagcacggcccggcttcgggtgcggggctccgt
acggggcgtggcgcggggctcgccgtgccgggcggggggtggcggcaggtgggggtgccgggcgg
ggcggggccgcctcgggccggggagggctcgggggaggggcgcggcggcccccggagcgccggcg
gctgtcgaggcgcggcgagccgcagccattgccttttatggtaatcgtgcgagagggcgcaggga
cttcctttgtcccaaatctgtgcggagccgaaatctgggaggcgccgccgcaccccctctagcgg
gcgcggggcgaagcggtgcggcgccggcaggaaggaaatgggcggggagggccttcgtgcgtcgc
cgcgccgccgtccccttctccctctccagcctcggggctgtccgcggggggacggctgccttcgg
gggggacggggcagggcggggttcggcttctggcgtgtgaccggcggctctagagcctctgctaa
ccatgttcatgccttcttctttttcctacagctcctgggcaacgtgctggttattgtgctgtctc
atcattttggcaaagaattgattaattcgagcgaacgcgtcgccaccatggtgagcaagggcgag
gagctgttcaccggggtggtgcccatcctggtcgagctggacggcgacgtaaacggccacaagtt
cagcgtgtccggcgagggcgagggcgatgccacctacggcaagctgaccctgaagttcatctgca
ccaccggcaagctgcccgtgccctggcccaccctcgtgaccaccctgacctacggcgtgcagtgc
ttcagccgctaccccgaccacatgaagcagcacgacttcttcaagtccgccatgcccgaaggcta
cgtccaggagcgcaccatcttcttcaaggacgacggcaactacaagacccgcgccgaggtgaagt
tcgagggcgacaccctggtgaaccgcatcgagctgaagggcatcgacttcaaggaggacggcaac
atcctggggcacaagctggagtacaactacaacagccacaacgtctatatcatggccgacaagca
gaagaacggcatcaaggtgaacttcaagatccgccacaacatcgaggacggcagcgtgcagctcg
ccgaccactaccagcagaacacccccatcggcgacggccccgtgctgctgcccgacaaccactac
ctgagcacccagtccgccctgagcaaagaccccaacgagaagcgcgatcacatggtcctgctgga
gttcgtgaccgccgccgggatcactctcggcatggacgagctgtacaagtaagcggccgcactcc
tcaggtgcaggctgcctatcagaaggtggtggctggtgtggccaatgccctggctcacaaaAttc
gatatcaagcttatcgataatcaacctctggattacaaaatttgtgaaagattgactggtattct
taactatgttgctccttttacgctatgtggatacgctgctttaatgcctttgtatcatgctattg
cttcccgtatggctttcattttctcctccttgtataaatcctggttgctgtctctttatgaggag
ttgtggcccgttgtcaggcaacgtggcgtggtgtgcactgtgtttgctgacgcaacccccactgg
ttggggcattgccaccacctgtcagctcctttccgggactttcgctttccccctccctattgcca
cggcggaactcatcgccgcctgccttgcccgctgctggacaggggctcggctgttgggcactgac
aattccgtggtgttgtcggggaaatcatcgtcctttccttggctgctcgcctgtgttgccacctg
gattctgcgcgggacgtccttctgctacgtcccttcggccctcaatccagcggaccttccttccc
gcggcctgctgccggctctgcggcctcttccgcgtcttcgccttcgccctcagacgagtcggatc
tccctttgggccgcctccccgcatcgataccgtcgacccgggcggccgcttcgagcagacatgat
aagatacattgatgagtttggacaaaccacaactagaatgcagtgaaaaaaatgctttatttgtg
aaatttgtgatgctattgctttatttgtaaccattataagctgcaataaacaagttaacaacaac
aattgcattcattttatgtttcaggttcagggggagatgtgggaggttttttaaagcaagtaaaa
cctctacaaatgtggtaaaatcgataaggatcttcctagagcatggctacgtagataagtagcat
ggcgggttaatcattaactacaaggaacccctagtgatggagttggccactccctctctgcgcgc
tcgctcgctcactgaggccgggcgaccaaaggtcgcccgacgcccgggctttgcccgggcggcct
cagtgagcgagcgagcgcgcagctgcattaatgaatcggccaacgcgcggggagaggcggtttgc
gtattgggcgcttaccactgagatctttttccctctgccaaaaattatggggacatcatgaagcc
ccttgagcatctgacttctggctaataaaggaaatttattttcattgcaatagtgtgttggaatt
ttttgtgtctctcactcggaaggacatggtgtggaaagtccccaggctccccagcaggcagaagt
atgcaaagcatgcatctcaattagtcagcaaccaaattaaagtaacccataacttcgtatagcat
acattatacgaagttatgaagttcctattctctagaaagtataggaacttctagtcacctatttc
agcatactacgcgcgta
CAG-GFP-ITR msDNA
SEQ ID NO: 20
tacgcgcgtagtatgctgaaataggtgactagaagttcctatactttctagagaataggaacttc
ataacttcgtataatgtatgctatacgaagttatgggttactttaatttggttgctgactaattg
agatgcatgctttgcatacttctgcctgctggggagcctggggactttccacacctggttgctga
ctaattgagatgcatgctttgcatacttctgcctgctggggagcctggggactttccacaccttt
ccagtcgacattgattattgactagttattaatagtaatcaattacggggtcattagttcatagc
ccatatatggagttccgcgttacataacttacggtaaatggcccgcctggctgaccgcccaacga
cccccgcccattgacgtcaataatgacgtatgttcccatagtaacgccaatagggactttccatt
gacgtcaatgggtggagtatttacggtaaactgcccacttggcagtacatcaagtgtatcatatg
ccaagtacgccccctattgacgtcaatgacggtaaatggcccgcctggcattatgcccagtacat
gaccttatgggactttcctacttggcagtacatctacgtattagtcatcgctattaccatggtcg
aggtgagccccacgttctgcttcactctccccatctcccccccctccccacccccaattttgtat
ttatttattttttaattattttgtgcagcgatgggggcggggggggggggggcgcgcgccaggcg
gggcggggcggggcgaggggcggggcggggcgaggcggaaaggtgcggcggcagccaatcagagc
ggcgcgctccgaaagtttccttttatggcgaggcggcggcggcggcggccctataaaaagcgaag
cgcgcggcgggcgggagtcgctgcgttgccttcgccccgtgccccgctccgcgccgcctcgcgcc
gcccgccccggctctgactgaccgcgttactcccacaggtgagcgggcgggacggcccttctcct
ccgggctgtaattagcgcttggtttaatgacggctcgtttcttttctgtggctgcgtgaaagcct
taaagggctccgggagggccctttgtgcgggggggagcggctcggggggtgcgtgcgtgtgtgtg
tgcgtggggagcgccgcgtgcggctccgcgctgcccggcggctgtgagcgctgcgggcgcggcgc
ggggctttgtgcgctccgcagtgtgcgcgaggggagcgcggccgggggcggtgccccgcggtgcg
gggggggctgcgaggggaacaaaggctgcgtgcggggtgtgtgcgtgggggggtgagcagggggt
gtgggcgcggcggtcgggctgtaacccccccctgcacccccctccccgagttgctgagcacggcc
cggcttcgggtgcggggctccgtacggggcgtggcgcggggctcgccgtgccgggcggggggtgg
cggcaggtgggggtgccgggcggggcggggccgcctcgggccggggagggctcgggggaggggcg
cggcggcccccggagcgccggcggctgtcgaggcgcggcgagccgcagccattgccttttatggt
aatcgtgcgagagggcgcagggacttcctttgtcccaaatctgtgcggagccgaaatctgggagg
cgccgccgcaccccctctagcgggcgcggggcgaagcggtgcggcgccggcaggaaggaaatggg
cggggagggccttcgtgcgtcgccgcgccgccgtccccttctccctctccagcctcggggctgtc
cgcggggggacggctgccttcgggggggacggggcagggcggggttcggcttctggcgtgtgacc
ggcggctctagagcctctgctaaccatgttcatgccttcttctttttcctacagctcctgggcaa
cgtgctggttattgtgctgtctcatcattttggcaaagaattgattaattcgagcgaacgcgtcg
ccaccatggtgagcaagggcgaggagctgttcaccggggtggtgcccatcctggtcgagctggac
ggcgacgtaaacggccacaagttcagcgtgtccggcgagggcgagggcgatgccacctacggcaa
gctgaccctgaagttcatctgcaccaccggcaagctgcccgtgccctggcccaccctcgtgacca
ccctgacctacggcgtgcagtgcttcagccgctaccccgaccacatgaagcagcacgacttcttc
aagtccgccatgcccgaaggctacgtccaggagcgcaccatcttcttcaaggacgacggcaacta
caagacccgcgccgaggtgaagttcgagggcgacaccctggtgaaccgcatcgagctgaagggca
tcgacttcaaggaggacggcaacatcctggggcacaagctggagtacaactacaacagccacaac
gtctatatcatggccgacaagcagaagaacggcatcaaggtgaacttcaagatccgccacaacat
cgaggacggcagcgtgcagctcgccgaccactaccagcagaacacccccatcggcgacggccccg
tgctgctgcccgacaaccactacctgagcacccagtccgccctgagcaaagaccccaacgagaag
cgcgatcacatggtcctgctggagttcgtgaccgccgccgggatcactctcggcatggacgagct
gtacaagtaagcggccgcactcctcaggtgcaggctgcctatcagaaggtggtggctggtgtggc
caatgccctggctcacaaaAttcgatatcaagcttatcgataatcaacctctggattacaaaatt
tgtgaaagattgactggtattcttaactatgttgctccttttacgctatgtggatacgctgcttt
aatgcctttgtatcatgctattgcttcccgtatggctttcattttctcctccttgtataaatcct
ggttgctgtctctttatgaggagttgtggcccgttgtcaggcaacgtggcgtggtgtgcactgtg
tttgctgacgcaacccccactggttggggcattgccaccacctgtcagctcctttccgggacttt
cgctttccccctccctattgccacggcggaactcatcgccgcctgccttgcccgctgctggacag
gggctcggctgttgggcactgacaattccgtggtgttgtcggggaaatcatcgtcctttccttgg
ctgctcgcctgtgttgccacctggattctgcgcgggacgtccttctgctacgtcccttcggccct
caatccagcggaccttccttcccgcggcctgctgccggctctgcggcctcttccgcgtcttcgcc
ttcgccctcagacgagtcggatctccctttgggccgcctccccgcatcgataccgtcgacccggg
cggccgcttcgagcagacatgataagatacattgatgagtttggacaaaccacaactagaatgca
gtgaaaaaaatgctttatttgtgaaatttgtgatgctattgctttatttgtaaccattataagct
gcaataaacaagttaacaacaacaattgcattcattttatgtttcaggttcagggggagatgtgg
gaggttttttaaagcaagtaaaacctctacaaatgtggtaaaatcgataaggatcttcctagagc
atggctacgtagataagtagcatggcgggttaatcattaactacaaggaacccctagtgatggag
ttggccactccctctctgcgcgctcgctcgctcactgaggccgggcgaccaaaggtcgcccgacg
cccgggctttgcccgggcggcctcagtgagcgagcgagcgcgcagctgcattaatgaatcggcca
acgcgcggggagaggcggtttgcgtattgggcgcttaccactgagatctttttccctctgccaaa
aattatggggacatcatgaagccccttgagcatctgacttctggctaataaaggaaatttatttt
cattgcaatagtgtgttggaattttttgtgtctctcactcggaaggacatggtgtggaaagtccc
caggctccccagcaggcagaagtatgcaaagcatgcatctcaattagtcagcaaccaaattaaag
taacccataacttcgtatagcatacattatacgaagttatgaagttcctattctctagaaagtat
aggaacttctagtcacctatttcagcatactacgcgcgta
AAV-2 rep
SEQ ID NO: 21
atgccggggttttacgagattgtgattaaggtccccagcgaccttgacgagcatctgcccggcat
ttctgacagctttgtgaactgggtggccgagaaggaatgggagttgccgccagattctgacatgg
atctgaatctgattgagcaggcacccctgaccgtggccgagaagctgcagcgcgactttctgacg
gaatggcgccgtgtgagtaaggccccggaggcccttttctttgtgcaatttgagaagggagagag
ctacttccacatgcacgtgctcgtggaaaccaccggggtgaaatccatggttttgggacgtttcc
tgagtcagattcgcgaaaaactgattcagagaatttaccgcgggatcgagccgactttgccaaac
tggttcgcggtcacaaagaccagaaatggcgccggaggcgggaacaaggtggtggatgagtgcta
catccccaattacttgctccccaaaacccagcctgagctccagtgggcgtggactaatatggaac
agtatttaagcgcctgtttgaatctcacggagcgtaaacggttggtggcgcagcatctgacgcac
gtgtcgcagacgcaggagcagaacaaagagaatcagaatcccaattctgatgcgccggtgatcag
atcaaaaacttcagccaggtacatggagctggtcgggtggctcgtggacaaggggattacctcgg
agaagcagtggatccaggaggaccaggcctcatacatctccttcaatgcggcctccaactcgcgg
tcccaaatcaaggctgccttggacaatgcgggaaagattatgagcctgactaaaaccgcccccga
ctacctggtgggccagcagcccgtggaggacatttccagcaatcggatttataaaattttggaac
taaacgggtacgatccccaatatgcggcttccgtctttctgggatgggccacgaaaaagttcggc
aagaggaacaccatctggctgtttgggcctgcaactaccgggaagaccaacatcgcggaggccat
agcccacactgtgcccttctacgggtgcgtaaactggaccaatgagaactttcccttcaacgact
gtgtcgacaagatggtgatctggtgggaggaggggaagatgaccgccaaggtcgtggagtcggcc
aaagccattctcggaggaagcaaggtgcgcgtggaccagaaatgcaagtcctcggcccagataga
cccgactcccgtgatcgtcacctccaacaccaacatgtgcgccgtgattgacgggaactcaacga
ccttcgaacaccagcagccgttgcaagaccggatgttcaaatttgaactcacccgccgtctggat
catgactttgggaaggtcaccaagcaggaagtcaaagactttttccggtgggcaaaggatcacgt
ggttgaggtggagcatgaattctacgtcaaaaagggtggagccaagaaaagacccgcccccagtg
acgcagatataagtgagcccaaacgggtgcgcgagtcagttgcgcagccatcgacgtcagacgcg
gaagcttcgatcaactacgcagacaggtaccaaaacaaatgttctcgtcacgtgggcatgaatct
gatgctgtttccctgcagacaatgcgagagaatgaatcagaattcaaatatctgcttcactcacg
gacagaaagactgtttagagtgctttcccgtgtcagaatctcaacccgtttctgtcgtcaaaaag
gcgtatcagaaactgtgctacattcatcatatcatgggaaaggtgccagacgcttgcactgcctg
cgatctggtcaatgtggatttggatgactgcatctttgaacaataa
AAV-2 cap
SEQ ID NO: 22
atggctgccgatggttatcttccagattggctcgaggacactctctctgaaggaataagacagtg
gtggaagctcaaacctggcccaccaccaccaaagcccgcagagcggcataaggacgacagcaggg
gtcttgtgcttcctgggtacaagtacctcggacccttcaacggactcgacaagggagagccggtc
aacgaggcagacgccgcggccctcgagcacgacaaagcctacgaccggcagctcgacagcggaga
caacccgtacctcaagtacaaccacgccgacgcggagtttcaggagcgccttaaagaagatacgt
cttttgggggcaacctcggacgagcagtcttccaggcgaaaaagagggttcttgaacctctgggc
ctggttgaggaacctgttaagacggctccgggaaaaaagaggccggtagagcactctcctgtgga
gccagactcctcctcgggaaccggaaaggcgggccagcagcctgcaagaaaaagattgaattttg
gtcagactggagacgcagactcagtacctgacccccagcctctcggacagccaccagcagccccc
tctggtctgggaactaatacgatggctacaggcagtggcgcaccaatggcagacaataacgaggg
cgccgacggagtgggtaattcctcgggaaattggcattgcgattccacatggatgggcgacagag
tcatcaccaccagcacccgaacctgggccctgcccacctacaacaaccacctctacaaacaaatt
tccagccaatcaggagcctcgaacgacaatcactactttggctacagcaccccttgggggtattt
tgacttcaacagattccactgccacttttcaccacgtgactggcaaagactcatcaacaacaact
ggggattccgacccaagagactcaacttcaagctctttaacattcaagtcaaagaggtcacgcag
aatgacggtacgacgacgattgccaataaccttaccagcacggttcaggtgtttactgactcgga
gtaccagctcccgtacgtcctcggctcggcgcatcaaggatgcctcccgccgttcccagcagacg
tcttcatggtgccacagtatggatacctcaccctgaacaacgggagtcaggcagtaggacgctct
tcattttactgcctggagtactttccttctcagatgctgcgtaccggaaacaactttaccttcag
ctacacttttgaggacgttcctttccacagcagctacgctcacagccagagtctggaccgtctca
tgaatcctctcatcgaccagtacctgtattacttgagcagaacaaacactccaagtggaaccacc
acgcagtcaaggcttcagttttctcaggccggagcgagtgacattcgggaccagtctaggaactg
gcttcctggaccctgttaccgccagcagcgagtatcaaagacatctgcggataacaacaacagtg
aatactcgtggactggagctaccaagtaccacctcaatggcagagactctctggtgaatccgggc
ccggccatggcaagccacaaggacgatgaagaaaagttttttcctcagagcggggttctcatctt
tgggaagcaaggctcagagaaaacaaatgtggacattgaaaaggtcatgattacagacgaagagg
aaatcaggacaaccaatcccgtggctacggagcagtatggttctgtatctaccaacctccagaga
ggcaacagacaagcagctaccgcagatgtcaacacacaaggcgttcttccaggcatggtctggca
ggacagagatgtgtaccttcaggggcccatctgggcaaagattccacacacggacggacattttc
acccctctcccctcatgggtggattcggacttaaacaccctcctccacagattctcatcaagaac
accccggtacctgcgaatccttcgaccaccttcagtgcggcaaagtttgcttccttcatcacaca
gtactccacgggacaggtcagcgtggagatcgagtgggagctgcagaaggaaaacagcaaacgct
ggaatcccgaaattcagtacacttccaactacaacaagtctgttaatgtggactttactgtggac
actaatggcgtgtattcagagcctcgccccattggcaccagatacctgactcgtaatctgtaa
PGL2-SS-CMV-Rep-Cap-SS plasmid
SEQ ID NO: 23
cccgggaggtaccgagctcttacgcgtgctagaattaaagtaacccaatcagcacacaattgcca
ttatacgcgcgtataatggactattgtgtgctgataaacctatttcagcatactacgcgcgtagt
atgctgaaataggtgactagaagttcctatactttctagagaataggaacttcataacttcgtat
aatgtatgctatacgaagttatgggttactttaatttggttgctgactaattgagatgcatgctt
tgcatacttctgcctgctggggagcctggggactttccacacctggttgctgactaattgagatg
catgctttgcatacttctgcctgctggggagcctggggactttccacacccctgattctgtggat
aaccgtattaccgccatgcattagttattaatagtaatcaattacggggtcattagttcatagcc
catatatggagttccgcgttacataacttacggtaaatggcccgcctggctgaccgcccaacgac
ccccgcccattgacgtcaataatgacgtatgttcccatagtaacgccaatagggactttccattg
acgtcaatgggtggagtatttacggtaaactgcccacttggcagtacatcaagtgtatcatatgc
caagtacgccccctattgacgtcaatgacggtaaatggcccgcctggcattatgcccagtacatg
accttatgggactttcctacttggcagtacatctacgtattagtcatcgctattaccatggtgat
gcggttttggcagtacatcaatgggcgtggatagcggtttgactcacggggatttccaagtctcc
accccattgacgtcaatgggagtttgttttggcaccaaaatcaacgggactttccaaaatgtcgt
aacaactccgccccattgacgcaaatgggcggtaggcgtgtacggtgggaggtctatataagcag
agctggtttagtgaaccgtcagatccgtttgaacgcgcagccgccatgccggggttttacgagat
tgtgattaaggtccccagcgaccttgacgagcatctgcccggcatttctgacagctttgtgaact
gggtggccgagaaggaatgggagttgccgccagattctgacatggatctgaatctgattgagcag
gcacccctgaccgtggccgagaagctgcagcgcgactttctgacggaatggcgccgtgtgagtaa
ggccccggaggcccttttctttgtgcaatttgagaagggagagagctacttccacatgcacgtgc
tcgtggaaaccaccggggtgaaatccatggttttgggacgtttcctgagtcagattcgcgaaaaa
ctgattcagagaatttaccgcgggatcgagccgactttgccaaactggttcgcggtcacaaagac
cagaaatggcgccggaggcgggaacaaggtggtggatgagtgctacatccccaattacttgctcc
ccaaaacccagcctgagctccagtgggcgtggactaatatggaacagtatttaagcgcctgtttg
aatctcacggagcgtaaacggttggtggcgcagcatctgacgcacgtgtcgcagacgcaggagca
gaacaaagagaatcagaatcccaattctgatgcgccggtgatcagatcaaaaacttcagccaggt
acatggagctggtcgggtggctcgtggacaaggggattacctcggagaagcagtggatccaggag
gaccaggcctcatacatctccttcaatgcggcctccaactcgcggtcccaaatcaaggctgcctt
ggacaatgcgggaaagattatgagcctgactaaaaccgcccccgactacctggtgggccagcagc
ccgtggaggacatttccagcaatcggatttataaaattttggaactaaacgggtacgatccccaa
tatgcggcttccgtctttctgggatgggccacgaaaaagttcggcaagaggaacaccatctggct
gtttgggcctgcaactaccgggaagaccaacatcgcggaggccatagcccacactgtgcccttct
acgggtgcgtaaactggaccaatgagaactttcccttcaacgactgtgtcgacaagatggtgatc
tggtgggaggaggggaagatgaccgccaaggtcgtggagtcggccaaagccattctcggaggaag
caaggtgcgcgtggaccagaaatgcaagtcctcggcccagatagacccgactcccgtgatcgtca
cctccaacaccaacatgtgcgccgtgattgacgggaactcaacgaccttcgaacaccagcagccg
ttgcaagaccggatgttcaaatttgaactcacccgccgtctggatcatgactttgggaaggtcac
caagcaggaagtcaaagactttttccggtgggcaaaggatcacgtggttgaggtggagcatgaat
tctacgtcaaaaagggtggagccaagaaaagacccgcccccagtgacgcagatataagtgagccc
aaacgggtgcgcgagtcagttgcgcagccatcgacgtcagacgcggaagcttcgatcaactacgc
agacaggtaccaaaacaaatgttctcgtcacgtgggcatgaatctgatgctgtttccctgcagac
aatgcgagagaatgaatcagaattcaaatatctgcttcactcacggacagaaagactgtttagag
tgctttcccgtgtcagaatctcaacccgtttctgtcgtcaaaaaggcgtatcagaaactgtgcta
cattcatcatatcatgggaaaggtgccagacgcttgcactgcctgcgatctggtcaatgtggatt
tggatgactgcatctttgaacaataaatgatttaaatcaggtatggctgccgatggttatcttcc
agattggctcgaggacactctctctgaaggaataagacagtggtggaagctcaaacctggcccac
caccaccaaagcccgcagagcggcataaggacgacagcaggggtcttgtgcttcctgggtacaag
tacctcggacccttcaacggactcgacaagggagagccggtcaacgaggcagacgccgcggccct
cgagcacgacaaagcctacgaccggcagctcgacagcggagacaacccgtacctcaagtacaacc
acgccgacgcggagtttcaggagcgccttaaagaagatacgtcttttgggggcaacctcggacga
gcagtcttccaggcgaaaaagagggttcttgaacctctgggcctggttgaggaacctgttaagac
ggctccgggaaaaaagaggccggtagagcactctcctgtggagccagactcctcctcgggaaccg
gaaaggcgggccagcagcctgcaagaaaaagattgaattttggtcagactggagacgcagactca
gtacctgacccccagcctctcggacagccaccagcagccccctctggtctgggaactaatacgat
ggctacaggcagtggcgcaccaatggcagacaataacgagggcgccgacggagtgggtaattcct
cgggaaattggcattgcgattccacatggatgggcgacagagtcatcaccaccagcacccgaacc
tgggccctgcccacctacaacaaccacctctacaaacaaatttccagccaatcaggagcctcgaa
cgacaatcactactttggctacagcaccccttgggggtattttgacttcaacagattccactgcc
acttttcaccacgtgactggcaaagactcatcaacaacaactggggattccgacccaagagactc
aacttcaagctctttaacattcaagtcaaagaggtcacgcagaatgacggtacgacgacgattgc
caataaccttaccagcacggttcaggtgtttactgactcggagtaccagctcccgtacgtcctcg
gctcggcgcatcaaggatgcctcccgccgttcccagcagacgtcttcatggtgccacagtatgga
tacctcaccctgaacaacgggagtcaggcagtaggacgctcttcattttactgcctggagtactt
tccttctcagatgctgcgtaccggaaacaactttaccttcagctacacttttgaggacgttcctt
tccacagcagctacgctcacagccagagtctggaccgtctcatgaatcctctcatcgaccagtac
ctgtattacttgagcagaacaaacactccaagtggaaccaccacgcagtcaaggcttcagttttc
tcaggccggagcgagtgacattcgggaccagtctaggaactggcttcctggaccctgttaccgcc
agcagcgagtatcaaagacatctgcggataacaacaacagtgaatactcgtggactggagctacc
aagtaccacctcaatggcagagactctctggtgaatccgggcccggccatggcaagccacaagga
cgatgaagaaaagttttttcctcagagcggggttctcatctttgggaagcaaggctcagagaaaa
caaatgtggacattgaaaaggtcatgattacagacgaagaggaaatcaggacaaccaatcccgtg
gctacggagcagtatggttctgtatctaccaacctccagagaggcaacagacaagcagctaccgc
agatgtcaacacacaaggcgttcttccaggcatggtctggcaggacagagatgtgtaccttcagg
ggcccatctgggcaaagattccacacacggacggacattttcacccctctcccctcatgggtgga
ttcggacttaaacaccctcctccacagattctcatcaagaacaccccggtacctgcgaatccttc
gaccaccttcagtgcggcaaagtttgcttccttcatcacacagtactccacgggacaggtcagcg
tggagatcgagtgggagctgcagaaggaaaacagcaaacgctggaatcccgaaattcagtacact
tccaactacaacaagtctgttaatgtggactttactgtggacactaatggcgtgtattcagagcc
tcgccccattggcaccagatacctgactcgtaatctgtaattgcttgttaatagcttacgcgtgg
ccgctcgagacggccggctgatcataatcagccataccacatttgtagaggttttacttgcttta
aaaaacctcccacacctccccctgaacctgaaacataaaatgaatgcaattgttgttgttaactt
gtttattgcagcttataatggttacaaataaagcaatagcatcacaaatttcacaaataaagcat
ttttttcactgcattctagttgtggtttgtccaaactcatcaatgtatcttaacgcaattcggct
tggtgtggaaagtccccaggctccccagcaggcagaagtatgcaaagcatgcatctcaattagtc
agcaaccaggtgtggaaagtccccaggctccccagcaggcagaagtatgcaaagcatgcatctca
attagtcagcaaccaaattaaagtaacccataacttcgtatagcatacattatacgaagttatga
agttcctattctctagaaagtataggaacttctagtcacctatttcagcatactacgcgcgtagt
atgctgaaataggtttatcagcacacaatagtccattatacgcgcgtataatggcaattgtgtgc
tgattgggttactttaatttggatccgtcgaccgatgcccttgagagccttcaacccagtcagct
ccttccggtgggcgcggggcatgactatcgtcgccgcacttatgactgtcttctttatcatgcaa
ctcgtaggacaggtgccggcagcgctcttccgcttcctcgctcactgactcgctgcgctcggtcg
ttcggctgcggcgagcggtatcagctcactcaaaggcggtaatacggttatccacagaatcaggg
gataacgcaggaaagaacatgtgagcaaaaggccagcaaaaggccaggaaccgtaaaaaggccgc
gttgctggcgtttttccataggctccgcccccctgacgagcatcacaaaaatcgacgctcaagtc
agaggtggcgaaacccgacaggactataaagataccaggcgtttccccctggaagctccctcgtg
cgctctcctgttccgaccctgccgcttaccggatacctgtccgcctttctcccttcgggaagcgt
ggcgctttctcatagctcacgctgtaggtatctcagttcggtgtaggtcgttcgctccaagctgg
gctgtgtgcacgaaccccccgttcagcccgaccgctgcgccttatccggtaactatcgtcttgag
tccaacccggtaagacacgacttatcgccactggcagcagccactggtaacaggattagcagagc
gaggtatgtaggcggtgctacagagttcttgaagtggtggcctaactacggctacactagaagaa
cagtatttggtatctgcgctctgctgaagccagttaccttcggaaaaagagttggtagctcttga
tccggcaaacaaaccaccgctggtagcggtggtttttttgtttgcaagcagcagattacgcgcag
aaaaaaaggatctcaagaagatcctttgatcttttctacggggtctgacgctcagtggaacgaaa
actcacgttaagggattttggtcatgagattatcaaaaaggatcttcacctagatccttttaaat
taaaaatgaagttttaaatcaatctaaagtatatatgagtaaacttggtctgacagttaccaatg
cttaatcagtgaggcacctatctcagcgatctgtctatttcgttcatccatagttgcctgactcc
ccgtcgtgtagataactacgatacgggagggcttaccatctggccccagtgctgcaatgataccg
cgagacccacgctcaccggctccagatttatcagcaataaaccagccagccggaagggccgagcg
cagaagtggtcctgcaactttatccgcctccatccagtctattaattgttgccgggaagctagag
taagtagttcgccagttaatagtttgcgcaacgttgttgccattgctacaggcatcgtggtgtca
cgctcgtcgtttggtatggcttcattcagctccggttcccaacgatcaaggcgagttacatgatc
ccccatgttgtgcaaaaaagcggttagctccttcggtcctccgatcgttgtcagaagtaagttgg
ccgcagtgttatcactcatggttatggcagcactgcataattctcttactgtcatgccatccgta
agatgcttttctgtgactggtgagtactcaaccaagtcattctgagaatagtgtatgcggcgacc
gagttgctcttgcccggcgtcaatacgggataataccgcgccacatagcagaactttaaaagtgc
tcatcattggaaaacgttcttcggggcgaaaactctcaaggatcttaccgctgttgagatccagt
tcgatgtaacccactcgtgcacccaactgatcttcagcatcttttactttcaccagcgtttctgg
gtgagcaaaaacaggaaggcaaaatgccgcaaaaaagggaataagggcgacacggaaatgttgaa
tactcatactcttcctttttcaatattattgaagcatttatcagggttattgtctcatgagcgga
tacatatttgaatgtatttagaaaaataaacaaataggggttccgcgcacatttccccgaaaagt
gccacctgacgcgccctgtagcggcgcattaagcgcggcgggtgtggtggttacgcgcagcgtga
ccgctacacttgccagcgccctagcgcccgctcctttcgctttcttcccttcctttctcgccacg
ttcgccggctttccccgtcaagctctaaatcgggggctccctttagggttccgatttagtgcttt
acggcacctcgaccccaaaaaacttgattagggtgatggttcacgtagtgggccatcgccctgat
agacggtttttcgccctttgacgttggagtccacgttctttaatagtggactcttgttccaaact
ggaacaacactcaaccctatctcggtctattcttttgatttataagggattttgccgatttcggc
ctattggttaaaaaatgagctgatttaacaaaaatttaacgcgaattttaacaaaatattaacgc
ttacaatttgccattcgccattcaggctgcgcaactgttgggaagggcgatcggtgcgggcctct
tcgctattacgccagcccaagctaccatgataagtaagtaatattaaggtacgtggaggttttac
ttgctttaaaaaacctcccacacctccccctgaacctgaaacataaaatgaatgcaattgttgtt
gttaacttgtttattgcagcttataatggttacaaataaagcaatagcatcacaaatttcacaaa
taaagcatttttttcactgcattctagttgtggtttgtccaaactcatcaatgtatcttatggta
ctgtaactgagctaacataa
PGL2-SS-CMV-Rep-Cap-SS msDNA
SEQ ID NO: 24
tacgcgcgtagtatgctgaaataggtgactagaagttcctatactttctagagaataggaacttc
ataacttcgtataatgtatgctatacgaagttatgggttactttaatttggttgctgactaattg
agatgcatgctttgcatacttctgcctgctggggagcctggggactttccacacctggttgctga
ctaattgagatgcatgctttgcatacttctgcctgctggggagcctggggactttccacacccct
gattctgtggataaccgtattaccgccatgcattagttattaatagtaatcaattacggggtcat
tagttcatagcccatatatggagttccgcgttacataacttacggtaaatggcccgcctggctga
ccgcccaacgacccccgcccattgacgtcaataatgacgtatgttcccatagtaacgccaatagg
gactttccattgacgtcaatgggtggagtatttacggtaaactgcccacttggcagtacatcaag
tgtatcatatgccaagtacgccccctattgacgtcaatgacggtaaatggcccgcctggcattat
gcccagtacatgaccttatgggactttcctacttggcagtacatctacgtattagtcatcgctat
taccatggtgatgcggttttggcagtacatcaatgggcgtggatagcggtttgactcacggggat
ttccaagtctccaccccattgacgtcaatgggagtttgttttggcaccaaaatcaacgggacttt
ccaaaatgtcgtaacaactccgccccattgacgcaaatgggcggtaggcgtgtacggtgggaggt
ctatataagcagagctggtttagtgaaccgtcagatccgtttgaacgcgcagccgccatgccggg
gttttacgagattgtgattaaggtccccagcgaccttgacgagcatctgcccggcatttctgaca
gctttgtgaactgggtggccgagaaggaatgggagttgccgccagattctgacatggatctgaat
ctgattgagcaggcacccctgaccgtggccgagaagctgcagcgcgactttctgacggaatggcg
ccgtgtgagtaaggccccggaggcccttttctttgtgcaatttgagaagggagagagctacttcc
acatgcacgtgctcgtggaaaccaccggggtgaaatccatggttttgggacgtttcctgagtcag
attcgcgaaaaactgattcagagaatttaccgcgggatcgagccgactttgccaaactggttcgc
ggtcacaaagaccagaaatggcgccggaggcgggaacaaggtggtggatgagtgctacatcccca
attacttgctccccaaaacccagcctgagctccagtgggcgtggactaatatggaacagtattta
agcgcctgtttgaatctcacggagcgtaaacggttggtggcgcagcatctgacgcacgtgtcgca
gacgcaggagcagaacaaagagaatcagaatcccaattctgatgcgccggtgatcagatcaaaaa
cttcagccaggtacatggagctggtcgggtggctcgtggacaaggggattacctcggagaagcag
tggatccaggaggaccaggcctcatacatctccttcaatgcggcctccaactcgcggtcccaaat
caaggctgccttggacaatgcgggaaagattatgagcctgactaaaaccgcccccgactacctgg
tgggccagcagcccgtggaggacatttccagcaatcggatttataaaattttggaactaaacggg
tacgatccccaatatgcggcttccgtctttctgggatgggccacgaaaaagttcggcaagaggaa
caccatctggctgtttgggcctgcaactaccgggaagaccaacatcgcggaggccatagcccaca
ctgtgcccttctacgggtgcgtaaactggaccaatgagaactttcccttcaacgactgtgtcgac
aagatggtgatctggtgggaggaggggaagatgaccgccaaggtcgtggagtcggccaaagccat
tctcggaggaagcaaggtgcgcgtggaccagaaatgcaagtcctcggcccagatagacccgactc
ccgtgatcgtcacctccaacaccaacatgtgcgccgtgattgacgggaactcaacgaccttcgaa
caccagcagccgttgcaagaccggatgttcaaatttgaactcacccgccgtctggatcatgactt
tgggaaggtcaccaagcaggaagtcaaagactttttccggtgggcaaaggatcacgtggttgagg
tggagcatgaattctacgtcaaaaagggtggagccaagaaaagacccgcccccagtgacgcagat
ataagtgagcccaaacgggtgcgcgagtcagttgcgcagccatcgacgtcagacgcggaagcttc
gatcaactacgcagacaggtaccaaaacaaatgttctcgtcacgtgggcatgaatctgatgctgt
ttccctgcagacaatgcgagagaatgaatcagaattcaaatatctgcttcactcacggacagaaa
gactgtttagagtgctttcccgtgtcagaatctcaacccgtttctgtcgtcaaaaaggcgtatca
gaaactgtgctacattcatcatatcatgggaaaggtgccagacgcttgcactgcctgcgatctgg
tcaatgtggatttggatgactgcatctttgaacaataaatgatttaaatcaggtatggctgccga
tggttatcttccagattggctcgaggacactctctctgaaggaataagacagtggtggaagctca
aacctggcccaccaccaccaaagcccgcagagcggcataaggacgacagcaggggtcttgtgctt
cctgggtacaagtacctcggacccttcaacggactcgacaagggagagccggtcaacgaggcaga
cgccgcggccctcgagcacgacaaagcctacgaccggcagctcgacagcggagacaacccgtacc
tcaagtacaaccacgccgacgcggagtttcaggagcgccttaaagaagatacgtcttttgggggc
aacctcggacgagcagtcttccaggcgaaaaagagggttcttgaacctctgggcctggttgagga
acctgttaagacggctccgggaaaaaagaggccggtagagcactctcctgtggagccagactcct
cctcgggaaccggaaaggcgggccagcagcctgcaagaaaaagattgaattttggtcagactgga
gacgcagactcagtacctgacccccagcctctcggacagccaccagcagccccctctggtctggg
aactaatacgatggctacaggcagtggcgcaccaatggcagacaataacgagggcgccgacggag
tgggtaattcctcgggaaattggcattgcgattccacatggatgggcgacagagtcatcaccacc
agcacccgaacctgggccctgcccacctacaacaaccacctctacaaacaaatttccagccaatc
aggagcctcgaacgacaatcactactttggctacagcaccccttgggggtattttgacttcaaca
gattccactgccacttttcaccacgtgactggcaaagactcatcaacaacaactggggattccga
cccaagagactcaacttcaagctctttaacattcaagtcaaagaggtcacgcagaatgacggtac
gacgacgattgccaataaccttaccagcacggttcaggtgtttactgactcggagtaccagctcc
cgtacgtcctcggctcggcgcatcaaggatgcctcccgccgttcccagcagacgtcttcatggtg
ccacagtatggatacctcaccctgaacaacgggagtcaggcagtaggacgctcttcattttactg
cctggagtactttccttctcagatgctgcgtaccggaaacaactttaccttcagctacacttttg
aggacgttcctttccacagcagctacgctcacagccagagtctggaccgtctcatgaatcctctc
atcgaccagtacctgtattacttgagcagaacaaacactccaagtggaaccaccacgcagtcaag
gcttcagttttctcaggccggagcgagtgacattcgggaccagtctaggaactggcttcctggac
cctgttaccgccagcagcgagtatcaaagacatctgcggataacaacaacagtgaatactcgtgg
actggagctaccaagtaccacctcaatggcagagactctctggtgaatccgggcccggccatggc
aagccacaaggacgatgaagaaaagttttttcctcagagcggggttctcatctttgggaagcaag
gctcagagaaaacaaatgtggacattgaaaaggtcatgattacagacgaagaggaaatcaggaca
accaatcccgtggctacggagcagtatggttctgtatctaccaacctccagagaggcaacagaca
agcagctaccgcagatgtcaacacacaaggcgttcttccaggcatggtctggcaggacagagatg
tgtaccttcaggggcccatctgggcaaagattccacacacggacggacattttcacccctctccc
ctcatgggtggattcggacttaaacaccctcctccacagattctcatcaagaacaccccggtacc
tgcgaatccttcgaccaccttcagtgcggcaaagtttgcttccttcatcacacagtactccacgg
gacaggtcagcgtggagatcgagtgggagctgcagaaggaaaacagcaaacgctggaatcccgaa
attcagtacacttccaactacaacaagtctgttaatgtggactttactgtggacactaatggcgt
gtattcagagcctcgccccattggcaccagatacctgactcgtaatctgtaattgcttgttaata
gcttacgcgtggccgctcgagacggccggctgatcataatcagccataccacatttgtagaggtt
ttacttgctttaaaaaacctcccacacctccccctgaacctgaaacataaaatgaatgcaattgt
tgttgttaacttgtttattgcagcttataatggttacaaataaagcaatagcatcacaaatttca
caaataaagcatttttttcactgcattctagttgtggtttgtccaaactcatcaatgtatcttaa
cgcaattcggcttggtgtggaaagtccccaggctccccagcaggcagaagtatgcaaagcatgca
tctcaattagtcagcaaccaggtgtggaaagtccccaggctccccagcaggcagaagtatgcaaa
gcatgcatctcaattagtcagcaaccaaattaaagtaacccataacttcgtatagcatacattat
acgaagttatgaagttcctattctctagaaagtataggaacttctagtcacctatttcagcatac
tacgcgcgta
Helper Sequences msDNA
SEQ ID NO: 25
tacgcgcgtagtatgctgaaataggtgactagaagttcctatactttctagagaataggaacttc
ataacttcgtataatgtatgctatacgaagttatgggttactttaatttggttgctgactaattg
agatgcatgctttgcatacttctgcctgctggggagcctggggactttccacacctggttgctga
ctaattgagatgcatgctttgcatacttctgcctgctggggagcctggggactttccacaccagt
acttctcgacagaagcaccatgtccttgggtccggcctgctgaatgcgcaggcggtcggccatgc
cccaggcttcgttttgacatcggcgcaggtctttgtagtagtcttgcatgagcctttctaccggc
acttcttcttctccttcctcttgtcctgcatctcttgcatctatcgctgcggcggcggcggagtt
tggccgtaggtggcgccctcttcctcccatgcgtgtgaccccgaagcccctcatcggctgaagca
gggctaggtcggcgacaacgcgctcggctaatatggcctgctgcacctgcgtgagggtagactgg
aagtcatccatgtccacaaagcggtggtatgcgcccgtgttgatggtgtaagtgcagttggccat
aacggaccagttaacggtctggtgacccggctgcgagagctcggtgtacctgagacgcgagtaag
ccctcgagtcaaatacgtagtcgttgcaagtccgcaccaggtactggtatcccaccaaaaagtgc
ggcggcggctggcggtagaggggccagcgtagggtggccggggctccgggggcgagatcttccaa
cataaggcgatgatatccgtagatgtacctggacatccaggtgatgccggcggcggtggtggagg
cgcgcggaaagtcgcggacgcggttccagatgttgcgcagcggcaaaaagtgctccatggtcggg
acgctctggccggtcaggcgcgcgcaatcgttgacgctctagcgtgcaaaaggagagcctgtaag
cgggcactcttccgtggtctggtggataaattcgcaagggtatcatggcggacgaccggggttcg
agccccgtatccggccgtccgccgtgatccatgcggttaccgcccgcgtgtcgaacccaggtgtg
cgacgtcagacaacgggggagtgctccttttggcttccttccaggcgcggcggctgctgcgctag
cttttttggccactggccgcgcgcagcgtaagcggttaggctggaaagcgaaagcattaagtggc
tcgctccctgtagccggagggttattttccaagggttgagtcgcgggacccccggttcgagtctc
ggaccggccggactgcggcgaacgggggtttgcctccccgtcatgcaagaccccgcttgcaaatt
cctccggaaacagggacgagccccttttttgcttttcccagatgcatccggtgctgcggcagatg
cgcccccctcctcagcagcggcaagagcaagagcagcggcagacatgcagggcaccctcccctcc
tcctaccgcgtcaggaggggcgacatccgcggttgacgcggcagcagatggtgattacgaacccc
cgcggcgccgggcccggcactacctggacttggaggagggcgagggcctggcgcggctaggagcg
ccctctcctgagcggcacccaagggtgcagctgaagcgtgatacgcgtgaggcgtacgtgccgcg
gcagaacctgtttcgcgaccgcgagggagaggagcccgaggagatgcgggatcgaaagttccacg
cagggcgcgagctgcggcatggcctgaatcgcgagcggttgctgcgcgaggaggactttgagccc
gacgcgcgaaccgggattagtcccgcgcgcgcacacgtggcggccgccgacctggtaaccgcata
cgagcagacggtgaaccaggagattaactttcaaaaaagctttaacaaccacgtgcgtacgcttg
tggcgcgcgaggaggtggctataggactgatgcatctgtgggactttgtaagcgcgctggagcaa
aacccaaatagcaagccgctcatggcgcagctgttccttatagtgcagcacagcagggacaacga
ggcattcagggatgcgctgctaaacatagtagagcccgagggccgctggctgctcgatttgataa
acatcctgcagagcatagtggtgcaggagcgcagcttgagcctggctgacaaggtggccgccatc
aactattccatgcttagcctgggcaagttttacgcccgcaagatataccataccccttacgttcc
catagacaaggaggtaaagatcgaggggttctacatgcgcatggcgctgaaggtgcttaccttga
gcgacgacctgggcgtttatcgcaacgagcgcatccacaaggccgtgagcgtgagccggcggcgc
gagctcagcgaccgcgagctgatgcacagcctgcaaagggccctggctggcacgggcagcggcga
tagagaggccgagtcctactttgacgcgggcgctgacctgcgctgggccccaagccgacgcgccc
tggaggcagctggggccggacctgggctggcggtggcacccgcgcgcgctggcaacgtcggcggc
gtggaggaatatgacgaggacgatgagtacgagccagaggacggcgagtactaagcggtgatgtt
tctgatcagatgatgcaagacgcaacggacccggcggtgcgggcggcgctgcagagccagccgtc
cggccttaactccacggacgactggcgccaggtcatggaccgcatcatgtcgctgactgcgcgca
atcctgacgcgttccggcagcagccgcaggccaaccggctctccgcaattctggaagcggtggtc
ccggcgcgcgcaaaccccacgcacgagaaggtgctggcgatcgtaaacgcgctggccgaaaacag
ggccatccggcccgacgaggccggcctggtctacgacgcgctgcttcagcgcgtggctcgttaca
acagcggcaacgtgcagaccaacctggaccggctggtgggggatgtgcgcgaggccgtggcgcag
cgtgagcgcgcgcagcagcagggcaacctgggctccatggttgcactaaacgccttcctgagtac
acagcccgccaacgtgccgcggggacaggaggactacaccaactttgtgagcgcactgcggctaa
tggtgactgagacaccgcaaagtgaggtgtaccagtctgggccagactattttttccagaccagt
agacaaggcctgcagaccgtaaacctgagccaggctttcaaaaacttgcaggggctgtggggggt
gcgggctcccacaggcgaccgcgcgaccgtgtctagcttgctgacgcccaactcgcgcctgttgc
tgctgctaatagcgcccttcacggacagtggcagcgtgtcccgggacacatacctaggtcacttg
ctgacactgtaccgcgaggccataggtcaggcgcatgtggacgagcatactttccaggagattac
aagtgtcagccgcgcgctggggcaggaggacacgggcagcctggaggcaaccctaaactacctgc
tgaccaaccggcggcagaagatcccctcgttgcacagtttgcaccctttggcgcatcccattctc
cagtaactttatgtccatgggcgcactcacagacctgggccaaaaccttctctacgccaactccg
cccacgcgctagacatgacttttgaggtggatcccatggacgagcccacccttctttatgttttg
tttgaagtctttgacgtggtccgtgtgcaccagccgcaccgcggcgtcatcgaaaccgtgtacct
gcgcacgcccttctcggccggcaacgccacaacataaagaagcaagcaacatcaacaacagctgc
cgccatgggctccagtgagcaggaactgaaagccattgtcaaagatcttggttgtgggccatatt
ttttgggcacctatgacaagcgctttccaggctttgtttctccacacaagctcgcctgcgccata
gtcaatacggccggtcgcgagactgggggcgtacactggatggcctttgcctggaacccgcactc
aaaaacatgctacctctttgagccctttggcttttctgaccagcgactcaagcaggtttaccagt
ttgagtacgagtcactcctgcgccgtagcgccattgcttcttcccccgaccgctgtataacgctg
gaaaagtccacccaaagcgtacaggggcccaactcggccgcctgtggactattctgctgcatgtt
tctccacgcctttgccaactggccccaaactcccatggatcacaaccccaccatgaaccttatta
ccggggtacccaactccatgctcaacagtccccaggtacagcccaccctgcgtcgcaaccaggaa
cagctctacagcttcctggagcgccactcgccctacttccgcagccacagtgcgcagattaggag
cgccacttctttttgtcacttgaaaaacatgtaaaaataatgtactagagacactttcaataaag
gcaaatgcttttatttgtacactctcgggtgattatttacccccacccttgccgtctgcgccgtt
taaaaatcaaaggggttctgccgcgcatcgctatgcgccactggcagggacacgttgcgatactg
gtgtttagtgctccacttaaactcaggcacaaccatccgcggcagctcggtgaagttttcactcc
acaggctgcgcaccatcaccaacgcgtttagcaggtcgggcgccgatatcttgaagtcgcagttg
gggcctccgccctgcgcgcgcgagttgcgatacacagggttgcagcactggaacactatcagcgc
cgggtggtgcacgctggccagcacgctcttgtcggagatcagatccgcgtccaggtcctccgcgt
tgctcagggcgaacggagtcaactttggtagctgccttcccaaaaagggcgcgtgcccaggcttt
gagttgcactcgcaccgtagtggcatcaaaaggtgaccgtgcccggtctgggcgttaggatacag
cgcctgcataaaagccttgatctgcttaaaagccacctgagcctttgcgccttcagagaagaaca
tgccgcaagacttgccggaaaactgattggccggacaggccgcgtcgtgcacgcagcaccttgcg
tcggtgttggagatctgcaccacatttcggccccaccggttcttcacgatcttggccttgctaga
ctgctccttcagcgcgcgctgcccgttttcgctcgtcacatccatttcaatcacgtgctccttat
ttatcataatgcttccgtgtagacacttaagctcgccttcgatctcagcgcagcggtgcagccac
aacgcgcagcccgtgggctcgtgatgcttgtaggtcacctctgcaaacgactgcaggtacgcctg
caggaatcgccccatcatcgtcacaaaggtcttgttgctggtgaaggtcagctgcaacccgcggt
gctcctcgttcagccaggtcttgcatacggccgccagagcttccacttggtcaggcagtagtttg
aagttcgcctttagatcgttatccacgtggtacttgtccatcagcgcgcgcgcagcctccatgcc
cttctcccacgcagacacgatcggcacactcagcgggttcatcaccgtaatttcactttccgctt
cgctgggctcttcctcttcctcttgcgtccgcataccacgcgccactgggtcgtcttcattcagc
cgccgcactgtgcgcttacctcctttgccatgcttgattagcaccggtgggttgctgaaacccac
catttgtagcgccacatcttctctttcttcctcgctgtccacgattacctctggtgatggcgggc
gctcgggcttgggagaagggcgcttctttttcttcttgggcgcaatggccaaatccgccgccgag
gtcgatggccgcgggctgggtgtgcgcggcaccagcgcgtcttgtgatgagtcttcctcgtcctc
ggactcgatacgccgcctcatccgcttttttgggggcgcccggggaggcggcggcgacggggacg
gggacgacacgtcctccatggttgggggacgtcgcgccgcaccgcgtccgcgctcgggggtggtt
tcgcgctgctcctcttcccgactggccatttccttctcctataggcagaaaaagatcatggagtc
agtcgagaagaaggacagcctaaccgccccctctgagttcgccaccaccgcctccaccgatgccg
ccaacgcgcctaccaccttccccgtcgaggcacccccgcttgaggaggaggaagtgattatcgag
caggacccaggttttgtaagcgaagacgacgaggaccgctcagtaccaacagaggataaaaagca
agaccaggacaacgcagaggcaaacgaggaacaagtcgggcggggggacgaaaggcatggcgact
acctagatgtgggagacgacgtgctgttgaagcatctgcagcgccagtgcgccattatctgcgac
gcgttgcaagagcgcagcgatgtgcccctcgccatagcggatgtcagccttgcctacgaacgcca
cctattctcaccgcgcgtaccccccaaacgccaagaaaacggcacatgcgagcccaacccgcgcc
tcaacttctaccccgtatttgccgtgccagaggtgcttgccacctatcacatctttttccaaaac
tgcaagatacccctatcctgccgtgccaaccgcagccgagcggacaagcagctggccttgcggca
gggcgctgtcatacctgatatcgcctcgctcaacgaagtgccaaaaatctttgagggtcttggac
gcgacgagaagcgcgcggcaaacgctctgcaacaggaaaacagcgaaaatgaaagtcactctgga
gtgttggtggaactcgagggtgacaacgcgcgcctagccgtactaaaacgcagcatcgaggtcac
ccactttgcctacccggcacttaacctaccccccaaggtcatgagcacagtcatgagtgagctga
tcgtgcgccgtgcgcagcccctggagagggatgcaaatttgcaagaacaaacagaggagggccta
cccgcagttggcgacgagcagctagcgcgctggcttcaaacgcgcgagcctgccgacttggagga
gcgacgcaaactaatgatggccgcagtgctcgttaccgtggagcttgagtgcatgcagcggttct
ttgctgacccggagatgcagcgcaagctagaggaaacattgcactacacctttcgacagggctac
gtacgccaggcctgcaagatctccaacgtggagctctgcaacctggtctcctaccttggaatttt
gcacgaaaaccgccttgggcaaaacgtgcttcattccacgctcaagggcgaggcgcgccgcgact
acgtccgcgactgcgtttacttatttctatgctacacctggcagacggccatgggcgtttggcag
cagtgcttggaggagtgcaacctcaaggagctgcagaaactgctaaagcaaaacttgaaggacct
atggacggccttcaacgagcgctccgtggccgcgcacctggcggacatcattttccccgaacgcc
tgcttaaaaccctgcaacagggtctgccagacttcaccagtcaaagcatgttgcagaactttagg
aactttatcctagagcgctcaggaatcttgcccgccacctgctgtgcacttcctagcgactttgt
gcccattaagtaccgcgaatgccctccgccgctttggggccactgctaccttctgcagctagcca
actaccttgcctaccactctgacataatggaagacgtgagcggtgacggtctactggagtgtcac
tgtcgctgcaacctatgcaccccgcaccgctccctggtttgcaattcgcagctgcttaacgaaag
tcaaattatcggtacctttgagctgcagggtccctcgcctgacgaaaagtccgcggctccggggt
tgaaactcactccggggctgtggacgtcggcttaccttcgcaaatttgtacctgaggactaccac
gcccacgagattaggttctacgaagaccaatcccgcccgcctaatgcggagcttaccgcctgcgt
cattacccagggccacattcttggccaattgcaagccatcaacaaagcccgccaagagtttctgc
tacgaaagggacggggggtttacttggacccccagtccggcgaggagctcaacccaatccccccg
ccgccgcagccctatcagcagcagccgcgggcccttgcttcccaggatggcacccaaaaagaagc
tgcagctgccgccgccacccacggacgaggaggaatactgggacagtcaggcagaggaggttttg
gacgaggaggaggaggacatgatggaagactgggagagcctagacgaggaagcttccgaggtcga
agaggtgtcagacgaaacaccgtcaccctcggtcgcattcccctcgccggcgccccagaaatcgg
caaccggttccagcatggctacaacctccgctcctcaggcgccgccggcactgcccgttcgccga
cccaaccgtagatgggacaccactggaaccagggccggtaagtccaagcagccgccgccgttagc
ccaagagcaacaacagcgccaaggctaccgctcatggcgcgggcacaagaacgccatagttgctt
gcttgcaagactgtgggggcaacatctccttcgcccgccgctttcttctctaccatcacggcgtg
gccttcccccgtaacatcctgcattactaccgtcatctctacagcccatactgcaccggcggcag
cggcagcaacagcagcggccacacagaagcaaaggcgaccggatagcaagactctgacaaagccc
aagaaatccacagcggcggcagcagcaggaggaggagcgctgcgtctggcgcccaacgaacccgt
atcgacccgcgagcttagaaacaggatttttcccactctgtatgctatatttcaacagagcaggg
gccaagaacaagagctgaaaataaaaaacaggtctctgcgatccctcacccgcagctgcctgtat
cacaaaagcgaagatcagcttcggcgcacgctggaagacgcggaggctctcttcagtaaatactg
cgcgctgactcttaaggactagtttcgcgccctttctcaaatttaagcgcgaaaactacgtcatc
tccagcggccacacccggcgccagcacctgttgtcagcgccattatgagcaaggaaattcccacg
ccctacatgtggagttaccagccacaaatgggacttgcggctggagctgcccaagactactcaac
ccgaataaactacatgagcgcgggaccccacatgatatcccgggtcaacggaatacgcgcccacc
gaaaccgaattctcctggaacaggcggctattaccaccacacctcgtaataaccttaatccccgt
agttggcccgctgccctggtgtaccaggaaagtcccgctcccaccactgtggtacttcccagaga
cgcccaggccgaagttcagatgactaactcaggggcgcagcttgcgggcggctttcgtcacaggg
tgcggtcgcccgggcagggtataactcacctgacaatcagagggcgaggtattcagctcaacgac
gagtcggtgagctcctcgcttggtctccgtccggacgggacatttcagatcggcggcgccggccg
ctcttcattcacgcctcgtcaggcaatcctaactctgcagacctcgtcctctgagccgcgctctg
gaggcattggaactctgcaatttattgaggagtttgtgccatcggtctactttaaccccttctcg
ggacctcccggccactatccggatcaatttattcctaactttgacgcggtaaaggactcggcgga
cggctacgactgaatgttaagtggagaggcagagcaactgcgcctgaaacacctggtccactgtc
gccgccacaagtgctttgcccgcgactccggtgagttttgctactttgaattgcccgaggatcat
atcgagggcccggcgcacggcgtccggcttaccgcccagggagagcttgcccgtagcctgattcg
ggagtttacccagcgccccctgctagttgagcgggacaggggaccctgtgttctcactgtgattt
gcaactgtcctaaccctggattacatcaagatcctctagttaattaactagagtacccggggatc
ttattccctttaactaataaaaaaaaataataaagcatcacttacttaaaatcagttagcaaatt
tctgtccagtttattcagcagcacctccttgccctcctcccagctctggtattgcagcttcctcc
tggctgcaaactttctccacaatctaaatggaatgtcagtttcctcctgttcctgtccatccgca
cccactatcttcatgttgttgcagatgaagcgcgcaagaccgtctgaagataccttcaaccccgt
gtatccatatgacacggaaaccggtcctccaactgtgccttttcttactcctccctttgtatccc
ccaatgggtttcaagagagtccccctggggtactctctttgcgcctatccgaacctctagttacc
tccaatggcatgcttgcgctcaaaatgggcaacggcctctctctggacgaggccggcaaccttac
ctcccaaaatgtaaccactgtgagcccacctctcaaaaaaaccaagtcaaacataaacctggaaa
tatctgcacccctcacagttacctcagaagccctaactgtggctgccgccgcacctctaatggtc
gcgggcaacacactcaccatgcaatcacaggccccgctaaccgtgcacgactccaaacttagcat
tgccacccaaggacccctcacagtgtcagaaggaaagctagccctgcaaacatcaggccccctca
ccaccaccgatagcagtacccttactatcactgcctcaccccctctaactactgccactggtagc
ttgggcattgacttgaaagagcccatttatacacaaaatggaaaactaggactaaagtacggggc
tcctttgcatgtaacagacgacctaaacactttgaccgtagcaactggtccaggtgtgactatta
ataatacttccttgcaaactaaagttactggagccttgggttttgattcacaaggcaatatgcaa
cttaatgtagcaggaggactaaggattgattctcaaaacagacgccttatacttgatgttagtta
tccgtttgatgctcaaaaccaactaaatctaagactaggacagggccctctttttataaactcag
cccacaacttggatattaactacaacaaaggcctttacttgtttacagcttcaaacaattccaaa
aagcttgaggttaacctaagcactgccaaggggttgatgtttgacgctacagccatagccattaa
tgcaggagatgggcttgaatttggttcacctaatgcaccaaacacaaatcccctcaaaacaaaaa
ttggccatggcctagaatttgattcaaacaaggctatggttcctaaactaggaactggccttagt
tttgacagcacaggtgccattacagtaggaaaaaataataatgataagctaactttgtggaccac
accagctccatctcctaactgtagactaaatgcagagaaagatgctaaactcactttggtcttaa
caaaatgtggcagtcaaatacttgctacagtttcagttttggctgttaaaggcagtttggctcca
atatctggaacagttcaaagtgctcatcttattataagatttgacgaaaatggagtgctactaaa
caattccttcctggacccagaatattggaactttagaaatggagatcttactgaaggcacagcct
atacaaacgctgttggatttatgcctaacctatcagcttatccaaaatctcacggtaaaactgcc
aaaagtaacattgtcagtcaagtttacttaaacggagacaaaactaaacctgtaacactaaccat
tacactaaacggtacacaggaaacaggagacacaactccaagtgcatactctatgtcattttcat
gggactggtctggccacaactacattaatgaaatatttgccacatcctcttacactttttcatac
attgcccaagaataaagaatcgtttgtgttatgtttcaacgtgtttatttttcaattgcagaaaa
tttcaagtcatttttcattcagtagtatagccccaccaccacatagcttatacagatcaccgtac
cttaatcaaactcacagaaccctagtattcaacctgccacctccctcccaacacacagagtacac
agtcctttctccccggctggccttaaaaagcatcatatcatgggtaacagacatattcttaggtg
ttatattccacacggtttcctgtcgagccaaacgctcatcagtgatattaataaactccccgggc
agctcacttaagttcatgtcgctgtccagctgctgagccacaggctgctgtccaacttgcggttg
cttaacgggcggcgaaggagaagtccacgcctacatgggggtagagtcataatcgtgcatcagga
tagggcggtggtgctgcagcagcgcgcgaataaactgctgccgccgccgctccgtcctgcaggaa
tacaacatggcagtggtctcctcagcgatgattcgcaccgcccgcagcataaggcgccttgtcct
ccgggcacagcagcgcaccctgatctcacttaaatcagcacagtaactgcagcacagcaccacaa
tattgttcaaaatcccacagtgcaaggcgctgtatccaaagctcatggcggggaccacagaaccc
acgtggccatcataccacaagcgcaggtagattaagtggcgacccctcataaacacgctggacat
aaacattacctcttttggcatgttgtaattcaccacctcccggtaccatataaacctctgattaa
acatggcgccatccaccaccatcctaaaccagctggccaaaacctgcccgccggctatacactgc
agggaaccgggactggaacaatgacagtggagagcccaggactcgtaaccatggatcatcatgct
cgtcatgatatcaatgttggcacaacacaggcacacgtgcatacacttcctcaggattacaagct
cctcccgcgttagaaccatatcccagggaacaacccattcctgaatcagcgtaaatcccacactg
cagggaagacctcgcacgtaactcacgttgtgcattgtcaaagtgttacattcgggcagcagcgg
atgatcctccagtatggtagcgcgggtttctgtctcaaaaggaggtagacgatccctactgtacg
gagtgcgccgagacaaccgagatcgtgttggtcgtagtgtcatgccaaatggaacgccggacgta
gtcatatttcctgaagcaaaaccaggtgcgggcgtgacaaacagatctgcgtctccggtctcgcc
gcttagatcgctctgtgtagtagttgtagtatatccactctctcaaagcatccaggcgccccctg
gcttcgggttctatgtaaactccttcatgcgccgctgccctgataacatccaccaccgcagaata
agccacacccagccaacctacacattcgttctgcgagtcacacacgggaggagcgggaagagctg
gaagaaccatgtttttttttttattccaaaagattatccaaaacctcaaaatgaagatctattaa
gtgaacgcgctcccctccggtggcgtggtcaaactctacagccaaagaacagataatggcatttg
taagatgttgcacaatggcttccaaaaggcaaacggccctcacgtccaagtggacgtaaaggcta
aacccttcagggtgaatctcctctataaacattccagcaccttcaaccatgcccaaataattctc
atctcgccaccttctcaatatatctctaagcaaatcccgaatattaagtccggccattgtaaaaa
tctgctccagagcgccctccaccttcagcctcaagcagcgaatcatgattgcaaaaattcaggtt
cctcacagacctgtataagattcaaaagcggaacattaacaaaaataccgcgatcccgtaggtcc
cttcgcagggccagctgaacataatcgtgcaggtctgcacggaccagcgcggccacttccccgcc
aggaaccatgacaaaagaacccacactgattatgacacgcatactcggagctatgctaaccagcg
tagccccgatgtaagcttgttgcatgggcggcgatataaaatgcaaggtgctgctcaaaaaatca
ggcaaagcctcgcgcaaaaaagaaagcacatcgtagtcatgctcatgcagataaaggcaggtaag
ctccggaaccaccacagaaaaagacaccatttttctctcaaacatgtctgcgggtttctgcataa
acacaaaataaaataacaaaaaaacatttaaacattagaagcctgtcttacaacaggaaaaacaa
cccttataagcataagacggactacggccatgccggcgtgaccgtaaaaaaactggtcaccgtga
ttaaaaagcaccaccgacagctcctcggtcatgtccggagtcataatgtaagactcggtaaacac
atcaggttgattcacatcggtcagtgctaaaaagcgaccgaaatagcccgggggaatacataccc
gcaggcgtagagacaacattacagcccccataggaggtataacaaaattaataggagagaaaaac
acataaacacctgaaaaaccctcctgcctaggcaaaatagcaccctcccgctccagaacaacata
cagcgcttccacagcggcagccataacagtcagccttaccagtaaaaaagaaaacctattaaaaa
aacaccactcgacacggcaccagctcaatcagtcacagtgtaaaaaagggccaagtgcagagcga
gtatatataggactaaaaaatgacgtaacggttaaagtccacaaaaaacacccagaaaaccgcac
gcgaacctacgcccagaaacgaaagccaaaaaacccacaacttcctcaaatcgtcacttccgttt
tcccacgttacgtcacttcccattttaagaaaactacaattcccaacacatacaagttactccgc
cctaaaacctacgtcacccgccccgttcccacgccccgcgccacgtcacaaactccaccccctca
ttatcatattggcttcaatccaaaataatcatcaataatataccttattttggattgaagccaat
atgataatgagggggtggagtttgtgacgtggcgcggggcgtgggaacggggcgggtgacgtagt
agtgtggcggaagtgtgatgttgcaagtgtggcggaacacatgtaagcgacggatgtggcaaaag
tgacgtttttggtgtgcgccggatccacaggacggggtgtggaaagtccccaggctccccagcag
gcagaagtatgcaaagcatgcatctcaattagtcagcaaccaaattaaagtaacccataacttcg
tatagcatacattatacgaagttatgaagttcctattctctagaaagtataggaacttctagtca
cctatttcagcatactacgcgcgta
ampR forward primer
SEQ ID NO: 26
atcccgtattgacgcc
ampR reverse primer
SEQ ID NO: 27
cgctcgtcgtttggta
F1 ori forward primer
SEQ ID NO: 28
gcgtgatggacagact
F1 ori reverse primer
SEQ ID NO: 29
gtcgaggtgccgtaaag
ori forward primer
SEQ ID NO: 30
taccgggttggactca
ori reverse primer
SEQ ID NO: 31
cccgacaggactataaag
SCAR forward primer
SEQ ID NO: 32
aacccataattgtgagcg
SCAR reverse primer
SEQ ID NO: 33
cattaaggtcgggaaaatgc
kanR forward primer
SEQ ID NO: 34
gccctgaatgaactgc
kanR reverse primer
SEQ ID NO: 35
ccatccgagtacgtgc
SSeq
SEQ ID NO: 36
taaagtaacccaatcagcacacaattgccattatacgcgcgtataatggactattgtgtgctgat
aaacctatttcagcatactacgcgcgtagtatgctgaaataggtgactagaagttcctatacttt
ctagagaataggaacttcataacttcgtataatgtatgctatacgaagttatgggttactttaat
ttggttgctgactaattgagatgcatgctttgcatacttctgcctgctggggagcctggggactt
tccacacctggttgctgactaattgagatgcatgctttgcatacttctgcctgctggggagcctg
gggactttccacacc
SSeq after Tel recombination
SEQ ID NO: 37
cgcgtagtatgctgaaataggtgactagaagttcctatactttctagagaataggaacttcataa
cttcgtataatgtatgctatacgaagttatgggttactttaatttggttgctgactaattgagat
gcatgctttgcatacttctgcctgctggggagcctggggactttccacacctggttgctgactaa
ttgagatgcatgctttgcatacttctgcctgctggggagcctggggactttccacacc
AAV artificial 5′ ITR (+ strand)
SEQ ID NO: 38
ttggccactccctctctgcgcgctcgctcgctcactgaggccgcccgggcaaagcccgggcgtcg
ggcgacctttggtcgcccggcctcagtgagcgagcgagcgcgcagagagggagtggccaactcca
tcactaggggttcct
AAV artificial 3′ ITR (+ strand)
SEQ ID NO: 39
aggaacccctagtgatggagttggccactccctctctgcgcgctcgctcgctcactgaggccggg
cgaccaaaggtcgcccgacgcccgggctttgcccgggcggcctcagtgagcgagcgagcgcgcag
agagggagtggccaa
5′ spacer between SSeq and ITR
SEQ ID NO: 40
cctgattctgtggataaccgtatta
3′ spacer between SSeq and ITR
SEQ ID NO: 41
acgcaattcggctt
pITR2Cis msDNA precursor plasmid
SEQ ID NO: 42
cccgggaggtaccgagctcttacgcgtgctagaattaaagtaacccaatcagcacacaattgcca
ttatacgcgcgtataatggactattgtgtgctgataaacctatttcagcatactacgcgcgtagt
atgctgaaataggtgactagaagttcctatactttctagagaataggaacttcataacttcgtat
aatgtatgctatacgaagttatgggttactttaatttggttgctgactaattgagatgcatgctt
tgcatacttctgcctgctggggagcctggggactttccacacctggttgctgactaattgagatg
catgctttgcatacttctgcctgctggggagcctggggactttccacacccctgattctgtggat
aaccgtattattggccactccctctctgcgcgctcgctcgctcactgaggccgcccgggcaaagc
ccgggcgtcgggcgacctttggtcgcccggcctcagtgagcgagcgagcgcgcagagagggagtg
gccaactccatcactaggggttcctctatattaccctgttatccctagcgtaactgacattgatt
attgactagttattaatagtaatcaattacggggtcattagttcatagcccatatatggagttcc
gcgttacataacttacggtaaatggcccgcctggctgaccgcccaacgacccccgcccattgacg
tcaataatgacgtatgttcccatagtaacgccaatagggactttccattgacgtcaatgggtgga
gtatttacggtaaactgcccacttggcagtacatcaagtgtatcatatgccaagtacgcccccta
ttgacgtcaatgacggtaaatggcccgcctggcattatgcccagtacatgaccttatgggacttt
cctacttggcagtacatctacgtattagtcatcgctattaccatggtcgaggtgagccccacgtt
ctgcttcactctccccatctcccccccctccccacccccaattttgtatttatttattttttaat
tattttgtgcagcgatgggggcggggggggggggggggcgcgcgccaggcggggcggggcggggc
gaggggcggggcggggcgaggcggagaggtgcggcggcagccaatcagagcggcgcgctccgaaa
gtttccttttatggcgaggcggcggcggcggcggccctataaaaagcgaagcgcgcggcgggcgg
ggagtcgctgcgacgctgccttcgccccgtgccccgctccgccgccgcctcgcgccgcccgcccc
ggctctgactgaccgcgttactcccacaggtgagcgggcgggacggcccttctcctccgggctgt
aattagcgcttggtttaatgacggcttgtttcttttctgtggctgcgtgaaagccttgaggggct
ccgggagggccctttgtgcggggggagcggctcggggggtgcgtgcgtgtgtgtgtgcgtgggga
gcgccgcgtgcggctccgcgctgcccggcggctgtgagcgctgcgggcgcggcgcggggctttgt
gcgctccgcagtgtgcgcgaggggagcgcggccgggggcggtgccccgcggtgcggggggggctg
cgaggggaacaaaggctgcgtgcggggtgtgtgcgtgggggggtgagcagggggtgtgggcgcgt
cggtcgggctgcaaccccccctgcacccccctccccgagttgctgagcacggcccggcttcgggt
gcggggctccgtacggggcgtggcgcggggctcgccgtgccgggcggggggtggcggcaggtggg
ggtgccgggcggggcggggccgcctcgggccggggagggctcgggggaggggcgcggcggccccc
ggagcgccggcggctgtcgaggcgcggcgagccgcagccattgccttttatggtaatcgtgcgag
agggcgcagggacttcctttgtcccaaatctgtgcggagccgaaatctgggaggcgccgccgcac
cccctctagcgggcgcggggcgaagcggtgcggcgccggcaggaaggaaatgggcggggagggcc
ttcgtgcgtcgccgcgccgccgtccccttctccctctccagcctcggggctgtccgcggggggac
ggctgccttcgggggggacggggcagggcggggttcggcttctggcgtgtgaccggcggctctag
agcctctgctaaccatgttcatgccttcttctttttcctacagctcctgggcaacgtgctggtta
ttgtgctgtctcatcattttggcaaagaattcttcgaaagatctgctagcttaattaacccggtc
gccaccatggtgagcaagggcgaggagctgttcaccggggtggtgcccatcctggtcgagctgga
cggcgacgtaaacggccacaagttcagcgtgtccggcgagggcgagggcgatgccacctacggca
agctgaccctgaagttcatctgcaccaccggcaagctgcccgtgccctggcccaccctcgtgacc
accctgacctacggcgtgcagtgcttcagccgctaccccgaccacatgaagcagcacgacttctt
caagtccgccatgcccgaaggctacgtccaggagcgcaccatcttcttcaaggacgacggcaact
acaagacccgcgccgaggtgaagttcgagggcgacaccctggtgaaccgcatcgagctgaagggc
atcgacttcaaggaggacggcaacatcctggggcacaagctggagtacaactacaacagccacaa
cgtctatatcatggccgacaagcagaagaacggcatcaaggtgaacttcaagatccgccacaaca
tcgaggacggcagcgtgcagctcgccgaccactaccagcagaacacccccatcggcgacggcccc
gtgctgctgcccgacaaccactacctgagcacccagtccgccctgagcaaagaccccaacgagaa
gcgcgatcacatggtcctgctggagttcgtgaccgccgccgggatcactctcggcatggacgagc
tgtacaagtaaagcggccaaatcgtacgcctaggtgatcaagatctgctagcttaattaacccgg
gactagtgcggccgctcgagcatgcatctagagggccctattctatagtgtcacctaaatgctag
agctcgctgatcagcctcgactgtgccttctagttgccagccatctgttgtttgcccctcccccg
tgccttccttgaccctggaaggtgccactcccactgtcctttcctaataaaatgaggaaattgca
tcgcattgtctgagtaggtgtcattctattctggggggtggggtggggcaggacagcaaggggga
ggattgggaagacaatagcaggcatgctggggagctagagtctacttcgctaccttaggaccgtt
atagttacgaggaacccctagtgatggagttggccactccctctctgcgcgctcgctcgctcact
gaggccgggcgaccaaaggtcgcccgacgcccgggctttgcccgggcggcctcagtgagcgagcg
agcgcgcagagagggagtggccaaacgcaattcggcttggtgtggaaagtccccaggctccccag
caggcagaagtatgcaaagcatgcatctcaattagtcagcaaccaggtgtggaaagtccccaggc
tccccagcaggcagaagtatgcaaagcatgcatctcaattagtcagcaaccaaattaaagtaacc
cataacttcgtatagcatacattatacgaagttatgaagttcctattctctagaaagtataggaa
cttctagtcacctatttcagcatactacgcgcgtagtatgctgaaataggtttatcagcacacaa
tagtccattatacgcgcgtataatggcaattgtgtgctgattgggttactttaatttggatccgt
cgaccgatgcccttgagagccttcaacccagtcagctccttccggtgggcgcggggcatgactat
cgtcgccgcacttatgactgtcttctttatcatgcaactcgtaggacaggtgccggcagcgctct
tccgcttcctcgctcactgactcgctgcgctcggtcgttcggctgcggcgagcggtatcagctca
ctcaaaggcggtaatacggttatccacagaatcaggggataacgcaggaaagaacatgtgagcaa
aaggccagcaaaaggccaggaaccgtaaaaaggccgcgttgctggcgtttttccataggctccgc
ccccctgacgagcatcacaaaaatcgacgctcaagtcagaggtggcgaaacccgacaggactata
aagataccaggcgtttccccctggaagctccctcgtgcgctctcctgttccgaccctgccgctta
ccggatacctgtccgcctttctcccttcgggaagcgtggcgctttctcatagctcacgctgtagg
tatctcagttcggtgtaggtcgttcgctccaagctgggctgtgtgcacgaaccccccgttcagcc
cgaccgctgcgccttatccggtaactatcgtcttgagtccaacccggtaagacacgacttatcgc
cactggcagcagccactggtaacaggattagcagagcgaggtatgtaggcggtgctacagagttc
ttgaagtggtggcctaactacggctacactagaagaacagtatttggtatctgcgctctgctgaa
gccagttaccttcggaaaaagagttggtagctcttgatccggcaaacaaaccaccgctggtagcg
gtggtttttttgtttgcaagcagcagattacgcgcagaaaaaaaggatctcaagaagatcctttg
atcttttctacggggtctgacgctcagtggaacgaaaactcacgttaagggattttggtcatgag
attatcaaaaaggatcttcacctagatccttttaaattaaaaatgaagttttaaatcaatctaaa
gtatatatgagtaaacttggtctgacagttaccaatgcttaatcagtgaggcacctatctcagcg
atctgtctatttcgttcatccatagttgcctgactccccgtcgtgtagataactacgatacggga
gggcttaccatctggccccagtgctgcaatgataccgcgagacccacgctcaccggctccagatt
tatcagcaataaaccagccagccggaagggccgagcgcagaagtggtcctgcaactttatccgcc
tccatccagtctattaattgttgccgggaagctagagtaagtagttcgccagttaatagtttgcg
caacgttgttgccattgctacaggcatcgtggtgtcacgctcgtcgtttggtatggcttcattca
gctccggttcccaacgatcaaggcgagttacatgatcccccatgttgtgcaaaaaagcggttagc
tccttcggtcctccgatcgttgtcagaagtaagttggccgcagtgttatcactcatggttatggc
agcactgcataattctcttactgtcatgccatccgtaagatgcttttctgtgactggtgagtact
caaccaagtcattctgagaatagtgtatgcggcgaccgagttgctcttgcccggcgtcaatacgg
gataataccgcgccacatagcagaactttaaaagtgctcatcattggaaaacgttcttcggggcg
aaaactctcaaggatcttaccgctgttgagatccagttcgatgtaacccactcgtgcacccaact
gatcttcagcatcttttactttcaccagcgtttctgggtgagcaaaaacaggaaggcaaaatgcc
gcaaaaaagggaataagggcgacacggaaatgttgaatactcatactcttcctttttcaatatta
ttgaagcatttatcagggttattgtctcatgagcggatacatatttgaatgtatttagaaaaata
aacaaataggggttccgcgcacatttccccgaaaagtgccacctgacgcgccctgtagcggcgca
ttaagcgcggcgggtgtggtggttacgcgcagcgtgaccgctacacttgccagcgccctagcgcc
cgctcctttcgctttcttcccttcctttctcgccacgttcgccggctttccccgtcaagctctaa
atcgggggctccctttagggttccgatttagtgctttacggcacctcgaccccaaaaaacttgat
tagggtgatggttcacgtagtgggccatcgccctgatagacggtttttcgccctttgacgttgga
gtccacgttctttaatagtggactcttgttccaaactggaacaacactcaaccctatctcggtct
attcttttgatttataagggattttgccgatttcggcctattggttaaaaaatgagctgatttaa
caaaaatttaacgcgaattttaacaaaatattaacgcttacaatttgccattcgccattcaggct
gcgcaactgttgggaagggcgatcggtgcgggcctcttcgctattacgccagcccaagctaccat
gataagtaagtaatattaaggtacgtggaggttttacttgctttaaaaaacctcccacacctccc
cctgaacctgaaacataaaatgaatgcaattgttgttgttaacttgtttattgcagcttataatg
gttacaaataaagcaatagcatcacaaatttcacaaataaagcatttttttcactgcattctagt
tgtggtttgtccaaactcatcaatgtatcttatggtactgtaactgagctaacataa
ITR2Cis msDNA
SEQ ID NO: 43
cgcgtagtatgctgaaataggtgactagaagttcctatactttctagagaataggaacttcataa
cttcgtataatgtatgctatacgaagttatgggttactttaatttggttgctgactaattgagat
gcatgctttgcatacttctgcctgctggggagcctggggactttccacacctggttgctgactaa
ttgagatgcatgctttgcatacttctgcctgctggggagcctggggactttccacacccctgatt
ctgtggataaccgtattattggccactccctctctgcgcgctcgctcgctcactgaggccgcccg
ggcaaagcccgggcgtcgggcgacctttggtcgcccggcctcagtgagcgagcgagcgcgcagag
agggagtggccaactccatcactaggggttcctctatattaccctgttatccctagcgtaactga
cattgattattgactagttattaatagtaatcaattacggggtcattagttcatagcccatatat
ggagttccgcgttacataacttacggtaaatggcccgcctggctgaccgcccaacgacccccgcc
cattgacgtcaataatgacgtatgttcccatagtaacgccaatagggactttccattgacgtcaa
tgggtggagtatttacggtaaactgcccacttggcagtacatcaagtgtatcatatgccaagtac
gccccctattgacgtcaatgacggtaaatggcccgcctggcattatgcccagtacatgaccttat
gggactttcctacttggcagtacatctacgtattagtcatcgctattaccatggtcgaggtgagc
cccacgttctgcttcactctccccatctcccccccctccccacccccaattttgtatttatttat
tttttaattattttgtgcagcgatgggggcggggggggggggggggcgcgcgccaggcggggcgg
ggcggggcgaggggcggggcggggcgaggcggagaggtgcggcggcagccaatcagagcggcgcg
ctccgaaagtttccttttatggcgaggcggcggcggcggcggccctataaaaagcgaagcgcgcg
gcgggcggggagtcgctgcgacgctgccttcgccccgtgccccgctccgccgccgcctcgcgccg
cccgccccggctctgactgaccgcgttactcccacaggtgagcgggcgggacggcccttctcctc
cgggctgtaattagcgcttggtttaatgacggcttgtttcttttctgtggctgcgtgaaagcctt
gaggggctccgggagggccctttgtgcggggggagcggctcggggggtgcgtgcgtgtgtgtgtg
cgtggggagcgccgcgtgcggctccgcgctgcccggcggctgtgagcgctgcgggcgcggcgcgg
ggctttgtgcgctccgcagtgtgcgcgaggggagcgcggccgggggcggtgccccgcggtgcggg
gggggctgcgaggggaacaaaggctgcgtgcggggtgtgtgcgtgggggggtgagcagggggtgt
gggcgcgtcggtcgggctgcaaccccccctgcacccccctccccgagttgctgagcacggcccgg
cttcgggtgcggggctccgtacggggcgtggcgcggggctcgccgtgccggggcgggggtggcgg
caggtgggggtgccgggcggggcggggccgcctcgggccggggagggctcgggggaggggcgcgg
cggcccccggagcgccggcggctgtcgaggcgcggcgagccgcagccattgccttttatggtaat
cgtgcgagagggcgcagggacttcctttgtcccaaatctgtgcggagccgaaatctgggaggcgc
cgccgcaccccctctagcgggcgcggggcgaagcggtgcggcgccggcaggaaggaaatgggcgg
ggagggccttcgtgcgtcgccgcgccgccgtccccttctccctctccagcctcggggctgtccgc
ggggggacggctgccttcgggggggacggggcagggcggggttcggcttctggcgtgtgaccggc
ggctctagagcctctgctaaccatgttcatgccttcttctttttcctacagctcctgggcaacgt
gctggttattgtgctgtctcatcattttggcaaagaattcttcgaaagatctgctagcttaatta
acccggtcgccaccatggtgagcaagggcgaggagctgttcaccggggtggtgcccatcctggtc
gagctggacggcgacgtaaacggccacaagttcagcgtgtccggcgagggcgagggcgatgccac
ctacggcaagctgaccctgaagttcatctgcaccaccggcaagctgcccgtgccctggcccaccc
tcgtgaccaccctgacctacggcgtgcagtgcttcagccgctaccccgaccacatgaagcagcac
gacttcttcaagtccgccatgcccgaaggctacgtccaggagcgcaccatcttcttcaaggacga
cggcaactacaagacccgcgccgaggtgaagttcgagggcgacaccctggtgaaccgcatcgagc
tgaagggcatcgacttcaaggaggacggcaacatcctggggcacaagctggagtacaactacaac
agccacaacgtctatatcatggccgacaagcagaagaacggcatcaaggtgaacttcaagatccg
ccacaacatcgaggacggcagcgtgcagctcgccgaccactaccagcagaacacccccatcggcg
acggccccgtgctgctgcccgacaaccactacctgagcacccagtccgccctgagcaaagacccc
aacgagaagcgcgatcacatggtcctgctggagttcgtgaccgccgccgggatcactctcggcat
ggacgagctgtacaagtaaagcggccaaatcgtacgcctaggtgatcaagatctgctagcttaat
taacccgggactagtgcggccgctcgagcatgcatctagagggccctattctatagtgtcaccta
aatgctagagctcgctgatcagcctcgactgtgccttctagttgccagccatctgttgtttgccc
ctcccccgtgccttccttgaccctggaaggtgccactcccactgtcctttcctaataaaatgagg
aaattgcatcgcattgtctgagtaggtgtcattctattctggggggtggggtggggcaggacagc
aagggggaggattgggaagacaatagcaggcatgctggggagctagagtctacttcgctacctta
ggaccgttatagttacgaggaacccctagtgatggagttggccactccctctctgcgcgctcgct
cgctcactgaggccgggcgaccaaaggtcgcccgacgcccgggctttgcccgggcggcctcagtg
agcgagcgagcgcgcagagagggagtggccaaacgcaattcggcttcgcgtagtatgctgaaata
ggtgactagaagttcctatactttctagagaataggaacttcataacttcgtataatgtatgcta
tacgaagttatgggttactttaatttggttgctgactaattgagatgcatgctttgcatacttct
gcctgctggggagcctggggactttccacacctggttgctgactaattgagatgcatgctttgca
tacttctgcctgctggggagcctggggactttccacacc
pITR2Cis no SSeq plasmid control
SEQ ID NO: 44
ccaatgatatcggaaagaacatgtgagcaaaaggccagcaaaaggccaggttggccactccctct
ctgcgcgctcgctcgctcactgaggccgcccgggcaaagcccgggcgtcgggcgacctttggtcg
cccggcctcagtgagcgagcgagcgcgcagagagggagtggccaactccatcactaggggttcct
ctatattaccctgttatccctagcgtaactgacattgattattgactagttattaatagtaatca
attacggggtcattagttcatagcccatatatggagttccgcgttacataacttacggtaaatgg
cccgcctggctgaccgcccaacgacccccgcccattgacgtcaataatgacgtatgttcccatag
taacgccaatagggactttccattgacgtcaatgggtggagtatttacggtaaactgcccacttg
gcagtacatcaagtgtatcatatgccaagtacgccccctattgacgtcaatgacggtaaatggcc
cgcctggcattatgcccagtacatgaccttatgggactttcctacttggcagtacatctacgtat
tagtcatcgctattaccatggtcgaggtgagccccacgttctgcttcactctccccatctccccc
ccctccccacccccaattttgtatttatttattttttaattattttgtgcagcgatgggggcggg
gggggggggggggcgcgcgccaggcggggcggggcggggcgaggggcgggggcgggcgaggcgga
gaggtgcggcggcagccaatcagagcggcgcgctccgaaagtttccttttatggcgaggcggcgg
cggcggcggccctataaaaagcgaagcgcgcgggcgggcgggagtcgctgcgacgctgccttcgc
cccgtgccccgctccgccgccgcctcgcgccgcccgccccggctctgactgaccgcgttactccc
acaggtgagcgggcgggacggcccttctcctccgggctgtaattagcgcttggtttaatgacggc
ttgtttcttttctgtggctgcgtgaaagccttgaggggctccgggagggccctttgtgcgggggg
agcggctcggggggtgcgtgcgtgtgtgtgtgcgtggggagcgccgcgtgcggctccgcgctgcc
cggcggctgtgagcgctgcgggcgcggcgcggggctttgtgcgctccgcagtgtgcgcgagggga
gcgcggccgggggcggtgccccgcggtgcggggggggctgcgaggggaacaaaggctgcgtgcgg
ggtgtgtgcgtgggggggtgagcagggggtgtgggcgcgtcggtcgggctgcaaccccccctgca
cccccctccccgagttgctgagcacggcccggcttcgggtgcggggctccgtacggggcgtggcg
cggggctcgccgtgccgggcggggggtggcggcaggtgggggtgccgggcggggcggggccgcct
cgggccggggagggctcgggggaggggcgcggcggcccccggagcgccggcggctgtcgaggcgc
ggcgagccgcagccattgccttttatggtaatcgtgcgagagggcgcagggacttcctttgtccc
aaatctgtgcggagccgaaatctgggaggcgccgccgcaccccctctagcgggcgcggggcgaag
cggtgcggcgccggcaggaaggaaatgggcggggagggccttcgtgcgtcgccgcgccgccgtcc
ccttctccctctccagcctcggggctgtccgcggggggacggctgccttcgggggggacggggca
gggcggggttcggcttctggcgtgtgaccggcggctctagagcctctgctaaccatgttcatgcc
ttcttctttttcctacagctcctgggcaacgtgctggttattgtgctgtctcatcattttggcaa
agaattcttcgaaagatctgctagcttaattaacccggtcgccaccatggtgagcaagggcgagg
agctgttcaccggggtggtgcccatcctggtcgagctggacggcgacgtaaacggccacaagttc
agcgtgtccggcgagggcgagggcgatgccacctacggcaagctgaccctgaagttcatctgcac
caccggcaagctgcccgtgccctggcccaccctcgtgaccaccctgacctacggcgtgcagtgct
tcagccgctaccccgaccacatgaagcagcacgacttcttcaagtccgccatgcccgaaggctac
gtccaggagcgcaccatcttcttcaaggacgacggcaactacaagacccgcgccgaggtgaagtt
cgagggcgacaccctggtgaaccgcatcgagctgaagggcatcgacttcaaggaggacggcaaca
tcctggggcacaagctggagtacaactacaacagccacaacgtctatatcatggccgacaagcag
aagaacggcatcaaggtgaacttcaagatccgccacaacatcgaggacggcagcgtgcagctcgc
cgaccactaccagcagaacacccccatcggcgacggccccgtgctgctgcccgacaaccactacc
tgagcacccagtccgccctgagcaaagaccccaacgagaagcgcgatcacatggtcctgctggag
ttcgtgaccgccgccgggatcactctcggcatggacgagctgtacaagtaaagcggccaaatcgt
acgcctaggtgatcaagatctgctagcttaattaacccgggactagtgcggccgctcgagcatgc
atctagagggccctattctatagtgtcacctaaatgctagagctcgctgatcagcctcgactgtg
ccttctagttgccagccatctgttgtttgcccctcccccgtgccttccttgaccctggaaggtgc
cactcccactgtcctttcctaataaaatgaggaaattgcatcgcattgtctgagtaggtgtcatt
ctattctggggggtggggtggggcaggacagcaagggggaggattgggaagacaatagcaggcat
gctggggagctagagtctacttcgctaccttaggaccgttatagttacgaggaacccctagtgat
ggagttggccactccctctctgcgcgctcgctcgctcactgaggccgggcgaccaaaggtcgccc
gacgcccgggctttgcccgggcggcctcagtgagcgagcgagcgcgcagagagggagtggccaaa
tcggaaagaacatgtgagcaaaaggccagcaaaaggccaggaaccgtaaaaaggccgcgttgctg
gcgtttttccataggctccgcccccctgacgagcatcacaaaaatcgacgctcaagtcagaggtg
gcgaaacccgacaggactataaagataccaggcgtttccccctggaagctccctcgtgcgctctc
ctgttccgaccctgccgcttaccggatacctgtccgcctttctcccttcgggaagcgtggcgctt
tctcatagctcacgctgtaggtatctcagttcggtgtaggtcgttcgctccaagctgggctgtgt
gcacgaaccccccgttcagcccgaccgctgcgccttatccggtaactatcgtcttgagtccaacc
cggtaagacacgacttatcgccactggcagcagccactggtaacaggattagcagagcgaggtat
gtaggcggtgctacagagttcttgaagtggtggcctaactacggctacactagaagaacagtatt
tggtatctgcgctctgctgaagccagttaccttcggaaaaagagttggtagctcttgatccggca
aacaaaccaccgctggtagcggtggtttttttgtttgcaagcagcagattacgcgcagaaaaaaa
ggatctcaagaagatcctttgatcttttctacggggtctgacgctcagtggaacgaaaactcacg
ttaagggattttggtcatgagattatcaaaaaggatcttcacctagatccttttaaattaaaaat
gaagttttaaatcaatctaaagtatatatgagtaaacttggtctgacagttaccaatgcttaatc
agtgaggcacctatctcagcgatctgtctatttcgttcatccatagttgcctgactccccgtcgt
gtagataactacgatacgggagggcttaccatctggccccagtgctgcaatgataccgcgagacc
cacgctcaccggctccagatttatcagcaataaaccagccagccggaagggccgagcgcagaagt
ggtcctgcaactttatccgcctccatccagtctattaattgttgccgggaagctagagtaagtag
ttcgccagttaatagtttgcgcaacgttgttgccattgctacaggcatcgtggtgtcacgctcgt
cgtttggtatggcttcattcagctccggttcccaacgatcaaggcgagttacatgatcccccatg
ttgtgcaaaaaagcggttagctccttcggtcctccgatcgttgtcagaagtaagttggccgcagt
gttatcactcatggttatggcagcactgcataattctcttactgtcatgccatccgtaagatgct
tttctgtgactggtgagtactcaaccaagtcattctgagaatagtgtatgcggcgaccgagttgc
tcttgcccggcgtcaatacgggataataccgcgccacatagcagaactttaaaagtgctcatcat
tggaaaacgttcttcggggcgaaaactctcaaggatcttaccgctgttgagatccagttcgatgt
aacccactcgtgcacccaactgatcttcagcatcttttactttcaccagcgtttctgggtgagca
aaaacaggaaggcaaaatgccgcaaaaaagggaataagggcgacacggaaatgttgaatactcat
actcttcctttttcaatattattgaagcatttatcagggttattgtctcatgagcggatacatat
ttgaatgtatttagaaaaataaacaaataggggttccgcgcacatttccccgaaaagtgccacct
gacgtc
AAV-2 rep
SEQ ID NO: 45
atgccggggttttacgagattgtgattaaggtccccagcgaccttgacgagcatctgcccggcat
ttctgacagctttgtgaactgggtggccgagaaggaatgggagttgccgccagattctgacatgg
atctgaatctgattgagcaggcacccctgaccgtggccgagaagctgcagcgcgactttctgacg
gaatggcgccgtgtgagtaaggccccggaggcccttttctttgtgcaatttgagaagggagagag
ctacttccacatgcacgtgctcgtggaaaccaccggggtgaaatccatggttttgggacgtttcc
tgagtcagattcgcgaaaaactgattcagagaatttaccgcgggatcgagccgactttgccaaac
tggttcgcggtcacaaagaccagaaatggcgccggaggcgggaacaaggtggtggatgagtgcta
catccccaattacttgctccccaaaacccagcctgagctccagtgggcgtggactaatatggaac
agtatttaagcgcctgtttgaatctcacggagcgtaaacggttggtggcgcagcatctgacgcac
gtgtcgcagacgcaggagcagaacaaagagaatcagaatcccaattctgatgcgccggtgatcag
atcaaaaacttcagccaggtacatggagctggtcgggtggctcgtggacaaggggattacctcgg
agaagcagtggatccaggaggaccaggcctcatacatctccttcaatgcggcctccaactcgcgg
tcccaaatcaaggctgccttggacaatgcgggaaagattatgagcctgactaaaaccgcccccga
ctacctggtgggccagcagcccgtggaggacatttccagcaatcggatttataaaattttggaac
taaacgggtacgatccccaatatgcggcttccgtctttctgggatgggccacgaaaaagttcggc
aagaggaacaccatctggctgtttgggcctgcaactaccgggaagaccaacatcgcggaggccat
agcccacactgtgcccttctacgggtgcgtaaactggaccaatgagaactttcccttcaacgact
gtgtcgacaagatggtgatctggtgggaggaggggaagatgaccgccaaggtcgtggagtcggcc
aaagccattctcggaggaagcaaggtgcgcgtggaccagaaatgcaagtcctcggcccagataga
cccgactcccgtgatcgtcacctccaacaccaacatgtgcgccgtgattgacgggaactcaacga
ccttcgaacaccagcagccgttgcaagaccggatgttcaaatttgaactcacccgccgtctggat
catgactttgggaaggtcaccaagcaggaagtcaaagactttttccggtgggcaaaggatcacgt
ggttgaggtggagcatgaattctacgtcaaaaagggtggagccaagaaaagacccgcccccagtg
acgcagatataagtgagcccaaacgggtgcgcgagtcagttgcgcagccatcgacgtcagacgcg
gaagcttcgatcaactacgcagacaggtaccaaaacaaatgttctcgtcacgtgggcatgaatct
gatgctgtttccctgcagacaatgcgagagaatgaatcagaattcaaatatctgcttcactcacg
gacagaaagactgtttagagtgctttcccgtgtcagaatctcaacccgtttctgtcgtcaaaaag
gcgtatcagaaactgtgctacattcatcatatcatgggaaaggtgccagacgcttg
AAV-5 cap
SEQ ID NO: 46
atgtcttttgttgatcaccctccagattggttggaagaagttggtgaaggtcttcgcgagttttt
gggccttgaagcgggcccaccgaaaccaaaacccaatcagcagcatcaagatcaagcccgtggtc
ttgtgctgcctggttataactatctcggacccggaaacggtctcgatcgaggagagcctgtcaac
agggcagacgaggtcgcgcgagagcacgacatctcgtacaacgagcagcttgaggcgggagacaa
cccctacctcaagtacaaccacgcggacgccgagtttcaggagaagctcgccgacgacacatcct
tcgggggaaacctcggaaaggcagtctttcaggccaagaaaagggttctcgaaccttttggcctg
gttgaagagggtgctaagacggcccctaccggaaagcggatagacgaccactttccaaaaagaaa
gaaggctcggaccgaagaggactccaagccttccacctcgtcagacgccgaagctggacccagcg
gatcccagcagctgcaaatcccagcccaaccagcctcaagtttgggagctgatacaatgtctgcg
ggaggtggcggcccattgggcgacaataaccaaggtgccgatggagtgggcaatgcctcgggaga
ttggcattgcgattccacgtggatgggggacagagtcgtcaccaagtccacccgaacctgggtgc
tgcccagctacaacaaccaccagtaccgagagatcaaaagcggctccgtcgacggaagcaacgcc
aacgcctactttggatacagcaccccctgggggtactttgactttaaccgcttccacagccactg
gagcccccgagactggcaaagactcatcaacaactactggggcttcagaccccggtccctcagag
tcaaaatcttcaacattcaagtcaaagaggtcacggtgcaggactccaccaccaccatcgccaac
aacctcacctccaccgtccaagtgtttacggacgacgactaccagctgccctacgtcgtcggcaa
cgggaccgagggatgcctgccggccttccctccgcaggtctttacgctgccgcagtacggttacg
cgacgctgaaccgcgacaacacagaaaatcccaccgagaggagcagcttcttctgcctagagtac
tttcccagcaagatgctgagaacgggcaacaactttgagtttacctacaactttgaggaggtgcc
cttccactccagcttcgctcccagtcagaacctgttcaagctggccaacccgctggtggaccagt
acttgtaccgcttcgtgagcacaaataacactggcggagtccagttcaacaagaacctggccggg
agatacgccaacacctacaaaaactggttcccggggcccatgggccgaacccagggctggaacct
gggctccggggtcaaccgcgccagtgtcagcgccttcgccacgaccaataggatggagctcgagg
gcgcgagttaccaggtgcccccgcagccgaacggcatgaccaacaacctccagggcagcaacacc
tatgccctggagaacactatgatcttcaacagccagccggcgaacccgggcaccaccgccacgta
cctcgagggcaacatgctcatcaccagcgagagcgagacgcagccggtgaaccgcgtggcgtaca
acgtcggcgggcagatggccaccaacaaccagagctccaccactgcccccgcgaccggcacgtac
aacctccaggaaatcgtgcccggcagcgtgtggatggagagggacgtgtacctccaaggacccat
ctgggccaagatcccagagacgggggcgcactttcacccctctccggccatgggcggattcggac
tcaaacacccaccgcccatgatgctcatcaagaacacgcctgtgcccggaaatatcaccagcttc
tcggacgtgcccgtcagcagcttcatcacccagtacagcaccgggcaggtcaccgtggagatgga
gtgggagctcaagaaggaaaactccaagaggtggaacccagagatccagtacacaaacaactaca
acgacccccagtttgtggactttgccccggacagcaccggggaatacagaaccaccagacctatc
ggaacccgataccttacccgacccctttaa
AAV-9 cap
SEQ ID NO: 47
atggctgccgatggttatcttccagattggctcgaggacaaccttagtgaaggaattcgcgagtg
gtgggctttgaaacctggagcccctcaacccaaggcaaatcaacaacatcaagacaacgctcgag
gtcttgtgcttccgggttacaaataccttggacccggcaacggactcgacaagggggagccggtc
aacgcagcagacgcggcggccctcgagcacgacaaggcctacgaccagcagctcaaggccggaga
caacccgtacctcaagtacaaccacgccgacgccgagttccaggagcggctcaaagaagatacgt
cttttgggggcaacctcgggcgagcagtcttccaggccaaaaagaggcttcttgaacctcttggt
ctggttgaggaagcggctaagacggctcctggaaagaagaggcctgtagagcagtctcctcagga
accggactcctccgcgggtattggcaaatcgggtgcacagcccgctaaaaagagactcaatttcg
gtcagactggcgacacagagtcagtcccagaccctcaaccaatcggagaacctcccgcagccccc
tcaggtgtgggatctcttacaatggcttcaggtggtggcgcaccagtggcagacaataacgaagg
tgccgatggagtgggtagttcctcgggaaattggcattgcgattcccaatggctgggggacagag
tcatcaccaccagcacccgaacctgggccctgcccacctacaacaatcacctctacaagcaaatc
tccaacagcacatctggaggatcttcaaatgacaacgcctacttcggctacagcaccccctgggg
gtattttgacttcaacagattccactgccacttctcaccacgtgactggcagcgactcatcaaca
acaactggggattccggcctaagcgactcaacttcaagctcttcaacattcaggtcaaagaggtt
acggacaacaatggagtcaagaccatcgccaataaccttaccagcacggtccaggtcttcacgga
ctcagactatcagctcccgtacgtgctcgggtcggctcacgagggctgcctcccgccgttcccag
cggacgttttcatgattcctcagtacgggtatctgacgcttaatgatggaagccaggccgtgggt
cgttcgtccttttactgcctggaatatttcccgtcgcaaatgctaagaacgggtaacaacttcca
gttcagctacgagtttgagaacgtacctttccatagcagctacgctcacagccaaagcctggacc
gactaatgaatccactcatcgaccaatacttgtactatctctcaaagactattaacggttctgga
cagaatcaacaaacgctaaaattcagtgtggccggacccagcaacatggctgtccagggaagaaa
ctacatacctggacccagctaccgacaacaacgtgtctcaaccactgtgactcaaaacaacaaca
gcgaatttgcttggcctggagcttcttcttgggctctcaatggacgtaatagcttgatgaatcct
ggacctgctatggccagccacaaagaaggagaggaccgtttctttcctttgtctggatctttaat
ttttggcaaacaaggaactggaagagacaacgtggatgcggacaaagtcatgataaccaacgaag
aagaaattaaaactactaacccggtagcaacggagtcctatggacaagtggccacaaaccaccag
agtgcccaagcacaggcgcagaccggctgggttcaaaaccaaggaatacttccgggtatggtttg
gcaggacagagatgtgtacctgcaaggacccatttgggccaaaattcctcacacggacggcaact
ttcacccttctccgctgatgggagggtttggaatgaagcacccgcctcctcagatcctcatcaaa
aacacacctgtacctgcggatcctccaacggccttcaacaaggacaagctgaactctttcatcac
ccagtattctactggccaagtcagcgtggagatcgagtgggagctgcagaaggaaaacagcaagc
gctggaacccggagatccagtacacttccaactattacaagtctaataatgttgaatttgctgtt
aatactgaaggtgtatatagtgaaccccgccccattggcaccagatacctgactcgtaatctgta
a
Rep2/Cap2 msDNA
SEQ ID NO: 48
cgcgtagtatgctgaaataggtgactagaagttcctatactttctagagaataggaacttcataa
cttcgtataatgtatgctatacgaagttatgggttactttaatttggttgctgactaattgagat
gcatgctttgcatacttctgcctgctggggagcctggggactttccacacctggttgctgactaa
ttgagatgcatgctttgcatacttctgcctgctggggagcctggggactttccacaccggaactt
ctgatctattcgagctcggtacccctagagtcctgtattagaggtcacgtgagtgttttgcgaca
ttttgcgacaccatgtggtcacgctgggtatttaagcccgagtgagcacgcagggtctccatttt
gaagcgggaggtttgaacgcgcagccgccatgccggggttttacgagattgtgattaaggtcccc
agcgaccttgacgagcatctgcccggcatttctgacagctttgtgaactgggtggccgagaagga
atgggagttgccgccagattctgacatggatctgaatctgattgagcaggcacccctgaccgtgg
ccgagaagctgcagcgcgactttctgacggaatggcgccgtgtgagtaaggccccggaggccctt
ttctttgtgcaatttgagaagggagagagctacttccacatgcacgtgctcgtggaaaccaccgg
ggtgaaatccatggttttgggacgtttcctgagtcagattcgcgaaaaactgattcagagaattt
accgcgggatcgagccgactttgccaaactggttcgcggtcacaaagaccagaaatggcgccgga
ggcgggaacaaggtggtggatgagtgctacatccccaattacttgctccccaaaacccagcctga
gctccagtgggcgtggactaatatggaacagtatttaagcgcctgtttgaatctcacggagcgta
aacggttggtggcgcagcatctgacgcacgtgtcgcagacgcaggagcagaacaaagagaatcag
aatcccaattctgatgcgccggtgatcagatcaaaaacttcagccaggtacatggagctggtcgg
gtggctcgtggacaaggggattacctcggagaagcagtggatccaggaggaccaggcctcataca
tctccttcaatgcggcctccaactcgcggtcccaaatcaaggctgccttggacaatgcgggaaag
attatgagcctgactaaaaccgcccccgactacctggtgggccagcagcccgtggaggacatttc
cagcaatcggatttataaaattttggaactaaacgggtacgatccccaatatgcggcttccgtct
ttctgggatgggccacgaaaaagttcggcaagaggaacaccatctggctgtttgggcctgcaact
accgggaagaccaacatcgcggaggccatagcccacactgtgcccttctacgggtgcgtaaactg
gaccaatgagaactttcccttcaacgactgtgtcgacaagatggtgatctggtgggaggagggga
agatgaccgccaaggtcgtggagtcggccaaagccattctcggaggaagcaaggtgcgcgtggac
cagaaatgcaagtcctcggcccagatagacccgactcccgtgatcgtcacctccaacaccaacat
gtgcgccgtgattgacgggaactcaacgaccttcgaacaccagcagccgttgcaagaccggatgt
tcaaatttgaactcacccgccgtctggatcatgactttgggaaggtcaccaagcaggaagtcaaa
gactttttccggtgggcaaaggatcacgtggttgaggtggagcatgaattctacgtcaaaaaggg
tggagccaagaaaagacccgcccccagtgacgcagatataagtgagcccaaacgggtgcgcgagt
cagttgcgcagccatcgacgtcagacgcggaagcttcgatcaactacgcagacaggtaccaaaac
aaatgttctcgtcacgtgggcatgaatctgatgctgtttccctgcagacaatgcgagagaatgaa
tcagaattcaaatatctgcttcactcacggacagaaagactgtttagagtgctttcccgtgtcag
aatctcaacccgtttctgtcgtcaaaaaggcgtatcagaaactgtgctacattcatcatatcatg
ggaaaggtgccagacgcttgcactgcctgcgatctggtcaatgtggatttggatgactgcatctt
tgaacaataaatgatttaaatcaggtatggctgccgatggttatcttccagattggctcgaggac
actctctctgaaggaataagacagtggtggaagctcaaacctggcccaccaccaccaaagcccgc
agagcggcataaggacgacagcaggggtcttgtgcttcctgggtacaagtacctcggacccttca
acggactcgacaagggagagccggtcaacgaggcagacgccgcggccctcgagcacgacaaagcc
tacgaccggcagctcgacagcggagacaacccgtacctcaagtacaaccacgccgacgcggagtt
tcaggagcgccttaaagaagatacgtcttttgggggcaacctcggacgagcagtcttccaggcga
aaaagagggttcttgaacctctgggcctggttgaggaacctgttaagacggctccgggaaaaaag
aggccggtagagcactctcctgtggagccagactcctcctcgggaaccggaaaggcgggccagca
gcctgcaagaaaaagattgaattttggtcagactggagacgcagactcagtacctgacccccagc
ctctcggacagccaccagcagccccctctggtctgggaactaatacgatggctacaggcagtggc
gcaccaatggcagacaataacgagggcgccgacggagtgggtaattcctcgggaaattggcattg
cgattccacatggatgggcgacagagtcatcaccaccagcacccgaacctgggccctgcccacct
acaacaaccacctctacaaacaaatttccagccaatcaggagcctcgaacgacaatcactacttt
ggctacagcaccccttgggggtattttgacttcaacagattccactgccacttttcaccacgtga
ctggcaaagactcatcaacaacaactggggattccgacccaagagactcaacttcaagctcttta
acattcaagtcaaagaggtcacgcagaatgacggtacgacgacgattgccaataaccttaccagc
acggttcaggtgtttactgactcggagtaccagctcccgtacgtcctcggctcggcgcatcaagg
atgcctcccgccgttcccagcagacgtcttcatggtgccacagtatggatacctcaccctgaaca
acgggagtcaggcagtaggacgctcttcattttactgcctggagtactttccttctcagatgctg
cgtaccggaaacaactttaccttcagctacacttttgaggacgttcctttccacagcagctacgc
tcacagccagagtctggaccgtctcatgaatcctctcatcgaccagtacctgtattacttgagca
gaacaaacactccaagtggaaccaccacgcagtcaaggcttcagttttctcaggccggagcgagt
gacattcgggaccagtctaggaactggcttcctggaccctgttaccgccagcagcgagtatcaaa
gacatctgcggataacaacaacagtgaatactcgtggactggagctaccaagtaccacctcaatg
gcagagactctctggtgaatccgggcccggccatggcaagccacaaggacgatgaagaaaagttt
tttcctcagagcggggttctcatctttgggaagcaaggctcagagaaaacaaatgtggacattga
aaaggtcatgattacagacgaagaggaaatcaggacaaccaatcccgtggctacggagcagtatg
gttctgtatctaccaacctccagagaggcaacagacaagcagctaccgcagatgtcaacacacaa
ggcgttcttccaggcatggtctggcaggacagagatgtgtaccttcaggggcccatctgggcaaa
gattccacacacggacggacattttcacccctctcccctcatgggtggattcggacttaaacacc
ctcctccacagattctcatcaagaacaccccggtacctgcgaatccttcgaccaccttcagtgcg
gcaaagtttgcttccttcatcacacagtactccacgggacaggtcagcgtggagatcgagtggga
gctgcagaaggaaaacagcaaacgctggaatcccgaaattcagtacacttccaactacaacaagt
ctgttaatgtggactttactgtggacactaatggcgtgtattcagagcctcgccccattggcacc
agatacctgactcgtaatctgtaattgcttgttaatcaataaaccgtttaattcgtttcagttga
actttggtctctgcgtatttctttcttatctagtttccatggctacgtagataagtagcatggcg
ggttaatcattaactacagcccgggcgtttaaacagcgggcggaggggtggagtcgtgacgtgaa
ttacgtcatagggttagggagagtcctgtattagaggtcacgtgagtgttttgcgacattttgcg
acaccatgtggtcacgctgggtatttaagcccgagtgagcacgcagggtctccattttgagcggg
aggtttgaacgagcgctggcgcgctcactggccgtcgttttacaacgcgcgtagtatgctgaaat
aggtgactagaagttcctatactttctagagaataggaacttcataacttcgtataatgtatgct
atacgaagttatgggttactttaatttggttgctgactaattgagatgcatgctttgcatacttc
tgcctgctggggagcctggggactttccacacctggttgctgactaattgagatgcatgctttgc
atacttctgcctgctggggagcctggggactttccacacc
Rep2/Cap5 msDNA
SEQ ID NO: 49
cgcgtagtatgctgaaataggtgactagaagttcctatactttctagagaataggaacttcataa
cttcgtataatgtatgctatacgaagttatgggttactttaatttggttgctgactaattgagat
gcatgctttgcatacttctgcctgctggggagcctggggactttccacacctggttgctgactaa
ttgagatgcatgctttgcatacttctgcctgctggggagcctggggactttccacaccggaactt
ctgatctattcgagctcggtacccctagagtcctgtattagaggtcacgtgagtgttttgcgaca
ttttgcgacaccatgtggtcacgctgggtatttaagcccgagtgagcacgcagggtctccatttt
gaagcgggaggtttgaacgcgcagccgccatgccggggttttacgagattgtgattaaggtcccc
agcgaccttgacgagcatctgcccggcatttctgacagctttgtgaactgggtggccgagaagga
atgggagttgccgccagattctgacatggatctgaatctgattgagcaggcacccctgaccgtgg
ccgagaagctgcagcgcgactttctgacggaatggcgccgtgtgagtaaggccccggaggccctt
ttctttgtgcaatttgagaagggagagagctacttccacatgcacgtgctcgtggaaaccaccgg
ggtgaaatccatggttttgggacgtttcctgagtcagattcgcgaaaaactgattcagagaattt
accgcgggatcgagccgactttgccaaactggttcgcggtcacaaagaccagaaatggcgccgga
ggcgggaacaaggtggtggatgagtgctacatccccaattacttgctccccaaaacccagcctga
gctccagtgggcgtggactaatatggaacagtatttaagcgcctgtttgaatctcacggagcgta
aacggttggtggcgcagcatctgacgcacgtgtcgcagacgcaggagcagaacaaagagaatcag
aatcccaattctgatgcgccggtgatcagatcaaaaacttcagccaggtacatggagctggtcgg
gtggctcgtggacaaggggattacctcggagaagcagtggatccaggaggaccaggcctcataca
tctccttcaatgcggcctccaactcgcggtcccaaatcaaggctgccttggacaatgcgggaaag
attatgagcctgactaaaaccgcccccgactacctggtgggccagcagcccgtggaggacatttc
cagcaatcggatttataaaattttggaactaaacgggtacgatccccaatatgcggcttccgtct
ttctgggatgggccacgaaaaagttcggcaagaggaacaccatctggctgtttgggcctgcaact
accgggaagaccaacatcgcggaggccatagcccacactgtgcccttctacgggtgcgtaaactg
gaccaatgagaactttcccttcaacgactgtgtcgacaagatggtgatctggtgggaggagggga
agatgaccgccaaggtcgtggagtcggccaaagccattctcggaggaagcaaggtgcgcgtggac
cagaaatgcaagtcctcggcccagatagacccgactcccgtgatcgtcacctccaacaccaacat
gtgcgccgtgattgacgggaactcaacgaccttcgaacaccagcagccgttgcaagaccggatgt
tcaaatttgaactcacccgccgtctggatcatgactttgggaaggtcaccaagcaggaagtcaaa
gactttttccggtgggcaaaggatcacgtggttgaggtggagcatgaattctacgtcaaaaaggg
tggagccaagaaaagacccgcccccagtgacgcagatataagtgagcccaaacgggtgcgcgagt
cagttgcgcagccatcgacgtcagacgcggaagcttcgatcaactacgcagacaggtaccaaaac
aaatgttctcgtcacgtgggcatgaatctgatgctgtttccctgcagacaatgcgagagaatgaa
tcagaattcaaatatctgcttcactcacggacagaaagactgtttagagtgctttcccgtgtcag
aatctcaacccgtttctgtcgtcaaaaaggcgtatcagaaactgtgctacattcatcatatcatg
ggaaaggtgccagacgcttgcactgcctgcgatctggtcaatgtggatttggatgactgcatctt
tgaacaataaatgatttaaatcaggtatgtcttttgttgatcaccctccagattggttggaagaa
gttggtgaaggtcttcgcgagtttttgggccttgaagcgggcccaccgaaaccaaaacccaatca
gcagcatcaagatcaagcccgtggtcttgtgctgcctggttataactatctcggacccggaaacg
gtctcgatcgaggagagcctgtcaacagggcagacgaggtcgcgcgagagcacgacatctcgtac
aacgagcagcttgaggcgggagacaacccctacctcaagtacaaccacgcggacgccgagtttca
ggagaagctcgccgacgacacatccttcgggggaaacctcggaaaggcagtctttcaggccaaga
aaagggttctcgaaccttttggcctggttgaagagggtgctaagacggcccctaccggaaagcgg
atagacgaccactttccaaaaagaaagaaggctcggaccgaagaggactccaagccttccacctc
gtcagacgccgaagctggacccagcggatcccagcagctgcaaatcccagcccaaccagcctcaa
gtttgggagctgatacaatgtctgcgggaggtggcggcccattgggcgacaataaccaaggtgcc
gatggagtgggcaatgcctcgggagattggcattgcgattccacgtggatgggggacagagtcgt
caccaagtccacccgaacctgggtgctgcccagctacaacaaccaccagtaccgagagatcaaaa
gcggctccgtcgacggaagcaacgccaacgcctactttggatacagcaccccctgggggtacttt
gactttaaccgcttccacagccactggagcccccgagactggcaaagactcatcaacaactactg
gggcttcagaccccggtccctcagagtcaaaatcttcaacattcaagtcaaagaggtcacggtgc
aggactccaccaccaccatcgccaacaacctcacctccaccgtccaagtgtttacggacgacgac
taccagctgccctacgtcgtcggcaacgggaccgagggatgcctgccggccttccctccgcaggt
ctttacgctgccgcagtacggttacgcgacgctgaaccgcgacaacacagaaaatcccaccgaga
ggagcagcttcttctgcctagagtactttcccagcaagatgctgagaacgggcaacaactttgag
tttacctacaactttgaggaggtgcccttccactccagcttcgctcccagtcagaacctgttcaa
gctggccaacccgctggtggaccagtacttgtaccgcttcgtgagcacaaataacactggcggag
tccagttcaacaagaacctggccgggagatacgccaacacctacaaaaactggttcccggggccc
atgggccgaacccagggctggaacctgggctccggggtcaaccgcgccagtgtcagcgccttcgc
cacgaccaataggatggagctcgagggcgcgagttaccaggtgcccccgcagccgaacggcatga
ccaacaacctccagggcagcaacacctatgccctggagaacactatgatcttcaacagccagccg
gcgaacccgggcaccaccgccacgtacctcgagggcaacatgctcatcaccagcgagagcgagac
gcagccggtgaaccgcgtggcgtacaacgtcggcgggcagatggccaccaacaaccagagctcca
ccactgcccccgcgaccggcacgtacaacctccaggaaatcgtgcccggcagcgtgtggatggag
agggacgtgtacctccaaggacccatctgggccaagatcccagagacgggggcgcactttcaccc
ctctccggccatgggcggattcggactcaaacacccaccgcccatgatgctcatcaagaacacgc
ctgtgcccggaaatatcaccagcttctcggacgtgcccgtcagcagcttcatcacccagtacagc
accgggcaggtcaccgtggagatggagtgggagctcaagaaggaaaactccaagaggtggaaccc
agagatccagtacacaaacaactacaacgacccccagtttgtggactttgccccggacagcaccg
gggaatacagaaccaccagacctatcggaacccgataccttacccgacccctttaatgcttgtta
atcaataaactacgtagataagtagcatggcgggttaatcattaactacagcccgggcgtttaaa
cagcgggcggaggggtggagtcgtgacgtgaattacgtcatagggttagggagagtcctgtatta
gaggtcacgtgagtgttttgcgacattttgcgacaccatgtggtcacgctgggtatttaagcccg
agtgagcacgcagggtctccattttgagcgggaggtttgaacgagcgctggcgcgctcactggcc
gtcgttttacaacgcgcgtagtatgctgaaataggtgactagaagttcctatactttctagagaa
taggaacttcataacttcgtataatgtatgctatacgaagttatgggttactttaatttggttgc
tgactaattgagatgcatgctttgcatacttctgcctgctggggagcctggggactttccacacc
tggttgctgactaattgagatgcatgctttgcatacttctgcctgctggggagcctggggacttt
ccacacc
Rep2/Cap9 msDNA
SEQ ID NO: 50
cgcgtagtatgctgaaataggtgactagaagttcctatactttctagagaataggaacttcataa
cttcgtataatgtatgctatacgaagttatgggttactttaatttggttgctgactaattgagat
gcatgctttgcatacttctgcctgctggggagcctggggactttccacacctggttgctgactaa
ttgagatgcatgctttgcatacttctgcctgctggggagcctggggactttccacaccggaactt
ctgatctattcgagctcggtacccctagagtcctgtattagaggtcacgtgagtgttttgcgaca
ttttgcgacaccatgtggtcacgctgggtatttaagcccgagtgagcacgcagggtctccatttt
gaagcgggaggtttgaacgcgcagccgccatgccggggttttacgagattgtgattaaggtcccc
agcgaccttgacgagcatctgcccggcatttctgacagctttgtgaactgggtggccgagaagga
atgggagttgccgccagattctgacatggatctgaatctgattgagcaggcacccctgaccgtgg
ccgagaagctgcagcgcgactttctgacggaatggcgccgtgtgagtaaggccccggaggccctt
ttctttgtgcaatttgagaagggagagagctacttccacatgcacgtgctcgtggaaaccaccgg
ggtgaaatccatggttttgggacgtttcctgagtcagattcgcgaaaaactgattcagagaattt
accgcgggatcgagccgactttgccaaactggttcgcggtcacaaagaccagaaatggcgccgga
ggcgggaacaaggtggtggatgagtgctacatccccaattacttgctccccaaaacccagcctga
gctccagtgggcgtggactaatatggaacagtatttaagcgcctgtttgaatctcacggagcgta
aacggttggtggcgcagcatctgacgcacgtgtcgcagacgcaggagcagaacaaagagaatcag
aatcccaattctgatgcgccggtgatcagatcaaaaacttcagccaggtacatggagctggtcgg
gtggctcgtggacaaggggattacctcggagaagcagtggatccaggaggaccaggcctcataca
tctccttcaatgcggcctccaactcgcggtcccaaatcaaggctgccttggacaatgcgggaaag
attatgagcctgactaaaaccgcccccgactacctggtgggccagcagcccgtggaggacatttc
cagcaatcggatttataaaattttggaactaaacgggtacgatccccaatatgcggcttccgtct
ttctgggatgggccacgaaaaagttcggcaagaggaacaccatctggctgtttgggcctgcaact
accgggaagaccaacatcgcggaggccatagcccacactgtgcccttctacgggtgcgtaaactg
gaccaatgagaactttcccttcaacgactgtgtcgacaagatggtgatctggtgggaggagggga
agatgaccgccaaggtcgtggagtcggccaaagccattctcggaggaagcaaggtgcgcgtggac
cagaaatgcaagtcctcggcccagatagacccgactcccgtgatcgtcacctccaacaccaacat
gtgcgccgtgattgacgggaactcaacgaccttcgaacaccagcagccgttgcaagaccggatgt
tcaaatttgaactcacccgccgtctggatcatgactttgggaaggtcaccaagcaggaagtcaaa
gactttttccggtgggcaaaggatcacgtggttgaggtggagcatgaattctacgtcaaaaaggg
tggagccaagaaaagacccgcccccagtgacgcagatataagtgagcccaaacgggtgcgcgagt
cagttgcgcagccatcgacgtcagacgcggaagcttcgatcaactacgcagacaggtaccaaaac
aaatgttctcgtcacgtgggcatgaatctgatgctgtttccctgcagacaatgcgagagaatgaa
tcagaattcaaatatctgcttcactcacggacagaaagactgtttagagtgctttcccgtgtcag
aatctcaacccgtttctgtcgtcaaaaaggcgtatcagaaactgtgctacattcatcatatcatg
ggaaaggtgccagacgcttgcactgcctgcgatctggtcaatgtggatttggatgactgcatctt
tgaacaataaatgatttaaatcaggtatggctgccgatggttatcttccagattggctcgaggac
aaccttagtgaaggaattcgcgagtggtgggctttgaaacctggagcccctcaacccaaggcaaa
tcaacaacatcaagacaacgctcgaggtcttgtgcttccgggttacaaataccttggacccggca
acggactcgacaagggggagccggtcaacgcagcagacgcggcggccctcgagcacgacaaggcc
tacgaccagcagctcaaggccggagacaacccgtacctcaagtacaaccacgccgacgccgagtt
ccaggagcggctcaaagaagatacgtcttttgggggcaacctcgggcgagcagtcttccaggcca
aaaagaggcttcttgaacctcttggtctggttgaggaagcggctaagacggctcctggaaagaag
aggcctgtagagcagtctcctcaggaaccggactcctccgcgggtattggcaaatcgggtgcaca
gcccgctaaaaagagactcaatttcggtcagactggcgacacagagtcagtcccagaccctcaac
caatcggagaacctcccgcagccccctcaggtgtgggatctcttacaatggcttcaggtggtggc
gcaccagtggcagacaataacgaaggtgccgatggagtgggtagttcctcgggaaattggcattg
cgattcccaatggctgggggacagagtcatcaccaccagcacccgaacctgggccctgcccacct
acaacaatcacctctacaagcaaatctccaacagcacatctggaggatcttcaaatgacaacgcc
tacttcggctacagcaccccctgggggtattttgacttcaacagattccactgccacttctcacc
acgtgactggcagcgactcatcaacaacaactggggattccggcctaagcgactcaacttcaagc
tcttcaacattcaggtcaaagaggttacggacaacaatggagtcaagaccatcgccaataacctt
accagcacggtccaggtcttcacggactcagactatcagctcccgtacgtgctcgggtcggctca
cgagggctgcctcccgccgttcccagcggacgttttcatgattcctcagtacgggtatctgacgc
ttaatgatggaagccaggccgtgggtcgttcgtccttttactgcctggaatatttcccgtcgcaa
atgctaagaacgggtaacaacttccagttcagctacgagtttgagaacgtacctttccatagcag
ctacgctcacagccaaagcctggaccgactaatgaatccactcatcgaccaatacttgtactatc
tctcaaagactattaacggttctggacagaatcaacaaacgctaaaattcagtgtggccggaccc
agcaacatggctgtccagggaagaaactacatacctggacccagctaccgacaacaacgtgtctc
aaccactgtgactcaaaacaacaacagcgaatttgcttggcctggagcttcttcttgggctctca
atggacgtaatagcttgatgaatcctggacctgctatggccagccacaaagaaggagaggaccgt
ttctttcctttgtctggatctttaatttttggcaaacaaggaactggaagagacaacgtggatgc
ggacaaagtcatgataaccaacgaagaagaaattaaaactactaacccggtagcaacggagtcct
atggacaagtggccacaaaccaccagagtgcccaagcacaggcgcagaccggctgggttcaaaac
caaggaatacttccgggtatggtttggcaggacagagatgtgtacctgcaaggacccatttgggc
caaaattcctcacacggacggcaactttcacccttctccgctgatgggagggtttggaatgaagc
acccgcctcctcagatcctcatcaaaaacacacctgtacctgcggatcctccaacggccttcaac
aaggacaagctgaactctttcatcacccagtattctactggccaagtcagcgtggagatcgagtg
ggagctgcagaaggaaaacagcaagcgctggaacccggagatccagtacacttccaactattaca
agtctaataatgttgaatttgctgttaatactgaaggtgtatatagtgaaccccgccccattggc
accagatacctgactcgtaatctgtaattgcttgttaatcaataaaccgtttaattcgtttcagt
tgaactttggtctctgcgaagggcgaattcgtttaaacctgcaggactagaggtcctgtattaga
ggtcacgtgagtgttttgcgacattttgcgacaccatgtggtcacgctgggtatttaagcccgag
tgagcacgcagggtctccattttgaagcgggaggtttgaacgcgcagccgccaagccgaattctg
cagatatccatcacactggcggccgctcgactagagcggccgccaccgcggtggagctccagctt
ttgttccctttagtgagggttaattgcgcgcttggcgtaatcatggtcatagctgcgcgtagtat
gctgaaataggtgactagaagttcctatactttctagagaataggaacttcataacttcgtataa
tgtatgctatacgaagttatgggttactttaatttggttgctgactaattgagatgcatgctttg
catacttctgcctgctggggagcctggggactttccacacctggttgctgactaattgagatgca
tgctttgcatacttctgcctgctggggagcctggggactttccacacc
Helper msDNA
SEQ ID NO: 51
cgcgtagtatgctgaaataggtgactagaagttcctatactttctagagaataggaacttcataa
cttcgtataatgtatgctatacgaagttatgggttactttaatttggttgctgactaattgagat
gcatgctttgcatacttctgcctgctggggagcctggggactttccacacctggttgctgactaa
ttgagatgcatgctttgcatacttctgcctgctggggagcctggggactttccacaccgatccgg
cgcacaccaaaaacgtcacttttgccacatccgtcgcttacatgtgttccgccacacttgcaaca
tcacacttccgccacactactacgtcacccgccccgttcccacgccccgcgccacgtcacaaact
ccaccccctcattatcatattggcttcaatccaaaataatcatcaataatataccttattttgga
ttgaagccaatatgataatgagggggtggagtttgtgacgtggcgcggggcgtgggaacggggcg
ggtgacgtaggttttagggcggagtaacttgtatgtgttgggaattgtagttttcttaaaatggg
aagtgacgtaacgtgggaaaacggaagtgacgatttgaggaagttgtgggttttttggctttcgt
ttctgggcgtaggttcgcgtgcggttttctgggtgttttttgtggactttaaccgttacgtcatt
ttttagtcctatatatactcgctctgcacttggcccttttttacactgtgactgattgagctggt
gccgtgtcgagtggtgtttttttaataggttttcttttttactggtaaggctgactgttatggct
gccgctgtggaagcgctgtatgttgttctggagcgggagggtgctattttgcctaggcaggaggg
tttttcaggtgtttatgtgtttttctctcctattaattttgttatacctcctatgggggctgtaa
tgttgtctctacgcctgcgggtatgtattcccccgggctatttcggtcgctttttagcactgacc
gatgtgaatcaacctgatgtgtttaccgagtcttacattatgactccggacatgaccgaggagct
gtcggtggtgctttttaatcacggtgaccagtttttttacggtcacgccggcatggccgtagtcc
gtcttatgcttataagggttgtttttcctgttgtaagacaggcttctaatgtttaaatgtttttt
tgttattttattttgtgtttatgcagaaacccgcagacatgtttgagagaaaaatggtgtctttt
tctgtggtggttccggagcttacctgcctttatctgcatgagcatgactacgatgtgctttcttt
tttgcgcgaggctttgcctgattttttgagcagcaccttgcattttatatcgccgcccatgcaac
aagcttacatcggggctacgctggttagcatagctccgagtatgcgtgtcataatcagtgtgggt
tcttttgtcatggttcctggcggggaagtggccgcgctggtccgtgcagacctgcacgattatgt
tcagctggccctgcgaagggacctacgggatcgcggtatttttgttaatgttccgcttttgaatc
ttatacaggtctgtgaggaacctgaatttttgcaatcatgattcgctgcttgaggctgaaggtgg
agggcgctctggagcagatttttacaatggccggacttaatattcgggatttgcttagagatata
ttgagaaggtggcgagatgagaattatttgggcatggttgaaggtgctggaatgtttatagagga
gattcaccctgaagggtttagcctttacgtccacttggacgtgagggccgtttgccttttggaag
ccattgtgcaacatcttacaaatgccattatctgttctttggctgtagagtttgaccacgccacc
ggaggggagcgcgttcacttaatagatcttcattttgaggttttggataatcttttggaataaaa
aaaaaaacatggttcttccagctcttcccgctcctcccgtgtgtgactcgcagaacgaatgtgta
ggttggctgggtgtggcttattctgcggtggtggatgttatcagggcagcggcgcatgaaggagt
ttacatagaacccgaagccagggggcgcctggatgctttgagagagtggatatactacaactact
acacagagcgatctaagcggcgagaccggagacgcagatctgtttgtcacgcccgcacctggttt
tgcttcaggaaatatgactacgtccggcgttccatttggcatgacactacgaccaacacgatctc
ggttgtctcggcgcactccgtacagtagggatcgtctacctccttttgagacagaaacccgcgct
accatactggaggatcatccgctgctgcccgaatgtaacactttgacaatgcacaacgtgagtta
cgtgcgaggtcttccctgcagtgtgggatttacgctgattcaggaatgggttgttccctgggata
tggttctaacgcgggaggagcttgtaatcctgaggaagtgtatgcacgtgtgcctgtgttgtgcc
aacattgatatcatgacgagcatgatgatccatggttacgagtcctgggctctccactgtcattg
ttccagtcccggttccctgcagtgtatagccggcgggcaggttttggccagctggtttaggatgg
tggtggatggcgccatgtttaatcagaggtttatatggtaccgggaggtggtgaattacaacatg
ccaaaagaggtaatgtttatgtccagcgtgtttatgaggggtcgccacttaatctacctgcgctt
gtggtatgatggccacgtgggttctgtggtccccgccatgagctttggatacagcgccttgcact
gtgggattttgaacaatattgtggtgctgtgctgcagttactgtgctgatttaagtgagatcagg
gtgcgctgctgtgcccggaggacaaggcgccttatgctgcgggcggtgcgaatcatcgctgagga
gaccactgccatgttgtattcctgcaggacggagcggcggcggcagcagtttattcgcgcgctgc
tgcagcaccaccgccctatcctgatgcacgattatgactctacccccatgtaggcgtggacttct
ccttcgccgcccgttaagcaaccgcaagttggacagcagcctgtggctcagcagctggacagcga
catgaacttaagtgagctgcccggggagtttattaatatcactgatgagcgtttggctcgacagg
aaaccgtgtggaatataacacctaagaatatgtctgttacccatgatatgatgctttttaaggcc
agccggggagaaaggactgtgtactctgtgtgttgggagggaggtggcaggttgaatactagggt
tctgtgagtttgattaaggtacggtgatctgtataagctatgtggtggtggggctatactactga
atgaaaaatgacttgaaattttctgcaattgaaaaataaacacgttgaaacataacacaaacgat
tctttattcttgggcaatgtatgaaaaagtgtaagaggatgtggcaaatatttcattaatgtagt
tgtggccagaccagtcccatgaaaatgacatagagtatgcacttggagttgtgtctcctgtttcc
tgtgtaccgtttagtgtaatggttagtgttacaggtttagttttgtctccgtttaagtaaacttg
actgacaatgttacttttggcagttttaccgtgagattttggataagctgataggttaggcataa
atccaacagcgtttgtataggctgtgccttcagtaagatctccatttctaaagttccaatattct
gggtccaggaaggaattgtttagtagcactccattttcgtcaaatcttataataagatgagcact
ttgaactgttccagatattggagccaaactgcctttaacagccaaaactgaaactgtagcaagta
tttgactgccacattttgttaagaccaaagtgagtttagcatctttctctgcatttagtctacag
ttaggagatggagctggtgtggtccacaaagttagcttatcattatttttgtttcctactgtaat
ggcacctgtgctgtcaaaactaaggccagttcctagtttaggaaccatagccttgtttgaatcaa
attctaggccatggccaatttttgttttgaggggatttgtgtttggtgcattaggtgaaccaaat
tcaagcccatctcctgcattaatggctatggctgtagcgtcaaacatcaaccccttggcagtgct
taggttaacctcaagctttttggaattgtttgaagctgtaaacaagtaaaggcctttgttgtagt
taatatccaagttgtgggctgagtttataaaaagagggccctgtcctagtcttagatttagttgg
ttttgagcatcaaacggataactaacatcaagtataaggcgtctgttttgagaatcaatccttag
tcctcctgctacattaagttgcatattgccttgtgaatcaaaacccaaggctccagtaactttag
tttgcaaggaagtattattaatagtcacacctggaccagttgctacggtcaaagtgtttaggtcg
tctgttacatgcaaaggagccccgtactttagtcctagttttccattttgtgtataaatgggctc
tttcaagtcaatgcccaagctaccagtggcagtagttagagggggtgaggcagtgatagtaaggg
tactgctatcggtggtggtgagggggcctgatgtttgcagggctagctttccttctgacactgtg
aggggtccttgggtggcaatgctaagtttggagtcgtgcacggttagcggggcctgtgattgcat
ggtgagtgtgttgcccgcgaccattagaggtgcggcggcagccacagttagggcttctgaggtaa
ctgtgaggggtgcagatatttccaggtttatgtttgacttggtttttttgagaggtgggctcaca
gtggttacattttgggaggtaaggttgccggcctcgtccagagagaggccgttgcccattttgag
cgcaagcatgccattggaggtaactagaggttcggataggcgcaaagagagtaccccagggggac
tctcttgaaacccattgggggatacaaagggaggagtaagaaaaggcacagttggaggaccggtt
tccgtgtcatatggatacacggggttgaaggtatcttcagacggtcttgcgcgcttcatctgcaa
caacatgaagatagtgggtgcggatggacaggaacaggaggaaactgacattccatttagattgt
ggagaaagtttgcagccaggaggaagctgcaataccagagctgggaggagggcaaggaggtgctg
ctgaataaactggacagaaatttgctaactgattttaagtaagtgatgctttattattttttttt
attagttaaagggaataagatccccgggtactctagttaattaactagaggatcttgatgtaatc
cagggttaggacagttgcaaatcacagtgagaacacagggtcccctgtcccgctcaactagcagg
gggcgctgggtaaactcccgaatcaggctacgggcaagctctccctgggcggtaagccggacgcc
gtgcgccgggccctcgatatgatcctcgggcaattcaaagtagcaaaactcaccggagtcgcggg
caaagcacttgtggcggcgacagtggaccaggtgtttcaggcgcagttgctctgcctctccactt
aacattcagtcgtagccgtccgccgagtcctttaccgcgtcaaagttaggaataaattgatccgg
atagtggccgggaggtcccgagaaggggttaaagtagaccgatggcacaaactcctcaataaatt
gcagagttccaatgcctccagagcgcggctcagaggacgaggtctgcagagttaggattgcctga
cgaggcgtgaatgaagagcggccggcgccgccgatctgaaatgtcccgtccggacggagaccaag
cgaggagctcaccgactcgtcgttgagctgaatacctcgccctctgattgtcaggtgagttatac
cctgcccgggcgaccgcaccctgtgacgaaagccgcccgcaagctgcgcccctgagttagtcatc
tgaacttcggcctgggcgtctctgggaagtaccacagtggtgggagcgggactttcctggtacac
cagggcagcgggccaactacggggattaaggttattacgaggtgtggtggtaatagccgcctgtt
ccaggagaattcggtttcggtgggcgcgtattccgttgacccgggatatcatgtggggtcccgcg
ctcatgtagtttattcgggttgagtagtcttgggcagctccagccgcaagtcccatttgtggctg
gtaactccacatgtagggcgtgggaatttccttgctcataatggcgctgacaacaggtgctggcg
ccgggtgtggccgctggagatgacgtagttttcgcgcttaaatttgagaaagggcgcgaaactag
tccttaagagtcagcgcgcagtatttactgaagagagcctccgcgtcttccagcgtgcgccgaag
ctgatcttcgcttttgtgatacaggcagctgcgggtgagggatcgcagagacctgttttttattt
tcagctcttgttcttggcccctgctctgttgaaatatagcatacagagtgggaaaaatcctgttt
ctaagctcgcgggtcgatacgggttcgttgggcgccagacgcagcgctcctcctcctgctgctgc
cgccgctgtggatttcttgggctttgtcagagtcttgctatccggtcgcctttgcttctgtgtgg
ccgctgctgttgctgccgctgccgccggtgcagtatgggctgtagagatgacggtagtaatgcag
gatgttacgggggaaggccacgccgtgatggtagagaagaaagcggcgggcgaaggagatgttgc
ccccacagtcttgcaagcaagcaactatggcgttcttgtgcccgcgccatgagcggtagccttgg
cgctgttgttgctcttgggctaacggcggcggctgcttggacttaccggccctggttccagtggt
gtcccatctacggttgggtcggcgaacgggcagtgccggcggcgcctgaggagcggaggttgtag
ccatgctggaaccggttgccgatttctggggcgccggcgaggggaatgcgaccgagggtgacggt
gtttcgtctgacacctcttcgacctcggaagcttcctcgtctaggctctcccagtcttccatcat
gtcctcctcctcctcgtccaaaacctcctctgcctgactgtcccagtattcctcctcgtccgtgg
gtggcggcggcagctgcagcttctttttgggtgccatcctgggaagcaagggcccgcggctgctg
ctgatagggctgcggcggcggggggattgggttgagctcctcgccggactgggggtccaagtaaa
ccccccgtccctttcgtagcagaaactcttggcgggctttgttgatggcttgcaattggccaaga
atgtggccctgggtaatgacgcaggcggtaagctccgcattaggcgggcgggattggtcttcgta
gaacctaatctcgtgggcgtggtagtcctcaggtacaaatttgcgaaggtaagccgacgtccaca
gccccggagtgagtttcaaccccggagccgcggacttttcgtcaggcgagggaccctgcagctca
aaggtaccgataatttgactttcgttaagcagctgcgaattgcaaaccagggagcggtgcggggt
gcataggttgcagcgacagtgacactccagtagaccgtcaccgctcacgtcttccattatgtcag
agtggtaggcaaggtagttggctagctgcagaaggtagcagtggccccaaagcggcggagggcat
tcgcggtacttaatgggcacaaagtcgctaggaagtgcacagcaggtggcgggcaagattcctga
gcgctctaggataaagttcctaaagttctgcaacatgctttgactggtgaagtctggcagaccct
gttgcagggttttaagcaggcgttcggggaaaatgatgtccgccaggtgcgcggccacggagcgc
tcgttgaaggccgtccataggtccttcaagttttgctttagcagtttctgcagctccttgaggtt
gcactcctccaagcactgctgccaaacgcccatggccgtctgccaggtgtagcatagaaataagt
aaacgcagtcgcggacgtagtcgcggcgcgcctcgcccttgagcgtggaatgaagcacgttttgc
ccaaggcggttttcgtgcaaaattccaaggtaggagaccaggttgcagagctccacgttggagat
cttgcaggcctggcgtacgtagccctgtcgaaaggtgtagtgcaatgtttcctctagcttgcgct
gcatctccgggtcagcaaagaaccgctgcatgcactcaagctccacggtaacgagcactgcggcc
atcattagtttgcgtcgctcctccaagtcggcaggctcgcgcgtttgaagccagcgcgctagctg
ctcgtcgccaactgcgggtaggccctcctctgtttgttcttgcaaatttgcatccctctccaggg
gctgcgcacggcgcacgatcagctcactcatgactgtgctcatgaccttggggggtaggttaagt
gccgggtaggcaaagtgggtgacctcgatgctgcgttttagtacggctaggcgcgcgttgtcacc
ctcgagttccaccaacactccagagtgactttcattttcgctgttttcctgttgcagagcgtttg
ccgcgcgcttctcgtcgcgtccaagaccctcaaagatttttggcacttcgttgagcgaggcgata
tcaggtatgacagcgccctgccgcaaggccagctgcttgtccgctcggctgcggttggcacggca
ggataggggtatcttgcagttttggaaaaagatgtgataggtggcaagcacctctggcacggcaa
atacggggtagaagttgaggcgcgggttgggctcgcatgtgccgttttcttggcgtttggggggt
acgcgcggtgagaataggtggcgttcgtaggcaaggctgacatccgctatggcgaggggcacatc
gctgcgctcttgcaacgcgtcgcagataatggcgcactggcgctgcagatgcttcaacagcacgt
cgtctcccacatctaggtagtcgccatgcctttcgtccccccgcccgacttgttcctcgtttgcc
tctgcgttgtcctggtcttgctttttatcctctgttggtactgagcggtcctcgtcgtcttcgct
tacaaaacctgggtcctgctcgataatcacttcctcctcctcaagcgggggtgcctcgacgggga
aggtggtaggcgcgttggcggcatcggtggaggcggtggtggcgaactcagagggggcggttagg
ctgtccttcttctcgactgactccatgatctttttctgcctataggagaaggaaatggccagtcg
ggaagaggagcagcgcgaaaccacccccgagcgcggacgcggtgcggcgcgacgtcccccaacca
tggaggacgtgtcgtccccgtccccgtcgccgccgcctccccgggcgcccccaaaaaagcggatg
aggcggcgtatcgagtccgaggacgaggaagactcatcacaagacgcgctggtgccgcgcacacc
cagcccgcggccatcgacctcggcggcggatttggccattgcgcccaagaagaaaaagaagcgcc
cttctcccaagcccgagcgcccgccatcaccagaggtaatcgtggacagcgaggaagaaagagaa
gatgtggcgctacaaatggtgggtttcagcaacccaccggtgctaatcaagcatggcaaaggagg
taagcgcacagtgcggcggctgaatgaagacgacccagtggcgcgtggtatgcggacgcaagagg
aagaggaagagcccagcgaagcggaaagtgaaattacggtgatgaacccgctgagtgtgccgatc
gtgtctgcgtgggagaagggcatggaggctgcgcgcgcgctgatggacaagtaccacgtggataa
cgatctaaaggcgaacttcaaactactgcctgaccaagtggaagctctggcggccgtatgcaaga
cctggctgaacgaggagcaccgcgggttgcagctgaccttcaccagcaacaagacctttgtgacg
atgatggggcgattcctgcaggcgtacctgcagtcgtttgcagaggtgacctacaagcatcacga
gcccacgggctgcgcgttgtggctgcaccgctgcgctgagatcgaaggcgagcttaagtgtctac
acggaagcattatgataaataaggagcacgtgattgaaatggatgtgacgagcgaaaacgggcag
cgcgcgctgaaggagcagtctagcaaggccaagatcgtgaagaaccggtggggccgaaatgtggt
gcagatctccaacaccgacgcaaggtgctgcgtgcacgacgcggcctgtccggccaatcagtttt
ccggcaagtcttgcggcatgttcttctctgaaggcgcaaaggctcaggtggcttttaagcagatc
aaggcttttatgcaggcgctgtatcctaacgcccagaccgggcacggtcaccttttgatgccact
acggtgcgagtgcaactcaaagcctgggcacgcgccctttttgggaaggcagctaccaaagttga
ctccgttcgccctgagcaacgcggaggacctggacgcggatctgatctccgacaagagcgtgctg
gccagcgtgcaccacccggcgctgatagtgttccagtgctgcaaccctgtgtatcgcaactcgcg
cgcgcagggcggaggccccaactgcgacttcaagatatcggcgcccgacctgctaaacgcgttgg
tgatggtgcgcagcctgtggagtgaaaacttcaccgagctgccgcggatggttgtgcctgagttt
aagtggagcactaaacaccagtatcgcaacgtgtccctgccagtggcgcatagcgatgcgcggca
gaacccctttgatttttaaacggcgcagacggcaagggtgggggtaaataatcacccgagagtgt
acaaataaaagcatttgcctttattgaaagtgtctctagtacattatttttacatgtttttcaag
tgacaaaaagaagtggcgctcctaatctgcgcactgtggctgcggaagtagggcgagtggcgctc
caggaagctgtagagctgttcctggttgcgacgcagggtgggctgtacctggggactgttgagca
tggagttgggtaccccggtaataaggttcatggtggggttgtgatccatgggagtttggggccag
ttggcaaaggcgtggagaaacatgcagcagaatagtccacaggcggccgagttgggcccctgtac
gctttgggtggacttttccagcgttatacagcggtcgggggaagaagcaatggcgctacggcgca
ggagtgactcgtactcaaactggtaaacctgcttgagtcgctggtcagaaaagccaaagggctca
aagaggtagcatgtttttgagtgcgggttccaggcaaaggccatccagtgtacgcccccagtctc
ggtccgagactcgaaccgggggtcccgcgactcaacccttggaaaataaccctccggctacaggg
agcgagccacttaatgctttcgctttccagcctaaccgcttacgctgcgcgcggccagtggccaa
aaaagctagcgcagcagccgccgcgcctggaaggaagccaaaaggagcactcccccgttgtctga
cgtcgcacacctgggttcgacacgcgggcggtaaccgcatggatcacggcggacggccggatacg
gggctcgaaccccggtcgtccgccatgatacccttgcgaatttatccaccagaccacggaagagt
gcccgcttacaggctctccttttgcacgctagagcgtcaacgattgcgcgcgcctgaccggccag
agcgtcccgaccatggagcactttttgccgctgcgcaacatctggaaccgcgtccgcgactttcc
gcgcgcctccaccaccgccgccggcatcacctggatgtccaggtacatctacggatatcatcgcc
ttatgttggaagatctcgcccccggagccccggccaccctacgctggcccctctaccgccagccg
ccgccgcactttttggtgggataccagtacctggtgcggacttgcaacgactacgtatttgactc
gagggcttactcgcgtctcaggtacaccgagctctcgcagccgggtcaccagaccgttaactggt
ccgttatggccaactgcacttacaccatcaacacgggcgcataccaccgctttgtggacatggat
gacttccagtctaccctcacgcaggtgcagcaggccatattagccgagcgcgttgtcgccgacct
agccctgcttcagccgatgaggggcttcggggtcacacgcatgggaggaagagggcgccacctac
ggccaaactccgccgccgccgcagcgatagatgcaagagatgcaggacaagaggaaggagaagaa
gaagtgccggtagaaaggctcatgcaagactactacaaagacctgcgccgatgtcaaaacgaagc
ctggggcatggccgaccgcctgcgcattcagcaggccggacccaaggacatggtgcttctgtcga
cggtatcgataagcttgatatcgaattcctgcagcccgggggatccgcgtagtatgctgaaatag
gtgactagaagttcctatactttctagagaataggaacttcataacttcgtataatgtatgctat
acgaagttatgggttactttaatttggttgctgactaattgagatgcatgctttgcatacttctg
cctgctggggagcctggggactttccacacctggttgctgactaattgagatgcatgctttgcat
acttctgcctgctggggagcctggggactttccacacc

What is claimed is:

1. An expression vector comprising:
(a) a first sequence comprising an inverted terminal repeat (ITR) and a multiple cloning site (MCS), wherein the ITR flanks at least one side of the MCS, and wherein the ITR comprises a sequence for adeno-associated virus (AAV) replication and an AAV packaging signal,
(b) a target sequence for a first recombinase flanking each side of the first sequence, and
(c) one or more additional target sequences for one or more additional recombinases integrated within non-binding regions of the target sequence for the first recombinase,
wherein the expression vector is for producing a bacterial sequence-free vector having linear covalently closed ends.

2. The expression vector of claim 1, wherein the ITR flanks only one side of the MCS.

3. The expression vector of claim 1, wherein the ITR flanks each side of the MCS.

4. The expression vector of any one of claims 1 to 3, further comprising a spacer sequence between the target sequence for the first recombinase and the first sequence.

5. The expression vector of claim 4, wherein the spacer sequence is 10 to 500 nucleotides.

6. The expression vector of any one of claims 1 to 3, further comprising an expression cassette comprising an AAV replication (rep) gene and an AAV capsid (cap) gene flanked on one side by the target sequence for the first recombinase and flanked on the other side by the first sequence.

7. An expression vector comprising:
(a) a first sequence comprising an ITR and an expression cassette comprising a nucleic acid sequence of interest, wherein the ITR flanks at least one side of the expression cassette comprising the nucleic acid sequence of interest, and wherein the ITR comprises a sequence for AAV replication and an AAV packaging signal,
(b) a target sequence for a first recombinase flanking each side of the first sequence, and
(c) one or more additional target sequences for one or more additional recombinases integrated within non-binding regions of the target sequence for the first recombinase,
wherein the expression vector is for producing a bacterial sequence-free vector having linear covalently closed ends.

8. The expression vector of claim 7, wherein the ITR flanks only one side of the expression cassette comprising the nucleic acid sequence of interest.

9. The expression vector of claim 8, further comprising a spacer sequence between the target sequence for the first recombinase and the first sequence.

10. The expression vector of claim 9, wherein the spacer sequence is 10 to 500 nucleotides.

11. The expression vector of any one of claims 8 to 10, further comprising an expression cassette comprising an AAV rep gene and an AAV cap gene flanked on one side by the target sequence for the first recombinase and flanked on the other side by the first sequence.

12. The expression vector of claim 7, wherein the ITR flanks each side of the expression cassette comprising the nucleic acid sequence of interest.

13. The expression vector of claim 12, further comprising a spacer sequence between the target sequence for the first recombinase and the first sequence.

14. The expression vector of claim 13, wherein the spacer sequence is 10 to 500 nucleotides.

15. The expression vector of any one of claims 12 to 14, further comprising an expression cassette comprising an AAV rep gene and an AAV cap gene flanked on one side by the target sequence for the first recombinase and flanked on the other side by the first sequence.

16. A expression vector comprising:
(a) a first sequence comprising an ITR and a palindromic sequence, wherein the ITR flanks each side of the palindromic sequence, wherein the palindromic sequence comprises an expression cassette comprising a nucleic acid sequence of interest and a complement of the expression cassette, and wherein the ITR comprises a sequence for AAV replication and an AAV packaging signal,
(b) a target sequence for a first recombinase flanking each side of the first sequence, and
(c) one or more additional target sequences for one or more additional recombinases integrated within non-binding regions of the target sequence for the first recombinase,
wherein the expression vector is for producing a bacterial sequence-free vector having linear covalently closed ends.

17. The expression vector of claim 16, wherein the complement is separated from the expression cassette comprising the nucleic acid sequence of interest by a non-complementary spacer sequence.

18. The expression vector of claim 16 or 17, further comprising a spacer sequence between the target sequence for the first recombinase and the first sequence.

19. The expression vector of claim 18, wherein the spacer sequence is 10 to 500 nucleotides.

20. The expression vector of any one of claims 16 to 19, further comprising an expression cassette comprising an AAV rep gene and an AAV cap gene flanked on one side by the target sequence for the first recombinase and flanked on the other side by the first sequence.

21. An expression vector comprising:
(a) a first sequence comprising a portion of an expression cassette comprising a nucleic acid sequence of interest flanked on one side by a splicing sequence,
(b) an ITR flanking each side of the first sequence, wherein the ITR comprises a sequence for AAV replication and an AAV packaging signal,
(c) a target sequence for a first recombinase flanking each ITR, and
(d) one or more additional target sequences for one or more additional recombinases integrated within non-binding regions of the target sequence for the first recombinase,
wherein the expression vector is for producing a bacterial sequence-free vector having linear covalently closed ends.

22. The expression vector of claim 21, wherein the portion of the expression cassette comprises a 5′ portion that in combination with the remaining portion of the expression cassette provides the complete sequence of the expression cassette, and the splicing sequence flanks the 3′ end of the 5′ portion.

23. The expression vector of claim 21, wherein the portion of the expression cassette comprises a 3′ portion that in combination with the remaining portion of the expression cassette provides the complete sequence of the expression cassette, and the splicing sequence flanks the 5′ end of the 3′ portion.

24. The expression vector of any one of claims 21 to 23, further comprising a spacer sequence between the target sequence for the first recombinase and the first sequence.

25. The expression vector of claim 24, wherein the spacer sequence is 10 to 500 nucleotides.

26. The expression vector of any one of claims 1 to 25, wherein the sequence for AAV replication comprises an AAV ITR Replication (Rep) protein binding element (RBE) and terminal resolution site (TRS).

27. The expression vector of any one of claims 1 to 26, wherein the AAV packaging signal comprises an AAV ITR D-sequence.

28. An expression vector comprising:
(a) an expression cassette comprising an AAV Rep gene and an AAV Cap gene,
(b) a target sequence for a first recombinase flanking each side of the expression cassette, and
(c) one or more additional target sequences for one or more additional recombinases integrated within non-binding regions of the target sequence for the first recombinase
wherein the expression vector is for producing a bacterial sequence-free vector having linear covalently closed ends.

29. An expression vector comprising:
(a) an expression cassette comprising one or more helper virus genes for production of AAV,
(b) a target sequence for a first recombinase flanking each side of the expression cassette, and
(c) one or more additional target sequences for one or more additional recombinases integrated within non-binding regions of the target sequence for the first recombinase,
wherein the expression vector is for producing a bacterial sequence-free vector having linear covalently closed ends.

30. The expression vector of claim 29, wherein the one or more helper virus genes are from an adenovirus, a herpesvirus, a retrovirus, a poxvirus, and/or a lentivirus.

31. The expression vector of claim 30, wherein the one or more helper virus genes comprise an adenovirus Early 4 (E4) gene, adenovirus Early 2A (E2A) gene, and adenovirus Viral Associated (VA) gene.

32. The expression vector of any one of claims 1 to 31, wherein the target sequence for the first recombinase and the one or more additional target sequences for the one or more additional recombinases are selected from the group consisting of the PY54 pal site, the N15 telRL site, and the qK02 telRL site.

33. The expression vector of claim 32, wherein the expression vector comprises each of the target sequences.

34. The expression vector of claim 32, wherein the expression vector comprises the Tel recombinase pal site and the telRL recombinase target binding sequence integrated within the pal site.

35. The expression vector of any one of claims 1 to 31, wherein the target sequence for the first recombinase is the phage PY54 Tel 142 base pair target site.

36. A vector production system comprising recombinant cells designed to encode at least a first recombinase under the control of an inducible promoter, wherein the cells comprise the expression vector of any one of claims 7 to 35.

37. The vector production system of claim 36, wherein the inducible promoter is thermally-regulated, chemically-regulated, IPTG regulated, glucose-regulated, arabinose inducible, T7 polymerase regulated, cold-shock inducible, pH inducible, or combinations thereof.

38. The vector production system of claim 36 or 37, wherein the first recombinase is selected from TelN and Tel, and the expression vector incorporates the target sequence for at least the first recombinase.

39. The vector production system of any one of claims 36 to 38, wherein the recombinant cells have been further designed to encode a nuclease genome editing system, and wherein the expression vector further comprises a backbone sequence containing a cleavage site for the nuclease genome editing system.

40. The vector production system of claim 39, wherein the nuclease genome editing system is a CRISPR nuclease system comprising a Cas nuclease and gRNA, and the expression vector comprises a target sequence for the gRNA within the backbone sequence.

41. A method of producing a bacterial sequence-free vector having linear covalently closed ends comprising incubating the vector production system of any one of claims 36 to 38 under suitable conditions for expression of the first recombinase.

42. A method of producing a bacterial sequence-free vector having linear covalently closed ends comprising incubating the vector production system of claim 39 or 40 under suitable conditions for expression of the first recombinase and the nuclease genome editing system.

43. The method of claim 41 or 42, further comprising harvesting the bacterial sequence-free vector.

44. A bacterial sequence-free vector produced by the method of any of claims 41 to 43.

45. The bacterial sequence-free vector of claim 44, wherein the bacterial sequence-free vector is produced from the expression vector of any one of claims 8 to 10.

46. The bacterial sequence-free vector of claim 44, wherein the bacterial sequence-free vector is produced from the expression vector of claim 11.

47. The bacterial sequence-free vector of claim 44, wherein the bacterial sequence-free vector is produced from the expression vector of any one of claims 12 to 14.

48. The bacterial sequence-free vector of claim 44, wherein the bacterial sequence-free vector is produced from the expression vector of claim 15.

49. The bacterial sequence-free vector of claim 44, wherein the bacterial sequence-free vector is produced from the expression vector of any one of claims 16 to 19.

50. The bacterial sequence-free vector of claim 44, wherein the bacterial sequence-free vector is produced from the expression vector of claim 20.

51. The bacterial sequence-free vector of claim 44, wherein the bacterial sequence-free vector is produced from the expression vector of any one of claims 21 to 25.

52. The bacterial sequence-free vector of claim 42, wherein the bacterial sequence-free vector is produced from the expression vector of claim 28.

53. The bacterial sequence-free vector of claim 40, wherein the bacterial sequence-free vector is produced from the expression vector of any one of claims 29 to 31.

54. A method for producing a single-stranded AAV, wherein the method comprises:
(a) transfecting cells that are capable of producing an AAV with:
i. the bacterial sequence-free vector of claim 47,
ii. the bacterial sequence-free vector of claim 52 or an expression vector comprising an expression cassette comprising an AAV rep gene and an AAV cap gene, and
iii. the bacterial sequence-free vector of claim 53 or an expression vector comprising an expression cassette comprising one or more helper virus genes for production of AAV, and
(b) incubating the cells under conditions suitable for production of an AAV.

55. A method for producing a single-stranded AAV, wherein the method comprises:
(a) transfecting cells that are capable of producing an AAV with:
i. the bacterial sequence-free vector of claim 48,
ii. the bacterial sequence-free vector of claim 53 or an expression vector comprising an expression cassette comprising one or more helper virus genes for production of AAV, and
(b) incubating the cells under conditions suitable for production of an AAV.

56. A method for producing a single-stranded AAV, wherein the method comprises:
(a) transfecting cells that are capable of producing an AAV with the bacterial sequence-free vector of claim 47, wherein each of an AAV rep gene, an AAV cap gene, and one or more helper virus genes for production of AAV is encoded by the cells or a vector,
(b) incubating the cells under conditions suitable for expression of the rep gene, the cap gene, and the one or more helper virus genes and for production of an AAV.

57. A method for producing a self-complementary AAV, wherein the method comprises:
(a) transfecting cells that are capable of producing an AAV with:
i. the bacterial sequence-free vector of claim 45,
ii. the bacterial sequence-free vector of claim 52 or an expression vector comprising an expression cassette comprising an AAV rep gene and an AAV cap gene, and
iii. the bacterial sequence-free vector of claim 53 or an expression vector comprising an expression cassette comprising one or more helper virus genes for production of AAV, and
(b) incubating the cells under conditions suitable for production of an AAV.

58. A method for producing a self-complementary AAV, wherein the method comprises:
(a) transfecting cells that are capable of producing an AAV with:
iv. the bacterial sequence-free vector of claim 46,
V. the bacterial sequence-free vector of claim 53 or an expression vector comprising an expression cassette comprising one or more helper virus genes for production of AAV, and
(b) incubating the cells under conditions suitable for production of an AAV.

59. A method for producing a self-complementary AAV, wherein the method comprises:
(a) transfecting cells that are capable of producing an AAV with:
i. the bacterial sequence-free vector of claim 49,
ii. the bacterial sequence-free vector of claim 52 or an expression vector comprising an expression cassette comprising an AAV rep gene and an AAV cap gene, and
iii. the bacterial sequence-free vector of claim 53 or an expression vector comprising an expression cassette comprising one or more helper virus genes for production of AAV, and
(b) incubating the cells under conditions suitable for production of an AAV.

60. A method for producing a self-complementary AAV, wherein the method comprises:
(a) transfecting cells that are capable of producing an AAV with:
i. the bacterial sequence-free vector of claim 50,
ii. the bacterial sequence-free vector of claim 53 or an expression vector comprising an expression cassette comprising one or more helper virus genes for production of AAV, and
(b) incubating the cells under conditions suitable for production of an AAV.

61. A method for producing a self-complementary AAV, wherein the method comprises:
(a) transfecting cells that are capable of producing an AAV with the bacterial sequence-free vector of claim 45 or 49, wherein each of an AAV rep gene, an AAV cap gene, and one or more helper virus genes for production of AAV is encoded by the cells or a vector, and
(b) incubating the cells under conditions suitable for expression of the rep gene, the cap gene, and the one or more helper virus genes and for production of an AAV.

62. The method of any one of claims 54 to 61, wherein the cells are HEK293T cells.

63. The method of any one of claims 54 to 62, further comprising harvesting the AAV.

64. An AAV produced by the method of any one of claims 54 to 63.

65. A pharmaceutical composition comprising the AAV of claim 64.

66. A method of treating a disease or disorder in a subject in need thereof, comprising administering the AAV of claim 64 or the pharmaceutical composition of claim 63 to the subject.


SEQUENCE LISTING (51 sequences)

SEQ ID NO: 1 - DNA, 145 bp, synthetic construct
  source 1..145: mol_type = other DNA; note = AAV-2 wild-type 5' ITR (+ strand)
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 2 - DNA, 145 bp, synthetic construct
  source 1..145: mol_type = other DNA; note = AAV-2 wild-type 3' ITR (+ strand)
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 3 - DNA, 41 bp, synthetic construct
  source 1..41: mol_type = other DNA; note = AAV-2 wild-type ITR A-sequence (+ strand)
ttggccactccctctctgcgcgctcgctcgctcactgaggc

SEQ ID NO: 4 - DNA, 41 bp, synthetic construct
  source 1..41: mol_type = other DNA; note = AAV-2 wild-type ITR A'-sequence (+ strand)
gcctcagtgagcgagcgagcgcgcagagagggagtggccaa

SEQ ID NO: 5 - DNA, 10 bp, synthetic construct
  source 1..10: mol_type = other DNA; note = AAV-2 wild-type ITR B-sequence (+ strand)
cgggcgacca

SEQ ID NO: 6 - DNA, 11 bp, synthetic construct
  source 1..11: mol_type = other DNA; note = AAV-2 wild-type ITR B'-sequence (+ strand)
aaggtcgcccg

SEQ ID NO: 7 - DNA, 10 bp, synthetic construct
  source 1..10: mol_type = other DNA; note = AAV-2 wild-type ITR C-sequence (+ strand)
cgcccgggct

SEQ ID NO: 8 - DNA, 11 bp, synthetic construct
  source 1..11: mol_type = other DNA; note = AAV-2 wild-type ITR C'-sequence (+ strand)
ttgcccgggcg

SEQ ID NO: 9 - DNA, 20 bp, synthetic construct
  source 1..20: mol_type = other DNA; note = AAV-2 wild-type 5' ITR D-sequence (+ strand)
ctccatcactaggggttcct

SEQ ID NO: 10 - DNA, 20 bp, synthetic construct
  source 1..20: mol_type = other DNA; note = AAV-2 wild-type 3' ITR D-sequence (+ strand)
aggaacccctagtgatggag

SEQ ID NO: 11 - DNA, 16 bp, synthetic construct
  source 1..16: mol_type = other DNA; note = AAV-2 wild-type ITR A-RBS (+ strand)
gcgcgctcgctcgctc

SEQ ID NO: 12 - DNA, 16 bp, synthetic construct
  source 1..16: mol_type = other DNA; note = AAV-2 wild-type ITR A'-RBS (+ strand)
gagcgagcgagcgcgc

SEQ ID NO: 13-15 - skipped (3 entries)

SEQ ID NO: 16 - DNA, 130 bp, synthetic construct
  source 1..130: mol_type = other DNA; note = AAV artificial 5' ITR (+ strand)
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 17 - DNA, 130 bp, synthetic construct
  source 1..130: mol_type = other DNA; note = AAV artificial 3' ITR (+ strand)
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 18 - DNA, 7902 bp, synthetic construct
  source 1..7902: mol_type = other DNA; note = ITR-CAG-GFP-ITR plasmid
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacaccccaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcactcctcaggtgcaggctgcctatcagaaggtggtggctggtgtggccaatgccctggctcacaaaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgcttaccactgagatctttttccctctgccaaaaattatggggacatcatgaagccccttgagcatctgacttctggctaataaaggaaatttattttcattgcaatagtgtgttggaattttttgtgtctctcactcggaaggacatggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaattggtgtggaaagtccccaggctccccagcaggcagaagtatgcaaagcatgcatctcaattagtcagcaaccatggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttccagggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 19 - DNA, 4762 bp, synthetic construct
  source 1..4762: mol_type = other DNA; note = ITR-CAG-GFP-ITR msDNA
tacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacaccccaggctgcgcaactgttgggaagggcgatcggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagagtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcactcctcaggtgcaggctgcctatcagaaggtggtggctggtgtggccaatgccctggctcacaaaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgcttaccactgagatctttttccctctgccaaaaattatggggacatcatgaagccccttgagcatctgacttctggctaataaaggaaatttattttcattgcaatagtgtgttggaattttttgtgtctctcactcggaaggacatggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgta

SEQ ID NO: 20 - DNA, 4265 bp, synthetic construct
  source 1..4265: mol_type = other DNA; note = CAG-GFP-ITR msDNA
tacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacctttccagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggaaaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcactcctcaggtgcaggctgcctatcagaaggtggtggctggtgtggccaatgccctggctcacaaaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgcttaccactgagatctttttccctctgccaaaaattatggggacatcatgaagccccttgagcatctgacttctggctaataaaggaaatttattttcattgcaatagtgtgttggaattttttgtgtctctcactcggaaggacatggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgta

SEQ ID NO: 21 - DNA, 1866 bp, synthetic construct
  source 1..1866: mol_type = other DNA; note = AAV-2 rep
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 22 - DNA, 2208 bp, synthetic construct
  source 1..2208: mol_type = other DNA; note = AAV-2 cap
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 23 - DNA, 8600 bp, synthetic construct
  source 1..8600: mol_type = other DNA; note = PGL2-SS-CMV-Rep-Cap-SS plasmid
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatagcttacgcgtggccgctcgagacggccggctgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcaattcggcttggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 24 - DNA, 5535 bp, synthetic construct
  source 1..5535: mol_type = other DNA; note = PGL2-SS-CMV-Rep-Cap-SS msDNA
tacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgattctgtggataaccgtattaccgccatgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatagcttacgcgtggccgctcgagacggccggctgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcaattcggcttggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgta

SEQ ID NO: 25 - DNA, 15885 bp, synthetic construct
  source 1..15885: mol_type = other DNA; note = Helper Sequences msDNA
tacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacaccagtacttctcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggatccacaggacggggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgta

SEQ ID NO: 26 - DNA, 16 bp, synthetic construct
  source 1..16: mol_type = other DNA; note = ampR forward primer
atcccgtattgacgcc

SEQ ID NO: 27 - DNA, 16 bp, synthetic construct
  source 1..16: mol_type = other DNA; note = ampR reverse primer
cgctcgtcgtttggta

SEQ ID NO: 28 - DNA, 16 bp, synthetic construct
  source 1..16: mol_type = other DNA; note = F1 ori forward primer
gcgtgatggacagact

SEQ ID NO: 29 - DNA, 17 bp, synthetic construct
  source 1..17: mol_type = other DNA; note = F1 ori reverse primer
gtcgaggtgccgtaaag

SEQ ID NO: 30 - DNA, 16 bp, synthetic construct
  source 1..16: mol_type = other DNA; note = ori forward primer
taccgggttggactca

SEQ ID NO: 31 - DNA, 18 bp, synthetic construct
  source 1..18: mol_type = other DNA; note = ori reverse primer
cccgacaggactataaag

SEQ ID NO: 32 - DNA, 18 bp, synthetic construct
  source 1..18: mol_type = other DNA; note = SCAR forward primer
aacccataattgtgagcg

SEQ ID NO: 33 - DNA, 20 bp, synthetic construct
  source 1..20: mol_type = other DNA; note = SCAR reverse primer
cattaaggtcgggaaaatgc

SEQ ID NO: 34 - DNA, 16 bp, synthetic construct
  source 1..16: mol_type = other DNA; note = kanR forward primer
gccctgaatgaactgc

SEQ ID NO: 35 - DNA, 16 bp, synthetic construct
  source 1..16: mol_type = other DNA; note = kanR reverse primer
ccatccgagtacgtgc

SEQ ID NO: 36 - DNA, 340 bp, synthetic construct
  source 1..340: mol_type = other DNA; note = SSeq
taaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacc

SEQ ID NO: 37 - DNA, 253 bp, synthetic construct
  source 1..253: mol_type = other DNA; note = SSeq after Tel recombination
cgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacc

SEQ ID NO: 38 - DNA, 145 bp, synthetic construct
  source 1..145: mol_type = other DNA; note = AAV artificial 5' ITR (+ strand)
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 39 - DNA, 145 bp, synthetic construct; identical to SEQ ID NO: 2
  source 1..145: mol_type = other DNA; note = AAV artificial 3' ITR (+ strand)

SEQ ID NO: 40 - DNA, 25 bp, synthetic construct
  source 1..25: mol_type = other DNA; note = 5' spacer between SSeq and ITR
cctgattctgtggataaccgtatta

SEQ ID NO: 41 - DNA, 14 bp, synthetic construct
  source 1..14: mol_type = other DNA; note = 3' spacer between SSeq and ITR
acgcaattcggctt

SEQ ID NO: 42 - DNA, 6817 bp, synthetic construct
  source 1..6817: mol_type = other DNA; note = pITR2Cis msDNA precursor plasmid
cccgggaggtaccgagctcttacgcgtgctagaattaaagtaacccaatcagcacacaattgccattatacgcgcgtataatggactattgtgtgctgataaacctatttcagcatactacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgattctgtggataaccgtattattggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctctatattaccctgttatccctagcgtaactgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcttcgaaagatctgctagcttaattaacccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaaatcgtacgcctaggtgatcaagatctgctagcttaattaacccgggactagtgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagagtctacttcgctaccttaggaccgttatagttacgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaacgcaattcggcttggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccaaattaaagtaacccataacttcgtatagcatacattatacgaagttatgaagttcctattctctagaaagtataggaacttctagtcacctatttcagcatactacgcgcgtagtatgctgaaataggtttatcagcacacaatagtccattatacgcgcgtataatggcaattgtgtgctgattgggttactttaatttggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagcccaagctaccatgataagtaagtaatattaaggtacgtggaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatggtactgtaactgagctaacataa

SEQ ID NO: 43 - DNA, 3744 bp, synthetic construct
  source 1..3744: mol_type = other DNA; note = ITR2Cis msDNA
cgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacccctgattctgtggataaccgtattattggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctctatattaccctgttatccctagcgtaactgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcttcgaaagatctgctagcttaattaacccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaaatcgtacgcctaggtgatcaagatctgctagcttaattaacccgggactagtgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagagtctacttcgctaccttaggaccgttatagttacgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaacgcaattcggcttcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacc

SEQ ID NO: 44 - DNA, 5076 bp, synthetic construct
  source 1..5076: mol_type = other DNA; note = pITR2Cis no SSeq plasmid control
ccaatgatatcggaaagaacatgtgagcaaaaggccagcaaaaggccaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctctatattaccctgttatccctagcgtaactgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcttcgaaagatctgctagcttaattaacccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccaaatcgtacgcctaggtgatcaagatctgctagcttaattaacccgggactagtgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagctagagtctacttcgctaccttaggaccgttatagttacgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 45 - DNA, 1811 bp, synthetic construct
  source 1..1811: mol_type = other DNA; note = AAV-2 rep
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttg

SEQ ID NO: 46 - DNA, 2175 bp, synthetic construct
  source 1..2175: mol_type = other DNA; note = AAV-5 cap
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 47 - DNA, 2211 bp, synthetic construct
  source 1..2211: mol_type = other DNA; note = AAV-9 cap
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 48 - DNA, 5110 bp, synthetic construct
  source 1..5110: mol_type = other DNA; note = Rep2/Cap2 msDNA
cgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacaccggaacttctgatctattcgagctcggtacccctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggagagtcctgtattagaggtcacgtgagtgttttgcgacattttgcgacaccatgtggtcacgctgggtatttaagcccgagtgagcacgcagggtctccattttgagcgggaggtttgaacgagcgctggcgcgctcactggccgtcgttttacaacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacc

SEQ ID NO: 49 - DNA, 5012 bp, synthetic construct
  source 1..5012: mol_type = other DNA; note = Rep2/Cap5 msDNA
cgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacaccggaacttctgatctattcgagctcggtacccctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatgcttgttaatcaataaactacgtagataagtagcatggcgggttaatcattaactacagcccgggcgtttaaacagcgggcggaggggtggagtcgtgacgtgaattacgtcatagggttagggagagtcctgtattagaggtcacgtgagtgttttgcgacattttgcgacaccatgtggtcacgctgggtatttaagcccgagtgagcacgcagggtctccattttgagcgggaggtttgaacgagcgctggcgcgctcactggccgtcgttttacaacgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacc

SEQ ID NO: 50 - DNA, 5118 bp, synthetic construct
  source 1..5118: mol_type = other DNA; note = Rep2/Cap9 msDNA
cgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacaccggaacttctgatctattcgagctcggtacccctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgcgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacc

SEQ ID NO: 51 - DNA, 13233 bp, synthetic construct
  source 1..13233: mol_type = other DNA; note = Helper msDNA
cgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacaccgatccggcgcacaccaaaaacgtcacttttgccacatccgtcgcttacatgtgttccgccacacttgcaacatcacacttccgccacactactacgtcacccgccccgttcccacgccccgcgccacgtcacaaactccaccccctcattatcatattggcttcaatccaaaataatcatcaataatataccttattttggattgaagccaatatgataatgagggggtggagtttgtgacgtggcgcggggcgtgggaacggggcgggtgacgtaggttttagggcggagtaacttgtatgtgttgggaattgtagttttcttaaaatgggaagtgacgtaacgtgggaaaacggaagtgacgatttgaggaagttgtgggttttttggctttcgtttctgggcgtaggttcgcgtgcggttttctgggtgttttttgtggactttaaccgttacgtcattttttagtcctatatatactcgctctgcacttggcccttttttacactgtgactgattgagctggtgccgtgtcgagtggtgtttttttaataggttttcttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgtgaatcaacctgatgtgtttaccgagtcttacattatgactccggacatgaccgaggagctgtcggtggtgctttttaatcacggtgaccagtttttttacggtcacgccggcatggccgtagtccgtcttatgcttataagggttgtttttcctgttgtaagacaggcttctaatgtttaaatgtttttttgttattttattttgtgtttatgcagaaacccgcagacatgtttgagagaaaaatggtgtctttttctgtggtggttccggagcttacctgcctttatctgcatgagcatgactacgatgtgctttcttttttgcgcgaggctttgcctgattttttgagcagcaccttgcattttatatcgccgcccatgcaacaagcttacatcggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagatatattgagaaggtggcgagatgagaattatttgggcatggttgaaggtgctggaatgtttatagaggagattcaccctgaagggtttagcctttacgtccacttggacgtgagggccgtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgatctaagcggcgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgtctacctccttttgagacagaaacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctaacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgtatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgccttatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttctccttcgccgcccgttaagcaaccgcaagttggacagcagcctgtggctcagcagctggacagcgacatgaacttaagtgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatctgtataagctatgtggtggtggggctatactactgaatgaaaaatgacttgaaattttctgcaattgaaaaataaacacgttgaaacataacacaaacgattctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatctgcaacaacatgaagatagtgggtgcggatggacaggaacaggaggaaactgacattccatttagattgtggagaaagtttgcagccaggaggaagctgcaataccagagctgggaggagggcaaggaggtgctgctgaataaactggacagaaatttgctaactgattttaagtaagtgatgctttattatttttttttattagttaaagggaataagatccccgggtactctagttaattaactagaggatcttgatgtaatccagggttaggacagttgcaaatcacagtgagaacacagggtcccctgtcccgctcaactagcagggggcgctgggtaaactcccgaatcaggctacgggcaagctctccctgggcggtaagccggacgccgtgcgccgggccctcgatatgatcctcgggcaattcaaagtagcaaaactcaccggagtcgcgggcaaagcacttgtggcggcgacagtggaccaggtgtttcaggcgcagttgctctgcctctccacttaacattcagtcgtagccgtccgccgagtcctttaccgcgtcaaagttaggaataaattgatccggatagtggccgggaggtcccgagaaggggttaaagtagaccgatggcacaaactcctcaataaattgcagagttccaatgcctccagagcgcggctcagaggacgaggtctgcagagttaggattgcctgacgaggcgtgaatgaagagcggccggcgccgccgatctgaaatgtcccgtccggacggagaccaagcgaggagctcaccgactcgtcgttgagctgaatacctcgccctctgattgtcaggtgagttataccctgcccgggcgaccgcaccctgtgacgaaagccgcccgcaagctgcgcccctgagttagtcatctgaacttcggcctgggcgtctctgggaagtaccacagtggtgggagcgggactttcctggtacaccagggcagcgggccaactacggggattaaggttattacgaggtgtggtggtaatagccgcctgttccaggagaattcggtttcggtgggcgcgtattccgttgacccgggatatcatgtggggtcccgcgctcatgtagtttattcgggttgagtagtcttgggcagctccagccgcaagtcccatttgtggctggtaactccacatgtagggcgtgggaatttccttgctcataatggcgctgacaacaggtgctggcgccgggtgtggccgctggagatgacgtagttttcgcgcttaaatttgagaaagggcgcgaaactagtccttaagagtcagcgcgcagtatttactgaagagagcctccgcgtcttccagcgtgcgccgaagctgatcttcgcttttgtgatacaggcagctgcgggtgagggatcgcagagacctgttttttattttcagctcttgttcttggcccctgctctgttgaaatatagcatacagagtgggaaaaatcctgtttctaagctcgcgggtcgatacgggttcgttgggcgccagacgcagcgctcctcctcctgctgctgccgccgctgtggatttcttgggctttgtcagagtcttgctatccggtcgcctttgcttctgtgtggccgctgctgttgctgccgctgccgccggtgcagtatgggctgtagagatgacggtagtaatgcaggatgttacgggggaaggccacgccgtgatggtagagaagaaagcggcgggcgaaggagatgttgcccccacagtcttgcaagcaagcaactatggcgttcttgtgcccgcgccatgagcggtagccttggcgctgttgttgctcttgggctaacggcggcggctgcttggacttaccggccctggttccagtggtgtcccatctacggttgggtcggcgaacgggcagtgccggcggcgcctgaggagcggaggttgtagccatgctggaaccggttgccgatttctggggcgccggcgaggggaatgcgaccgagggtgacggtgtttcgtctgacacctcttcgacctcggaagcttcctcgtctaggctctcccagtcttccatcatgtcctcctcctcctcgtccaaaacctcctctgcctgactgtcccagtattcctcctcgtccgtgggtggcggcggcagctgcagcttctttttgggtgccatcctgggaagcaagggcccgcggctgctgctgatagggctgcggcggcggggggattgggttgagctcctcgccggactgggggtccaagtaaaccccccgtccctttcgtagcagaaactcttggcgggctttgttgatggcttgcaattggccaagaatgtggccctgggtaatgacgcaggcggtaagctccgcattaggcgggcgggattggtcttcgtagaacctaatctcgtgggcgtggtagtcctcaggtacaaatttgcgaaggtaagccgacgtccacagccccggagtgagtttcaaccccggagccgcggacttttcgtcaggcgagggaccctgcagctcaaaggtaccgataatttgactttcgttaagcagctgcgaattgcaaaccagggagcggtgcggggtgcataggttgcagcgacagtgacactccagtagaccgtcaccgctcacgtcttccattatgtcagagtggtaggcaaggtagttggctagctgcagaaggtagcagtggccccaaagcggcggagggcattcgcggtacttaatgggcacaaagtcgctaggaagtgcacagcaggtggcgggcaagattcctgagcgctctaggataaagttcctaaagttctgcaacatgctttgactggtgaagtctggcagaccctgttgcagggttttaagcaggcgttcggggaaaatgatgtccgccaggtgcgcggccacggagcgctcgttgaaggccgtccataggtccttcaagttttgctttagcagtttctgcagctccttgaggttgcactcctccaagcactgctgccaaacgcccatggccgtctgccaggtgtagcatagaaataagtaaacgcagtcgcggacgtagtcgcggcgcgcctcgcccttgagcgtggaatgaagcacgttttgcccaaggcggttttcgtgcaaaattccaaggtaggagaccaggttgcagagctccacgttggagatcttgcaggcctggcgtacgtagccctgtcgaaaggtgtagtgcaatgtttcctctagcttgcgctgcatctccgggtcagcaaagaaccgctgcatgcactcaagctccacggtaacgagcactgcggccatcattagtttgcgtcgctcctccaagtcggcaggctcgcgcgtttgaagccagcgcgctagctgctcgtcgccaactgcgggtaggccctcctctgtttgttcttgcaaatttgcatccctctccaggggctgcgcacggcgcacgatcagctcactcatgactgtgctcatgaccttggggggtaggttaagtgccgggtaggcaaagtgggtgacctcgatgctgcgttttagtacggctaggcgcgcgttgtcaccctcgagttccaccaacactccagagtgactttcattttcgctgttttcctgttgcagagcgtttgccgcgcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcggtccgagactcgaaccgggggtcccgcgactcaacccttggaaaataaccctccggctacagggagcgagccacttaatgctttcgctttccagcctaaccgcttacgctgcgcgcggccagtggccaaaaaagctagcgcagcagccgccgcgcctggaaggaagccaaaaggagcactcccccgttgtctgacgtcgcacacctgggttcgacacgcgggcggtaaccgcatggatcacggcggacggccggatacggggctcgaaccccggtcgtccgccatgatacccttgcgaatttatccaccagaccacggaagagtgcccgcttacaggctctccttttgcacgctagagcgtcaacgattgcgcgcgcctgaccggccagagcgtcccgaccatggagcactttttgccgctgcgcaacatctggaaccgcgtccgcgactttccgcgcgcctccaccaccgccgccggcatcacctggatgtccaggtacatctacggatatcatcgccttatgttggaagatctcgcccccggagccccggccaccctacgctggcccctctaccgccagccgccgccgcactttttggtgggataccagtacctggtgcggacttgcaacgactacgtatttgactcgagggcttactcgcgtctcaggtacaccgagctctcgcagccgggtcaccagaccgttaactggtccgttatggccaactgcacttacaccatcaacacgggcgcataccaccgctttgtggacatggatgacttccagtctaccctcacgcaggtgcagcaggccatattagccgagcgcgttgtcgccgacctagccctgcttcagccgatgaggggcttcggggtcacacgcatgggaggaagagggcgccacctacggccaaactccgccgccgccgcagcgatagatgcaagagatgcaggacaagaggaaggagaagaagaagtgccggtagaaaggctcatgcaagactactacaaagacctgcgccgatgtcaaaacgaagcctggggcatggccgaccgcctgcgcattcagcaggccggacccaaggacatggtgcttctgtcgacggtatcgataagcttgatatcgaattcctgcagcccgggggatccgcgtagtatgctgaaataggtgactagaagttcctatactttctagagaataggaacttcataacttcgtataatgtatgctatacgaagttatgggttactttaatttggttgctgactaattgagatgcatgctttgcatacttctgcctgctggggagcctggggactttccacacctggttgctgactaattgagatgcatgctttgcatacttctgcctgctggggagcctggggactttccacacc